SEQUENCE LISTING (613 sequences)

SEQ ID NO: 1 - DNA, 23 bp, Artificial Sequence
  Synthetic Construct
taatacgactcactatagggaat

SEQ ID NO: 2 - RNA, 49 nt, Artificial Sequence
  Synthetic Construct
uuuccccugaugaguccgugaggacgaaacgaguaagcucgucgggaaa

SEQ ID NO: 3 - RNA, 20 nt, Artificial Sequence
  Synthetic Construct
gggaaaaaaaugccgucggu

SEQ ID NO: 4 - RNA, 20 nt, Artificial Sequence
  Synthetic Construct
accgacggcaaaaaaaaaaa

SEQ ID NO: 5 - RNA, 464 nt, Artificial Sequence
  Synthetic Construct
gagggcccggaaaccuggcccugucuucuugacgagcauuccuaggggucuuuccccucucgccaaaggaaugcaaggucuguugaaugucgugaaggaagcaguuccucuggaagcuucuugaagacaaacaacgucuguagcgacccuuugcaggcagcggaaccccccaccuggcgacaggugccucugcggccaaaagccacguguauaagauacaccugcaaaggcggcacaaccccagugccacguugugaguuggauaguuguggaaagagucaaauggcucuccucaagcguauucaacaaggggcugaaggaugcccagaagguaccccauuguaugggaucugaucuggggccucggugcacaugcuuuacauguguuuagucgagguuaaaaaaacgucuaggccccccgaaccacggggacgugguuuuccuuugaaaaacacgaugauaau

SEQ ID NO: 6 - RNA, 516 nt, Artificial Sequence
  Synthetic Construct
auggucuucacacucgaagauuucguuggggacuggcgacagacagccggcuacaaccuggaccaaguccuugaacagggagguguguccaguuuguuucagaaucucgggguguccguaacuccgauccaaaggauuguccugagcggugaaaaugggcugaagaucgacauccaugucaucaucccguaugaaggucugagcggcgaccaaaugggccagaucgaaaaaauuuuuaaggugguguacccuguggaugaucaucacuuuaaggugauccugcacuauggcacacugguaaucgacgggguuacgccgaacaugaucgacuauuucggacggccguaugaaggcaucgccguguucgacggcaaaaagaucacuguaacagggacccuguggaacggcaacaaaauuaucgacgagcgccugaucaaccccgacggcucccugcuguuccgaguaaccaucaacggagugaccggcuggcggcugugcgaacgcauucuggcguaa

SEQ ID NO: 7 - RNA, 68 nt, Artificial Sequence
  Synthetic Construct
ggccggcauggucccagccuccucgcuggcgccggcugggcaacaugcuucggcauggcgaaugggac

SEQ ID NO: 8 - DNA, 1160 bp, Artificial Sequence
  Synthetic Construct
taatacgactcactatagggaattttcccctgatgagtccgtgaggacgaaacgagtaagctcgtcgggaaaaaaatgccgtcggtgagggcccggaaacctggccctgtcttcttgacgagcattcctaggggtctttcccctctcgccaaaggaatgcaaggtctgttgaatgtcgtgaaggaagcagttcctctggaagcttcttgaagacaaacaacgtctgtagcgaccctttgcaggcagcggaaccccccacctggcgacaggtgcctctgcggccaaaagccacgtgtataagatacacctgcaaaggcggcacaaccccagtgccacgttgtgagttggatagttgtggaaagagtcaaatggctctcctcaagcgtattcaacaaggggctgaaggatgcccagaaggtaccccattgtatgggatctgatctggggcctcggtgcacatgctttacatgtgtttagtcgaggttaaaaaaacgtctaggccccccgaaccacggggacgtggttttcctttgaaaaacacgatgataatgccaccatggtcttcacactcgaagatttcgttggggactggcgacagacagccggctacaacctggaccaagtccttgaacagggaggtgtgtccagtttgtttcagaatctcggggtgtccgtaactccgatccaaaggattgtcctgagcggtgaaaatgggctgaagatcgacatccatgtcatcatcccgtatgaaggtctgagcggcgaccaaatgggccagatcgaaaaaatttttaaggtggtgtaccctgtggatgatcatcactttaaggtgatcctgcactatggcacactggtaatcgacggggttacgccgaacatgatcgactatttcggacggccgtatgaaggcatcgccgtgttcgacggcaaaaagatcactgtaacagggaccctgtggaacggcaacaaaattatcgacgagcgcctgatcaaccccgacggctccctgctgttccgagtaaccatcaacggagtgaccggctggcggctgtgcgaacgcattctggcgtaaaccgacggcaaaaaaaaaaaggccggcatggtcccagcctcctcgctggcgccggctgggcaacatgcttcggcatggcgaatgggac

SEQ ID NO: 9 - RNA, 1143 nt, Artificial Sequence
  Synthetic Construct
gggaauuuuccccugaugaguccgugaggacgaaacgaguaagcucgucgggaaaaaaaugccgucggugagggcccggaaaccuggcccugucuucuugacgagcauuccuaggggucuuuccccucucgccaaaggaaugcaaggucuguugaaugucgugaaggaagcaguuccucuggaagcuucuugaagacaaacaacgucuguagcgacccuuugcaggcagcggaaccccccaccuggcgacaggugccucugcggccaaaagccacguguauaagauacaccugcaaaggcggcacaaccccagugccacguugugaguuggauaguuguggaaagagucaaauggcucuccucaagcguauucaacaaggggcugaaggaugcccagaagguaccccauuguaugggaucugaucuggggccucggugcacaugcuuuacauguguuuagucgagguuaaaaaaacgucuaggccccccgaaccacggggacgugguuuuccuuugaaaaacacgaugauaaugccaccauggucuucacacucgaagauuucguuggggacuggcgacagacagccggcuacaaccuggaccaaguccuugaacagggagguguguccaguuuguuucagaaucucgggguguccguaacuccgauccaaaggauuguccugagcggugaaaaugggcugaagaucgacauccaugucaucaucccguaugaaggucugagcggcgaccaaaugggccagaucgaaaaaauuuuuaaggugguguacccuguggaugaucaucacuuuaaggugauccugcacuauggcacacugguaaucgacgggguuacgccgaacaugaucgacuauuucggacggccguaugaaggcaucgccguguucgacggcaaaaagaucacuguaacagggacccuguggaacggcaacaaaauuaucgacgagcgccugaucaaccccgacggcucccugcuguuccgaguaaccaucaacggagugaccggcuggcggcugugcgaacgcauucuggcguaaaccgacggcaaaaaaaaaaaggccggcauggucccagccuccucgcuggcgccggcugggcaacaugcuucggcauggcgaaugggac

SEQ ID NO: 10 - RNA, 1026 nt, Artificial Sequence
  Synthetic Construct
gggaaaaaaaugccgucggugagggcccggaaaccuggcccugucuucuugacgagcauuccuaggggucuuuccccucucgccaaaggaaugcaaggucuguugaaugucgugaaggaagcaguuccucuggaagcuucuugaagacaaacaacgucuguagcgacccuuugcaggcagcggaaccccccaccuggcgacaggugccucugcggccaaaagccacguguauaagauacaccugcaaaggcggcacaaccccagugccacguugugaguuggauaguuguggaaagagucaaauggcucuccucaagcguauucaacaaggggcugaaggaugcccagaagguaccccauuguaugggaucugaucuggggccucggugcacaugcuuuacauguguuuagucgagguuaaaaaaacgucuaggccccccgaaccacggggacgugguuuuccuuugaaaaacacgaugauaaugccaccauggucuucacacucgaagauuucguuggggacuggcgacagacagccggcuacaaccuggaccaaguccuugaacagggagguguguccaguuuguuucagaaucucgggguguccguaacuccgauccaaaggauuguccugagcggugaaaaugggcugaagaucgacauccaugucaucaucccguaugaaggucugagcggcgaccaaaugggccagaucgaaaaaauuuuuaaggugguguacccuguggaugaucaucacuuuaaggugauccugcacuauggcacacugguaaucgacgggguuacgccgaacaugaucgacuauuucggacggccguaugaaggcaucgccguguucgacggcaaaaagaucacuguaacagggacccuguggaacggcaacaaaauuaucgacgagcgccugaucaaccccgacggcucccugcuguuccgaguaaccaucaacggagugaccggcuggcggcugugcgaacgcauucuggcguaaaccgacggcaaaaaaaaaaa

SEQ ID NO: 11 - RNA, 228 nt, Artificial Sequence
  Synthetic Construct
aucuaaguuagguuguaaacauauuagagacguuguucauuuggaagaguuacgcgagucuuugugugauguagcuaguaaccuaaauaauugugcguauuuuucacaguuagaugaggccguugccgagguucauaagaccgcgguaggcgguucguuugcuuuuuguaguauaauuaaauauuugucagauaagagauuguuuagagauuuguucuuuguuugaua

SEQ ID NO: 12 - RNA, 121 nt, Artificial Sequence
  Synthetic Construct
uagcuuauuuggguucuuuaucacuguccugauuaaggcugaaucagaacaccaucacuuccacucacacgacaguaguaagacacagaacauaguaguuaauacuggaaaguaacccacc

SEQ ID NO: 13 - RNA, 147 nt, Artificial Sequence
  Synthetic Construct
agcacaacauuucaaccagaaacacuagccgaagcaaauccauuccacaagcaccuggugggaucaucucaucaucagaaaccaagagagagauuccguguccgcuuguuguaguagauugugaggacugaggaccgagaagcagcc

SEQ ID NO: 14 - RNA, 1653 nt, Artificial Sequence
  Synthetic Construct
auggaagacgccaaaaacauaaagaaaggcccggcgccauucuauccgcuggaagauggaaccgcuggagagcaacugcauaaggcuaugaagagauacgcccugguuccuggaacaauugcuuuuacagaugcacauaucgagguggacaucacuuacgcugaguacuucgaaauguccguucgguuggcagaagcuaugaaacgauaugggcugaauacaaaucacagaaucgucguaugcagugaaaacucucuucaauucuuuaugccgguguugggcgcguuauuuaucggaguugcaguugcgcccgcgaacgacauuuauaaugaacgugaauugcucaacaguaugggcauuucgcagccuaccgugguguucguuuccaaaaagggguugcaaaaaauuuugaacgugcaaaaaaagcucccaaucauccaaaaaauuauuaucauggauucuaaaacggauuaccagggauuucagucgauguacacguucgucacaucucaucuaccucccgguuuuaaugaauacgauuuugugccagaguccuucgauagggacaagacaauugcacugaucaugaacuccucuggaucuacuggucugccuaaaggugucgcucugccucauagaacugccugcgugagauucucgcaugccagagauccuauuuuuggcaaucaaaucauuccggauacugcgauuuuaaguguuguuccauuccaucacgguuuuggaauguuuacuacacucggauauuugauauguggauuucgagucgucuuaauguauagauuugaagaagagcuguuucugaggagccuucaggauuacaagauucaaagugcgcugcuggugccaacccuauucuccuucuucgccaaaagcacucugauugacaaauacgauuuaucuaauuuacacgaaauugcuucugguggcgcuccccucucuaaggaagucggggaagcgguugccaagagguuccaucugccagguaucaggcaaggauaugggcucacugagacuacaucagcuauucugauuacacccgagggggaugauaaaccgggcgcggucgguaaaguuguuccauuuuuugaagcgaagguuguggaucuggauaccgggaaaacgcugggcguuaaucaaagaggcgaacugugugugagagguccuaugauuauguccgguuauguaaacaauccggaagcgaccaacgccuugauugacaaggauggauggcuacauucuggagacauagcuuacugggacgaagacgaacacuucuucaucguugaccgccugaagucucugauuaaguacaaaggcuaucagguggcucccgcugaauuggaauccaucuugcuccaacaccccaacaucuucgacgcuggugucgcaggucuucccgacgaugacgccggugaacuucccgccgccguuguuguuuuggagcacggaaagacgaugacggaaaaagagaucguggauuacgucgccagucaaguaacaaccgcgaaaaaguugcgcggaggaguuguguuuguggacgaaguaccgaaaggucuuaccggaaaacucgacgcaagaaaaaucagagagauccucauaaaggccaagaagggcggaaagaucgccguguaa

SEQ ID NO: 15 - RNA, 790 nt, Artificial Sequence
  Synthetic Construct
gggaaaaaaaugccgucgguaucuaaguuagguuguaaacauauuagagacguuguucauuuggaagaguuacgcgagucuuugugugauguagcuaguaaccuaaauaauugugcguauuuuucacaguuagaugaggccguugccgagguucauaagaccgcgguaggcgguucguuugcuuuuuguaguauaauuaaauauuugucagauaagagauuguuuagagauuuguucuuuguuugauagccaccauggucuucacacucgaagauuucguuggggacuggcgacagacagccggcuacaaccuggaccaaguccuugaacagggagguguguccaguuuguuucagaaucucgggguguccguaacuccgauccaaaggauuguccugagcggugaaaaugggcugaagaucgacauccaugucaucaucccguaugaaggucugagcggcgaccaaaugggccagaucgaaaaaauuuuuaaggugguguacccuguggaugaucaucacuuuaaggugauccugcacuauggcacacugguaaucgacgggguuacgccgaacaugaucgacuauuucggacggccguaugaaggcaucgccguguucgacggcaaaaagaucacuguaacagggacccuguggaacggcaacaaaauuaucgacgagcgccugaucaaccccgacggcucccugcuguuccgaguaaccaucaacggagugaccggcuggcggcugugcgaacgcauucuggcguaaaccgacggcaaaaaaaaaaa

SEQ ID NO: 16 - RNA, 1876 nt, Artificial Sequence
  Synthetic Construct
gggaaaaaaaugccgucgguagcacaacauuucaaccagaaacacuagccgaagcaaauccauuccacaagcaccuggugggaucaucucaucaucagaaaccaagagagagauuccguguccgcuuguuguaguagauugugaggacugaggaccgagaagcagccgccaccaaugagagaccuuuuaaaaggucucuaaugauggaggacgccaagaacaucaagaagggccccgcccccuucuacccccuggaggacggcaccgccggcgagcagcugcacaaggccaugaagcgguacgcccuggugcccggcaccaucgccuucaccgacgcccacaucgagguggacaucaccuacgccgaguacuucgagaugagcgugcggcuggccgaggccaugaagcgguacggccugaacaccaaccaccggaucguggugugcagcgagaacagccugcaguucuucaugcccgugcugggcgcccuguucaucggcguggccguggcccccgccaacgacaucuacaacgagcgggagcugcugaacagcaugggcaucagccagcccaccgugguguucgugagcaagaagggccugcagaagauccugaacgugcagaagaagcugcccaucauccagaagaucaucaucauggacagcaagaccgacuaccagggcuuccagagcauguacaccuucgugaccagccaccugccccccggcuucaacgaguacgacuucgugcccgagagcuucgaccgggacaagaccaucgcccugaucaugaacagcagcggcagcaccggccugcccaagggcguggcccugccccaccggaccgccugcgugcgguucagccacgcccgggaccccaucuucggcaaccagaucauccccgacaccgccauccugagcguggugcccuuccaccacggcuucggcauguucaccacccugggcuaccugaucugcggcuuccggguggugcugauguaccgguucgaggaggagcuguuccugcggagccugcaggacuacaagauccagagcgcccugcuggugcccacccuguucagcuucuucgccaagagcacccugaucgacaaguacgaccugagcaaccugcacgagaucgccagcggcggcgccccccugagcaaggaggugggcgaggccguggccaagcgguuccaccugcccggcauccggcagggcuacggccugaccgagaccaccagcgccauccugaucacccccgagggcgacgacaagcccggcgccgugggcaagguggugcccuucuucgaggccaaggugguggaccuggacaccggcaagacccugggcgugaaccagcggggcgagcugugcgugcggggccccaugaucaugagcggcuacgugaacaaccccgaggccaccaacgcccugaucgacaaggacggcuggcugcacagcggcgacaucgccuacugggacgaggacgagcacuucuucaucguggaccggcugaagagccugaucaaguacaagggcuaccagguggcccccgccgagcuggagagcauccugcugcagcaccccaacaucuucgacgccggcguggccggccugcccgacgacgacgccggcgagcugcccgccgccgugguggugcuggagcacggcaagaccaugaccgagaaggagaucguggacuacguggccagccaggugaccaccgccaagaagcugcggggcggcgugguguucguggacgaggugcccaagggccugaccggcaagcuggacgcccggaagauccgggagauccugaucaaggccaagaagggcggcaagaucgccgugugaaccgacggcaaaaaaaaaaa

SEQ ID NO: 17 - DNA, 49 bp, Artificial Sequence
  Synthetic Construct
tttcccctgatgagtccgtgaggacgaaacgagtaagctcgtcgggaaa

SEQ ID NO: 18 - DNA, 20 bp, Artificial Sequence
  Synthetic Construct
gggaaaaaaatgccgtcggt

SEQ ID NO: 19 - DNA, 20 bp, Artificial Sequence
  Synthetic Construct
accgacggcaaaaaaaaaaa

SEQ ID NO: 20 - DNA, 464 bp, Artificial Sequence
  Synthetic Construct
gagggcccggaaacctggccctgtcttcttgacgagcattcctaggggtctttcccctctcgccaaaggaatgcaaggtctgttgaatgtcgtgaaggaagcagttcctctggaagcttcttgaagacaaacaacgtctgtagcgaccctttgcaggcagcggaaccccccacctggcgacaggtgcctctgcggccaaaagccacgtgtataagatacacctgcaaaggcggcacaaccccagtgccacgttgtgagttggatagttgtggaaagagtcaaatggctctcctcaagcgtattcaacaaggggctgaaggatgcccagaaggtaccccattgtatgggatctgatctggggcctcggtgcacatgctttacatgtgtttagtcgaggttaaaaaaacgtctaggccccccgaaccacggggacgtggttttcctttgaaaaacacgatgataat

SEQ ID NO: 21 - DNA, 516 bp, Artificial Sequence
  Synthetic Construct
atggtcttcacactcgaagatttcgttggggactggcgacagacagccggctacaacctggaccaagtccttgaacagggaggtgtgtccagtttgtttcagaatctcggggtgtccgtaactccgatccaaaggattgtcctgagcggtgaaaatgggctgaagatcgacatccatgtcatcatcccgtatgaaggtctgagcggcgaccaaatgggccagatcgaaaaaatttttaaggtggtgtaccctgtggatgatcatcactttaaggtgatcctgcactatggcacactggtaatcgacggggttacgccgaacatgatcgactatttcggacggccgtatgaaggcatcgccgtgttcgacggcaaaaagatcactgtaacagggaccctgtggaacggcaacaaaattatcgacgagcgcctgatcaaccccgacggctccctgctgttccgagtaaccatcaacggagtgaccggctggcggctgtgcgaacgcattctggcgtaa

SEQ ID NO: 22 - DNA, 68 bp, Artificial Sequence
  Synthetic Construct
ggccggcatggtcccagcctcctcgctggcgccggctgggcaacatgcttcggcatggcgaatgggac

SEQ ID NO: 23 - RNA, 683 nt, Artificial Sequence
  Synthetic Construct
gggaaaaaaaugccgucgguuagcuuauuuggguucuuuaucacuguccugauuaaggcugaaucagaacaccaucacuuccacucacacgacaguaguaagacacagaacauaguaguuaauacuggaaaguaacccaccgccaccauggucuucacacucgaagauuucguuggggacuggcgacagacagccggcuacaaccuggaccaaguccuugaacagggagguguguccaguuuguuucagaaucucgggguguccguaacuccgauccaaaggauuguccugagcggugaaaaugggcugaagaucgacauccaugucaucaucccguaugaaggucugagcggcgaccaaaugggccagaucgaaaaaauuuuuaaggugguguacccuguggaugaucaucacuuuaaggugauccugcacuauggcacacugguaaucgacgggguuacgccgaacaugaucgacuauuucggacggccguaugaaggcaucgccguguucgacggcaaaaagaucacuguaacagggacccuguggaacggcaacaaaauuaucgacgagcgccugaucaaccccgacggcucccugcuguuccgaguaaccaucaacggagugaccggcuggcggcugugcgaacgcauucuggcguaaaccgacggcaaaaaaaaaaa

SEQ ID NO: 24 - RNA, 61 nt, Unknown
  Unknown Organism
caugcucagcggucccaaguccgcaucaaagccugagggcugcaguaaagguacugagcug

SEQ ID NO: 25 - RNA, 76 nt, Unknown
  Unknown Organism
uuauuuagccgucuaaagucggcaaugaauugagauagcacccuguaaauuuucaggguguaaacaaacuaaauga

SEQ ID NO: 26 - RNA, 72 nt, Unknown
  Unknown Organism
uuaauugccgguugccaguccguuaaauugugagcaguccggccauugugccggauuaaacaaaccaauuaa

SEQ ID NO: 27 - RNA, 72 nt, Unknown
  Unknown Organism
uuaguuaacgguugcacguccgauaaauugugagcagucccggagcaauccgggauuaaacaaacuaacuaa

SEQ ID NO: 28 - RNA, 74 nt, Unknown
  Unknown Organism
ugauuuaggcguuccaaaccgccgcaaauugugaggacugcucgccaaaagcgggcaguaaacaaguuaaauca

SEQ ID NO: 29 - RNA, 80 nt, Unknown
  Unknown Organism
aauucuugcgguucaaaguccgcguaaaauccagaugacacauucccguaauaaacgggaguguguaaugaacaagaauu

SEQ ID NO: 30 - RNA, 73 nt, Unknown
  Unknown Organism
acacccaccuguuacaagucaggacagaagcagaguaacgguugcuuacgcaaccgguaaugcuacugggugu

SEQ ID NO: 31 - RNA, 74 nt, Unknown
  Unknown Organism
caauaaagcgguuacaagcccgcaaaaauagcagaguaaugucgcgauagcgcggcauuaaugcagcuuuauug

SEQ ID NO: 32 - RNA, 72 nt, Unknown
  Unknown Organism
uguuuaaugcagccaugaguauuuaauacuaugaaggugauaagcuccuuguaaaguaaugcagaaucgaca

SEQ ID NO: 33 - RNA, 57 nt, Unknown
  Unknown Organism
gccguaaagccacuaugaccggguugcaagucccggcugcgauaggcugagcacggu

SEQ ID NO: 34 - RNA, 119 nt, Unknown
  Unknown Organism
guucuaaugcagccagcacgacuuugucauagauaaaauaucauuaauacacuauuuacacagauguaugcgauuacuagugcugggaguccuaagccuccauaaaugcagaagggaac

SEQ ID NO: 35 - RNA, 93 nt, Unknown
  Unknown Organism
ucuguaaccccaccaccguggacauccuggcagggauaauggccaggaugaucaugguggagguccaaaguccucaaaagaggggauggcaga

SEQ ID NO: 36 - RNA, 84 nt, Unknown
  Unknown Organism
acaauaaugcggccucgcuaccaauacgcauuuauuaguauugguaacgugacagucccaagccuguaaaacgcagaggguugu

SEQ ID NO: 37 - RNA, 57 nt, Unknown
  Unknown Organism
gucguaaugcagccguugccacgugccaagucguggauuagaaaugcagaggcggaa

SEQ ID NO: 38 - RNA, 61 nt, Unknown
  Unknown Organism
gguguaaugcgacucgcucacagagcgacagguucacaguccuacaaacgcagaugacacc

SEQ ID NO: 39 - RNA, 97 nt, Unknown
  Unknown Organism
agcuuaauacagguagauaagcaagcaaggugcggcuaucuacacggucccaacuccguaaagguuagagugacaacuaaucgaaguagagggagcu

SEQ ID NO: 40 - RNA, 52 nt, Unknown
  Unknown Organism
uaaauaaugucgccaauggagguaucaagcccucauaaagacagagauaaaa

SEQ ID NO: 41 - RNA, 73 nt, Unknown
  Unknown Organism
acguuaauguggcuguaugugugggugcacacacauacacuagucccaagccuagguaaacacagagggauug

SEQ ID NO: 42 - RNA, 62 nt, Unknown
  Unknown Organism
aaaguaaugcaacuacaagaaauuguaucggugacaaguccgagauaaaugcagagucauuu

SEQ ID NO: 43 - RNA, 56 nt, Unknown
  Unknown Organism
ucuguaaugaugccgauggcgguugcaagcccgcaggaagaaacucagagcacaga

SEQ ID NO: 44 - RNA, 55 nt, Unknown
  Unknown Organism
acuauaaucuugccaucgucaguuccaagccugagugagaaaaagagaggauagu

SEQ ID NO: 45 - RNA, 58 nt, Unknown
  Unknown Organism
acucuaacccagcggcaaucuuuugcccguguccgaagccacuaauggggacgggagu

SEQ ID NO: 46 - RNA, 55 nt, Unknown
  Unknown Organism
ccgcuaaccaugccguggccagucccaagccuggaugugaaaaugggaggggcgg

SEQ ID NO: 47 - RNA, 60 nt, Unknown
  Unknown Organism
uuuuuaaugaagccacagugaucacugugaggguccuaagccccuaauucagaagggaaa

SEQ ID NO: 48 - RNA, 68 nt, Unknown
  Unknown Organism
uguguaaugcuacuaugauagcacauugcgaaucauacggguugcaagucccucaagcagagcacacg

SEQ ID NO: 49 - RNA, 51 nt, Unknown
  Unknown Organism
uuuuuaacccagccagagacggucacaagcccgugaaauggggaguggaaa

SEQ ID NO: 50 - RNA, 58 nt, Unknown
  Unknown Organism
gcucuaauguggccacccgacaggguguguguuucaagccaccaacacagagaagagc

SEQ ID NO: 51 - RNA, 83 nt, Unknown
  Unknown Organism
gguguaacacggcuauagucaggcauuacaagauuaaguccugcuauaaaggucuaaagcccuuguaaacaguggauagcacu

SEQ ID NO: 52 - RNA, 57 nt, Unknown
  Unknown Organism
uguguaaugcgagcauuguauggucacaacuccauaauuaaaaacgcagagugcaca

SEQ ID NO: 53 - RNA, 56 nt, Unknown
  Unknown Organism
gcuuuaacacagccaaagaagguuccaagcccuuuagugaaauuguggaggaaagc

SEQ ID NO: 54 - RNA, 67 nt, Unknown
  Unknown Organism
aaaguaaugcagccgcccgccgcgcgcggggacgucgguagcaagcccguguaaugcagaguuuucu

SEQ ID NO: 55 - RNA, 61 nt, Unknown
  Unknown Organism
gcuuuaaugcggcccguuuugauacggcagguuacaagcccugguaaacgcagaguagagc

SEQ ID NO: 56 - RNA, 69 nt, Unknown
  Unknown Organism
uucguaaugcggccgugcugguaacguuccagcgcgacggucccaagcccgaaaaacgcagagggagaa

SEQ ID NO: 57 - RNA, 80 nt, Unknown
  Unknown Organism
ccgguaaugcggcacgcguggucacaagcccaccgcccuucguugagcggaaacguucacguugggacgcagagugacgg

SEQ ID NO: 58 - RNA, 53 nt, Unknown
  Unknown Organism
ggcuuaacucagccaacggcgguccaaagcccgcguguaaugaggauggagcc

SEQ ID NO: 59 - RNA, 66 nt, Unknown
  Unknown Organism
agcguaauguagccuaguccgacuuuggacuagaggguucacagccccuuuaauacagaugacgca

SEQ ID NO: 60 - RNA, 81 nt, Unknown
  Unknown Organism
gguguaaagcuacuaaacaggcaauacaaaaauaaguccuguuuaaagguucaaaguccuuguaaaaaagcugaugacacg

SEQ ID NO: 61 - RNA, 62 nt, Unknown
  Unknown Organism
ccucuaaugcggcccggcauggugccggacggugguaagcccgugcaaacgcagaaccuagg

SEQ ID NO: 62 - RNA, 52 nt, Unknown
  Unknown Organism
cucguaaugcggcgaaccgguggcaagcccggugguggacgcagagccagag

SEQ ID NO: 63 - RNA, 53 nt, Unknown
  Unknown Organism
uccucaaugcggcaagccggugacaagcccggcgguagacgcagagucaagga

SEQ ID NO: 64 - RNA, 62 nt, Unknown
  Unknown Organism
uuuguaauguggccuaaauuuuuauuuagaacguuccaagccguuaaaacacagaggacaaa

SEQ ID NO: 65 - RNA, 55 nt, Unknown
  Unknown Organism
cucuuaaaguugccuaagaacguugcaagccguuuuacgaaaaacugagcaagaa

SEQ ID NO: 66 - RNA, 71 nt, Unknown
  Unknown Organism
auuguaaugcagcauauagauguauuaacaccuauauagaguucaaagccucuacaaaugcagaugacaau

SEQ ID NO: 67 - RNA, 72 nt, Acyrthosiphon pisum
uuuuuaaucauaccaguagucuaauuuuuagauuacugacaguccuaagucuguaaaaaaugagaagggaaa

SEQ ID NO: 68 - RNA, 106 nt, Unknown
  Unknown Organism
aacucagcuagggagaguauaacauucauguugacgagaccuagacgaaacacagaggaaaauuauuaaucacuggauaguauuaguaaugacucuguguccauga

SEQ ID NO: 69 - RNA, 77 nt, Unknown
  Unknown Organism
uugucagcuaaggagacagaaaaauuaucuacugaugagacuuagccgaaaccaccucuuuuagggguggucuagau

SEQ ID NO: 70 - RNA, 85 nt, Unknown
  Unknown Organism
aagucagccaggagacuauaaaauucauacugaugagacuggacgaaauaccuaguaacaguuguacguuauuagguaucuauga

SEQ ID NO: 71 - RNA, 119 nt, Unknown
  Unknown Organism
aacucagcuagggagaguagcgagcauuacguaauacuacguauuacuccaauaacauugucacugaugagaccuagacgaaacuacgguaaacauuugcaucauacuguagucugaua

SEQ ID NO: 72 - RNA, 76 nt, Unknown
  Unknown Organism
aagacagccuaggagucuauaaaauaugugcugacgagacuaggacgaaacuauccucaguugaggauaguccacu

SEQ ID NO: 73 - RNA, 73 nt, Unknown
  Unknown Organism
aagacagucuaggagucuauaaaauuguuacugaagagacuagaacgaaacuucuuuaauuagaagucuaaca

SEQ ID NO: 74 - RNA, 63 nt, Unknown
  Unknown Organism
aacucaaccaggagaguauaaaauguuuacugaugagacuggacgaaaccaauagguuuaaac

SEQ ID NO: 75 - RNA, 74 nt, Unknown
  Unknown Organism
aagacauccaggagucuauaaaauagucacugaugagacuggacgaaaccucugcuauauguagaggucugauu

SEQ ID NO: 76 - RNA, 163 nt, Veillonella sp.
auugccugugaagguagugcauauuuuuauuauuagaucaucagaagaugacaagcauguggggcguaaguaguauuuuuaugcgggagaagaagaauggcaauuguucuaauuaguacugauaauugcaaauacuaugaucgugcggacguuaaaaucaugc

SEQ ID NO: 77 - RNA, 176 nt, Unknown
  Unknown Organism
uuauaauguuagcauaaauacaauaaaguuaaugcaguagaaauacugcgcucuuuaaggugagaauccuugacaagcauguggggcuuauaucuauucauacagagcaaguacguacgggaaagcuuaaaauacucaucuguaaaauaguauuagugcagacuuuaaaaucaugc

SEQ ID NO: 78 - RNA, 128 nt, Unknown
  Unknown Organism
acagaaaaagaagcuaaagaagcaagaaaguauuacugugagaaucaguaauaagcauguggggcuuaugucuuaucaaaaggguggccaacuuuuagauagcauuagugcggacguuaaaaucaugc

SEQ ID NO: 79 - RNA, 157 nt, Unknown
  Unknown Organism
acauuuugugguuuuaaggguuaauccuuaagguugauaaaccuugacaagccuauggggcuacuauaguauucucuuauuacggguaagaguaucaagcauaagcgaaauuccgugcuuauguaaugcuaaguuagugcagacuuaaaaauuaggc

SEQ ID NO: 80 - RNA, 170 nt, Unknown
  Unknown Organism
cuuguucgugagaauaggugcaauugccuaaaugaaugucuucagaagaugacaaaccuguggggcguaaguaauaaagagucugaaagauugcagauaagaguaugcacuuauuggcaauaugcauaccagaauaauuuauuaugaucgugcggacguuaaaaucaggu

SEQ ID NO: 81 - RNA, 159 nt, Unknown
  Unknown Organism
ucagucugugaagauagaguauacguccucagaagaugacaaaccuguggggcguaaguaaaugcauaucguauauuauucccuugaauacggcaauagcggguaauauccgagauacucguauuuguguuuauaaucgugcagacguuaaaaucaggu

SEQ ID NO: 82 - RNA, 78 nt, Fukomys damarensis
ggaggauaacagggggccacagcacaagcguucacgucgcagccccugucggauucugaggaaucugcgaauucugca

SEQ ID NO: 83 - RNA, 78 nt, Sus scrofa
agaggauaacuggcagccacaguagaagcauucacauugugguccaugucagauucuggugaauuugcaaauucugcu

SEQ ID NO: 84 - RNA, 78 nt, Alligator mississippiensis
uuuaugucacugggggccauagcggaaguguucauaucauggccccaaucggauuccaacaaaucugagaauucugcu

SEQ ID NO: 85 - RNA, 77 nt, Latimeria chalumnae
ggguacuauugggggaccguagcaggagcguucacaucgcggucccugucagacuauuacagucugcgaauccugcu

SEQ ID NO: 86 - RNA, 83 nt, Alligator mississippiensis
auugcagcuuagggggccauagcagaagcauucauguugcagccccugucagguaauagcugguaauaccugcuaauucugau

SEQ ID NO: 87 - RNA, 79 nt, Latimeria chalumnae
auuguuuauuuugggggccauagcagaaguguucaugucgcggccccugucagauucuuaugaaucugcaaauucugcu

SEQ ID NO: 88 - RNA, 78 nt, Latimeria chalumnae
uuacccacaacuggggccauagcagaagcguucaugucgcggccccugucauauucuuacaaaccugugaauucugcu

SEQ ID NO: 89 - RNA, 75 nt, Branchiostoma floridae
cgccacuacaugggggccacagaaggagcguucacgucgcggucccugucagguguucuaccugcggauccuucu

SEQ ID NO: 90 - RNA, 83 nt, Alligator sinensis
agcaguuggcuaggggucauaguagaaguguucaugccacaaccccugucagguaauaccuaguaauaccugcaaauucugcu

SEQ ID NO: 91 - RNA, 81 nt, Alligator mississippiensis
agaggucacaaguccgaggccgcggcagaagugcucacggcacgggcccugucagauuccagcgaaucugcaaauucugcu

SEQ ID NO: 92 - RNA, 77 nt, Alligator mississippiensis
cagggguugcaugaggccauagcaaaagcacucacagugcugcccugucagauuccaacaaaucugcaaauucugcu

SEQ ID NO: 93 - RNA, 83 nt, Alligator mississippiensis
aaugcuuugaugggggucauagcagaagcauuaauguugugaccccugucagguaauaccugauaauaccugugaauucugcu

SEQ ID NO: 94 - RNA, 78 nt, Latimeria chalumnae
ugcacaucuaugggggccuuagcagaagcauucacgucgcagccccugucggauucuuaagaauuugcgaauucugcu

SEQ ID NO: 95 - RNA, 78 nt, Alligator sinensis
caauuaagaugcagggccacagcagacauguuuauguuguggucccugucggauucuaaugaaucugagaauucugcu

SEQ ID NO: 96 - RNA, 75 nt, Strongylocentrotus purpuratus
acaguaaaaaaguggggccauugaaggagcguucacgucguggucccugucagaugaaaaucugcgaauccuuca

SEQ ID NO: 97 - RNA, 76 nt, Alligator mississippiensis
aguugcuauaacggccacaacagaaauguucacaucguggccccggucagauuccagcaaaucugcaaauucugcu

SEQ ID NO: 98 - RNA, 81 nt, Alligator mississippiensis
agagguuacaagugcaaggccagagcagaaguguucacagcauagcccuugucagauaccaaugaaucugugaauucugcu

SEQ ID NO: 99 - RNA, 78 nt, Latimeria chalumnae
agcuugcgaaugggggccauagcagaagaguucacgucgcggccccugucagaguucuacgaauuugcgaauucugcu

SEQ ID NO: 100 - RNA, 78 nt, Dasypus novemcinctus
auagaagauaauggggccacagcagaagcauucauguugcagcccuugugagauucaagugaaucugugaauucugcu

SEQ ID NO: 101 - RNA, 104 nt, Aedes aegypti
uacccagcaaauccuaucccuaccuccuuaagguacuggcugaaguacgaguaacuuuaggaaagaucggguaaccaaccccgguccaauucugacugagaagg

SEQ ID NO: 102 - RNA, 96 nt, Anopheles minimus
cacuggcaaaauccgaucccugccuccacguggcgcugcuggaugucgguuuuggugaggcuuaucaccucagccaagaccuaaccaaagggacgg

SEQ ID NO: 103 - RNA, 116 nt, Aedes albopictus
uucccaacaacuccuaucccuaccuccucgugacacucacuggaccgccagcuacuuuagacaagaucggauaacccacccugacggauaauuuggccguuggcugacagggcagg

SEQ ID NO: 104 - RNA, 133 nt, Aedes aegypti
ugcucagcaacuccuaucccuaccuccucgugguacugguacgaguaugggugguaccgguacgaguaaccuuggggaagaucggguaaccaaucccggggggggaacuuuggucguaugcagacagggaagg

SEQ ID NO: 105 - RNA, 123 nt, Aedes aegypti
uguccaguaacuccuaucccuaccuccccguggugccgccugggguacgaguaaucguaggcaacauuggguaaccaacccugacagggaaggcuccucucuucuguaugcugacagggaagg

SEQ ID NO: 106 - RNA, 109 nt, Aedes albopictus
ugcccagcaacucuuaucccuacuuccucgugguaccagccggaaacuacgagaaaccuaagggaagaucggguaaccacaaguguggcgggggcgcagagggggggag

SEQ ID NO: 107 - RNA, 92 nt, Aedes albopictus
ugcccagcaccuccuaucccugccuccacgcgguagggaagaucggguaaccaaccccggugagaaguuuggucguaggcugacagggaagg

SEQ ID NO: 108 - RNA, 106 nt, Aedes aegypti
ugcacagcaacuccuaucccuaucuccucgcgguacugaccgagguacgagcaaccuuagggaagaucggguucugcaaaccuagagcgucuguacauggaguagg

SEQ ID NO: 109 - RNA, 110 nt, Anopheles sinensis
uauucuugaacuccgaucccaaccuccucguggugcuagcugaaguaugaucuuggaacuuauuaaguucuucagcacauugugcaacgaucguauaccaauagggacgg

SEQ ID NO: 110 - RNA, 135 nt, Aedes aegypti
ugccuagcaacuccuaucccuaccucuucaugguacugcccgggguacuggccggaguauuagcaacucaagcaauuagagaagaucggguaacuaaccccggucucaacuuugaucguaugcugauauggaagg

SEQ ID NO: 111 - RNA, 99 nt, Aedes albopictus
ugcccagcaacuccaaucccuacauccgcgagguaccgguuguagacuacgagcaccgagcaaccggugguaacuuuggucguauucugacagggaagg

SEQ ID NO: 112 - RNA, 76 nt, Lutzomyia longipalpis
gguaauccaacuccuacuucaaccuccacguggugacaccugggcacccaauuuauuggguggcuaacugaagagg

SEQ ID NO: 113 - RNA, 113 nt, Aedes aegypti
ugcucagcagcuccuaucucuaccucgucgcauuacuggccgggguccgagcaaccuuauggaaaaucgccccaaccccgagggaaacuuuggucguaugcugacagggaagg

SEQ ID NO: 114 - RNA, 64 nt, Trichomalopsis sarcophagae
ggcguacaaaauccuaucgugcaaccuccccgugguguaugccggguuaugcuaaugcggaagg

SEQ ID NO: 115 - RNA, 76 nt, Acyrthosiphon pisum
ggucggugaaguccuacccccaccaccacguggugccgacuggaaacggaacuccgguuccagccaacgggggagg

SEQ ID NO: 116 - RNA, 99 nt, Aedes albopictus
ugcccagcaacuccuaucccuaccuccucgcgguaccggccggaaacuauaaggcaaucuagcgcucaucacccuucucucucaagcaaacacagaaga

SEQ ID NO: 117 - RNA, 95 nt, Anopheles minimus
cauuggcaaaauccuauuccuaccuccucguggugcugguggaugagggcaugcugagucucacuagcucaguaugucuuaacuaaaagggaagg

SEQ ID NO: 118 - RNA, 95 nt, Lepisosteus oculatus
ggcuggcaaaauccuaucaccaccuccucgcggugccagguggauacggcuggauacaacuggauacaacgacucguuggaacuaacggugaagg

SEQ ID NO: 119 - RNA, 96 nt, Aedes aegypti
uggccagcaccucccauccccaccuccuugugguacuggccaggguacgagcaaccaaucccgguggacacucuugucguaugcugacagggaaag

SEQ ID NO: 120 - RNA, 126 nt, Aedes aegypti
ugcccagcaacucuuaucccuaccuccucuuacuuccucgugguaauggccaggguacgagcaaccuuagggaagaucggauaaccaacccuggugagagcucucgucguaugcuggcagggaagg

SEQ ID NO: 121 - RNA, 98 nt, Aedes albopictus
uaaccaggaacuccuaucccuaccuccccgcgguacugaccgggauacgaucagucccaaucaccgugggaacuuuggucguaugcugacagggaagg

SEQ ID NO: 122 - RNA, 88 nt, Anopheles farauti
ggccggcaaagcccgacccccaccuccucguggugccggcuggaugcauaagacccuacccgucguggguugcagccaacgggggcgg

SEQ ID NO: 123 - RNA, 89 nt, Aedes aegypti
ugccaagaaacuccuccccaaccuccucgugguacuggccgggcuacgaguaaccuuggagaacuuuagucguaugaugacaagaaagg

SEQ ID NO: 124 - RNA, 97 nt, Aedes albopictus
ugcccagcaacucuuaucccuaccuccacgugguaccgcacagaaaaaaaaauauucauguaaaauucagcgacaaaucaugcacauaaagggaaug

SEQ ID NO: 125 - RNA, 130 nt, Aedes aegypti
ugcucaguaacuccuaucccugaccuccccgaggugccggcuggggugcgaacaacccaaagguugaaaggcgaauucacguagccuaaugagcucaaagcgaacucaggucgcaugcugacagggaagg

SEQ ID NO: 126 - RNA, 80 nt, Rhodnius prolixus
ugcucgguaaaaucugaucucuaccuccuugugguccuaccaggaccuuuuaccuacuaagaauaggccaacagagacgg

SEQ ID NO: 127 - RNA, 102 nt, Aedes albopictus
ugcccagcaacaccaaccccuaccuccgcggggcaccagccggacugcaugcggcuguaugcggacuacaugggaccuuuggucguaggcugacagggaagg

SEQ ID NO: 128 - RNA, 109 nt, Aedes albopictus
ugcccagcaacuccuaucccuaccuccucgugguaccggccggaaacuaugauuagcaucacggggaucaucaagaauaauuucggaccgcacaagcuaaaugggugag

SEQ ID NO: 129 - RNA, 97 nt, Anopheles albimanus
cgucucggaacaccuaucucuaccuccacguggugccugcuggauuauggugcaugcgacgguacagcucacaugaaccauauaccgacagagaagg

SEQ ID NO: 130 - RNA, 81 nt, Aedes albopictus
ugcccagcaacuccuaucccuaccuccucgugguacugguuggaaacuacgcuggaaucaacguccgaguuccagggaagg

SEQ ID NO: 131 - RNA, 82 nt, Anopheles albimanus
aacucggaacuccuauccucaccuccacguggugccggcuggaauaugauuguauuagucuaucauauacagacgaggaagg

SEQ ID NO: 132 - RNA, 108 nt, Aedes aegypti
ugcccagcaacuccuaucccuaccuccucgugguacuggccgggguacgaguuguugaucuaagcaaccggaaguccauguccaugaucaaagcacccauagaggaag

SEQ ID NO: 133 - RNA, 94 nt, Anopheles stephensi
ugcuuuagaacuccgaucucaaaccuccucguggugcuggcuggaggauaauuguugcacauuuuacacaacaauuauucacugauugagacgg

SEQ ID NO: 134 - RNA, 94 nt, Aedes aegypti
agcccagcaacuccuaucccuaccuccucgugguacuggccggcugcgaaaggccuggaaaaguuucagaaaauggagucgcuaaaaccgaagg

SEQ ID NO: 135 - RNA, 120 nt, Aedes aegypti
ugcccaauaauuccuaucccuaucuccccacgaugccgcccagaguacgaguaaucaucuuuccgaucuuuuccaguaaucaaccccggugagaccuuggucguaugcugacaagaaagg

SEQ ID NO: 136 - RNA, 134 nt, Aedes aegypti
ugcccagcaacuccuaucccuaccuccucgugguacuggccgggguacgaguaaccuuggggaaguaguaggaaguaguaggaaggaguaaccaacccccggugggaacuuuggucguaugcugacaggaaagg

SEQ ID NO: 137 - RNA, 80 nt, Tribolium castaneum
uccuggcaaaaaugcucuaaaccuccacgugguucuugcuggacaaauuaguuauuagcuaauuugaccaauuagagcaa

SEQ ID NO: 138 - RNA, 89 nt, Anopheles farauti
gccuuuggaacuccguuuucuaaccuccacguggugcuggcuggaauauggucuuuccuuuauggucgaucauauacaaauagaaacgg

SEQ ID NO: 139 - RNA, 105 nt, Aedes aegypti
uguucaucaacuccuaucccuaccuccucgcgguacuguccgggguacgagcaaccuuagagaagaucccgcaacggcuucguggcgcgagccgagaugugcagg

SEQ ID NO: 140 - RNA, 104 nt, Aedes albopictus
aacccaguaacuccgaucccuuccuucacgcggcgccggccggggugcgaccauccgaaagguagauuaagcuugaagcuuaggucguauguugacagggaagg

SEQ ID NO: 141 - RNA, 119 nt, Culex pipiens
uguucaguaacuccgauacccuggccuccccgcggcgcuggccgggauacuaguaaccauuggagagaucggguaaccaaccccggugggaacuaugguaguaugcugacaggguaagg

SEQ ID NO: 142 - RNA, 83 nt, Anopheles epiroticus
ggccgacaaaacccucucccaaccuccacguggugucggcuggaaagugccucauguaauguugcauuuaccaacugggaagg

SEQ ID NO: 143 - RNA, 104 nt, Anopheles albimanus
aaucucggaacuccuauccccaccuccucguggugccggcuggaauaugguagaugugcaugguauccgaccaauaucaucuuaccauauacagacggggaagg

SEQ ID NO: 144 - RNA, 92 nt, Aedes aegypti
ugccaagcaucuccuaucccuaccaucucgugguacuggccgugguacgagccucccagaugggaacgauggucguauggugacagcgaagg

SEQ ID NO: 145 - RNA, 55 nt, Folsomia candida
aacaaauauacgggugcccccguacugaugaggccauggcaggccgaaauuugug

SEQ ID NO: 146 - RNA, 55 nt, Paenibacillus wynnii
cuugcuuauggacucaguucacugacgagcucgugagauucgagcgaaaaguauc

SEQ ID NO: 147 - RNA, 60 nt, Agaricus bisporus
gucggauuagggcagcgguuaagcccucugaugagccccuucgcaagggcgaaauccgca

SEQ ID NO: 148 - RNA, 58 nt, Eucalyptus grandis
aauuaguugggaguugaugcugcucuccugaugaggccauagcaggccgaaaccaguu

SEQ ID NO: 149 - RNA, 58 nt, Eucalyptus grandis
aauugguugggagcuaaugcuauucuccugacgaggccauggcaggcugaaacuauuu

SEQ ID NO: 150 - RNA, 75 nt, Habropoda laboriosa
guggcgucuggggcauggaccggcuacaucagccucacugaugagucuguggucggucucgagacgaaacgcuuc

SEQ ID NO: 151 - RNA, 57 nt, Desulfobulbus sp.
gugaugucugcggcugaaucugccgcacugacgagcccauccagggcgaaacaucca

SEQ ID NO: 152 - RNA, 58 nt, Orchesella cincta
gacgcgucuagaagugaagcccuucuacugaugagguuauggcagaccgaaacgcaaa

SEQ ID NO: 153 - RNA, 58 nt, Helianthus annuus
cacuaguugagaguugucgcugguuuccugaugaguccaaggcaagacaaaaccagua

SEQ ID NO: 154 - RNA, 56 nt, Citreicella sp.
cccagguacccggauguguuuuccgggcugaugaguccgugaggacgaaaccuggg

SEQ ID NO: 155 - RNA, 67 nt, Chlorocebus sabaeus
auucagucaggaguuuuuucugcugaugaguuccuggucuugcuaacuucaaagaacgaagcugcag

SEQ ID NO: 156 - RNA, 60 nt, Fomitopsis pinicola
ggacggucggggcagcggguaagcccccugacgaggacuuucgcagguccgaaaccgcug

SEQ ID NO: 157 - RNA, 75 nt, Trichomalopsis sarcophagae
ugcgcgucugaggcaggguuaccaucggaugccuuacugacgaguccacgaugguaaccugggacgaaacgcaac

SEQ ID NO: 158 - RNA, 60 nt, “Vitis vinifera,”
aacuggucaagagcuggagucauuccccugaugaauccaugaaucaggaugaaaccaguu

SEQ ID NO: 159 - RNA, 57 nt, Candidatus Uhrbacteria
uuuuugucuuuagauacaguaucuaaacugaugaguccuguaaggacgaaacaaaag

SEQ ID NO: 160 - RNA, 61 nt, Asian citrus
aacgcgucuuaggcugcucucaggugcuagcugaugaguuccaacaagaacgaaacgcguc

SEQ ID NO: 161 - RNA, 79 nt, Dufourea novaeangliae
caggcgucugggguuggggucgucuaccgucagucccacugacgaaucuugguugacgauucucgagacgaaacgccau

SEQ ID NO: 162 - RNA, 58 nt, Eucalyptus grandis
aacuggucaggagcuuaugcuaccauccuaaugaggccaugguaggccgaaaccaguu

SEQ ID NO: 163 - RNA, 58 nt, Citrus clementina
cacugguugggaacugaagccguucuccugacgagcccacgguagggcgaaaccaguc

SEQ ID NO: 164 - RNA, 56 nt, Echinostoma caproni
cuggagugauauuugcugauauuuacugaugagcuccaauaagagcgaaacucgag

SEQ ID NO: 165 - RNA, 58 nt, “Vitis vinifera,”
aacuaguugggagcuagagccauuccccuuaugaguccauggcaagacgaaaccaguc

SEQ ID NO: 166 - RNA, 68 nt, Candidatus Uhrbacteria
accacuucugccguugaguacggcacugaugaguccauucgauuguaaacagcaggacgaaaaguaaa

SEQ ID NO: 167 - RNA, 55 nt, Candidatus Taylorbacteria
cguugcucucggaauguguauuccgacugaugaguccaaaaggacgaaagcagaa

SEQ ID NO: 168 - RNA, 56 nt, Candidatus Omnitrophica
cggcuguuucccgauguguuaucgggacugaugaguccgaaaggacgaaacagcgu

SEQ ID NO: 169 - RNA, 58 nt, Rhizobium phage
aauagguacggggcugaugcugccccgcugaugaggccaagcuauggccgaaaccauc

SEQ ID NO: 170 - RNA, 58 nt, Eucalyptus grandis
aacuggucgagaguugaugucgcucucuugacgaggccauggcaggucgaaaccaauu

SEQ ID NO: 171 - RNA, 58 nt, Octopus bimaculoides
aaugagucaagugacgcgaacaucucugaugagacccucaaaaaggucgaaauucgau

SEQ ID NO: 172 - RNA, 57 nt, Perkinsus marinus
ggugugucuggcgccguuagccacugaugagucccuguggugaggacgaaacacuac

SEQ ID NO: 173 - RNA, 56 nt, Trachymyrmex cornetzi
uauaugucaguuugcguuugcucugaggagggcucaggaaugagccgaaacaugua

SEQ ID NO: 174 - RNA, 55 nt, Parcubacteria (Giovannonibacteria)
ccacuguccuagaguguguacucuagcugaugagucggaaacgacgaaacagaaa

SEQ ID NO: 175 - RNA, 53 nt, Octopus bimaculoides
ccgaagucgagcugucuuaauugaugaggcgaaggaaaaugccgaaacuacgc

SEQ ID NO: 176 - RNA, 74 nt, Dufourea novaeangliae
cccgcgucuaaggcagggucugcuagaaaagccuuacugacgaguccacuagcaugcccaggacgaaacgcucc

SEQ ID NO: 177 - RNA, 60 nt, Schistosoma rodhaini
uggauguauauucaugauauaggauugcugaugagucccaaagauaggacgaaacaaccg

SEQ ID NO: 178 - RNA, 55 nt, Pleurotus ostreatus
uuuguguugggaggugugugccucuccugaugaauccaaaaggacgaaacacauu

SEQ ID NO: 179 - RNA, 99 nt, Melipona quadrifasciata
agggcgucugggguaggagucacugccaucaaaacaccccccuccccccccccccccccacugaugagucuaggcagcgacuccgagacgaaacgcauc

SEQ ID NO: 180 - RNA, 58 nt, Ipomoea nil
aacuagucgggagcuauugacguuccccugaugagcccaugacgggacaaaaccaguu

SEQ ID NO: 181 - RNA, 52 nt, Punctularia strigosozonata
gcucggucaucucgggcagaacccugaugagccuauaaaggcgaaacagggc

SEQ ID NO: 182 - RNA, 62 nt, Melipona quadrifasciata
caagcguuuuggggccagccccacugaugagucuaggcagcgacuccaagacgaaacgcauc

SEQ ID NO: 183 - RNA, 55 nt, Mycobacterium obuense
cugcucuccagggucacccugcugacgagcccgugaaagucgggcgaaagagccc

SEQ ID NO: 184 - RNA, 74 nt, Candidatus Giovannonibacteria
gaacgcucgcgagaugugugucucgccugaugagcccgccaaaggcgggcaaguccaaaaggacgaaagcgugu

SEQ ID NO: 185 - RNA, 54 nt, Trichobilharzia regenti
aaugcauccaguacauccacuggcugacgaguccgagauaagacgaaaugcaug

SEQ ID NO: 186 - RNA, 59 nt, Schistosoma rodhaini
gacaugucugggaugcagguacauccaacugacgagucccaaauacgacgaaacaugca

SEQ ID NO: 187 - RNA, 69 nt, Aedes albopictus
ucaaagucuugacgaaaggccaacgggccaaaacgucaacugaugaguccuugauggacgaaacuuugu

SEQ ID NO: 188 - RNA, 55 nt, Candidatus Lloydbacteria
uugcuguagagaagugcaugcuucuccugacgagucggaaacgacgaaacagcac

SEQ ID NO: 189 - RNA, 110 nt, Bacterium SM23_31
agcagagaccgggaagggauucucuuauuaugaaaauauugaaaauagcaugaaacacuaaaccccggggauccucccgguaaugcagccguagccggucacaagcccgg

SEQ ID NO: 190 - RNA, 58 nt, Clostridium thermocellum
uccagagugacggaacgacucuuccuccgguaaugcgguggcccggucacaaguccgg

SEQ ID NO: 191 - RNA, 60 nt, Candidate division
cgcagagaggggcuaggccauaggcuuagcucuaaugcggcauaccggucucaagcccgg

SEQ ID NO: 192 - RNA, 51 nt, Balearica pavonina
ugcagauggaauaauuuaaugcaacuguaguuacucagguuccaaguccug

SEQ ID NO: 193 - RNA, 63 nt, Spirochaetes bacterium
ugcagagggggccgggacgcgcgaagcgacucggccuaaugcacaggccggucccaaguccgg

SEQ ID NO: 194 - RNA, 57 nt, Clostridium asparagiforme
cgcagagcaacggggcagcaaugccccgguaaugcgggggaacgguugcaaccccgu

SEQ ID NO: 195 - RNA, 65 nt, [Clostridium] hungatei
ugcagaugggcggccuuauggccguuaaugcgcucccggauaccgggaacccguccaaagccggg

SEQ ID NO: 196 - RNA, 60 nt, Strongylocentrotus purpuratus
agggagggagggguauuggaaccaaaccucuuaaccaaccgucgcccgucccaagucggg

SEQ ID NO: 197 - RNA, 56 nt, Desulfosporosinus sp.
cgcagagugaccgcccaucgcgggcggguaaugcggcuagccggucacaagcccgg

SEQ ID NO: 198 - RNA, 70 nt, Clostridium sp.
cgcagagcagcggagaaacugacuucguuaaugcggccugacguuuuucgucugacgguugcaagcccgc

SEQ ID NO: 199 - RNA, 65 nt, Unknown
  Unknown Organism
ugcagaugggcgccuucgggcguuaaugcgcugaaaccaaagguuccaccagguccaaaguccug

SEQ ID NO: 200 - RNA, 59 nt, Unknown
  Unknown Organism
ggugagcggccccgcccguaaggacggggacuaaaccacaaguccggucgcaaguccgg

SEQ ID NO: 201 - RNA, 71 nt, Ruminococcaceae bacterium
ugcagagugagaaagcucauuaccguuuggugaugggcuuuuguaaugcagagcgccggucacaaucccgg

SEQ ID NO: 202 - RNA, 56 nt, Unknown
  Unknown Organism
cgcagaugacggugccaccacggcaccguaaugcgacaagcagguuccaaucccug

SEQ ID NO: 203 - RNA, 72 nt, Unknown
  Unknown Organism
ugaugaggggcggggggccagagaccccccguuaaaucgccaugucaaccgacaugcuggucccaagcccag

SEQ ID NO: 204 - RNA, 61 nt, Unknown
  Unknown Organism
agugaggggaucgaucuaaacuacuggcuuguuucgugcaagucaccggucccaaguccgg

SEQ ID NO: 205 - RNA, 65 nt, Unknown
  Unknown Organism
cgcagagcacgcccuacggggcguaaugcggccucaccacuggggugagccaguugcaagccugg

SEQ ID NO: 206 - RNA, 63 nt, Unknown
  Unknown Organism
cgcagagggcagcccuucggggcuguaaugcacuccccaccuggggagcggucccaaguccgc

SEQ ID NO: 207 - RNA, 64 nt, Unknown
  Unknown Organism
cgcagagugacgggaggguuuaucggcccucccgguaaugcggcagcccgguucgcaagcccgg

SEQ ID NO: 208 - RNA, 55 nt, Unknown
  Unknown Organism
cgcagagugagccgggaaaccggcuuaaugcgggcagaggcggucacaaccccgc

SEQ ID NO: 209 - RNA, 219 nt, Naegleria sp.
cuguuauuggaauuugauaguugugcgaugggguucauaccuuaacugccaaaacgggaccccuuuugggguauaaaucuuguaaaaggauuauauuccguacuaaggauauuugauaauauccggaaugucuagagacuacacggcaagccaauuggugguaugaauggauagucccuaguuuuuuuuaccaucuagguaucccauacaaaaugguaa

SEQ ID NO: 210 - RNA, 196 nt, Didymium iridis
uuuugguuggguugggaaguaucauggcuaaucaccaugaugcaaucggguugaacacuuaauuggguuaaaacggugggggacgaucccguaacauccguccuaacggcgacagacugcacggcccugccucuuagguguguucaaugaacagucguuccgaaaggaagcauccgguaucccaagacaaucaaau

SEQ ID NO: 211 - RNA, 200 nt, Naegleria sp.
cuguuauugaaggacguucuagagugcgaugggguucauaccuuuaucugccaaaacgggaccucuguugagguauauauugaauauuccguacuaaggauuuaauccggaacgucuagagacuacacggcagaccauuguuggugguaugaauggauagucccuagugaaccaucuaggcaucccauacaaaaugguua

SEQ ID NO: 212 - RNA, 209 nt, Heterolobosea sp.
cagcuguuuugauacaugcucgacuuucuuuuucucuugugcaaugggguuuaugaguuaauuagccaaaacgggaccuuaaaaagguguaaguaaccguacuaaguucguaagaacggaaugucuagagacuacacggcugagcgauuuagcucucauaaauggauaguccucaguauaccaucugagcaucccauacaaaaugguua

SEQ ID NO: 213 - RNA, 76 nt, Eubacterium ruminantium
agucgucagagcgacuauaaauaggcuuuaggcucugagcgugccgaccgucaauaaaaggcggucagcgguagca

SEQ ID NO: 214 - RNA, 71 nt, Anopheles gambiae
acucgacuaagcgaguauaaaaagguuucaagcuuagagcguugauagggauaaaaaccuaucagguaaca

SEQ ID NO: 215 - RNA, 71 nt, Tsukamurella phage
ccucgucagggcgagguuaaauagccgcauaggcccugagcguccccgccccacaagggcggggggacggg

SEQ ID NO: 216 - RNA, 65 nt, Paenibacillus sp.
agucggcuuggcgacuauaaauaggcuuuuggccaagcgcgggcucccaacucgggaguauagca

SEQ ID NO: 217 - RNA, 69 nt, Paenibacillus naphthalenovorans
acucgugccagcgaguuuaaauagaccaauaggcuggcagcguuccacucauaaagaguggaggaggua

SEQ ID NO: 218 - RNA, 71 nt, Ruminococcus sp.
aguggucacagccacuauaaacagggcuuuaagcugugagcguugaccgucacaacggcggucagguaguc

SEQ ID NO: 219 - RNA, 69 nt, Clostridium sp.
aguagucauggcuacuauaaauagagacuuaagccaugagcguucccaucuuugugaugggaggugucu

SEQ ID NO: 220 - RNA, 69 nt, Gordonia phage
cgucgucugagcgacguuaaauagccguuaggcucagagcgguacaccuccccuauucucgggguuggg

SEQ ID NO: 221 - RNA, 66 nt, Cellulophaga phage
agccguugcagcggcauaaaauagguuauuaggcugcaagcguucgcccuuaauugggcgguguua

SEQ ID NO: 222 - RNA, 65 nt, Sphingobacterium sp.
agucguuugagcgacuuaaaauagguuuuaagcucaaagcgccccgauaauaaucgggaguaaca

SEQ ID NO: 223 - RNA, 70 nt, Blautia sp.
agagguugcaaccucuauaaauagggcuuuaaguugcaagcguucccgcuggaaacagugggagauagcc

SEQ ID NO: 224 - RNA, 66 nt, Lachnospiraceae bacterium
agccgucccaacggcucuaaaaaguccauuaaguugggagcguccggcagaaaugccgggguugga

SEQ ID NO: 225 - RNA, 75 nt, Clostridiales bacterium
gcucgucugggcgaggguaaauaguaauuaggcccagagcgucuuggcuggcagaucugccggucggggguuuag

SEQ ID NO: 226 - RNA, 64 nt, Brevibacillus phage
uaguguugcggcacuuacaagcccauuaagccgcaagcguuagcccuuccggggcuagguuggg

SEQ ID NO: 227 - RNA, 74 nt, Gordonia phage
acacgacuggacguguauaaauaggcguuagguccagugcgggugaugguauugaguauuuuggaaucggugcc

SEQ ID NO: 228 - RNA, 64 nt, Bacillus glycinifermentans
agucguggcggcaacauuaaacaggcauuaagccgccagcauuccccuuauuggggagguugca

SEQ ID NO: 229 - RNA, 69 nt, Bacillus glycinifermentans
ggacgugacggcggcucaaaaaagugcauuaagccgcaagaguuuccccguuuuugggggaagguuuca

SEQ ID NO: 230 - RNA, 80 nt, Ethanoligenens harbinense
caccguggcggcgguguaaaacaaacauuaagccgccagcgucccggaacaaggcauuuuccgauucuccggggguugca

SEQ ID NO: 231 - RNA, 70 nt, Clostridiales bacterium
gcucgucugggcgaggauaaacagcuauuaagcccagagcguucugagucuuuaagauucggagguuuag

SEQ ID NO: 232 - RNA, 69 nt, Bacillus phage
agucgugugagcgacuauaaacaggcuuuaggcucacagcgucgcgggguuuaucccccguggguagca

SEQ ID NO: 233 - RNA, 70 nt, Sphingobacterium sp.
aguggauugcgccacuuuaaaaagguuuuaagcguaaagcguugcaagguuuugagccuugcagguaaca

SEQ ID NO: 234 - RNA, 64 nt, Bacillus glycinifermentans
acucgucacagcgaguauaaagaggcauuaggcugugagcguuccccgucauggggagguugca

SEQ ID NO: 235 - RNA, 67 nt, Clostridiales sp.
acacguugcgccguguauaaauagccaguuagggcgcaagcgucccggcauuuugccgggggucugg

SEQ ID NO: 236 - RNA, 65 nt, Alistipes sp.
agccguucggguggcuauaaauagaccuuaggcccgaagcguggcggcaccugccgccgguggua

SEQ ID NO: 237 - RNA, 78 nt, Streptococcus sobrinus
agucguuguggcgacuauaaccaagcucuuuaagccacaagcguugcugaugagguuucauaacaucagcagguagag

SEQ ID NO: 238 - RNA, 67 nt, Paenibacillus elgii
acugguucgagccaguaaaaaaaggccgauaagcucgaagcguuccacucuuagaguggaggaggca

SEQ ID NO: 239 - RNA, 66 nt, Unknown
  Unknown Organism
agucguuagggcgacuauaaacagacauuaagcccuaagcguccccuacuagcuagggggguugua

SEQ ID NO: 240 - RNA, 69 nt, Unknown
  Unknown Organism
agucgguagagcgacuuuaaaaaggcauuaggcucuacgcguuccaggaggaaacuccuggagguuguu

SEQ ID NO: 241 - RNA, 64 nt, Erysipelotrichaceae bacterium
auucgacuagacgaguauaaauaggugucaggucuagugcggcaggguucuucccugcaucaua

SEQ ID NO: 242 - RNA, 69 nt, Erysipelotrichaceae bacterium
aaucgacuaggcgauuuuaaauagguguuaagccuagugcgguaagagguauaacccucuugcgucacg

SEQ ID NO: 243 - RNA, 70 nt, Unknown
  Unknown Organism
gcuggucacggccaguauaaacagacauuaagccgugagcgucuccuguucugugaacgggaggguugua

SEQ ID NO: 244 - RNA, 66 nt, Unknown
  Unknown Organism
acucguuagggcgaguauaaauagccauuaggcccuaagcgucaaugauaagcucauuggguugga

SEQ ID NO: 245 - RNA, 66 nt, Unknown
  Unknown Organism
agucguuugggcgacuauaaacagacgaauaagcccaaagcguuuccucguaagaggaaggacgga

SEQ ID NO: 246 - RNA, 66 nt, Unknown
  Unknown Organism
agucgucugagcgacuauaaacagaguuuuaggcucagagcgccuccccuucgggggaggguacua

SEQ ID NO: 247 - RNA, 79 nt, Atta colombica
acucgacuagacgaguauaaacuacauuaagccuagugcguuauagccguaaauaagaaguaaacggcuauagguugua

SEQ ID NO: 248 - RNA, 70 nt, Paenibacillus polymyxa
guucgucugagcgaacgcaaacaggccauuaagcucagagcguucacuggauucguccagugagauuggc

SEQ ID NO: 249 - RNA, 71 nt, Unknown
  Unknown Organism
acuggacuacgccaguauaaauaggcauuaagcguagugcguuccaauguugugaaacaucggagguuguu

SEQ ID NO: 250 - RNA, 66 nt, Unknown
  Unknown Organism
agucgucuaagcgacucuaaaaaggcuuuaagcuuagagcguucgcccauauugggcgagguugua

SEQ ID NO: 251 - RNA, 73 nt, Unknown
  Unknown Organism
acugguugcggccaguauaaauagucuuuaagccgcaagcguguccuggaguuaaucuuccagggcgguagca

SEQ ID NO: 252 - RNA, 69 nt, Unknown
  Unknown Organism
agucgacuaagcgacucuaaacagcauuuaggcuuagugcguuccccugcucacgcgggggagguaugg

SEQ ID NO: 253 - RNA, 78 nt, Lysinibacillus sphaericus
acucgacuaagcgaguauaaacaggcauuaggcuuagagcguucucacguuaucugaaugaugaugugagagguugca

SEQ ID NO: 254 - RNA, 55 nt, Unknown
  Unknown Organism
acucgacagggcgaggcuaaauagcauuuaggcccugagcggcucccuucgggag

SEQ ID NO: 255 - RNA, 58 nt, Unknown
  Unknown Organism
gcucggugcggcgagccuaaauagugccuuaggccgcacgcguuaugcauagguggca

SEQ ID NO: 256 - RNA, 66 nt, Unknown
  Unknown Organism
acagguuugcgccuguauaaauagacauuaagcgcaaagcgucccgcaauuguugcggggguugua

SEQ ID NO: 257 - RNA, 67 nt, Tolumonas auensis
aagcgaaacaggccccggagggccugucugccggagguggugcuccgguacugaugagcagccuagc

SEQ ID NO: 258 - RNA, 69 nt, Mycobacterium vanbaalenii
ugccgaaacgccgacucgggucggcgucccugggagguggcauucucaggcugaugauggcugccgcag

SEQ ID NO: 259 - RNA, 77 nt, Shewanella oneidensis
aagcgaaacaagcaaggcgcuuaggugccuugccugucugcucggcgugguugccgagcacugaugagcagccaaag

SEQ ID NO: 260 - RNA, 87 nt, Desulfobacteraceae bacterium
augcgaaaccgcgaucauuuugccgccauuggcaaggugaucgcggucaucagggugcggcgauccugaucugaugagcagccaaga

SEQ ID NO: 261 - RNA, 70 nt, Desulfovibrio alkalitolerans
aagcgaaaccgcccugagugggcggucguuccggagagacggcgaccggggccugaugagccagccgaau

SEQ ID NO: 262 - RNA, 77 nt, Streptomyces phage
augcgaaacaucucgccggcuggaccggugaggugucggcccagggcgguuccuggguccugacgaugcaaccggga

SEQ ID NO: 263 - RNA, 71 nt, Thermoplasmatales archaeon
agccgaaacaggggucugugcgccccuguccaccauggguggugccauggugccgaugaugguagccacaa

SEQ ID NO: 264 - RNA, 70 nt, Paenibacillus sp.
agccgaaacgccucgcgauaggaggcgucgcggggauauggccuaccccgccugaugauggcaggccgga

SEQ ID NO: 265 - RNA, 74 nt, Neptunomonas antarctica
ccgcgaaacgcccacaccuuaacgggacgggcgucuauccagcguggcaacuggguacugaugagcagccacua

SEQ ID NO: 266 - RNA, 67 nt, Ethanoligenens harbinense
agccgaaacggggugaaagcccuguccgcuggggauggccuccucgcgcugaugauggcaggccaac

SEQ ID NO: 267 - RNA, 84 nt, Rhodobacterales bacterium
augcgaaaccgcauccggggcggcgugugccccgggugccggucggccgggcgugguggcccgguccugaugaugcagccggag

SEQ ID NO: 268 - RNA, 68 nt, Pirellula sp.
agccgaaacgcgguagcgauccgcgucgccgaucggugguucgaucggccugacgauggcagccaacc

SEQ ID NO: 269 - RNA, 74 nt, Devosia sp.
uugcgaaacgccucccggcuccggcugggggcgucguccacgggucgcgccgugggccugaugagcagcgacac

SEQ ID NO: 270 - RNA, 77 nt, Verrucomicrobiaceae bacterium
ugccgaaacggcuuccucgugccccgaggugccguccugccgggcugagcucccagcagcugaugaggcagcucccu

SEQ ID NO: 271 - RNA, 68 nt, Saccharothrix sp.
cggcgaaaccgccuccccggaggcgguccacgggauuggcauucccgugcugaggaugccugccgagc

SEQ ID NO: 272 - RNA, 69 nt, Marinomonas sp.
uagcgaagcgcggcuagguauagccgcgucaaucucguguaguggcuagauacugaugagcagcuaaaa

SEQ ID NO: 273 - RNA, 75 nt, Ruegeria sp.
augcgaaaccgucccgguguucacgccgggauggucaucggggcguggugaccccggucugaugagcagccagaa

SEQ ID NO: 274 - RNA, 67 nt, Beggiatoa sp.
aaccgaaacuccccucacggggaguccgaccgggauuaaucacccggcgcugaugaggcagauuccu

SEQ ID NO: 275 - RNA, 65 nt, Streptomyces phage
ugccgaaacacccuucggggugucggggugggguggcgcucaccuccugacgauggcagccacga

SEQ ID NO: 276 - RNA, 69 nt, Lachnoclostridium sp.
agccgaaacggucaguaaugaccgucagccgggaagugacugccccggcucugaugauggcaggucaug

SEQ ID NO: 277 - RNA, 66 nt, Herbaspirillum seropedicae
agccgaaacauccucaaagggugucucucagagguggccuccugagacugaugauggcuggcugug

SEQ ID NO: 278 - RNA, 71 nt, Moritella viscosa
aagcgaaacacgucuuagugauaagucgugucuacucagcguugugguugaguacugaugagcagcaacuu

SEQ ID NO: 279 - RNA, 67 nt, Fervidicella metallireducens
aaccgaaacaaggguaugucccuugucugcugaggauaaccucucagcacugaugagguagguuaaa

SEQ ID NO: 280 - RNA, 67 nt, Saccharothrix sp.
cggcgaaaccguccgguguggacggucccgagggcuggcaucccucggcugaugaugccugccaaga

SEQ ID NO: 281 - RNA, 61 nt, Streptomyces phage
aggcgaaacgccgugaggcguccggccgggugguacccggucgcugaugagccagccugcu

SEQ ID NO: 282 - RNA, 67 nt, Ethanoligenens harbinense
agccgaaacgggacuuugguccugucugccgggaauggccgcccggcacugaggauggcaggcugcu

SEQ ID NO: 283 - RNA, 66 nt, Oscillibacter sp.
agccgaaacgcccuccggggcgucaucggggggagcccucccccggucugaagauggcagggcacg

SEQ ID NO: 284 - RNA, 69 nt, Subdoligranulum sp.
agccgaaacagcccugcggggcugucgugcgggggcugaccgccccgugccugaugauggcaggucaag

SEQ ID NO: 285 - RNA, 69 nt, Acinetobacter baumannii
aagcgaaacacaggcauucgugccugugucuacuggaugucgugauccaguacugaugagcagcgauag

SEQ ID NO: 286 - RNA, 66 nt, Streptomyces hygroscopicus
ugccgaaaccccuuggugaggggucguuccgggguggugcccggagccugacgacggcagccgccc

SEQ ID NO: 287 - RNA, 70 nt, Ruminococcus callidus
agccgaaacagcggcagagagccgcugucugccggaacuggucuaccggcacugaugauggcagaccgga

SEQ ID NO: 288 - RNA, 65 nt, Saccharothrix sp.
aggcgaaacccggcuggcaccggguccguagggcuggcaucccugcgcugaugagccugccaacg

SEQ ID NO: 289 - RNA, 67 nt, Blautia sp.
agccgaaacggggaacuuaccccguccgcugcgggaucgccucccggcgcugaugaggcaggcgaga

SEQ ID NO: 290 - RNA, 78 nt, Rhodovulum sp.
ccgcgaaaccccgccaggcccaucggucuggcggcggucggccgggcgugguggcccgacccugaugagcagccggag

SEQ ID NO: 291 - RNA, 81 nt, Geobacteraceae bacterium
augcgaaacgaucauuuugccggcgucgacaaaaugaucgucaucccggcguggcggccggggucugaugagcagccgcgg

SEQ ID NO: 292 - RNA, 68 nt, Streptomyces yokosukanensis
cggcgaaacccgcuggugaggcgggucgcgaagcgguggugcgcuucgccugaugaugccagccagca

SEQ ID NO: 293 - RNA, 84 nt, Endozoicomonas sp.
uugcgaaacacucccgccguaccuguccccacaggugggagugucaguccaguguggugacugggcucugaugagcagccaaag

SEQ ID NO: 294 - RNA, 68 nt, Chloroflexi bacterium
agccgaaacgggggcaucggcccccgucgucccgggcaguccacugggaccugacgaggcaaagcgcg

SEQ ID NO: 295 - RNA, 72 nt, Shewanella oneidensis
aagcgaaacccgccccauucauggggcgcggucugucuaauguagugauuaggcacugaugagcagcuaacc

SEQ ID NO: 296 - RNA, 66 nt, Streptomyces phage
aggcgaaaccacccgagaggguggucggaccgggcgguucccgguuccugacgaugccaaccacug

SEQ ID NO: 297 - RNA, 69 nt, Desulfofustis glycolicus
aggcgaaacgccggggugacccggcgucgucggagggugaugccuccggccugacgaugccaguuacag

SEQ ID NO: 298 - RNA, 68 nt, Desulfovibrio sp.
aggcgaaaccguucuccucggagcggucggccgggugugguggcccggcccugaugagccagccgcuc

SEQ ID NO: 299 - RNA, 76 nt, Desulfuromonas soudanensis
aagcgaaacgaccacccccccagggggguagucgucgcucggggguggugccccgggccugaugaugcagccaagu

SEQ ID NO: 300 - RNA, 69 nt, Marinomonas sp.
aagcgaaacauggcucguuguagccgugucuauucagcguaguggcuggguacugaugagcagcuaaaa

SEQ ID NO: 301 - RNA, 57 nt, Faecalibacterium prausnitzii
gcggacacuuucaagggcugcaccgcugccgcaaaagcaacccuaugccaccgcccc

SEQ ID NO: 302 - RNA, 54 nt, Faecalibacterium prausnitzii
acggaugccuugacgggccgcaccgaacgaaaagcgacccgaucccacaccccg

SEQ ID NO: 303 - RNA, 53 nt, Faecalibacterium prausnitzii
acggauacucuagccggguugcaccguucaaagcagcccagccccagccgcaa

SEQ ID NO: 304 - RNA, 54 nt, Faecalibacterium prausnitzii
ugggauacccuagcagggccgcaccccagaaaagcggccccgccccacacccgg

SEQ ID NO: 305 - RNA, 53 nt, Faecalibacterium sp.
ccggauauuuuggcagggcugcaccgggcaaagcaaccccgccccacuacccc

SEQ ID NO: 306 - RNA, 53 nt, Unknown
  Unknown Organism
gcggacaccucagcagggccgcaccggacaaagcggccccgccccaccgccca

SEQ ID NO: 307 - RNA, 57 nt, Faecalibacterium sp.
gcggaugcccuggcagggucgcaccgcucaaacaaagcggccccgccccauaacccc

SEQ ID NO: 308 - RNA, 52 nt, Unknown
  Unknown Organism
ucggacacucuggcagggcaagcaccguauagcagccccgaccaacuacccc

SEQ ID NO: 309 - RNA, 54 nt, Unknown
  Unknown Organism
ccggaagcccuggcagggugcgcaccggauaaagcggcccuaccucaccggcac

SEQ ID NO: 310 - RNA, 247 nt, Unknown
  Unknown Organism
aaaagcaccucuuaaauagugauccguaaaaugagguucauauaaaauuuuucacuauaugcuggaaaaucuuaaagcuuuaaguaccucaaugguaacaaucuuaaagauauuacaauagacaaucagcaggaaaccaacauaauucuauuauuuuuaguaggauccucagagacuacacgugaaacaccguauuuuuauuaagaauacgcugaagauauaguccgccccacuucgaaagaugugg

SEQ ID NO: 311 - RNA, 262 nt, Candidatus Taylorbacteria
cuguuauaguucuguuaaugcaauaaaauguaaaaacauuuugauaaacuaaauaaacaauaaucaguacacuauucaaagcugccucgcucuguaguaauacaaagcagguccgcaccauggcuauaugcggggaagucuguaauuugcagaucauccgcagggaaguucuaaaauuuuuuuuagaaccccucagagaccacacgccaugcuccagcugguuuguaccagcuggaugaagauaugguccuucguuaaagag

SEQ ID NO: 312 - RNA, 428 nt, Candidatus Yanofskybacteria
caaggcggcuuguuacuugccgcaggggccauugagaagcaauucucaauagcaaauucgacuauaugcuggaaacuccgccaguaucucuagguacuaugauauuaugauauaucauagugaaaauccuaagagugaugcggacaaucagcaggcaaccccgcucaaauuuuuggaaauaaaaaaguggauuucugggauucaggauccggccucgauuagagguugccauccuuucggaaggaugcccugaaaugcuucccacgcaaaauccacuuucaugcuaguauaucaaauaaauaaugcacuugucaaguguuuguuucuaaaaauuugagcgggagaguccucaacgacugaaagucgaaccaguuauauuucaaaaaaaugacuggaugauacagucugaacuuauaggcgacuauaag

SEQ ID NO: 313 - RNA, 501 nt, Trichoplax adhaerens
aagauuaaauaauauaaguuuuugacuuuuaccuccggcacuuuuuuuacuauuagguuccucuuuaguagaacaaggagcggguacaggguggacgguugaaaggccgcccgaguuagugaugacuuggugaaaauuuugcucaaugcgagaacauccucaaaaaaaaggugcuuuggcucauugauuaacccuaaaaagguaccuuuugauggccccaugcaaaaauccuuuuuuacgccgaaggcgccguggacaacucgccgggggcccaagccuaugggccccucagagacuaaaugcagaauaucuucuauuuuuugauaggcgccgggccccuuaacgggcgccgaaggcgcccaaugggagccaacgaccgauggcgccauaggcgccgaaggcgccgauagaaauaaagggcccgaagcgaccgauucaccaaucggucgcuucggccgauggaagauaaaggaauaguccgauccgacucuaaagggucgg

SEQ ID NO: 314 - RNA, 307 nt, Galerina marginata
uugccuggguuuucuuaauugaauucccgaauuuaaaugcuaguccaaguuaaaacuugggcaagaccuucaaacugacggggaacuccuaaagcuucagacaccaagccuuauuccgaaaggguaggguggccagguuaauagccucggguaugguaaaagaucugaagauauuacaauggacaauccgcagccaaggcccuaacgaaguguuucacuucuaugggacagguucagagacuagauggaggucggucucauguaaaugaggcuuaagguauaguccggcuucaagugaaaacuuguu

SEQ ID NO: 315 - RNA, 215 nt, Sclerotinia sclerotiorum
uuauauuuuuauuacuaaaaaaaaaagggaaaaaacagcaaauaaaaaaacuucuucuuacuaauugcuggaaacuccuguuuaauaggacaaucagcaggagccugcuguauauguuuauacaguaggguucuucagagacuacacguaagauauccuagaaucauuaaauaaauaggauaaagauauaguccgcucuuaauagaaauauuaag

SEQ ID NO: 316 - RNA, 564 nt, Gossypium arboreum
cuacggacuuaauuggauugagccuugguauggaaaccuacuaagugauaacuuucaaauucagagaaacccuggaaugaaaaaugggcaauccugagccaaauccuauuauuuuauuauuuuacgaaaauaaacaugaacaaagguucagcaagcgagaauaagaaaaaaaggaaaggauaggugcagagacucaauggaagcuauucuaacaaaugggguugacuguugguaaaggaauccuuauaucgaauaucgaaacucuagaaaggaugcaagauauaccuauuuuuuuuauagguauacuaaugaaaaacuaucucaaaaaagacguaccgaacccguauuuuuuuuuuuauuucuauuauaugcaauaucaauuuauauuuauaugaaaauaugaaaaauaaaaagaauuguugugaaucgauuccaaguugaagaaagaaucgaauagaauagucauuaaucaaaucauucacuccauagucugauaaaucuuuugaaaaacugauuaaucggacgagaauaaagauagagucccguucuacaugucaauaucaa

SEQ ID NO: 317 - RNA, 227 nt, Acanthamoeba polyphaga
auuccuuauugguuccuaaguauauaucgaaagguauauaugguaauaguuaaucacuauuagaggaaaaauaucaauaaggucauagucaauccgcagcaaagcuccuaaacccguuaugcuagggcauggagaauguucaacgacuaaacggaugugggcaugaaggaauuagcacuuccuaaugauugcuuaagauauagucuaaacccaccagugaugguguu

SEQ ID NO: 318 - RNA, 370 nt, Bacillus koreensis
uucgugacguagauuaugcuuuagcugcguaagcaguaacaagcacagucguccuagcugguaacggcuagagaucauaauugggugaauugcuggaaaccccuuagagcuuucuuccccacagcggaguuggaaacgacagacgcgauggguuuaagaagaagagagauugggcaaucagcagccgagcuccuguuccgaaaggauggagaagguucaacgacuaggauagaccaucuaaaagcuaaagcucaagaugaugaaauccauaggugaagcaguagaucaucacuacugugaauccgaagugcccaaccccuaccgaauacggagggugaagauauagucuagucauuuaugaaaguaaaug

SEQ ID NO: 319 - RNA, 282 nt, Corchorus olitorius
ccacggacuuaauuaaauuggauugagcuuuggcaugguaaccuacuaagugauaacuuucaaauucagagaaauccuggaaugaaaaaugggcaauccugagucaaauccuauuauuucacgaaaauaaacaaagguucagcaggggagacaucuuuaacagcugccaaugaaucuccaauauauuugguaauuuccuacuuauaguaguuaaagaagcugaauaacaagcauuuuaagguagaagagugcugaccugugagguuaguggaggucgugugg

SEQ ID NO: 320 - RNA, 214 nt, Omnitrophica WOR_2
agcgggcugugcucaagcgcagcuuccacaggaaacugugguugacaaagcaggagaauugcuggaagccccucugggguaaucagcagccgagcccgucauuauuuuggcgggaagguucagagacuauguaccugccucccgaaacgcaauguccgccuauggcggaaagucgugggagaagauauaguccaaguccgauaguaauaucggg

SEQ ID NO: 321 - RNA, 243 nt, Rhizopus oryzae
guucggagauuuguggaguucaccacggguagguaauaagcccccucauuauuagauggggauaaucucacuauaugcccgaaacuccuaaagcccaauuuacggaaaccgugauaauaauugggauaauacaauggacaaugggcaggaaacagaaaauuuauucuggcuccucagagacuacaugugaaacauucauuuuaaugaaugaagauauagucccauccaugacgagauucaugg

SEQ ID NO: 322 - RNA, 251 nt, Triticum urartu
auuugaauacaauaguauugagcccaaguaaaacuggaugaauugcagggaaaacuaaaaaugaauuuaguuaaucugcagcgaagcuauuaucggcaccuuauuaacuuaucaguuaauuuguagauaauagaacguucaacgacuaaucggugagcugugcuagcaauaauccggacacgagcguccagcagaaaauaauuaauauuuauuuucugaugaaauagucugaacaauggugugaaucauug

SEQ ID NO: 323 - RNA, 825 nt, Microbotryum lychnidis-dioicae
auuuuaggauucauuguuuggucuugguugggacucccucacagugauguggggguaagaaauuucgcuauuugcugaaacaguuuuacguuguuagguaucaagauauaguaaaauccuagcagcaauacucaaucagcagggagccgucacuuuauuaaugcggguucuucagagacuauacgcgagacaucuuggcauaacuuuacagccuucuauacccuuuuaaugguuauaucaacacuaugagucagcuuuuuucuaguuagccuuuuuuuuuggccugucguuuuugcucucuuuuuuagaaaaacagcccgcucauuauuuuugcuuuuauuuuucuuuuucuuuuuacucucuuuuucuaccggaggaguaaaaacagcagcgcuuauuuucacucgguagaaaauaaaggccaaauaagcgcccuuuuucuguuuuuauaaagcgccucgcccagccgagcugggcgcuucauucgcccagccgagcugcuuuucaucaaagaugaagcgcccagcucggcugggcgaggcgcuuaguuuuuauuuuugcuuuuuuuguccugcuuuuugauuaaucaaaaaagacaaaaacagcaaaaagcacaaaaaaggaaaaggcagcucggcugggcgaggaaaaagacaaaaaaauaaaaaaaaagggaaagagcauuuaacaccagacggauuacagcagauucgaucccuuuacaggcaaauuaaugucuguaacuaagaaucagcuaagacagaucaucucggccauaaagcgacaagaugaagauauaguccgaacuuuacucgcgagaguaag

SEQ ID NO: 324 - RNA, 269 nt, Candidatus Liberibacter
auaugugguuuauguuuguaaacuucauaauagguaaugaaaaaaaauuguggauguggcggaauagguagacgcagcagacuuaauguuauugggugcccauagagaaaucgauggaguagaacugcucaaauucggggaaagcuuuugcaaagcuaaucccgagccaaaucuuguuauucaagagagguguagagacuggacgggcagcaccuaaggcauuuaaaacguuauggugaagggacaguccagaccacaaacacugcaaa

SEQ ID NO: 325 - RNA, 324 nt, Tortispora caseinolytica
agcgggucguuuucugaaaggaaagcggcguugcugaaagcuagguucuaaaacguugggccagucgcgcugaaaggcgcggcuagucgugcauaugcacggcgacacugucaaauugcggcgacacccugagagcuucaaguaccaagcuagcgucgaaagacagcuaguggccgaguuaguaacccuggguacgguaaaaaccuugaagauugggcgagcacgcagccaaguccuacggcgcaagcuacggaugcaguucacagacuaaauggcagugggcgaaagcuuaagauauagucgggccucuggcgaaagccaggu

SEQ ID NO: 326 - RNA, 847 nt, Verticillium longisporum
  misc_feature (333)..(345): n is a, c, g, or u
  misc_feature (510)..(517): n is a, c, g, or u
  misc_feature (625)..(633): n is a, c, g, or u
aaaucggcgucauuugagacgaggacuuucgggcccgaaaggguguccaccaacgaggaccguagcacggcuuguguaccguagucuccucggaggcgacacccucaaauugcgggaaacuccuaaagcucacgcuccaaagccgucugugaaagcaguucgguggccagguuaauugccucggguauuggaacaacgcgugagaugcaacaauggacaauccgcagccaagccucuaagucucuugugacucugggugaacgugcuucacccaguuugcucaaggcgggaggacucacagaucgaaaccggagucacgaccucuggucaugnnnnnnnnnnnnncuccggugguucggcgucucgauucugcugaguccugguucgcgucccagagccaaacugccucuggcagcaccuagacggagacuuaagugccguagacggaggcuuaaguccccaacugccuaacaggcgguugguucugauucaggaccagccugagucacnnnnnnnnccagccugagucacgagagauauggggaagguucagagacuugacgggggugggugaauucacugcugcugcaacaauauaaauggggagagauccucuucuucuucnnnnnnnnnucuucuucuuccaacaaccaaaccaaaccacaacugaaccucaaacaagacccacaagcucuucaaaaugucccauucuucucccucuccuguucucgcuaacgggagcgaguaugucgugagggauggagguucgucuggucaggcauaaggaacgagaaugcaguggcgugguuugcuuaagauaaaguccgggcuuaugggaaaccauagg

SEQ ID NO: 327 - RNA, 365 nt, Rhizophagus irregularis
cuauaguuuuauaagcccugaagcuauagaugucuaucuggcuauaugcugggaaacccacuaacuuucuauuuaaguuaagaauauggugaaguggacaaucagcagguaacccuccuuagcaaaguagggaggcuaccucagagacuaaacgccagagccugcaguaugaauugcauucccucugggcuaaauuggaagggagucugggacacuaucuugccggguuaauagaaggagacggagcuauuauuguuucuucuaaaauaaccuuguucuuuuugauaaaaucccgguaaggugagucaaagcaugcuguucaaucugcagguaagauauaguccgauccaaauagugauguuugg

SEQ ID NO: 328 - RNA, 311 nt, Paludisphaera borealis
gcaggggacucaucaaccaaaaugguggcgccggagggcgaccuucggaugcgaaccgggugaauugcgggaaaccuaaaccucuguuuugaggcacggcgauccgcagccaagccuggccgggcuuugguggccaggaagguucagagacuagcggggugagucccaacgauaauccccgccucgagcgcccggccucccucgaaugcuucgaggcggucacgucaagcgguccgucaacgaccgccacgcaaccguuucgaucgucgcaggcgaggaugagauaguccaagccccguggaaacgcgggg

SEQ ID NO: 329 - RNA, 346 nt, Triticum aestivum
auucaucgauuagcugcuagauaauagcaugugacauuuuuagucgcuaagugguaacuuccaaauucagagaaacccuggaauuaaaaaagggcaauccugagccaaauccguguuuugagaaaacaagggguucucgaacuagaauacaaaggagaaggauagguguagagacucaauggaagcuguucuaacgaaucgaguuaauuacguuguguuguuaguggaauuccgaagugaguggcaucgugccuucuuuuagagcgggucacauccaauuucgauauggcucaccuuugaaucacuuguugguaauuauuccauagaaaugacuuauuaggauacg

SEQ ID NO: 330 - RNA, 322 nt, Exiguobacterium sibiricum
guaauuugauuucaccgggcgucugaucgaguaacugaucagagcaugacugggugaauugcuggaacuccuuagagccuugauguacgacaacguggcuggaaacggugagcgugaccguucugaaaaacgucaaggauuggacaaucagcagccaagcaccuguagggaaaccuuggugaagguucaacgacuaggauagacgaccuaauggagacuucugaugguuaugaaauccguacuccguaaacggcgggggaagcgcccagcuccuaguauaccuaggaugaagauauagucuuaucauuagcgaaaguuaaug

SEQ ID NO: 331 - RNA, 349 nt, Parasitella parasitica
auauuuggguaaacuauacacuugccccauauuaguuaauaacuaauaugcaaaucucacuauaugcuggaaacuccuuagagcuugcaauaccuagaucccuuuggguaucuuaccccaggcgcuacgcgcccggggcuagauggugacaauuuacaagauuggacaaucagcaggaaaccaaaggaauauuaauauuccaaguuucggcgggcccugcgggcccgccgucaccgaacccgcgcgcuuugcgcgcgggaaaaguaggaucuucagagacuacacgugagacauccuauaguauauuugacggaugaugauauaguccaaccuuuauugaaagaugaaa

SEQ ID NO: 332 - RNA, 306 nt, Spizellomyces punctatus
aagaccauguuaugcagugaucagcacgugcacuugcaaagaaaguaacauggauaggaucuucuggcucaacugcguguggcagagaucgucaaauuguucggggaagcccuuagagcucaagcuaccaaccauugguugaaagaccaguggggcccuuccuagggaugguaauaaugcuuugagauuggguaauccgcagccaagcuccuaaaacuugcuuagcaagucauggagaagguucaacgacuguaaggcguaccgcgcaagcggaauauacagucuagccccacgggaaacugugcc

SEQ ID NO: 333 - RNA, 302 nt, Lyngbya sp.
ggaaaaugguuaauauuagcccuuuauaucaguaaugauauaaaugcaccuccugaauugcugggaaacccuaaagcuguuuuaacgacaacauaacuagaaauagucagugugaacguuuaaaaauaaaacagaugaaacaaugggugaucagcagccgagauucuguuaaaugaaucagguucaacgacuauuccaaacggaaguacacucaagcgaguggaaguaggagguauccuguagucaaaucucuaaauuauuacaggauaaagauauagucuggucuuacaugaaaguguaag

SEQ ID NO: 334 - RNA, 288 nt, Sclerotinia sclerotiorum
guaagaggggaugcgaauagcauuccuuuagugaugagaucgcaacacugucaaauugcggggaguuccuaaagcucaggcuaccgccucaggugcugaaaagcccugaaggcaccaagguuagcaaccuuggguaugguaauaacgccuguagauacuacaauggaugauccgcagccaagcucuaacaaucuuuucacgauucacgagcgggguucaacgacuagacggcagugggccugcaaaacagguuuaagauauagucugcgccuagggaaaaaucccaag

SEQ ID NO: 335 - RNA, 305 nt, Marssonina brunnea
guuuguguuuuuaaauggugaauauugaauauuacaaucaacuccucgugauauaaauaaaagguaaugacauuagccccuucaaaucuuucuauaugcuggaaacucuuaaaggcuuaaguacuauauaaaauucauauuuaauuuuauaaguaaaaaucuuaaguauaucuagacaaucagcaggaaaccaacggauaauauagauuuauucuaguaggauccucagagacuacacgaaagagaugguauagcguaaagucuguaccauuaagacauaguccaauuuguuuguaauguaacau

SEQ ID NO: 336 - RNA, 213 nt, Parcubacteria (Yanofskybacteria)
ucgggcucauaaauaauugugaccuaccauaguaauguggcauggaaaaacucucuaaauugucuggaaaccccacucgccuaucagcgaagggcaaucagcagcgaaaccuuaagcucaucgaaagggacguucagagacuauaaugggagcacccguaaccguaacaaaaguugggugaugguauaguccgucacugcaaguaauugcaga

SEQ ID NO: 337 - RNA, 269 nt, Oscillatoria nigro-viridis
caaaagcccuagugacauagcagcucuauccgguaacggguacugaaaaaucgggugaauucaaggaaaccgcagcacuucggguggcgacaaucuugagccaagucuggcgaaaggcagcgguugcgaucgcaaguagccggaaaggugcagagacuagagaugaggagccuaaccaauaaaucucacagcgcccgacauccgacgacagaucgcacaaaugauuuguagggaugaugaaauaguccgcccccuucggaaacguuggg

SEQ ID NO: 338 - RNA, 292 nt, Pithovirus sibericum
ugacacgcauuugaucuugaauguguguugagcaagacccucaaauucagggaaaccccuaaagcuuuugaauaccaagcuuccagcgaaaguuggagguggccgcgaguaaaucucguaggguauggugaaaacgucaaaagauauccgggaaaccgguaaugggcaauccugagccaagcaaccgaaaugccguaugguagagguugaaggugcaacgacuugacgggggucggucagaaacgacaguuucaggcuuaagguaaagucuacuccuuagcgaaaguuaagg

SEQ ID NO: 339 - RNA, 221 nt, Diplodia seriata
agaagcauuuaacucaguugagcauauauucccacauaaugugcucauuaaaccaggcuguuugcugggaacucugccgcauuaaaccgguugacaaucagcaggaaccaagggguuuuuuaaaauccugauggguucuucagagacuauacgccuggcgcuuaauuauuaaagaaaaaaauuaaaugaugauauaguccuucuacuauugaaaaauugua

SEQ ID NO: 340 - RNA, 359 nt, Rhizoctonia solani
gcaccucgauaguaacaugucgaguuaaauuagaaauaauuuaugggaaauuggguuaauuucaagaaaaucuuuacacuccaaaauuuuuucuauauguuaagcuucaagcuuaacaaacccacuuacgguggguugccucuacuuuuucgggggugcccagcgaagcugggucacccgaugguuaaaaauuuuuggaguuaaagacaacuugaagcgaagcuaguucugacaauaagcuaauuauggaacuagaacguucaacgacuagugggugaguuuugucaacaauaaucccgccacgaaugcccaacaacuaaagucacagauagaaucggaauuugugauugaaacaauua

SEQ ID NO: 341 - RNA, 317 nt, Chaetomium thermophilum
agaaggaguuuucuauggucauccccauuaagggacuaacugacauuggccuaaacuguagugaaccuacgguuaaaaaccaucaaauugcgggaaaccccuaaaggaaucuuaaccaaguaaguaugguaacauaacuuauggcacagguaaugacucgugguaugguaaaaucaagauucauuauucaaugggcaauccgcagccaagugccaaauauaaaauauuugguaugcaguucaucgacuagacggugguugguauuauuaguuuuaauaaugcuuaagauauagucaacaccccccugaaagggugcg

SEQ ID NO: 342 - RNA, 216 nt, Limnohabitans sp.
gcagaggacucauauuucucaaaugugccuuacacguggaaacuguguaagggauggugucaaauucgaugaaaccuaaguguggcaacacauauggcaaugucgagcgaagcuuagugcgaaagcacuuugaacguguagagacuugacggcacccaccuaaguacagcgauguauauggugaaggcaaaguccagcgagugaugaaagucacac

SEQ ID NO: 343 - RNA, 334 nt, Staphylococcus sp.
uucaguguguagagaaaucugcacaucgugacaguacgacuguccaacaaagaaauugaauugcuugaaaacccuaaagccugcuugaccacaacguagagauaaucaaacucaagcgugaagguugcgaaacugcagaaaaaauaagcaggaugacauaagguuaaaaccuaaguguuuuuugcaaugggcaacuagcagccaagccuagaaauaggaagguucaacgacuauuccucuugagggaaguacacacaagcguguggaagugguuucgccguaauggauaaugccaacggaaaagauauagucugugcuuguaugaaaauacaag

SEQ ID NO: 344 - RNA, 383 nt, Parcubacteria (Uhrbacteria)
auagcgacauucuguauaaaucgucuuuucgucuaaaaauuguucaaucauaugauugaacucgaccgugcugucauaaaaucuggcuauaugcuggaacaucuggcaucucccaaccaucaggagaaugggagauuggagaaucucacacuacguaguaaacuacuuguaaaagauacgugaaaauguguugagugcagauaaccagcagggaagacuaagauaugacagcgucgauucauccaaaucuuguuuauacaaugaacaagucuggcaaguaucgacguuguaaacaucaucuaucuuagaacccucagagacuauacgccggacuccgauguccaucggagaagauauaguccgaaccgcauggcgacaugcag

SEQ ID NO: 345 - RNA, 262 nt, Leptolyngbya sp.
cugcggacuuagaaaacugagccuuaguggagaaaucugcuaaguggaagcucucaaacucagggaaaccuaagucuugguugguuacuugaccuucugagauauggcaauccugagccaagcccuucaaaaggcgaaaaauagaggguaaaguucauccuuuaucuuuucgauuucauccuuuugaagggaaggugcagaggcccgacgggagcuacccuaacgucaagucgaggguaaagggaggguccaauccucaaag

SEQ ID NO: 346 - RNA, 339 nt, Mortierella elongata
ucauauauucauaauauuaugaauguauauuaaugauuuaauuaggcauggccggguaauauaguaauauauuacuuucuuuucacuaucugcuggaacaccuuaagaguauuuaaaacuaguucugcaugcuuucuuuaaugaaaagcgguaggaaacagugacauuuaaauaauuaggcaaucagcaggaaaccaaagauaaaagggcuuaacuuuaagcauuaaacacuuuuauugaguaggauccucagagacuacacgugaaauacccuauuaagugauuauucuuaauuauuuaaggguaaagauauaguccaaccauuaacgaaaguuaaug

SEQ ID NO: 347 - RNA, 209 nt, Candidatus Gottesmanbacteria
acagaaggcucaacauaugggcuguccugaguuuaaucgaaggauagaauucggcuauaucggugaaacccuaagaccaacguccggguaauaccgaggaaagaucccgagcuugucgagggaaauccguagagacuauacgccgaaucccuccgaagcuuuugagcgaagggggaaagauauaguccgacacucucaguaaugggagg

SEQ ID NO: 348 - RNA, 258 nt, Enterococcus dispar
auauucgguuuguugaaaucccauauucaaugaccgauaaagaaauagaaaaagccauauuugaauuaacuaugccaaucaugagccaagccugucgggaaacugcaggaaggugcaacgacuagauaaauuaaccuaagcaaaagcagucauuuauuugauugcuuuuuuuguauggcgaaauauccacgagcgcuugauaccuuaacguuuaaggcgaagguaaugauauagucugaacuuauaggaaacuauaag

SEQ ID NO: 349 - RNA, 345 nt, Aureococcus anophagefferens
uuaguugcgugucuauugugcgcuagucgcaccguuccgcgaacugcacgggaacggcggcggcaacaucaucgaauugcugggaaaccucgauaggccggagcuacuaaaggcucggggaaacccgggucaaaucgagcuuagacgcucgaagugaaaaugcuucggauagaggcaaucagcagccaagcgccuaaagccgcguguauuacaguguauuacaguuggguauacauguauugcaagcggucacggugaagguccagagacuaaguggugaugggugucggcgcgguugaccgcgccgaugcuuaagauauaguccgccccucuugagagagagcu

SEQ ID NO: 350 - RNA, 272 nt, Tuber melanosporum
caaaaaguauagguaaacccucugcuaguuccuaaagggagcaaaaccaucaaauugcgggaacaucuuaaagcaauuuuuaaccaagcgagaacgguaacguauuucguggcgcagguaaugacucgcgguaagguaaaauaaaaauugauguacgaaaggaaauagaaaauccgcagccaaguucgaaauaaaauucgaaugcaguucaucgacuaaauguugguuggcgcaagcuuaaaauauagucagaccucaaucgaaagauuuag

SEQ ID NO: 351 - RNA, 203 nt, Candidatus Gracilibacteria
agauaugauuucugucaagggcuacuagagaaguaauuuucuagugaaaauccgucaaauucggggaaaccuucauuucuaguuauagaaauauggcaaucccgagccaagccuauuuauaggaagguguagagacuagauggcggacauccugauacucaggaugaagggauaguccagaccacgaacccgaaaggggcgau

SEQ ID NO: 352 - RNA, 207 nt, Synechocystis sp.
cuuggcaucucaucuugcaaaaaggggcugcgcaaaaggaaacuucugcgugauuaucucucaaauucggggaagccuuucaaaugguaaucccgagccaaaccuaggaaugcuugguguuucugggaagguguagagacuuaaugggagacacccuaacagaaaagcugagggugaagagaaaguccagaccacaaacugacagag

SEQ ID NO: 353 - RNA, 355 nt, Parcubacteria bacterium
auaaaugcguuuuauugugcgcgaauugucacagagaaguacaugcugguguaaucgcagccccgcguaggaaugcgcggagaaaaacugggugaauucggggaagcccagccccacuuuuugauaagaugccaccaguagauaaauaauuguguauuuauuugcugacguuaguacuagucauuaucgguuuugucagaaaguggggcuggguaaucccgagccaauaccugacggguaaaagugucaggaaagguguagagacuagcgggugagucccaacgauaaucccgccacgagcgcccagcgccuagaacaggcgaugagauaguccgucccuauugguaacaguagg

SEQ ID NO: 354 - RNA, 92 nt, Legionella feeleii
acggguuuacccccgaaucgagccuuguggcccuugccaagcaucauguauaugagcugcucgaauaacacuaaacacaauccuggguaaac

SEQ ID NO: 355 - RNA, 96 nt, Legionella shakespearei
cuugauuugccucaucauuucgagccuugcagcgcaagcuggauauccucuuugagugaaucgcucgauuaacacuaaaccaaagacagggcaaau

SEQ ID NO: 356 - RNA, 96 nt, Legionella waltersii
auugauuugccccccccguuggagccuuguggcguaagccugguaucgcuuuugagugagccgcucgaucaacacuaaaccaaagucagggcaagu

SEQ ID NO: 357 - RNA, 96 nt, Legionella jamestowniensis
acugauuugccccugaacugagccuugaggcacuacgccugguacugcaaccuugcaggccgcucuaccaacacuaaacaaaauaccagggcaaau

SEQ ID NO: 358 - RNA, 96 nt, Legionella fallonii
auugauuugcccccucuuugagcauuucggcuuuugccggguaucaauuuuuuggauugagccgcucgaccaacacuaaacacaaacagggcaaau

SEQ ID NO: 359 - RNA, 126 nt, Pteropus alecto
caagaaauguuucuugaccaguugccugcagcugaugagcuccaguaagagcgaaaccaguucucacuccacugaaacaauuuugaagugugaauugguccuguaguacugugucagaaacaacuc

SEQ ID NO: 360 - RNA, 126 nt, Pterocles gutturalis
agguuuaugauguuaaaccaguugccuacagcugaugagugccaggaagagcgaaaccaguucuguucuguuucaacaguuaugaaaaguaaggacugguccuguaguacuguccagcaucaaaau

SEQ ID NO: 361 - RNA, 206 nt, Anopheles dirus
uagaaaugcagcacugguacggguacggauccgacgccucucguaggauacuuaggcucuccguacccuacuccuacucaaaacguccccgacguacauauucguguuucuuaucccguuucucucgauuagugauagcguagugaucuguucacuggcaccgauagguaaaaauccuuucaaaauacuauacgaaacuaaaagac

SEQ ID NO: 362 - RNA, 150 nt, Anopheles minimus
uuguacuuaugcucugcaaugggguaggacccggaaccuuuugaagguuacacagguucuccuauucaacuccuuuucuacuacguauccaagcuuggauacaugggccaucuacauccccuggagugggcagaaacgaaacugggcuac

SEQ ID NO: 363 - RNA, 192 nt, Anopheles culicifacies
aguaaaauuucacugguaagggauggaucugaaaaccuaucgaaaaucaacaaaggcucuccauauucuacuccgacucaauagaaguccccgacguauagaacgguaaccugucucacuaaauaucugagcuuggguauauggagaaacccaacccuugggaagaugggcggcuagcuuccuuucuauccu

SEQ ID NO: 364 - RNA, 167 nt, Anopheles funestus
agcaauaccgcacugauauagauauggauucaaagucucuugaaggauaauauagguucuccgucccgacccuacuauacguccaugucguauauacauaucucuacaaauaucugagcuuggguauacgaggaaacccuggagacuagauguuccucaugcccugg

SEQ ID NO: 365 - RNA, 176 nt, Anopheles sinensis
uugaccauuuagucugaccaugggucugcaaaggaacuauaagcuauccucccccacuccuacucaaugcguccgcgaaguacagaacggcagcuugucgcuuaaauauccaagcuuggguacauggggaaacccacccccuugggcgaauggccggcaaggcugaauugagagga

SEQ ID NO: 366 - RNA, 162 nt, Anopheles atroparvus
gucuguguuggucugugaauggggcaggauccgacgccuccugaaggcuacauaggcucuccuaucuaacucauauucugguauguccaagccauacagaccguguacggguucaaucccaacccccugggaggaugggguugcacggcuaauguagaaggg

SEQ ID NO: 367 - RNA, 186 nt, Anopheles christyi
agcaauacuucgcugacacgggaauggauccgaagccuccagaaggcuaacauaggcucuccguugccuacuccuacuaaauauucacuacauuccuacagaacggcaacuuguuucucaauuauccaaacuugaugcaacaugcaaccccuugggaagauggaaggaauggcaaaauuaggcugg

SEQ ID NO: 368 - RNA, 181 nt, Anopheles dirus
uuuacauuaugaaacacaaaucuacaaucuucacgccugucgaaggaugcacaggcucuucuuacucuacuccuacucaaaacguucccgacuguaacaugucuauccgcauaucugagcuuggguauacgaggaaacgcaaccccuugggcgaauggaugauguggcuaauuugagugga

SEQ ID NO: 369 - RNA, 164 nt, Anopheles gambiae
aauaauguuuaaauucgaaacugacuuggaaaacccuguaauauagacucucucauccaacuucuauucuuagccgucauugguaacaugugucaccacauaugaguacuuagacuagauccaaacccuugggcggaugguggcauauggcgaaccaggagagg

SEQ ID NO: 370 - RNA, 201 nt, Anopheles arabiensis
uuaaguagcaaaugcaaucggauagguuucgaagccucucugagggauaauagaggcucuacuauucaacuucuaaucgaacacgacccuauucguguagagugguaacauguggauucggacuaguucgaagggucccaaagggaacacggacuaguuccaacuccucgcacagaugguggcauauggcgaaugaggcga

SEQ ID NO: 371 - RNA, 167 nt, Anopheles minimus
aucaaauuucugugaguuggugugguagaaauaccgcaggagaauacuccuacucaauacguccccggcguacagagugguaacaugucucuccaaauaucugagcuuggguauacgggaaaauccauccucuugggaggaugggugauauggcuaaauugagagga

SEQ ID NO: 372 - RNA, 208 nt, Anopheles melas
agcaccaaauuaucugcaaaugaguuaauauccgacaccuccuugaagguuaauauagacucucuuacucucuuacucuuucuccuauccugcgacguccguuucguauagugguaacauguaucauaguauauucaagcauggcugcacgggcccagucccaaccccuugggcggaugaugguacauacauggccaaccaggagggg

SEQ ID NO: 373 - RNA, 180 nt, Anopheles christyi
cucauuggcuggaucaggugaagcgggacuugucggagacauaggcucuccuauucaauucccauacggacacgucccaguucgugcagaguaguaacuuggaucaucgaauauccaagcuuggguacacgggcuuguuccaaccccuugggcggauggcucauggcaaaucaggagggc

SEQ ID NO: 374 - RNA, 140 nt, Anopheles maculatus
gucaguauaacacacuaguauugauauggguccgaagccuguccaaggauaauauaggcucuccauguacuccauauaucuuagauagagcauaagggaaaacucuuggagagauggguguuuuagcuaaauugagcggu

SEQ ID NO: 375 - RNA, 186 nt, Anopheles gambiae
auucuucugugcucugcaaugggauaggauccgaagcccuucugagggauaauauaggcucucuuauuuaacuccuacucggacaagucccuguucgugcagaguggcaacaugugucaucacauauucaagcuugagugcacggacuaguuccaaccccucgggaaacagagucguaauaaagga

SEQ ID NO: 376 - RNA, 148 nt, Anopheles sinensis
ugaaugaguuguucugcaaauggauugaaucacuugccccuaucccagggcaguaugaaggcguguucauuccuagauccuacucaacacguccacgucgugcagaaugguagcauuucauuacgaugaaaugacuaaguugagaggg

SEQ ID NO: 377 - RNA, 169 nt, Anopheles epiroticus
acuacuacuagcuucuugaaggguuuugaagccaggcucuucuacuucuacucuucuacaauuuguccuuaucguacagagcacuaacauauauucaaauaucugagcuuggcaaaacggcgaaacccaaucacaacuccauggaugaaagacauggaguuugagaggg

SEQ ID NO: 378 - RNA, 225 nt, Anopheles gambiae
cucuuugccucaaaaguuugccgaccgcugcucuagaagguuaacauaggcucuccacccccuacccuuacucaauacguucccgucguacggaugucccucaauuauucagauguccagauauccagauguacagauguccaaugucccucaauuauccaagcuuggguauaaggggaaaccuaaccccuugggcugauggauugcauggcuaaauuaagagga

SEQ ID NO: 379 - RNA, 175 nt, Anopheles christyi
agcaauaccccgcuagugcgggaauggauccaaagcuuccagaagaugaacauacgcucuccauuccauacuccuacucaauacguccucggcguacagaacaacaccauguaucucuauuauccaagcuuggguaaauggcgaaaaucauacaauuuggggagaugggaggcag

SEQ ID NO: 380 - RNA, 173 nt, Anopheles maculatus
aaaaacaaccaauugggccacgcaaaacgacuuuugcaacaugauacauuacaaauuggaacauaauaccugucguacauaauacugaaauaaacauaccaaauaucugagcuuggguauacggggaaacccaaccucuagggaaaaugggugauauggcuaaauuuaccgaa

SEQ ID NO: 381 - RNA, 188 nt, Anopheles melas
gguaguucuacuggcaaugguuggcagauucgaaaccucuagaagguuaacaaaggcucuccaucaccgacuucuacucaauacguccuugucguacagaaugguaacauguuucuuaauuauccaagcuuugguacacggggaaacccaaccccuuggacauugguugcauggcuaaauugagagga

SEQ ID NO: 382 - RNA, 121 nt, Anopheles stephensi
  misc_feature (105)..(121): n is a, c, g, or u
auuaaucuggcucuguuaaugggguaggaaccgaagcuccucucgggguuacacaggcucuccuacccaacuccuauuccgucacguccucgucguacagagugnnnnnnnnnnnnnnnnn

SEQ ID NO: 383 - RNA, 175 nt, Anopheles christyi
uacuucauauguauugcaauaagauaaguuccguagccccuuugagggauaauacaggcucuccaauucaacuccuauccgaaaacguccuaguucguacaaagauucgucaccgcuuuucuuguugaccuguucuaaccccuugggagguuggcgcaaggcuaaucaggagagu

SEQ ID NO: 384 - RNA, 167 nt, Anopheles minimus
ucuagcaaugguaagggaauggaucuggagccucucgaaggauaauaaagguucuauauaucauauuacuacucaacgguaacauguaucgccaaauacccugagcuugggaauaugaagaaauucaaccacucggcaggaugaggaauguugugaagcuuggaaga

SEQ ID NO: 385 - RNA, 222 nt, Anopheles stephensi
ccuaaaaguugcucuguuaaugaaauaggauccgagacuccuuucaggguuacacagggggguaggagagaguuucaggguaggagaguccuacccaacuccuauuccgucacguccucgucguacagagugguaacuuuucccaccauauaucuaagcuuggguacacggaccugucccaaccccuugggcggaugguggagaaaggcuaaacaggaggag

SEQ ID NO: 386 - RNA, 158 nt, Anopheles dirus
uagaaaugcagcacugguaaggguacggauccgacgccucucgaaggauaccuaggcucuccguacccuacuccuacucaaaacguccccgauuuuccuccugucuaaccuaagacgcguuccgcgagagaucuuagcuuaugguuaguuugguuggu

SEQ ID NO: 387 - RNA, 189 nt, Anopheles minimus
uucacauaggcucugcaauaagguagaacucggaaccuauugaaggucacacaggcucuccuacucaacucccauucugcaacguccucgucgugcagagugacgagaacuaccguauauccaauauugaguacacggacuagcuccaacccuuugaucggguagugaugcucggcaaaagaggagggc

SEQ ID NO: 388 - RNA, 193 nt, Anopheles gambiae
cccaguguuuuuccuuuuuccuguuuuuccagaaaccccucgagggauaguaccggcucuuccauccaacuccuauuccgacacguccucgucgugcagagugguagcaugugccaccauauauccuagguuggauacacgggcuagauccaacccuucgggcggaugguggcauaugacgaaccaggagggg

SEQ ID NO: 389 - RNA, 167 nt, Anopheles sinensis
ucuaguaguaguccuuuaacggguuggauccggcgccucgugaaggcuacuauaggcucuccuucucaaaaucuauucgaugcguccgcgacguacagaacgguaccuugucgcccaaauauccaucaguucauccucgagcggcuguccuccauguuaggagcggc

SEQ ID NO: 390 - RNA, 189 nt, Anopheles gambiae
agcaauacgucgcugauauggcaguggagccgaagccucuagaagguuaacaaaggcucaccauccuuuacucauauuccauacguccucaauguacagaucgguaacaugccucucaauuaucuaagcuuagcuauacgagguucuucaugacuguucuccauauuaggagcggcucccuccaagcgu

SEQ ID NO: 391 - RNA, 117 nt, Anopheles arabiensis
cucgacuugccguucgaacaccaugaucaggccacauauaggcuaauuuucuaugcuuagauauacggggaaacccaagcccuugggcggauggguuguauggcuaaauugaguauu

SEQ ID NO: 392 - RNA, 155 nt, Anopheles sinensis
aguaucguuaaaaccacauauuguccuuugcaaaccuuccguaccaaaauuacggagaguacagaacgguagccuguugccuaaauauucaagcuuggguacacggggaaacccacccccuugggcgaaugggcgacaaggccgaauugagacgg

SEQ ID NO: 393 - RNA, 136 nt, Anopheles merus
cugagacgaggauagauccaaagguagauccgaagaagguuaauaaaggaucuccaaccccuacaccaacuaaauauguccccguaguacggcccgguaacaugugguaacccucucaguggauguacagacccga

SEQ ID NO: 394 - RNA, 119 nt, Anopheles epiroticus
ucaaucaugugaaacuguuaugggaugggauccgaagccuccagaagauuccaucuaacuccuguuccgacacguccacgucgugcaggguggggcaacauggaccuaaccaggggguc

SEQ ID NO: 395 - RNA, 208 nt, Anopheles darlingi
gcaauugguugcucuacaaaugagguuaggauccgaagcacuaggcuaauuuaggcucuccuccccuaacuccuacucgucgcguccucgcggggcucauggguucugcucguguagagcgguaacaugccucccacacgucugagcuuggguacacggguaacaccaaccccuugggaagaugggggguauggcugagacgagaggg

SEQ ID NO: 396 - RNA, 172 nt, Anopheles stephensi
cguagcaauaaugcacuaguaagguauggaucugaagcuucuugaggguuaacaaaaccucuccauacgcuacuucgacucaauacgucguacgggcguacagaacucuccauacauaugaacuugggucauacggggaaacccaaccccuugggaagagagauagcgagcg

SEQ ID NO: 397 - RNA, 193 nt, Anopheles gambiae
aacauuuuuagaucgucagcauauaauaacaauuuaacaugaggaauagcaaacacgacaucguuaauaaauauaauaaauaguaaagguccuaaucaccacauugauaugugucaccacauauccaagcuuggguacacgggcuugacccaaccccuugggcggaugguggcacauggcgaaccaggagggg

SEQ ID NO: 398 - RNA, 153 nt, Anopheles dirus
ugcaacaaccauuucguacaugagggaaugaaaacaagaagagguggggugacaaacuuaaagaauuuuaacacuauaguagauaucugagcuuggguauacggggaaacccaaccccuugggaggauggaagacauggcuaauuugagagga

SEQ ID NO: 399 - RNA, 110 nt, Anopheles culicifacies
aucaaucguugcucuguaaguaggugugguacuguuaagauaagcucuccauaccguacuccuacucaauacguccucggcguacagagcgguaacaugucucuccaaau

SEQ ID NO: 400 - RNA, 201 nt, Anopheles dirus
cguaucuuucuacuauauaaugggauaagauccaauaccuuuugcaagcuaacgcaugcucuauuauucucuucugucgcgacacguccccaacguacagcuugguagcauauaugauuuauuuuccaagcuuggguaguccggacaaauggcaaucucaacguuggagaagagcuagacgauauacucaacuacuucaag

SEQ ID NO: 401 - RNA, 129 nt, Anopheles culicifacies
aaauggauccgaagccucucgaaggauaauugagggcuacuccuacuuacaugucucuacaaauaucugagcucggguauucagggaaauccaacccuuugggaguaugauacggcugaauugagagga

SEQ ID NO: 402 - RNA, 129 nt, Anopheles stephensi
gcuaguucugcagcuguugugguagcauuacugaagccucuugcagguuaacaaaggcucuccauacccuaccucgacucaauacguuccccugucguacaaaacgguagcaugucucaacggaaauga

SEQ ID NO: 403 - RNA, 190 nt, Anopheles minimus
accauaauauccugcuaacgggguagaauaugaagucgcuuaaagguuacacaggcucuccuacucaacccuuauucccccuagcauccucgucgugcagagcggcaacuugaaccaucgcauauccaagcuuggguacgcggguuaguuccccuugggcggauggugguguaugauuaaacaggaggga

SEQ ID NO: 404 - RNA, 183 nt, Anopheles merus
uucagaucaucugcucagcaauggaacaggauccgaaguuucccugaaugauaacauagguuauccgaucuaacucguauccagacaagugucaguucguuacaugugucaucacauauccaagcuuauguacacggaccuguugcaaccguucgggcggauuguugcagucauuugauugca

SEQ ID NO: 405 - RNA, 127 nt, Clostridium sp.
aacuaaguugacgaggaugagauuuaucgaauuuuuucggcggauaucucacguaaauagcacuagcguuaauaauuaacaaaacuacaaaguaauuuguaggacaaauuuaauuaugugcaaucua

SEQ ID NO: 406 - RNA, 159 nt, [Clostridium] neopropionicum
uugaaaaaugaagcgccagaaccgcagauagggcgguugacgagguagaagugaucgaauuuuucggcggaugcuucucgcccauucguucagggacgcaggucuuucuacaaauaggagaagguaauucuucugacaaagggaaaggcaacggacgaa

SEQ ID NO: 407 - RNA, 154 nt, Clostridium sp.
uaacauuaaauagcgcaugaacauaccuuucguaugugacgaggaugauaguuaucgaacauucagcggauacuaucacggugauuuagcaucgauauauauaguacaaaaaguaaaaucuuuaauuacuacaaaaacuauauaaucuaaauau

SEQ ID NO: 408 - RNA, 178 nt, Atribacteria bacterium
aauugaauacaagcgccagaacuuacucaauuugaagggcuaaguguuuugauaagguaaguugacgaggaaggaguuuaucgaaaauucggcggaugcuccuggguuggccagacccuuagaaaaccuguaaaacuugugaguaauugcaaggacagagagguuuuuauggcaaaau

SEQ ID NO: 409 - RNA, 150 nt, Clostridium sp.
uuauuuaaguaagcgccaggacauuuuuuuaauguugacgaggauagaacuuaucgaaauuuucggcggaugguucuagggaaugcuacuuccuaaaauauugucaaaaaauaauagcgauauuauaacaaaucaauauuacuagcugug

SEQ ID NO: 410 - RNA, 171 nt, Kurthia sp.
aaaacaguuauagcgccaguacugaagaaaucggacgacaaguaucuucaguagacgagguggaggaguaucgaaaguuucggcggaugccucccggucgacagcccgaucguaaguucaucuuuaaaauagugaagugauucauuagacaaagagaugaaauggcaaauc

SEQ ID NO: 411 - RNA, 148 nt, Mycobacterium abscessus
caauaucggauagcgccagaccugaacguucaggugacgaggagagagcuuaucgaagauucggcggguggcucuagggacugcacucuacagauaacaaagaaaaacuaauugugaaguuagaacaaagcgguuaucacgcagguag

SEQ ID NO: 412 - RNA, 189 nt, Shuttleworthia satelles
ucaaaaacuuuagcgccauguacccggccgcuuuucaucccucuguauuuuuggaggaacguuuuggcccggguugacgaggugucaggugaucgaagauucggcggaugccugaucgcggaugcggccgcgcauacaguugacaaaagauccgguaacggagagacaaagagacuguaaccguaugga

SEQ ID NO: 413 - RNA, 324 nt, Clostridium beijerinckii
aaucaauaaaaagcgccaggacuaaguggaauuuaguugaaagugaauuuuuacaggguugguuuuuauaaaaggaauuuaguuaauaguaagcuuugauauaagcuacgcgaauagcuaaugaaaucuuuuuaaaggaaacucgacucacguucguucgcugaguaaauucaacuauccaaaucgaagauuuggaguuucauuuacgucgacgagguuggggaguaucgaaacuucggcgggugccccacgguaucgcacuaccguaaacgacugguaaaacugugaagugauucacaggacaaauucagucugguguuaaaa

SEQ ID NO: 414 - RNA, 147 nt, Bacillus sp.
gaauucuuucuagcgccagaacuuugaauaguugacgaggauaguagugaucgaaaauucggcggaugcuacuacguagggaagaugcguuagguaugucuaaaaagcaaaaucgacauuguaacaacagauguaucacuuuucaga

SEQ ID NO: 415 - RNA, 155 nt, Desulfotomaculum reducens
uaaauaacugaagcgccagaaccuuuuacaaaguaaggguugacgaggagagggaguaucgauguuucggcggaugcccuccggcccaguugcggccguaaaagcagaacaaagcuggaagguaacuuucgguacaaaaacugcgggugacuaaa

SEQ ID NO: 416 - RNA, 148 nt, Mahella australiensis
uucgauauaaaagcgccggaacucauuuugaguugacgaggucaggguuuaucgauuuuucggcgggugcccugcggcauacggcugccgacaaagguuccacaaaagcaaaaagcgauuuuugcuacaaacgggacugggccaaaau

SEQ ID NO: 417 - RNA, 202 nt, Clostridium sp.
aguaaagcuauagcgccaugcaccugggucgggucgugaacaguaacggaugaaugaauaauaacuuacugcugacacagguuaacgagguggagagagaaucgaacauauucggcgggugcucucccaugcaguccgggcguguuuuauaccagaaaaauaugcggguaacugcaaaacaaagcugguauaaccggacaga

SEQ ID NO: 418 - RNA, 173 nt, Bacillus megaterium
agugaauacaaagcgccugaacuaaguaaagggacggaaacgacuuaguugacgaggaggagguuuaucgaaguaucggcggaugccucccgguuguugauuaucacggccgaaaacuugaugugaaaaacaaugaggugacuuauuggacaaaagcauugagaugauaauca

SEQ ID NO: 419 - RNA, 197 nt, Caldicellulosiruptor saccharolyticus
gguuuuuucaaagcgccaggaccucuggaaauuauagcgcuauugccuuugcgcauuuuuuccagagguugacgaggacuggggagaaucgagguuuucggcgggugccccagcgggguuuugccuuuuucccucgcaacuuucugcuacaaaccccggaaggcaacuucugggacaaaggcagaaagaaaaaaggc

SEQ ID NO: 420 - RNA, 159 nt, [Clostridium] cellulosi
uuaaauauuaaagcgccaggaccgauauaaaucgguugacgagguggggaguuaucgaaagauucggcgggugcuccuccggccguuaguguugcggucguuagcucaugcuacaaaaaacgcggguaaccgcgcaaaaaaaggcugagcauucagcgc

SEQ ID NO: 421 - RNA, 152 nt, Clostridium sp.
aguucauuuuuagcgccaggacagcguuugugcuguugacgagguuagaguuuaucgaaauauucggcggaugcucuagggcuuucuacgguccuuauaaauuagcaaaaccuagcagugauguuagaacagauauaauuuugaguaguuua

SEQ ID NO: 422 - RNA, 157 nt, Bacillus cereus
agagugauuaaagcgccagaacuacaaauuguguaguugacgaggaggaguuuuaucgagauuucggcggaugacucccgguuauucaucauaaccgcaagcuuuuauuuaaaucacugaggcgacuugguggacaaagauaaaagugugaugagag

SEQ ID NO: 423 - RNA, 209 nt, Staphylococcus aureus
aggaaacuuauagcgccugaacaaagcgcauacacgauuguagaggcauguauaaucagauacaugcugaaugaguguuaugaccuuuguugacgaggaggauaguuaucgaauuuucggcggaugcuaucccggauguggcccauucgaaguucaauguuuaaagcauauaggugacuguauguccaaagacguugaaauagccauaa

SEQ ID NO: 424 - RNA, 156 nt, Clostridium grantii
auauuauaguuagcgccagaaguaguggguaaagugcuacuugacgaggauggggaguaucgaaauuucggcggaugccccacgguauaacacuaucgauaaacauuggcaaagcaaagaagugauucuuuguacaaauucaauggagugugaauc

SEQ ID NO: 425 - RNA, 153 nt, Clostridium bartlettii
aguguuuauaaagcgccagaacuuaauuuuuuaaguugacgaggucagaguuaaucgaaauaucggcggaugcucugcggugugccaccaucgaaggauuucuacaaagggugaaagcaauuucacuacacaaaaagaaacccauguggauug

SEQ ID NO: 426 - RNA, 154 nt, Clostridium sp.
acacguucaaaagcgccgggacugcgugugcaguugacgaggcggaggaugaucgaaccauucggcgggugccuccuuuucgcgucgcgcggaaaagagccgaugcucaaaacccguucgcaaggacggcgacaaaagcacggcacgcgacaca

SEQ ID NO: 427 - RNA, 178 nt, Deinococcus radiodurans
cuucuucgggcagcgcaaggccccggcgacacgugaugucacaagccggggagacgagguggaggucagcgacuuuucugcggaugccuccaggccccggugaacgggccuacccggcgcgugcuuugccgcucugagucaaagacuccggcaggcagaaccacgcgcaagcccggcg

SEQ ID NO: 428 - RNA, 167 nt, Fictibacillus macauensis
gcaauaccacaagcgccugaacuauuuucgaccggaaugaaaauaguugacgaggaagaggaucaucgagauuucggcggaugccucucggaugacgucacauccguaagcuuuuauacaaaccagguaaggugacuuccuguacaaacauaaaaguagugacgaau

SEQ ID NO: 429 - RNA, 153 nt, Clostridium methylpentosum
ggguacaaccaagcgccaggcccggauacagcuccgggugacgagguggagagugaucgaacagaaucggcggaugcucucccggucccggcaggaggaucgucagaugguucacaaaagcgcguugcgcacaaaagagccaccccaucccgc

SEQ ID NO: 430 - RNA, 158 nt, Clostridium sp.
guuauuuuucaagcgccaggaccacuauuuucaggagguugacgaggucuagaguuaucgaaauauucggcggaugcucuaguggcuuuuacuuaaggucaucaguauuaauuaaaaacuaauaguaauauuagaacagaaguuaauauuuuaaguuu

SEQ ID NO: 431 - RNA, 159 nt, Sporobacter termitidis
uaaaauuuagaagcgccagacuucacgcagagcgcggaguugacgagggcgggguuuaucgaaguauucggcgggugccccgugcugcgguccaucagcauuacagguuuuacaaauccucaagcaauugacgggacaaaauaaaccugauugggccuu

SEQ ID NO: 432 - RNA, 165 nt, Listeria grayi
aauuuaauagaagcgccagaacugaucgaacgggguaucaguugacgaggaggagauuaaucgaguuuuuucggcgggagucucccgguuauucauguagccguuaugucugaguuacaaaacaagcaggcgacuguuuggacagaaagcuuagacgcaugaguu

SEQ ID NO: 433 - RNA, 157 nt, Oscillibacter sp.
cggcccaucacagcgccagcgcccgccgccagcggcgggacgacgaggggaagggaguaucgaaggauucggcggaugcccuuccgugcccacgggcgcguacacagccggcaaauaugcgggcaaccgcaaaacaagggggcugugaccgugaaga

SEQ ID NO: 434 - RNA, 155 nt, Thermoanaerobacterium saccharolyticum
aacugaauaaaagcgccugggcuuaggggaaacucuaaguugacgaggacaggguuaaucgaguuaucggcggaugcccugcggcuuccugcggccgauagagaaccgggaaaaccaugggugaccauuggcauagagcgguuugagcagggaua

SEQ ID NO: 435 - RNA, 171 nt, Gracilibacillus halophilus
uauuaauggaaagcgccagggcuauggugaacgaagaauccauagcugacgagguggagguuuuaucgaguuugaucggcggaugccucccgguuguugcaucucaaccgucacauuuuuauucgaaaacaugaagguaacuuuaugaacaagaauaaaaauaagaugccu

SEQ ID NO: 436 - RNA, 152 nt, Salinicoccus alkaliphilus
aaaaucggaauagcgcccgggcagcuuugcugcugacgaggaggacggucaucgaucaucggcggauaccgucccggacacugaaguguucguuacaccggauguuaaaacccgcaagcgauugcggggacagagcaccggucgauaauugu

SEQ ID NO: 437 - RNA, 168 nt, Butyricicoccus pullicaecorum
ugcauaucgauagcgccagaacugcggagcagcccguuucgcaguggacgagguaaggguugaucgaaaauucggcggaugacccuucggccgcgagugagccggucguuagcaggacguguaaaccccgggagugaucccggaaacagagcgucuucgcaugaccca

SEQ ID NO: 438 - RNA, 198 nt, Firmicutes bacterium
uccugcgcuuuugcgccuugucuugacgaaaaaucucucgccggcaggccggcggauauuuuucuuuuccgccgccgugggauaacgagguggggagcgaucgaaaauucggcggaugcuccuccgcaucguccggaugcgcacacagcucauaaauaugcgggagaccgcaaaacagaggggcugugaccggaugcg

SEQ ID NO: 439 - RNA, 168 nt, Microgenomates bacterium
cauuguuauauagcguguggcccccaucugcauuggcagcggggauacgaggagagggcuagcgaauaaucugcggguacccucaggccgaagccaagcugucgaccuuaagugugcucuuaaauccuucuggcaacagaagggacacgggagcuugcauacagcaua

SEQ ID NO: 440 - RNA, 174 nt, Pyramidobacter piscolens
gaacgaacggaagcgccaggacugaaccgcuucucacuccgcggcgacagucgacgaggacgaaagugaucgaaccauucggcggaugcuuucguggcgggcgaaggggagccaugaaccuccgucacaaaaccccggggcgacccgggaacaaacggcggaggcccuucaaug

SEQ ID NO: 441 - RNA, 172 nt, Bacillus sp.
uagauauacaaagcgccugaacuaagcgacggacgaaaccaugcuuaguugacgaggaggagguuuaucgaucuaucggcggaugccucccgguugccaaucacaaccgacuuuagggacaguuuaaagcauaaaggcaacuuuauguacaaagacuguuacuaugauugca

SEQ ID NO: 442 - RNA, 147 nt, Mycoplasma sp.
auuuuuguaauagcgcauggccuauuuaggugacgaggacuuacauugucgauuuaucagcggauguguaagcggagaaguuuuacuucguaaaauaccuuaaaaaaauaaaaucaaaauuauaacaaagagguauuuaauaaaaca

SEQ ID NO: 443 - RNA, 297 nt, Clostridium novyi
aauaauauuaaagcgccagaacuuaaguugaguaaaugagggaauaaaggaaacucaaacuagacguuugaguaaguucgacuaacuaaaucauagauuuaagaaguuaaggaaacucaaacuaaauguuugaguaaguucgccuuaaccaaaucguagauuuggaggcucacuuaaguugacgaggauggggaguaucgagaauucggcggaugucccacgguauuuauguacuaccgauaacaguuagcaaaucuaaaaagcgauuuuuagcacaaaucuaacuagguacaugau

SEQ ID NO: 444 - RNA, 228 nt, Paenibacillus antarcticus
uguauauuaaaagcgccagaacuugacuagcgcgggauguaagauggaccgaggguauggaugaguacgauccguuucggugccugauuacaacggcagaucaaguugacgagguggggguguucgaaauguucggcggggaccucccggugaugcaccagagccgugaaucauauacggaaaauaagcgggcgacugcucauacaacguagugaugggugcuaauua

SEQ ID NO: 445 - RNA, 224 nt, Candidatus Gottesmanbacteria
cagagaacauaagcguguggcccggcuaucauguuccucgccgagcucgucacaccagccgggcaagcccgguugaugcgagcgaagcgagcauaauaaccgggauacgaggaaagggcuuaucggauaaaucugcgggugcccuuugguaguacucucuaccacaacgguaaucagaaacuccaccgguaacggugggaacaaaugauuaucggcagagucgc

SEQ ID NO: 446 - RNA, 144 nt, Anaerococcus lactolyticus
aaaaacaacaaagcgccagaucccuuugggaugacgagggaggaaguuaucgaaaauucggcgggagcuuccuggguucacagccuuaguguuagacaaaagcagagagcaauuucugcgacagagaagacacaguggccucau

SEQ ID NO: 447 - RNA, 149 nt, Enterococcus sp.
aauauaggaauagcgccaggccuguuguuucaggugacgaggagagagcuuaucgaaacauucggcggauagcucuaggggcugcacucuacaagcuggaaauaaaaaauaauugcaaaauuauaacaaagauucagcuaagcagaaua

SEQ ID NO: 448 - RNA, 328 nt, Clostridium amylolyticum
uacaauauaaaagcgccaggacuagaguagagccaaauuaaaauuuggaacauaugauuaacuuaccaugacguauggagaaguuggagcauaugauaaaaggaaacuucuucuccgaugcuaaagcuuaaaaaauaagaaauaugaagcuuuagcaucuacgaagaagaaagcgaccgcgccaaauuaaaauuuagagccucugcuuuaguugacgaggauggggaguaucgagucuucggcgggugccccacgguagcgcacuaccguuaacgauugauaaagccgggaagugauuucuggaaacaacaucaaucagguguuaaaa

SEQ ID NO: 449 - RNA, 84 nt, Corchorus olitorius
gcaauaacuccgccuguggcugcacuaaauaccuaccucugcaaccacagccggucccaagcccggaaaaaggaggaggguugc

SEQ ID NO: 450 - RNA, 92 nt, Gossypium hirsutum
augauaacuccgccugugccauauugaaaccugagaguauauauccuuuucggcacugccggucccaagcccggauaaaggaggagggucau

SEQ ID NO: 451 - RNA, 74 nt, Astyanax mexicanus
uuaguaacucugccaaagugucucuuuuaaggucacuuugccggucccaagcccggauaaaagaggaggguuaa

SEQ ID NO: 452 - RNA, 81 nt, Cephalotus follicularis
gucauaacuccgccuguguagcaauauguauaaccauguguacacagccggucccaagcccggaugaaggaggagggugac

SEQ ID NO: 453 - RNA, 80 nt, Macleaya cordata
uuuuuaacuccgccaaugcaaauguuaugccauaacauuugcauugccggucccaagcccggguaaaggaggagggggaa

SEQ ID NO: 454 - RNA, 63 nt, Macleaya cordata
uuuuuaacuccgccaaugcauaugcauugccggucucaagcccggguaaaggaggagggagaa

SEQ ID NO: 455 - RNA, 57 nt, Unknown
  Unknown Organism
gcacuaauguagcucagaccugugacaagccaaggcuagaaaaauacagagucgugc

SEQ ID NO: 456 - RNA, 56 nt, Unknown
  Unknown Organism
ggccuaaugcagcauaguccugucacaagccaggcugaaaaaugcagagugaggca

SEQ ID NO: 457 - RNA, 85 nt, Unknown
  Unknown Organism
gaaacccgcuaggccgacagccucaccgcugccgcuggugcaagcccagccgccccagaccggggcgggcgcucauggguaacag

SEQ ID NO: 458 - RNA, 82 nt, Pygoscelis adeliae
uuaggccguuaccuacagcugaugagcuccaagaagagcgaaaccuuuuaagauagguccuguaguauuggccuaacaaucu

SEQ ID NO: 459 - RNA, 77 nt, Acanthisitta chloris
auuggcuguuagcugcugcucuugagcuccagaaagagcaacacugcuuagguccugcaguacuggccuacaggugu

SEQ ID NO: 460 - RNA, 109 nt, Chelonia mydas
uuaaccaguuaccuacagcugaugagcuccaggaagagcgaaaccaguuccgcccuguuucaguaguuaugaaaaguucggacugguccuguaguacuguccugcagca

SEQ ID NO: 461 - RNA, 82 nt, Struthio camelus
uuaggcuguuaccuauagcugaugagcuucaaaaugaaugaaaccacuuaaaauagguccuguagaacuauccuuagggcca

SEQ ID NO: 462 - RNA, 77 nt, Manacus vitellinus
ucaggccauugccuauaggugaugagcuccaugaagagugaaaccaguuagguccuguauugcuagccuaaugagca

SEQ ID NO: 463 - RNA, 83 nt, Chelonia mydas
uugggcuauagccuacagcuaaugagcuucaaaaaggagcaaaagcauuuaaaauaggcccuguaguauuagccuaaucuaau

SEQ ID NO: 464 - RNA, 78 nt, Chelonia mydas
uuaagacauugccuacagcugacaagcuccacaaagagcaaaaccagugaggauccuguaacacuguccuacagagcu

SEQ ID NO: 465 - RNA, 86 nt, Latimeria chalumnae
acguuccaguuaucuacagcugaugagcucaaaggagagcgaaaccggacauccuguccggucuuguaguacuggcuuaguugucu

SEQ ID NO: 466 - RNA, 77 nt, Picoides pubescens
cugcaccguuaccugcagccgaugagcuccaaaaagagcaaaaccagccagguccugcaguacuggcugcagccuuc

SEQ ID NO: 467 - RNA, 77 nt, Anas platyrhynchos
aaugucuguuacuugaagcugaugagcuccaaauagagcaaaaccauuuagguccuauaguacuggccuguaagcuu

SEQ ID NO: 468 - RNA, 111 nt, Apaloderma vittatum
uuaaaccaguuaccuacagcugaugagcuccggaaagagcaaaaccaguucuguucuauuucagcaguuaugaaugcuaagaacugguccuguaguaauguugaacaucga

SEQ ID NO: 469 - RNA, 77 nt, Pterocles gutturalis
uuaggccauuaucugcagcugaugagcuccaagaagagcaaaauccguuagguccuacaaugcuggccuaauacaca

SEQ ID NO: 470 - RNA, 77 nt, Gallus gallus
cuuggccauuaccugcagcuggugagcuccaaaaagagcggugccauuuaggucaugucauucuggccuauauguuu

SEQ ID NO: 471 - RNA, 77 nt, Ficedula albicollis
auugaauguuacuugcagcugaugggcuccaaaaagagagaagccccuuagguccuguaguacuggacuugaaacau

SEQ ID NO: 472 - RNA, 77 nt, Colius striatus
auuggcuguuacaugcagcuagugagcuccaaaaagagugaaaccacuuagcuccuguaauucuggcuuauaagugu

SEQ ID NO: 473 - RNA, 77 nt, Calypte anna
auuggccauuaucugcagcugaugagcuccaaaaagagcaggaccacuuggauccuuuaguacugcccuauaagugu

SEQ ID NO: 474 - RNA, 85 nt, Latimeria chalumnae
aauuucaguuaucugcagcugaugagcuuaaauaaaagcaaaacuggaauuaauuuccaguccugcaguacugguuaauaugccu

SEQ ID NO: 475 - RNA, 78 nt, Merops nubicus
auuguccacuaccugcagcugaugaaccucaaaaaagagcaaaacucccuagguccuguaguaguggccaguaagugu

SEQ ID NO: 476 - RNA, 80 nt, Latimeria chalumnae
caauccaguuaucuacagcugacgagcucuaaugagagcgaaacuggggaaccgguccugugguacuggcaugaaagaaa

SEQ ID NO: 477 - RNA, 77 nt, Egretta garzetta
auuggccacuacccgcagccggugagcucuaaaaagagugaaaccacuugggucuugugguauuggccgauaagcgu

SEQ ID NO: 478 - RNA, 83 nt, Alligator mississippiensis
gcaggccauuacuugcagcuaaugaguuccacagagaaugaaaccauuugaaauuggucccugaaguacuggccuagaaaacu

SEQ ID NO: 479 - RNA, 77 nt, Echinops telfairi
ccgagccguugccugcagcugaugagcuccaacaagagcgaaaccgaacagguccugcaguacggguggggucagca

SEQ ID NO: 480 - RNA, 52 nt, Chicory yellow mottle virus
caacagcgaagcgcgccagggaaacacaccaugugugguauauuaucuggca

SEQ ID NO: 481 - RNA, 52 nt, Arabis mosaic virus
caacagcgaagcggaacggcgaaacacaccuugugugguauauuacccguug

SEQ ID NO: 482 - RNA, 50 nt, Tobacco ringspot virus
aaacagagaagucaaccagagaaacacacguugugguauauuaccuggua

SEQ ID NO: 483 - RNA, 70 nt, Cyphomyrmex costatus
gauucucaacaaucgucuaccuccccguggugagaaucgggaaacauuucaaauaauggcuaaagacgau

SEQ ID NO: 484 - RNA, 73 nt, Crassostrea gigas
ccugcucaaaauccuacuuccaccuccccgcggcgagcagggggcaacggacauuuguccggcgaacggaaga

SEQ ID NO: 485 - RNA, 74 nt, Clostridium saccharolyticum-like
gcugcucgaaacuuugcacaccucuucguggugagcagcaggcaacgaucuuauggucggcuaagaaugcagag

SEQ ID NO: 486 - RNA, 75 nt, Rhizophagus irregularis
ccugaacgaagcuugccaccucuacgugguguucaggagaaacaguuguaaguuaauaacuggccaagagcaagc

SEQ ID NO: 487 - RNA, 74 nt, Camponotus floridanus
gauuuuccauuauauguuuaccuccacgcggugaaaaucgggcaacgucaaauaaauuggcggcaaaagaacgu

SEQ ID NO: 488 - RNA, 72 nt, Tulasnella calospora
ggggcucgaugaugcgcacaccuccccguggugagcccugucaacgucggcaaggacggccaagaugcgcau

SEQ ID NO: 489 - RNA, 72 nt, Trichomalopsis sarcophagae
gauguuuugacucauucaccuccacgcgguaaguaucgggauacguuguacaucaacggcuaagaaaugaga

SEQ ID NO: 490 - RNA, 69 nt, Faecalibacterium cf.
gcuguccgaaaaugcugccucuacguggcggacggcaggcaacggagcgugucuccggcuaaagcauga

SEQ ID NO: 491 - RNA, 73 nt, Hymenolepis nana
gggcaacguauacucauacaccuccacguggugcacccugggcaacguauauucauauggcaaaaaugucuau

SEQ ID NO: 492 - RNA, 75 nt, Clostridiales bacterium
gcuguuuggauaaucacacaccgaugcgagguuagcagcaggcaacacagcggaagcuauggcgaagaugcaaug

SEQ ID NO: 493 - RNA, 75 nt, Tulasnella calospora
gggguucgagcuguacgcguaccuccucguggugaacccugggcaacgcucugacggagcggcugaaucgcguac

SEQ ID NO: 494 - RNA, 68 nt, Cerapachys biroi
aacucuaaaacguccaccuccacgugguuagaguugggcaacguuaaacauuaacggcuaacggacga

SEQ ID NO: 495 - RNA, 69 nt, Clostridium clostridioforme
gccgcccauaggugcugccucugcguggcggguggcaggcaacggaggaguucuccggcuaaagcacug

SEQ ID NO: 496 - RNA, 78 nt, Heligmosomoides polygyrus
gcuucccgaugacggugccaccuccacguggugggaagcgggcaacggguuuggauuggcgcccggcuaagagcaccg

SEQ ID NO: 497 - RNA, 77 nt, Nasonia vitripennis
ggguuuucaaugaacguucaccuucacguggugaaacccgggcaacguuacauucagcagcggcuaagaacguucac

SEQ ID NO: 498 - RNA, 70 nt, Ruminococcus sp.
guuacgcaagaucaaagccuccucgcggcgcguagcgggcaacgaauuuucauucggcugauuugaucga

SEQ ID NO: 499 - RNA, 79 nt, Cyphomyrmex costatus
aguuuucgaaagucguucaccuccucguggugaaaacuggauaacguuuaaauaacugauaaacggcaaagaaacgaca

SEQ ID NO: 500 - RNA, 72 nt, Ruminococcus sp.
gccgcacaaaaucaaagccuccacguggcgugcggcggacaacggaugauugauccggcuaagauugauuga

SEQ ID NO: 501 - RNA, 77 nt, Caenorhabditis tropicalis
cuucuacacguacuucgccucuccguggcguagaagaggcaacacuccugggcaaccagaguggcuaagaaguacac

SEQ ID NO: 502 - RNA, 75 nt, Hymenolepis nana
ggggugcuagauagacuuaccuccacgcgguguacccugggcaacguauacucaucauacggcaaauaagucaau

SEQ ID NO: 503 - RNA, 75 nt, Anaerotruncus sp.
accgcacagggucaaagccuccacguggcgugcggugggcaacggacaacucgcucguccggcugauuugaccau

SEQ ID NO: 504 - RNA, 70 nt, Faecalibacterium prausnitzii
gccgcucagauaugcuaccucuccguggugagcagcaggcaacgagaguuuucucucggcuaaagcauau

SEQ ID NO: 505 - RNA, 75 nt, Trichomalopsis sarcophagae
gguucucuuucaucguucaccuccccguggugagaaccgggcaacacaacauuucagaguggcaaagaaacgauu

SEQ ID NO: 506 - RNA, 84 nt, Trichomalopsis sarcophagae
gauucucaaaagcguucaccuccucguggugagaaucgggcaacucugauguuuacgaauaaaucagaggcaaagaacgcguga

SEQ ID NO: 507 - RNA, 82 nt, Steinernema glaseri
ggaagacgacgagcuacaccuccacguggugucuuccgggcaacguuagggcuucuggguccuaacggcaaagacagcucua

SEQ ID NO: 508 - RNA, 83 nt, Oesophagostomum dentatum
gguccucauagcugccaccuccacguggugaggaccgggcaacguuggugcuucuggagccaccaacggcuaaggcagcgugg

SEQ ID NO: 509 - RNA, 71 nt, Gonapodya prolifera
ggagcgcgauggcccgcccaccucuacguggugcgcucuagaaacaccaguuugguggcugagaggcgggc

SEQ ID NO: 510 - RNA, 71 nt, Gonapodya prolifera
gggguacacggugacugccuccucguggcguaccccgggcaacguucgauuuucgaacggcugaagucacc

SEQ ID NO: 511 - RNA, 74 nt, Trichomalopsis sarcophagae
gauucucaauguuugcuaaccuccacguggugagaaucgggcaacguuuauuuauaaacggcaaagaggcaaua

SEQ ID NO: 512 - RNA, 82 nt, Clostridium sp.
gcuacucggacaaaucaaaaaauuacacaccucuucgugguaagcagcagacaacgauuuuaugaucggcgaagaugugaga

SEQ ID NO: 513 - RNA, 77 nt, Clostridiales bacterium
guugcucgaaugcgaaugaaucacacaccucuccguggugagcagcaggcaaugaaguuauaucauaaaauuuuuaa

SEQ ID NO: 514 - RNA, 76 nt, Cyphomyrmex costatus
gguuaucgauaagcguccaccuccucgcggugauaaccgggcaacguugaauucaucagcggcaaaggacgucuaa

SEQ ID NO: 515 - RNA, 74 nt, Ruminococcus sp.
gcugcucgaaaaaugcacaccgcuacgaggugagcagcagacaacacagcagagacuguggcuaagaggcaaga

SEQ ID NO: 516 - RNA, 72 nt, Hymenolepis nana
ggggugugagacagacuuaccucaacgugguacaccccaggcaacguauauuuaugcggcaaaaaaguuuau

SEQ ID NO: 517 - RNA, 75 nt, Caenorhabditis brenneri
cuucuucgacgguacuaaccucuacgcggugaagaagagacaacaguuucugaugaaacuggcuaauaaguacca

SEQ ID NO: 518 - RNA, 71 nt, Unknown
  Unknown Organism
gcuacucaaaaaaagacagccuccacgcggcgagcagcgggcaacgggaaagacccggcagauagucuuua

SEQ ID NO: 519 - RNA, 80 nt, Faecalibacterium sp.
uccgcucgaaacuuugcacaccucuacgcggugggcggcaggcaacacaguguguagaugcuguggcaaagaaugcaaga

SEQ ID NO: 520 - RNA, 76 nt, Ruminococcus sp.
gcugcucagaaaugcacacugcgacuggugagcaguaggugauguuuaucaaaggauaagcggcuaagauguagaa

SEQ ID NO: 521 - RNA, 75 nt, Unknown
  Unknown Organism
gggggacgaagucgaacugaacaccuccaucguggugucccccgggcaacgcuugcaaaagcggcuaacguucag

SEQ ID NO: 522 - RNA, 89 nt, Unknown
  Unknown Organism
gcugcucguaauaaucacacaccucuccguggugagcagcaggcaacgauuuaagaauguaugguucgaugaucggcgaagaugugcga

SEQ ID NO: 523 - RNA, 71 nt, Unknown
  Unknown Organism
acugcacaaaaccaacagccuucacgcggcgugcagugagcaacguauaguuuuauacggccaauguugaa

SEQ ID NO: 524 - RNA, 76 nt, Unknown
  Unknown Organism
gccgcuuuauauuuuguacaccucuacgugguaagcggcaggcaacguuuauuuuauagacggcuaagaugcaaaa

SEQ ID NO: 525 - RNA, 89 nt, Coprococcus comes
gcugcucgaaugaaucacacaccucuuuguggugaguagcaggcaacgaucuaagaaucagggauccggugaucggcugagaugugaag

SEQ ID NO: 526 - RNA, 78 nt, Unknown
  Unknown Organism
aucucacaacguuaaucgccuccucguggcgugagauggaaacagcauauuugcaaauauguuggcuaagauuaacag

SEQ ID NO: 527 - RNA, 45 nt, Schistosoma haematobium
ucccggcugacgagucucaaacagaacguaaugcgcguccuggau

SEQ ID NO: 528 - RNA, 49 nt, Schistosoma mattheei
auccaucugaugaauccuaaaauaggacgaaacaugcgucaaacuggau

SEQ ID NO: 529 - RNA, 45 nt, Schistosoma margrebowiei
auccaacugaugugucuuagguaaaacgaaacacgcguccuggau

SEQ ID NO: 530 - RNA, 45 nt, Schistosoma curassoni
acccuaaugaaaagugccaaauaguacgaaacuuaagucuagggu

SEQ ID NO: 531 - RNA, 216 nt, Schistosoma haematobium
auucagcugacgagucccaaugucguguuugaaugaacaaaugauuccuuuguacuuguugaaugcuugauuucgaauucuaaauacaguacauucgcuugugcuuaucuacuacuucuugauccaauuacguuauuucuggaauucuuaguucauacuauaacucaaagacuccuaauuaucacacccgaguaggaugaaacgcgcguccugaau

SEQ ID NO: 532 - RNA, 68 nt, Schistosoma rodhaini
  misc_feature (33)..(55): n is a, c, g, or u
auccagcugacgagucccgaauaggacaaaacnnnnnnnnnnnnnnnnnnnnnnngagcguucuggau

SEQ ID NO: 533 - RNA, 45 nt, Schistosoma rodhaini
auccagacgacgaguccaagacaggaccaaacgcgcuuuuuguau

SEQ ID NO: 534 - RNA, 45 nt, Schistosoma rodhaini
acccagaugaugagucucacauaaaacgaaacguacgucuuagau

SEQ ID NO: 535 - RNA, 49 nt, Trichobilharzia regenti
auccagaugacgagucccaggucgaacgaaaugcgcauccuggcuggau

SEQ ID NO: 536 - RNA, 46 nt, Schistosoma haematobium
auucuacugacgagucccaaacaggacgagauggauuuuauagaau

SEQ ID NO: 537 - RNA, 48 nt, Trichobilharzia regenti
auccggaugacgaguccaaaauagggugaaauacgcguaauccuggau

SEQ ID NO: 538 - RNA, 45 nt, Schistosoma haematobium
accuugcggacgaguaccaaauagcacgaaacccggguccagggu

SEQ ID NO: 539 - RNA, 45 nt, Schistosoma rodhaini
auccaucugacgagcccuaaauggggcgaaaugcacauccugcac

SEQ ID NO: 540 - RNA, 46 nt, Schistosoma mansoni
auccagccgaagaguucaaaauuuagacgaaaugugcguccaggau

SEQ ID NO: 541 - RNA, 45 nt, Schistosoma mansoni
guccagccgaugaguucgaaauaggaugaaacgcacguccugaau

SEQ ID NO: 542 - RNA, 45 nt, Schistosoma margrebowiei
auucaacugauggguucaaaauaggacggagcucgcguccugaau

SEQ ID NO: 543 - RNA, 48 nt, Schistosoma margrebowiei
aucuagcugacgugucucaaauagggugaaacgcgcaucaaacuggau

SEQ ID NO: 544 - RNA, 46 nt, Schistosoma rodhaini
gucuugcugaggagucccacaauuggacaaaacgaucguccaguac

SEQ ID NO: 545 - RNA, 45 nt, Schistosoma mattheei
auccagacgaugagucgcaaucaggacaaaacgcguguccugcau

SEQ ID NO: 546 - RNA, 314 nt, Schistosoma rodhaini
ucacugcugaggagucccacaacagggugaaacgaccauccagugcuuucagguucuccauagugguccagcuucaaucgacucaugauuucaacuguuaaaauacuaaaucuccacaaaaacccuucugauaauucauaauagaucagagggggguuuguggagaauuuaguauuuuaacaguugaaaucaugagucgauugaagcuagaccaucauugaaaaccugaaagcacuggacggccauuucguucuauuaugggaauccucagcagugcgcauccauaauaauaggacgaaacggccguccagugc

SEQ ID NO: 547 - RNA, 45 nt, Schistosoma margrebowiei
auucagcugacgaguguugaauaagacggaacgugcauccugaau

SEQ ID NO: 548 - RNA, 51 nt, Echinostoma caproni
gcacugcugacgagucccagacaggacgaaacaacaacaacuguccagugc

SEQ ID NO: 549 - RNA, 45 nt, Schistosoma curassoni
accuuggugacgagugucaaauaggacgaaacuuagguccaugau

SEQ ID NO: 550 - RNA, 46 nt, Trichobilharzia regenti
guaucaccgaagagucccaaacuaggacgaaacagcagucuaauac

SEQ ID NO: 551 - RNA, 45 nt, Schistosoma rodhaini
auucaacuaaugaaucccaaguagaacgaaacguacguccugaau

SEQ ID NO: 552 - RNA, 141 nt, Schistosoma mattheei
auucagcugacgagucccacuuagcuauugaguccugauaauuacuugcuugugcaauuucugaagagaacaucaacucugggauguaggcacauccugcugaugggucgcaaauaggacgaaacgcgcgucaaaccggau

SEQ ID NO: 553 - RNA, 45 nt, Schistosoma margrebowiei
aucuaucugacaaguccuaaauaggacuaaacgugcguucugaau

SEQ ID NO: 554 - RNA, 46 nt, Trichobilharzia regenti
gcaccgaugaagaguccuaaaauaggacgaaacggcugucuggcgc

SEQ ID NO: 555 - RNA, 45 nt, Schistosoma curassoni
accuagccgacgagucugaaauaggacaaaacguguguccuugau

SEQ ID NO: 556 - RNA, 46 nt, Octopus bimaculoides
uucuggcugacgaaacacaacaggucgaaacaccggugucccagaa

SEQ ID NO: 557 - RNA, 45 nt, Schistosoma mansoni
auuuagcugauguaucccaaacaaaacgaaacacacgucaugaau

SEQ ID NO: 558 - RNA, 45 nt, Opisthorchis viverrini
gcacugcugaugagcucuaauuagagcgaaacucgaguccagugc

SEQ ID NO: 559 - RNA, 45 nt, Schistosoma haematobium
auucaacugauaagucccaaacaggaugaaauaagcaucuugaau

SEQ ID NO: 560 - RNA, 49 nt, Schistosoma haematobium
gcauugcugaggagucccacaauaagacgaaacguccgucaaacaauac

SEQ ID NO: 561 - RNA, 214 nt, Schistosoma haematobium
auugcugaggagucccauacuaguauuuuaguauuuuagauugaauaaaacuucauaaacaaagauggauaguggcuagcaguggaauccaggacacgcguuucguccuauuugugacucguuagcuagaugguccugcauuucagaguugauguucacucuaggacucgaacccaguaccguucgcuacaaggacgaaacgcgcguccugaau

SEQ ID NO: 562 - RNA, 46 nt, Schistosoma margrebowiei
gcacuacugaggagucccagaacaaaaggaaacggccgucuagugu

SEQ ID NO: 563 - RNA, 46 nt, Schistosoma haematobium
acacugcugaagaguccuacaaugggacgaaacagccgucugguau

SEQ ID NO: 564 - RNA, 45 nt, Schistosoma haematobium
auccaacugacaaaucccaaacaggaugaaacgcacguccucuau

SEQ ID NO: 565 - RNA, 45 nt, Schistosoma rodhaini
auccaacugaugagugucaaauaggacaaaacucuaguucuguau

SEQ ID NO: 566 - RNA, 46 nt, Schistosoma curassoni
guauuguugaggagucgcauaccagggcgaaacggccguccaauac

SEQ ID NO: 567 - RNA, 45 nt, Schistosoma margrebowiei
auccgucugacaaguccuagauagaacgagacgcgcgucuuggau

SEQ ID NO: 568 - RNA, 55 nt, Schistosoma mansoni
auccaacugaugagucccaaauagaaccaaauaggacgaaaugcauguccuggau

SEQ ID NO: 569 - RNA, 108 nt, Trichobilharzia regenti
  misc_feature (21)..(85): n is a, c, g, or u
auccagcugaugagucccaannnnnnnnnnnnnnnnnnnnnnnnnnnnnnnnnnnnnnnnnnnnnnnnnnnnnnnnnnnnnnnnnaggacgaaaugugcaucuuggau

SEQ ID NO: 570 - RNA, 46 nt, Trichobilharzia regenti
aucgaaaugacgagucccaaauggaacgaaacccgugucuuuugau

SEQ ID NO: 571 - RNA, 151 nt, Unknown
  Unknown Organism
aauucauucgcaaaguaauuauucuaugaaaugcaaauuaucuucauauguugugaaacauagcuuaaccacguuaaaguauaauaauauaaguuagguaugcccuuauaaagacuuagguagcgcuaaggacuauauuauuauacuucuu

SEQ ID NO: 572 - DNA, 23 bp, Artificial
  synthetic
taatacgactcactatagggaat

SEQ ID NO: 573 - RNA, 49 nt, Artificial; identical to SEQ ID NO: 2
  synthetic

SEQ ID NO: 574 - RNA, 20 nt, Artificial
  synthetic
gggaaaaaaaugccgucggu

SEQ ID NO: 575 - RNA, 20 nt, Artificial
  synthetic
accgacggcaaaaaaaaaaa

SEQ ID NO: 576 - RNA, 464 nt, encephalomyocarditis virus; identical to SEQ ID NO: 5

SEQ ID NO: 577 - RNA, 516 nt, Artificial; identical to SEQ ID NO: 6
  synthetic

SEQ ID NO: 578 - RNA, 68 nt, Artificial; identical to SEQ ID NO: 7
  synthetic

SEQ ID NO: 579 - DNA, 1160 bp, Artificial; identical to SEQ ID NO: 8
  synthetic

SEQ ID NO: 580 - RNA, 1143 nt, Artificial; identical to SEQ ID NO: 9
  synthetic

SEQ ID NO: 581 - RNA, 1026 nt, Artificial; identical to SEQ ID NO: 10
  synthetic

SEQ ID NO: 582 - RNA, 228 nt, Unknown; identical to SEQ ID NO: 11
  viral

SEQ ID NO: 583 - RNA, 121 nt, Hibiscus chlorotic ringspot virus; identical to SEQ ID NO: 12

SEQ ID NO: 584 - RNA, 147 nt, Zea mays; identical to SEQ ID NO: 13

SEQ ID NO: 585 - RNA, 1653 nt, Artificial; identical to SEQ ID NO: 14
  synthetic

SEQ ID NO: 586 - RNA, 790 nt, Artificial; identical to SEQ ID NO: 15
  synthetic

SEQ ID NO: 587 - RNA, 1876 nt, Artificial; identical to SEQ ID NO: 16
  synthetic

SEQ ID NO: 588 - DNA, 49 bp, Artificial; identical to SEQ ID NO: 17
  synthetic

SEQ ID NO: 589 - DNA, 20 bp, Artificial
  synthetic
gggaaaaaaatgccgtcggt

SEQ ID NO: 590 - DNA, 20 bp, Artificial
  synthetic
accgacggcaaaaaaaaaaa

SEQ ID NO: 591 - DNA, 464 bp, encephalomyocarditis virus; identical to SEQ ID NO: 20

SEQ ID NO: 592 - DNA, 516 bp, Artificial; identical to SEQ ID NO: 21
  synthetic

SEQ ID NO: 593 - DNA, 68 bp, Unknown; identical to SEQ ID NO: 22
  viral

SEQ ID NO: 594 - RNA, 683 nt, Artificial; identical to SEQ ID NO: 23
  synthetic

SEQ ID NO: 595 - DNA, 47 bp, Artificial
  synthetic
ccggtcccaagcccggataaaatccgagggggcgggaaaccgcctaa

SEQ ID NO: 596 - DNA, 55 bp, Artificial
  synthetic
aacactgccaatgccggtcccaagcccggataaaagtggagggaaaggggatagt

SEQ ID NO: 597 - DNA, 27 bp, Artificial
  synthetic
ggatgtgttccctaggagggtgggtgt

SEQ ID NO: 598 - DNA, 34 bp, Artificial
  synthetic
acacctctccccctcccaggtactatcccctttc

SEQ ID NO: 599 - DNA, 53 bp, Artificial
  synthetic
ttggaccaatcgtggcgtgtcggcctgcttcggcaggcactggcgccgtccag

SEQ ID NO: 600 - DNA, 29 bp, Satellite tobacco necrosis virus
agtaaagacaggaaactttactgactaac

SEQ ID NO: 601 - DNA, 122 bp, Unknown
  viral
agatatcgacctgcctgaccaggctgagattgcgctagccggcgtagttggtatctctcgcgcaagcgggtttgaaggtgcggcctatcttaggggggtaaattgtaacttcgcacaaaggc

SEQ ID NO: 602 - DNA, 38 bp, Tobacco necrosis virus
gatacctaaccagtgtctcagtgattaagtaatcagct

SEQ ID NO: 603 - DNA, 140 bp, Barley yellow dwarf virus
gtgaagattgaccatctcacaaaagctgttacgtgcttgtaacacactacgcgcccgttttgtattcgggaagtagttgcgaaaacggtccccttattgcctgacaagctaagggccacccttctttccccaccgccatc

SEQ ID NO: 604 - DNA, 42 bp, Artificial
  synthetic
caaccacaacgctctaacgcaatcaatctacattatattaaa

SEQ ID NO: 605 - DNA, 619 bp, Satellite tobacco necrosis virus
tcccagaggttcacaatgttagtgatggggcgctgaaagatgcgtagctacccttctggagccacttcctggtggtaagcagaaatccaagggtacggtggtacggtggaaagcagtcccagctctgcattgggaaccggcttacacccagcttagggctaaagtgtactacttgctcatttgtagtctaaatgagacgttggcctcgacgtgtcgaggtggcctaagggattggaacccctgatggtcgtagtcgaatttcccgtgtttcattccgagtctcttggtcataatgccattagtaggtctagcactcaacgtaacttcaaagatatcctccttgcaacaagaatatgtgcgccgtctgtgtttaaagcggtatattaagtgcgccggcatatcgttgtttggaccagggccccacgccggttggtacccgggtggcttcccctcgttcacagggctttaggagatgataaggtatagttattagacaaatgcggacaaacctgaaaagctcgctagtggtgggctggccaagcgaagaacctcatccaggtatagttctacatgggaaatttggtaccatccaaacttctatgaagtcctcgactacccc

SEQ ID NO: 606 - DNA, 221 bp, Unknown
  viral
gacaaactctccgcctaatgtctgatgatgtgaggaacgtggactgtgatgtggtggtgcggtaccatggctggtcaccatggtaatgcgtagggcaacacagttcattaagactcactgatgatggcactaggcacggttcacccccatccttcgggagggctatagggggtgaccgggttacaccaccggaagaccggaacattgcctttgggcagccc

SEQ ID NO: 607 - DNA, 306 bp, Tobacco necrosis virus
ttgctttcatagatccgtcttccgggagacgttaagaagacacccgataaaaatagtaagtagagttgtggcgtgacaacccacaactgcatctctttgcttatctaattacaatatatgttgacgtacaagccggatcctgggaaacaggtttaacgggctcactgtggtggtgggccgtcgatacacttgtatgtgccccaatattggttgtcgataagcgtcctgacatggctccatgcaacagcatgggggggtccagagtcagtcccctctttatttacctagagatctctctaggaaccc

SEQ ID NO: 608 - DNA, 869 bp, Barley yellow dwarf virus
agtgaagacaacaccactagcacaaatcggatcctgggaaacaggcagaacttcggttcgtaagctcgggtaggccgtcaacctaccgccgtatcgtattgtgtttggccgatggaggatcttcacgttatcgccgtttgtattcttgctttgactgtgctctctggggtaggcgctgttttgagttgctgccgttggtgctgcagcaatccttttcctccctccctctcttctgttcaagcaaaagactctcgatctgtgcgagagacaatcaaaaatatcgagggagcttcggctcagtgaggggattaacgacccccagtaatggccggtcctggcggacataaataacccgctataggacgaagtggtagccaccactgatcaaatggcaaacatgcttctgtgttgtacactgccccggagcctaccgggtcaacaaggctatcccaccaacccgatgaaatgagggtggagtgagcggagtgggtgacttcgtgatgtacacccgatcgtcaggattgaagacgttaaaactcgacgacctggtacaagtcgttaaactgactcgggtggatacaccacacccggcccagcatgttggcatacccacgatacgaaacgtgggtctcttggagccactacctgtgatgcaaggtagggtatgagtcttagcaagctctgagccaggagatggacataaaccatagcaatccaacgtgtaaccgcaatggggcaaacaacaggtgaaccgtgtccacgggcctggttaccgaaaggaaagccagtatccaacacagcaatgtgttgggggtcacaccttcggggtactcttaacgctgacactcgaaagagcagttcggcaaccc

SEQ ID NO: 609 - protein, 183 aa, Cowpea mosaic virus
TTAACTCTGGTTTCATTAAATTTTCTTTAGTTTGAATTTACTGTTATTCGGTGTGCATTTCTATGTTTGGTGAGCGGTTTTCTGTGCTCAGAGTGTGTTTATTTTATGTAATTTAATTTCTTTGTGAGCTCCTGTTTAGCAGGTCGTCCCTTCAGCAAGGACACAAAAGATTTTAATTTTATT

SEQ ID NO: 610 - DNA, 41 bp, Arabidopsis thaliana
tcgcacaagaaaataaaagatttgtagaatcaactaagaaa

SEQ ID NO: 611 - DNA, 22 bp, Tobacco mosaic virus
acaattactatttacaattaca

SEQ ID NO: 612 - DNA, 137 bp, Turnip crinkle virus
ggtaatatatgctttctacaactctctctcactggtcctcctactttgtcatctgattcctgaaatcaaaccgattcacacatcctacaacacacacgactcatcgaagcagcaacacataagcatcaacactggaa

SEQ ID NO: 613 - DNA, 255 bp, Turnip crinkle virus
tacggtaatagtgtagtcttctcatcttagtagttagctctctcttatattaagaaaagaaaacaaaaacccccaggtcgctttattttgacctgtgttagggaccaaaaacggtggcagcactgtctagctgcgggcattagactggaaaactagtgctctttgggtaaccactaaaatcccgaaagggtgggctgtggtgaccttccgaactaaaagatagcctccctcctcgcgcgggggggggtcctgccc

(19) United States
(12) Patent Application Publication
(10) Pub. No.: US 2024/0263206 A1
(43) Pub. Date: Aug. 8, 2024

(54) COMPOSITIONS AND METHODS FOR PRODUCING CIRCULAR POLYRIBONUCLEOTIDES

(71) Applicant: FLAGSHIP PIONEERING INNOVATIONS VII, LLC, CAMBRIDGE, MA (US)

(72) Inventors: Barry Andrew Martin, Newport, RI (US); Swetha Srinivasa Murali, Somerville, MA (US); Yajie Niu, Lexington, MA (US); Derek Thomas Rothenheber, Somerville, MA (US); Michka Gabrielle Sharpe, Cambridge, MA (US); Andrew McKinley Shumaker, Somerville, MA (US)

(21) Appl. No.: 18/283,257

(22) PCT Filed: Mar. 25, 2022
(86) PCT No.: PCT/US2022/021854

§ 371 Date: Sep. 21, 2023

Related U.S. Application Data
(60) Provisional application No. 63/166,467, filed on Mar. 26, 2021.

(51) Int. Cl. C12P 19/34; C12N 9/00; C12N 15/113

(52) U.S. Cl. CPC C12P 19/34; C12N 9/93; C12N 15/113; C12N 2310/128

(57) ABSTRACT

The present disclosure relates, generally, to compositions and methods for producing, purifying, and using circular RNA.

### REFERENCE TO PRIORITY APPLICATION

This international patent application filed under the patent Cooperation Treaty claims benefit of U.S. provisional patent application Ser. No. 63/166,467, filed Mar. 26, 2021.

### INCORPORATION OF SEQUENCE LISTINGS

This application contains a Sequence Listing which has been submitted electronically in ASCII format and is hereby incorporated by reference in its entirety. Said ASCII copy, created on Mar. 16, 2022, is named VL70002WO1_ST25, and is 192,364 bytes in size Also incorporated herein by reference in its entirety is the Sequence listing filed in U.S. provisional patent application Ser. No. 63/166,467, created on Mar. 25, 2021, named 51484-003001_Sequence_Listing_3.25.21_ST25, and which is 166,651 bytes in size.

### BACKGROUND

Circular polyribonucleotides are a subclass of polyribonucleotides that exist as continuous loops. Endogenous circular polyribonucleotides are expressed ubiquitously in human tissues and cells. Most endogenous circular polyribonucleotides are generated through backsplicing and primarily fulfill noncoding roles. The use of synthetic circular polyribonucleotides, including protein-coding circular polyribonucleotides, has been suggested for a variety of therapeutic and engineering applications. There is a need for methods of producing, purifying, and using circular polyribonucleotides.

### SUMMARY

The disclosure provides compositions and methods for producing, purifying, and using circular RNA.

In a first aspect, the disclosure features a polyribonucleotide, e.g., a linear polyribonucleotide, including the following, operably linked in a 5′-to-3′ orientation: (A) a 5′ self-cleaving ribozyme; (B) a 5′ annealing region; (C) a polyribonucleotide cargo; (D) a 3′ annealing region; and (E) a 3′ self-cleaving ribozyme. The linear polyribonucleotide can include further elements, e.g., outside of or between any of elements (A), (B), (C), (D), and (E). For example, any of elements (A), (B), (C), (D), and/or (E) can be separated by a spacer sequence, as described herein.

In another aspect the disclosure provides a polyribonucleotide, e.g., linear polyribonucleotide having the formula 5′-(A)-(B)-(C)-(D)-(E)-3′, wherein: (A) includes a 5′ self-cleaving ribozyme; (B) includes a 5′ annealing region; (C) includes a polyribonucleotide cargo; (D) includes a 3′ annealing region; and (E) includes a 3′ self-cleaving ribozyme.

In some embodiments, the 5′ self-cleaving ribozyme is capable of self-cleavage at a site that is located within 10 ribonucleotides of the 3′ end of the 5′ self-cleaving ribozyme or that is located at the 3′ end of the 5′ self-cleaving ribozyme.

In some embodiments, the 5′ self-cleaving ribozyme is a ribozyme selected from Hammerhead, Hairpin, Hepatitis Delta Virus ribozyme (HDV), Varkud Satellite (VS), glmS ribozyme, Twister, Twister sister, Hatchet, and Pistol ribozymes. In some embodiments, the 5′ self-cleaving ribozyme is a Hammerhead ribozyme. In some embodiments, the 5′ self-cleaving ribozyme includes a region having at least 85%, 90%, 95%, %%, 97%, 98%, or 99% sequence identity with the nucleic acid sequence of SEQ ID NO: 1. In some embodiments, the 5′ self-cleaving ribozyme includes the nucleic acid sequence of SEQ ID NO: 2. In some embodiments, the 5′ self-cleaving ribozyme includes a nucleic acid sequence having at least 85%, 90%, 95%, 96%, 97%, 98%, or 99% sequence identity with any one of SEQ ID NOs: 24-571, or a catalytically-competent fragment thereof. In some embodiments, the 5′ self-cleaving ribozyme includes the nucleic acid sequence of any one of SEQ ID NOs: 24-571, or a catalytically-competent fragment thereof.

In some embodiments, the 3′ self-cleaving ribozyme is capable of self-cleavage at a site that is located within 10 ribonucleotides of the 5′ end of the 3′ self-cleaving ribozyme or that is located at the 5′ end of the 3′ self-cleaving ribozyme.

In some embodiments, the 3′ self-cleaving ribozyme is a ribozyme selected from Hammerhead, Hairpin, Hepatitis Delta Virus ribozyme (HDV), Varkud Satellite (VS), glmS ribozyme, Twister, Twister sister, Hatchet, and Pistol ribozymes. In some embodiments, the 3′ self-cleaving ribozyme is a hepatitis delta virus (HDV) ribozyme. In some embodiments, the 3′ self-cleaving ribozyme includes a region having at least 85%, 90%, 95%, 96%, 97%, 98%, or 99% sequence identity with the nucleic acid sequence of SEQ ID NO: 2. In some embodiments, the 3′ self-cleaving ribozyme includes the nucleic acid sequence of SEQ ID NO: 7. In some embodiments, the 3′ self-cleaving ribozyme includes a nucleic acid sequence having at least 85%, 90%, 95%, 96%, 97%, 98%, or 99% sequence identity with any one of SEQ ID NOs: 24-571, or a catalytically-competent fragment thereof. In some embodiments, the 3′ self-cleaving ribozyme includes the nucleic acid sequence of any one of SEQ ID NOs: 24-571, or a catalytically-competent fragment thereof.

In some embodiments, the 5′ self-cleaving ribozyme and of the 3′ self-cleaving ribozyme produce a ligase-compatible linear polyribonucleotide. In some embodiments, cleavage of the 5′ self-cleaving ribozyme produces a free 5′-hydroxyl group and cleavage of 3′ self-cleaving ribozyme produces a free 2′,3′-cyclic phosphate group.

In some embodiments, the 5′ and 3′ self-cleaving ribozymes share at least 80%, 85%, 90%, 95%, 98%, or 99% sequence identity. In some embodiments, the 5′ and 3′ self-cleaving ribozymes are from the same family of self-cleaving ribozymes. In some embodiments, the 5′ and 3′ self-cleaving ribozymes share 100% sequence identity.

In some embodiments, the 5′ and 3′ self-cleaving ribozymes share less than 100%, 99%, 95%, 90%, 85%, or 80% sequence identity. In some embodiments, the 5′ and 3′ self-cleaving ribozymes are not from the same family of self-cleaving ribozymes.

In some embodiments, the 5′ annealing region has 5 to 100 ribonucleotides (e.g., 5 to 80, 5 to 50, 5 to 30, 5 to 20, 10 to 100, 10 to 80, 10 to 50, or 10 to 30 ribonucleotides). In some embodiments, the 3′ annealing region has 5 to 100 ribonucleotides (e.g., 5 to 80, 5 to 50, 5 to 30, 5 to 20, 10 to 100, 10 to 80, 10 to 50, or 10 to 30 ribonucleotides).

In some embodiments, the 5′ annealing region and the 3′ annealing region each include a complementary region (e.g., forming a pair of complementary regions). In some embodiments, the 5′ annealing region includes a 5′ complementary region having between 5 and 50 ribonucleotides (e.g., 5-40, 5-30, 5-20, 5-10, 10-50, 10-40, 10-30, 10-20, or 20-50 ribonucleotides); and the 3′ annealing region includes a 3′ complementary region having between 5 and 50 ribonucleotides (e.g., 5-40, 5-30, 5-20, 5-10, 10-50, 10-40, 10-30, 10-20, or 20-50 ribonucleotides). In some embodiments, the 5′ complementary region and the 3′ complementary region have between 50% and 100% sequence complementarity (e.g., between 60%-100%, 70%-100%, 80%-100%, 90%-100%, or 100% sequence complementarity).

In some embodiments, the 5′ complementary region and the 3′ complementary region have a free energy of binding of less than −5 kcal/mol (e.g., less than −10 kcal/mol, less than −20 kcal/mol, or less than −30 kcal/mol). In some embodiments, the 5′ complementary region and the 3′ complementary region have a Tm of binding of at least 10° C., at least 15° C., at least 20° C., at least 30° C., at least 40° C., at least 50° C., at least 60° C., at least 70° C., at least 80° C., or at least 90° C. In some embodiments, the 5′ complementary region and the 3′ complementary region include no more than 10 mismatches, e.g., 10, 9, 8, 7, 6, 5, 4, 3, or 2 mismatches, or 1 mismatch. In some embodiments, the 5′ complementary region and the 3′ complementary region do not include any mismatches.

In some embodiments, the 5′ annealing region and the 3′ annealing region each include a non-complementary region. In some embodiments, the 5′ annealing region further includes a 5′ non-complementary region having between 5 and 50 ribonucleotides (e.g., 5-40, 5-30, 5-20, 5-10, 10-50, 10-40, 10-30, 10-20, or 20-50 ribonucleotides). In some embodiments, the 3′ annealing region further includes a 3′ non-complementary region having between 5 and 50 ribonucleotides (e.g., 5-40, 5-30, 5-20, 5-10, 10-50, 10-40, 10-30, 10-20, or 20-50 ribonucleotides). In some embodiments the 5′ non-complementary region is located 5′ to the 5′ complementary region (e.g., between the 5′ self-cleaving ribozyme and the 5′ complementary region). In some embodiments, the 3′ non-complementary region is located 3′ to the 3′ complementary region (e.g., between the 3′ complementary region and the 3′ self-cleaving ribozyme). In some embodiments, the 5′ non-complementary region and the 3′ non-complementary region have between 0% and 50% sequence complementarity (e.g., between 0%-40%, 0%-30%, 0%-20%, 0%-10%, or 0% sequence complementarity). In some embodiments, the 5′ non-complementary region and the 3′ non-complementary region have a free energy of binding of greater than −5 kcal/mol. In some embodiments, the 5′ complementary region and the 3′ complementary region have a Tm of binding of less than 10° C. In some embodiments, the 5′ non-complementary region and the 3′ non-complementary region include at least 1, 2, 3, 4, 5, 6, 7, 8, 9, or 10 mismatches. In some embodiments, the 5′ annealing region and the 3′ annealing region do not include any non-complementary region.

In some embodiments, the 5′ annealing region includes a region having at least 85%, 90%, 95%, 96%, 974, 98%, or 99% sequence identity with the nucleic acid sequence of SEQ ID NO: 3. In some embodiments, the 5′ annealing region includes the nucleic acid sequence of SEQ ID NO: 3. In some embodiments, the 3′ annealing region includes a region having at least 85%, 90%, 95%, 96%, 97%, 98%, or 99% sequence identity with the nucleic acid sequence of SEQ ID NO: 4. In some embodiments, the 3′ annealing region includes the nucleic acid sequence of SEQ ID NO: 4.

In some embodiments, the polyribonucleotide cargo includes an expression sequence encoding a polypeptide. In some embodiments, the polyribonucleotide cargo includes an IRES operably linked to an expression sequence encoding a polypeptide. In some embodiments, the polypeptide is a biologically active polypeptide. In some embodiments, the polypeptide is a therapeutic polypeptide, e.g., for a human or non-human animal. In some embodiments, the polypeptide is a polypeptide having a sequence encoded in the genome of a vertebrate (e.g., non-human mammal, reptile, bird, amphibian, or fish), invertebrate (e.g., insect, arachnid, nematode, or mollusk), plant (e.g., monocot, dicot, gymnosperm, eukaryotic alga), or microbe (e.g., bacterium, fungus, archaea, oomycete). In some embodiments, the polypeptide has a biological effect when contacted with a vertebrate, invertebrate, or plant, or when contacted with a vertebrate cell, invertebrate cell, microbial cell, or plant cell. In some embodiments, the polypeptide is a plant-modifying polypeptide. In some embodiments, the polypeptide increases the fitness of a vertebrate, invertebrate, or plant, or increases the fitness of a vertebrate cell, invertebrate cell, microbial cell, or plant cell when contacted therewith. In some embodiments, the polypeptide decreases the fitness of a vertebrate, invertebrate, or plant, or decreases the fitness of a vertebrate cell, invertebrate cell, microbial cell, or plant cell, when contacted therewith.

In some embodiments, the linear polyribonucleotide further includes a spacer region of at least 5 polyribonucleotides in length between the 5′ annealing region and the polyribonucleotide cargo. In some embodiments, the linear polyribonucleotide further includes a spacer region of between 5 and 1000 polyribonucleotides in length between the 5′ annealing region and the polyribonucleotide cargo. In some embodiments, the spacer region includes a polyA sequence. In some embodiments, the spacer region includes a polyA-C sequence.

In some embodiments, the linear polyribonucleotide is at least 1 kb. In some embodiments, the linear polyribonucleotide is 1 kb to 20 kb. In some embodiments, the linear polyribonucleotide is 100 to about 20,000 nucleotides. In some embodiments, the linear RNA is at least 100, 500, 550, 600, 650, 700, 750, 800, 850, 900, 950, 1,000, 1,100, 1,200, 1,300, 1,400, 1,500, 1,600 1,700, 1,800, 1,900, 2,000, 2,500, 3,000, 3,500, 4,000, 4,500, 5,000, 6,000, 7,000, 8,000, 9,000, or 10,000 nucleotides in size.

In another aspect, the disclosure provides a deoxyribonucleic acid including an RNA polymerase promoter operably linked to a sequence encoding a linear polyribonucleotide described herein. In some embodiments, the RNA polymerase promoter is heterologous to the sequence encoding the linear polyribonucleotide. In some embodiments, the RNA polymerase promoter is a T7 promoter, a T6 promoter, a T4 promoter, a T3 promoter, an SP3 promoter, or an SP6 promoter.

In another aspect, the disclosure provides a circular polyribonucleotide produced from a linear polyribonucleotide or from a deoxyribonucleic acid described herein.

In some embodiments, the circular polyribonucleotide is at least 1 kb. In some embodiments, the circular polyribonucleotide is 1 kb to 20 kb. In some embodiments, the circular polyribonucleotide is 100 to about 20,000 nucleotides. In some embodiments, the circular RNA is at least 100, 500, 550, 600, 650, 700, 750, 800, 850, 900, 950, 1,000, 1,100, 1,200, 1,300, 1,400, 1,500, 1,600 1,700, 1,800, 1,900, 2,000, 2,500, 3,000, 3,500, 4,000, 4,500 5,000, 6,000, 7,000, 8,000, 9,000, or 10,000 nucleotides in size.

In another aspect, the disclosure provides a method of producing a circular polyribonucleotide, the method including: providing a linear polyribonucleotide (e.g., a precursor linear polyribonucleotide described herein) wherein the linear polyribonucleotide is in solution (e.g., in solution in a cell free system) under conditions suitable for cleavage of the 5′ self-cleaving ribozyme and the 3′ self-cleaving ribozyme thereby producing a ligase-compatible linear polyribonucleotide; and contacting the ligase-compatible linear polyribonucleotide with a ligase under conditions suitable for ligation of the 5′ and 3′ ends of the ligase-compatible linear polyribonucleotide; thereby producing a circular polyribonucleotide.

In another aspect, the disclosure provides a method of producing a circular polyribonucleotide, the method including: providing a deoxyribonucleotide encoding the linear polyribonucleotide (e.g., a precursor linear polyribonucleotide described herein); transcribing the deoxyribonucleotide in a cell-free system (e.g., in vitro transcription) to produce the linear polyribonucleotide; wherein the transcribing occurs under conditions suitable for cleavage of the 5′ self-cleaving ribozyme and 3′ self-cleaving ribozyme thereby producing a ligase-compatible linear polyribonucleotide; optionally purifying the ligase-compatible linear polyribonucleotide; and contacting the ligase-compatible linear polyribonucleotide with a ligase under conditions suitable for ligation of the 5′ and 3′ ends of the ligase-compatible linear polyribonucleotide, thereby producing a circular polyribonucleotide.

In another aspect, the disclosure provides a method of producing a circular polyribonucleotide, the method including: providing a deoxyribonucleotide encoding the linear polyribonucleotide (e.g., a precursor linear polyribonucleotide described herein); transcribing the deoxyribonucleotide in a cell-free system (e.g., in vitro transcription) to produce the linear polyribonucleotide; wherein the transcribing occurs under conditions suitable for cleavage of the 5′ self-cleaving ribozyme and 3′ self-cleaving ribozyme thereby producing a ligase-compatible linear polyribonucleotide; and wherein the transcribing occurs in a solution including a ligase and under conditions suitable for ligation of the 5′ and 3′ ends of the ligase-compatible linear polyribonucleotide, thereby producing a circular polyribonucleotide.

In another aspect, the disclosure provides a method of producing a circular polyribonucleotide, the method including: providing a deoxyribonucleotide encoding a linear polyribonucleotide; transcribing the deoxyribonucleotide in a cell-free system (e.g., in vitro transcription) to produce the linear polyribonucleotide, wherein the transcribing occurs in a solution comprising a ligase and under conditions suitable for ligation of the 5′ and 3′ ends of the linear polyribonucleotide, thereby producing a circular polyribonucleotide. In some embodiments, the linear polyribonucleotide comprises a 5′ self-cleaving ribozyme and a 3′ self-cleaving ribozyme. In some embodiments, the linear polyribonucleotide comprises a 5′ split-intron and a 3′ split-intron (e.g., a self-splicing construct for producing a circular polyribonucleotide). In some embodiments, the linear polyribonucleotide comprises a 5′ annealing region and a 3′ annealing region.

In some embodiments, the linear polyribonucleotide is produced from a deoxyribonucleic acid, e.g., a deoxyribonucleic acid described herein, such as a DNA vector, a linearized DNA vector, or a cDNA. In some embodiments, the deoxyribonucleic acid includes an RNA polymerase promoter operably linked to a sequence encoding the linear polyribonucleotide. In embodiments, the RNA polymerase promoter is heterologous to the sequence encoding the linear polyribonucleotide. In some embodiments, the RNA polymerase promoter is a T7 promoter, a T6 promoter, a T4 promoter, a T3 promoter, an SP3 promoter, or an SP6 promoter. In some embodiments, the linear polyribonucleotide is transcribed from the deoxyribonucleic acid by transcription in a cell-free system (e.g., in vitro transcription).

In some embodiments, the ligase-compatible linear polyribonucleotide is substantially enriched or pure, e.g., it is purified prior to contacting the ligase-compatible linear polyribonucleotide with a ligase. In some embodiments, the ligase-compatible linear polyribonucleotide is purified by enzymatic purification or by chromatography.

In some embodiments, the transcription of the linear polyribonucleotide is performed in a solution including the ligase.

In some embodiments, the ligase is an RNA ligase. In some embodiments, the RNA ligase is a tRNA ligase. In some embodiments, the tRNA ligase is a T4 ligase, an RtcB ligase, a TRL-1 ligase, and Rnl1 ligase, an Rnl2 ligase, a LIG1 ligase, a LIG2 ligase a PNK/PNL ligase, a PF0027 ligase, a thpR ligT ligase, a ytlPor ligase, or a variant thereof (e.g., a mutational variant that retains ligase function). In some embodiments the tRNA ligase is a T4 ligase or an RtcB ligase.

In some embodiments, the RNA ligase is a plant RNA ligase or a variant thereof. In some embodiments, the RNA ligase is a chloroplast RNA ligase or a variant thereof. In embodiments, the RNA ligase is a eukaryotic algal RNA ligase or a variant thereof. In some embodiments, the RNA ligase is an RNA ligase from archaea or a variant thereof. In some embodiments, the RNA ligase is a bacterial RNA ligase or a variant thereof. In some embodiments, the RNA ligase is a eukaryotic RNA ligase or a variant thereof. In some embodiments, the RNA ligase is a viral RNA ligase or a variant thereof. In some embodiments, the RNA ligase is a mitochondrial RNA ligase or a variant thereof.

In some embodiments, the RNA ligase is a ligase described in Table 2, or a variant thereof.

In another aspect, the disclosure provides a method of delivering a polyribonucleotide cargo to a cell, the method including contacting the cell with a circular polyribonucleotide described herein.

In another aspect, the disclosure provides a method of expressing a polypeptide in a cell, the method including contacting a cell with a circular polyribonucleotide described herein (e.g., a circular polyribonucleotide produced by the methods described herein). In some embodiments, the cell is an isolated cell. In some embodiments, the cell is transfected with a circular polyribonucleotide described herein. In some embodiments the cell is in a subject and a circular polyribonucleotide described herein is administered to that subject.

In some embodiments, circular polyribonucleotides made as described herein are used as effectors in therapy and/or agriculture. For example, a circular polyribonucleotide made by the methods described herein (e.g., the cell-free methods described herein) can be administered to a subject (e.g., in a pharmaceutical, veterinary, or agricultural composition). In some embodiments, the subject is a vertebrate animal (e.g., mammal, bird, fish, reptile, or amphibian). In some embodiments, the subject is a human. In some embodiments, the subject is a non-human mammal. In some embodiments, the subject is a non-human mammal such as a non-human primate, ungulate, carnivore, rodent, or lagomorph. In some embodiments, the subject is a bird, reptile, or amphibian. In some embodiments, the subject is an invertebrate animal. In some embodiments, the subject is a plant or eukaryotic alga. In some embodiments, the subject is a plant, such as angiosperm plant (which can be a dicot or a monocot) or a gymnosperm plant (e.g., a conifer, a cycad, a gnetophyte, a Ginkgo), a fern, horsetail, clubmoss, or a bryophyte. In embodiments, the subject is a plant of agricultural or horticultural importance, such as a row crop, fruit, vegetable, tree, or ornamental plant. In some embodiments, a circular polyribonucleotide made by the methods described herein (e.g., the cell-free methods described herein) can be delivered to a cell.

### Definitions

To facilitate the understanding of this disclosure, a number of terms are defined below. Terms defined herein have meanings as commonly understood by a person of ordinary skill in the areas relevant to the disclosure. Terms such as “a”, “an”, and “the” are not intended to refer to only a singular entity but include the general class of which a specific example can be used for illustration. The terminology herein is used to describe specific embodiments, but their usage is not to be taken as limiting, except as outlined in the claims.

The term “and/or” where used herein is to be taken as specific disclosure of each of the multiple specified features or components with or without another. Thus, the term “and/or” as used in a phrase such as “A and/or B” herein is intended to include “A and B,” “A or B,” “A” (alone), and “B” (alone). Likewise, the term “and/or” as used in a phrase such as “A, B, and/or C” is intended to encompass each of the following embodiments: A, B, and C; A, B, or C; A or C; A or B; B or C; A and C; A and B; B and C; A (alone); B (alone); and C (alone).

As used herein, any values provided in a range of values include both the upper and lower bounds, and any values contained within the upper and lower bounds.

As used herein, the terms “circRNA” or “circular polyribonucleotide” or “circular RNA” or “circular polyribonucleotide molecule” or “circularized RNA” are used interchangeably and mean a polyribonucleotide molecule that has a structure having no free ends (i.e., no free 3′ and/or 5′ ends), for example a polyribonucleotide molecule that forms a circular or end-less structure through covalent or non-covalent bonds.

As used herein, the term “circularization efficiency” is a measurement of resultant circular polyribonucleotide versus its non-circular (linear) starting material.

The wording “compound, composition, product, etc. for treating, modulating, etc.” is to be understood to refer a compound, composition, product, etc. per se which is suitable for the indicated purposes of treating, modulating, etc. The wording “compound, composition, product, etc. for treating, modulating, etc.” additionally discloses that, as a preferred embodiment, such compound, composition, product, etc. is for use in treating, modulating, etc.

The wording “compound, composition, product, etc. for use in . . . ” or “use of a compound, composition, product, etc. in the manufacture of a medicament, pharmaceutical composition, veterinary composition, diagnostic composition, etc. for . . . ” indicates that such compounds, compositions, products, etc. are to be used in therapeutic methods which can be practiced on the human or animal body. They are considered as an equivalent disclosure of embodiments and claims pertaining to methods of treatment, etc. If an embodiment or a claim thus refers to “a compound for use in treating a human or animal being suspected to suffer from a disease”, this is considered to be also a disclosure of a “use of a compound in the manufacture of a medicament for treating a human or animal being suspected to suffer from a disease” or a “method of treatment by administering a compound to a human or animal being suspected to suffer from a disease”.

As used herein, the terms “disease,” “disorder,” and “condition” each refer to a state of sub-optimal health, for example, a state that is or would typically be diagnosed or treated by a medical professional.

By “heterologous” is meant to occur in a context other than in the naturally occurring (native) context. A “heterologous” polynucleotide sequence indicates that the polynucleotide sequence is being used in a way other than what is found in that sequence's native genome. For example, a “heterologous promoter” is used to drive transcription of a sequence that is not one that is natively transcribed by that promoter, thus, a “heterologous promoter” sequence is often included in an expression construct by means of recombinant nucleic acid techniques. The term “heterologous” is also used to refer to a given sequence that is placed in a non-naturally occurring relationship to another sequence; for example, a heterologous coding or non-coding nucleotide sequence is commonly inserted into a genome by genomic transformation techniques, resulting in a genetically modified or recombinant genome.

As used herein “increasing fitness” or “promoting fitness” of a subject refers to any favorable alteration in physiology, or of any activity carried out by a subject organism, as a consequence of administration of a peptide or polypeptide described herein, including, but not limited to, any one or more of the following desired effects: (1) increased tolerance of biotic or abiotic stress by about 10%, 20%, 30%, 40%, 50%, 60%, 70%, 80%, 90%, 95%, 99%, 100% or more; (2) increased yield or biomass by about 10%, 20%, 30%, 40%, 50%, 60%, 70%, 80%, 90%, 95%, 99%, 100% or more; (3) modified flowering time by about 10%, 20%, 30%, 40%, 50%, 60%, 70%, 80%, 90%, 95%, 99%, 100% or more; (4) increased resistance to pests or pathogens by about 10%, 20%, 30%, 40%, 50%, 60%, 70%, 80%, 90%, 95%, 99%, 100% or more, (4) increased resistance to herbicides by about 10%, 20%, 30%, 40%, 50%, 60%, 70%, 80%, 90%, 95%, 99%, 100% or more; (5) increasing a population of a subject organism (e.g., an agriculturally important insect) by about 10%, 20%, 30%, 40%, 50%, 60%, 70%, 80%, 90%, 95%, 99%, 100% or more; (6) increasing the reproductive rate of a subject organism (e.g., insect, e.g., bee or silkworm) by about 10%, 20%, 30%, 40%, 50%, 60%, 70%, 80%, 90%, 95%, 99%, 100% or more; (7) increasing the mobility of a subject organism (e.g., insect, e.g., bee or silkworm) by about 10%, 20%, 30%, 40%, 50%, 60%, 70%, 80%, 90%, 95%, 99%, 100% or more; (8) increasing the body weight of a subject organism (e.g., insect, e.g., bee or silkworm) by about 10%, 20%, 30%, 40%, 50%, 60%, 70%, 80%, 90%, 95%, 99%, 100% or more; (9) increasing the metabolic rate or activity of a subject organism (e.g., insect, e.g., bee or silkworm) by about 10%, 20%, 30%, 40%, 50%, 60%, 70%, 80%, 90%, 95%, 99%, 100% or more; (10) increasing pollination (e.g., number of plants pollinated in a given amount of time) by a subject organism (e.g., insect, e.g., bee or silkworm) by about 10%, 20%, 30%, 40%, 50%, 60%, 70%, 80%, 90%, 95%, 99%, 100% or more; (11) increasing production of subject organism (e.g., insect, e.g., bee or silkworm) byproducts (e.g., honey from a honeybee or silk from a silkworm) by about 10%, 20%, 30%, 40%, 50%, 60%, 70%, 80%, 90%, 95%, 99%, 100% or more; (12) increasing nutrient content of the subject organism (e.g., insect) (e.g., protein, fatty acids, or amino acids) by about 10%, 20%, 30%, 40%, 50%, 60%, 70%, 80%, 90%, 95%, 99%, 100% or more; or (13) increasing a subject organism's resistance to pesticides (e.g., a neonicotinoid (e.g., imidacloprid) or an organophosphorus insecticide (e.g., a phosphorothioate, e.g., fenitrothion)) by about 10%, 20%, 30%, 40%, 50%, 60%, 70%, 80%, 90%, 95%, 99%, 100% or more, (14) increasing health or reducing disease of a subject organism such as a human or non-human animal. An increase in host fitness can be determined in comparison to a subject organism to which the modulating agent has not been administered. Conversely, “decreasing fitness” of a subject refers to any unfavorable alteration in physiology, or of any activity carried out by a subject organism, as a consequence of administration of a peptide or polypeptide described herein, including, but not limited to, any one or more of the following intended effects: (1) decreased tolerance of biotic or abiotic stress by about 10%, 20%, 30%, 40%, 50%, 60%, 70%, 80%, 90%, 95%, 99%, 100% or more; (2) decreased yield or biomass by about 10%, 20%, 30%, 40%, 50%, 60%, 70%, 80%, 90%, 95%, 99%, 100% or more; (3) modified flowering time by about 10%, 20%, 30%, 40%, 50%, 60%, 70%, 80%, 90%, 95%, 99%, 100% or more; (4) decreased resistance to pests or pathogens by about 10%, 20%, 30%, 40%, 50%, 60%, 70%, 80%, 90%, 95%, 99%, 100% or more, (4) decreased resistance to herbicides by about 10%, 20%, 30%, 40%, 50%, 60%, 70%, 80%, 90%, 95%, 99%, 100% or more; (5) decreasing a population of a subject organism (e.g., an agriculturally important insect) by about 10%, 20%, 30%, 40%, 50%, 60%, 70%, 80%, 90%, 95%, 99%, 100% or more; (6) decreasing the reproductive rate of a subject organism (e.g., insect, e.g., bee or silkworm) by about 10%, 20%, 30%, 40%, 50%, 60%, 70%, 80%, 90%, 95%, 99%, 100% or more; (7) decreasing the mobility of a subject organism (e.g., insect, e.g., bee or silkworm) by about 10%, 20%, 30%, 40%, 50%, 60%, 70%, 80%, 90%, 95%, 99%, 100% or more; (8) decreasing the body weight of a subject organism (e.g., insect, e.g., bee or silkworm) by about 10%, 20%, 30%, 40%, 50%, 60%, 70%, 80%, 90%, 95%, 99%, 100% or more; (9) decreasing the metabolic rate or activity of a subject organism (e.g., insect, e.g., bee or silkworm) by about 10%, 20%, 30%, 40%, 50%, 60%, 70%, 80%, 90%, 95%, 99%, 100% or more; (10) decreasing pollination (e.g., number of plants pollinated in a given amount of time) by a subject organism (e.g., insect, e.g., bee or silkworm) by about 10%, 20%, 30%, 40%, 50%, 60%, 70%, 80%, 90%, 95%, 99%, 100% or more; (11) decreasing production of subject organism (e.g., insect, e.g., bee or silkworm) byproducts (e.g., honey from a honeybee or silk from a silkworm) by about 10%, 20%, 30%, 40%, 50%, 60%, 70%, 80%, 90%, 95%, 99%, 100% or more; (12) decreasing nutrient content of the subject organism (e.g., insect) (e.g., protein, fatty acids, or amino acids) by about 10%, 20%, 30%, 40%, 50%, 60%, 70%, 80%, 90%, 95%, 99%, 100% or more; or (13) decreasing a subject organism's resistance to pesticides (e.g., a neonicotinoid (e.g., imidacloprid) or an organophosphorus insecticide (e.g., a phosphorothioate, e.g., fenitrothion)) by about 10%, 20%, 30%, 40%, 50%, 60%, 70%, 80%, 90%, 95%, 99%, 100% or more, (14) decreasing health or reducing disease of a subject organism such as a human or non-human animal. A decrease in host fitness can be determined in comparison to a subject organism to which the modulating agent has not been administered. It will be apparent to one of skill in the art that certain changes in the physiology, phenotype, or activity of a subject, e.g., modification of flowering time in a plant, can be considered to increase fitness of the subject or to decrease fitness of the subject, depending on the context (e.g., to adapt to a change in climate or other environmental conditions). For example, a delay in flowering time (e.g., about 10%, 20%, 30%, 40%, 50%, 60%, 70%, 80%, 90%, 95%, 99%, 100% fewer plants in a population flowering at a given calendar date) can be a beneficial adaptation to later or cooler springtimes and thus be considered to increase a plant's fitness; conversely, the same delay in flowering time in the context of earlier or warmer springtimes can be considered to decrease a plant's fitness.

As used herein, the terms “linear RNA” or “linear polyribonucleotide” or “linear polyribonucleotide molecule” are used interchangeably and mean polyribonucleotide molecule having a 5′ and 3′ end. One or both of the 5′ and 3′ ends can be free ends or joined to another moiety. Linear RNA includes RNA that has not undergone circularization (e.g., is pre-circularized) and can be used as a starting material for circularization.

As used herein, the term “modified ribonucleotide” means a nucleotide with at least one modification to the sugar, the nucleobase, or the internucleoside linkage.

The term “pharmaceutical composition” is intended to also disclose that the circular or linear polyribonucleotide included within a pharmaceutical composition can be used for the treatment of the human or animal body by therapy.

The term “polynucleotide” as used herein means a molecule including one or more nucleic acid subunits, or nucleotides, and can be used interchangeably with “nucleic acid” or “oligonucleotide”. A polynucleotide can include one or more nucleotides selected from adenosine (A), cytosine (C), guanine (G), thymine (T) and uracil (U), or variants thereof. A nucleotide can include a nucleoside and at least 1, 2, 3, 4, 5, 6, 7, 8, 9, 10, or more phosphate (PO₃) groups. A nucleotide can include a nucleobase, a five-carbon sugar (either ribose or deoxyribose), and one or more phosphate groups. Ribonucleotides are nucleotides in which the sugar is ribose. Polyribonucleotides or ribonucleic acids, or RNA, can refer to macromolecules that include multiple ribonucleotides that are polymerized via phosphodiester bonds. Deoxyribonucleotides are nucleotides in which the sugar is deoxyribose.

As used herein, the term “polyribonucleotide cargo” herein includes any sequence including at least one polyribonucleotide. In embodiments, the polyribonucleotide cargo includes one or multiple expression sequences, wherein each expression sequence encodes a polypeptide. In embodiments, the polyribonucleotide cargo includes one or multiple noncoding sequences, such as a polyribonucleotide having regulatory or catalytic functions. In embodiments, the polyribonucleotide cargo includes a combination of expression and noncoding sequences. In embodiments, the polyribonucleotide cargo includes one or more polyribonucleotide sequence described herein, such as one or multiple regulatory elements, internal ribosomal entry site (IRES) elements, and/or spacer sequences.

As used herein, the elements of a nucleic acid are “operably connected” if they are positioned on the vector such that they can be transcribed to form a precursor RNA that can then be circularized into a circular RNA using the methods provided herein.

Polydeoxyribonucleotides or deoxyribonucleic acids, or DNA, means macromolecules that include multiple deoxyribonucleotides that are polymerized via phosphodiester bonds. A nucleotide can be a nucleoside monophosphate or a nucleoside polyphosphate. A nucleotide means a deoxyribonucleoside polyphosphate, such as, e.g., a deoxyribonucleoside triphosphate (dNTP), which can be selected from deoxyadenosine triphosphate (dATP), deoxycytidine triphosphate (dCTP), deoxyguanosine triphosphate (dGTP), uridine triphosphate (dUTP) and deoxythymidine triphosphate (dTTP) dNTPs, that include detectable tags, such as luminescent tags or markers (e.g., fluorophores). A nucleotide can include any subunit that can be incorporated into a growing nucleic acid strand. Such subunit can be an A, C, G, T, or U, or any other subunit that is specific to one or more complementary A, C, G, T or U, or complementary to a purine (i.e., A or G, or variant thereof) or a pyrimidine (i.e., C, T or U, or variant thereof). In some examples, a polynucleotide is deoxyribonucleic acid (DNA), ribonucleic acid (RNA), or derivatives or variants thereof. In some cases, a polynucleotide is a short interfering RNA (siRNA), a microRNA (miRNA), a plasmid DNA (pDNA), a short hairpin RNA (shRNA), small nuclear RNA (snRNA), messenger RNA (mRNA), precursor mRNA (pre-mRNA), antisense RNA (asRNA), to name a few, and encompasses both the nucleotide sequence and any structural embodiments thereof, such as single-stranded, double-stranded, triple-stranded, helical, hairpin, etc. In some cases, a polynucleotide molecule is circular. A polynucleotide can have various lengths. A nucleic acid molecule can have a length of at least about 10 bases, 20 bases, 30 bases, 40 bases, 50 bases, 100 bases, 200 bases, 300 bases, 400 bases, 500 bases, 1 kilobase (kb), 2 kb, 3, kb, 4 kb, 5 kb, 10 kb, 50 kb, or more. A polynucleotide can be isolated from a cell or a tissue. Embodiments of polynucleotides include isolated and purified DNA/RNA molecules, synthetic DNA/RNA molecules, and synthetic DNA/RNA analogs.

Embodiments of polynucleotides, e.g., polyribonucleotides or polydeoxyribonucleotides, include polynucleotides that contain one or more nucleotide variants, including nonstandard nucleotide(s), non-natural nucleotide(s), nucleotide analog(s) and/or modified nucleotides. Examples of modified nucleotides include, but are not limited to diaminopurine, 5-fluorouracil, 5-bromouracil, 5-chlorouracil, 5-iodouracil, hypoxanthine, xanthine, 4-acetylcytosine, 5-(carboxyhydroxylmethyl)uracil, 5-carboxymethylaminomethyl-2-thiouridine, 5-carboxymethylaminomethyluracil, dihydrouracil, beta-D-galactosylqueosine, inosine, N6-isopentenyladenine, 1-methylguanine, 1-methylinosine, 2,2-dimethylguanine, 2-methyladenine, 2-methylguanine, 3-methylcytosine, 5-methylcytosine, N6-adenine, 7-methylguanine, 5-methylaminomethyluracil, 5-methoxyaminomethyl-2-thiouracil, beta-D-mannosylqueosine, 5′-methoxycarboxymethyluracil, 5-methoxyuracil, 2-methylthio-D46-isopentenyladenine, uracil-5-oxyacetic acid (v), wybutoxosine, pseudouracil, queosine, 2-thiocytosine, 5-methyl-2-thiouracil, 2-thiouracil, 4-thiouracil, 5-methyluracil, uracil-5-oxyacetic acid methylester, uracil-5-oxyacetic acid (v), 5-methyl-2-thiouracil, 3-(3-amino-3-N-2-carboxypropyl) uracil, (acp3)w, 2,6-diaminopurine and the like. In some cases, nucleotides include modifications in their phosphate moieties, including modifications to a triphosphate moiety. Non-limiting examples of such modifications include phosphate chains of greater length (e.g., a phosphate chain having, 4, 5, 6, 7, 8, 9, 10 or more phosphate moieties) and modifications with thiol moieties (e.g., alpha-thiotriphosphate and beta-thiotriphosphates). In embodiments, nucleic acid molecules are modified at the base moiety (e.g., at one or more atoms that typically are available to form a hydrogen bond with a complementary nucleotide and/or at one or more atoms that are not typically capable of forming a hydrogen bond with a complementary nucleotide), sugar moiety or phosphate backbone. In embodiments, nucleic acid molecules contain amine-modified groups, such as amino allyl 1-dUTP (aa-dUTP) and aminohexylacrylamide-dCTP (aha-dCTP) to allow covalent attachment of amine reactive moieties, such as N-hydroxysuccinimide esters (NHS). Alternatives to standard DNA base pairs or RNA base pairs in the oligonucleotides of this disclosure can provide higher density in bits per cubic mm, higher safety (resistant to accidental or purposeful synthesis of natural toxins), easier discrimination in photo-programmed polymerases, or lower secondary structure. Such alternative base pairs compatible with natural and mutant polymerases for de novo and/or amplification synthesis are described in Betz K, Malyshev D A, Lavergne T, Welte W, Diederichs K, Dwyer T J, Ordoukhanian P, Romesberg F E, Marx A. Nat. Chem. Biol. 2012 July; 8(7):612-4, which is herein incorporated by reference for all purposes.

As used herein, “polypeptide” means a polymer of amino acid residues (natural or unnatural) linked together most often by peptide bonds. The term, as used herein, refers to proteins, polypeptides, and peptides of any size, structure, or function. Polypeptides can include gene products, naturally occurring polypeptides, synthetic polypeptides, homologs, orthologs, paralogs, fragments and other equivalents, variants, and analogs of the foregoing. A polypeptide can be a single molecule or a multi-molecular complex such as a dimer, trimer, or tetramer. They can also include single chain or multichain polypeptides such as antibodies or insulin and can be associated or linked. Most commonly disulfide linkages are found in multichain polypeptides. The term polypeptide can also apply to amino acid polymers in which one or more amino acid residues are an artificial chemical analogue of a corresponding naturally occurring amino acid.

As used herein, “precursor linear polyribonucleotide” or “precursor linear RNA” refers to a linear RNA molecule created by transcription in a cell-free system (e.g., in vitro transcription) (e.g., from a deoxyribonucleotide template provided herein). The precursor linear RNA is a linear RNA prior to cleavage of one or more self-cleaving ribozymes. Following cleavage of the one or more self-cleaving ribozymes, the linear RNA is referred to as a “ligase-compatible linear polyribonucleotide” or a “ligase compatible RNA.”

As used herein, the term “plant-modifying polypeptide” refers to a polypeptide that can alter the genetic properties (e.g., increase gene expression, decrease gene expression, or otherwise alter the nucleotide sequence of DNA or RNA), epigenetic properties, or biochemical or physiological properties of a plant in a manner that results in an increase or a decrease in plant fitness.

As used herein, the term “regulatory element” is a moiety, such as a nucleic acid sequence, that modifies expression of an expression sequence within the circular or linear polyribonucleotide.

As used herein, a “spacer” refers to any contiguous nucleotide sequence (e.g., of one or more nucleotides) that provides distance and/or flexibility between two adjacent polynucleotide regions.

As used herein, the term “sequence identity” is determined by alignment of two peptide or two nucleotide sequences using a global or local alignment algorithm. Sequences are referred to as “substantially identical” or “essentially similar” when they share at least a certain minimal percentage of sequence identity when optimally aligned (e.g., when aligned by programs such as GAP or BESTFIT using default parameters). GAP uses the Needleman and Wunsch global alignment algorithm to align two sequences over their entire length, maximizing the number of matches and minimizes the number of gaps. Generally, the GAP default parameters are used, with a gap creation penalty=50 (nucleotides)/8 (proteins) and gap extension penalty=3 (nucleotides)/2 (proteins). For nucleotides the default scoring matrix used is nwsgapdna, and for proteins the default scoring matrix is Blosum62 (Henikoff & Henikoff, 1992, PNAS 89, 915-919). Sequence alignments and scores for percentage sequence identity are determined, e.g., using computer programs, such as the GCG Wisconsin Package, Version 10.3, available from Accelrys Inc., 9685 Scranton Road, San Diego, CA 92121-3752 USA, or EmbossWin version 2.10.0 (using the program “needle”). Alternatively, or additionally, percent identity is determined by searching against databases, e.g., using algorithms such as FASTA, BLAST, etc. Sequence identity refers to the sequence identity over the entire length of the sequence.

As used herein, “structured” with regard to RNA refers to an RNA sequence that is predicted by the RNAFold software or similar predictive tools to form a structure (e.g., a hairpin loop) with itself or other sequences in the same RNA molecule.

As used herein, “ribozyme” refers to a catalytic RNA or catalytic region of RNA. A “self-cleaving ribozyme” is a ribozyme that is capable of catalyzing a cleavage reaction that occurs at a nucleotide site within or at the terminus of the ribozyme sequence itself.

As used herein, the term “subject” refers to an organism, such as an animal, plant, or microbe. In embodiments, the subject is a vertebrate animal (e.g., mammal, bird, fish, reptile, or amphibian). In embodiments, the subject is a human. In embodiments, the subject is a non-human mammal. In embodiments, the subject is a non-human mammal such as a non-human primate (e.g., monkeys, apes), ungulate (e.g., cattle, buffalo, bison, sheep, goat, pig, camel, llama, alpaca, deer, horses, donkeys), carnivore (e.g., dog, cat), rodent (e.g., rat, mouse), or lagomorph (e.g., rabbit). In embodiments, the subject is a bird, such as a member of the avian taxa Galliformes (e.g., chickens, turkeys, pheasants, quail), Anseriformes (e.g., ducks, geese), Paleaognathae (e.g., ostriches, emus), Columbiformes (e.g., pigeons, doves), or Psittaciformes (e.g., parrots). In embodiments, the subject is an invertebrate such as an arthropod (e.g., insects, arachnids, crustaceans), a nematode, an annelid, a helminth, or a mollusc. In embodiments, the subject is an invertebrate agricultural pest or an invertebrate that is parasitic on an invertebrate or vertebrate host. In embodiments, the subject is a plant, such as an angiosperm plant (which can be a dicot or a monocot) or a gymnosperm plant (e.g., a conifer, a cycad, a gnetophyte, a Ginkgo), a fern, horsetail, clubmoss, or a bryophyte. In embodiments, the subject is a eukaryotic alga (unicellular or multicellular). In embodiments, the subject is a plant of agricultural or horticultural importance, such as row crop plants, fruit-producing plants and trees, vegetables, trees, and ornamental plants including ornamental flowers, shrubs, trees, groundcovers, and turf grasses.

As used herein, the term “treat,” or “treating,” refers to a prophylactic or therapeutic treatment of a disease or disorder (e.g., an infectious disease, a cancer, a toxicity, or an allergic reaction) in a subject. The effect of treatment can include reversing, alleviating, reducing severity of, curing, inhibiting the progression of, reducing the likelihood of recurrence of the disease or one or more symptoms or manifestations of the disease or disorder, stabilizing (i.e., not worsening) the state of the disease or disorder, and/or preventing the spread of the disease or disorder as compared to the state and/or the condition of the disease or disorder in the absence of the therapeutic treatment. Embodiments include treating plants to control a disease or adverse condition caused by or associated with an invertebrate pest or a microbial (e.g., bacterial, fungal, or viral) pathogen. Embodiments include treating a plant to increase the plant's innate defense or immune capability to tolerate pest or pathogen pressure.

As used herein, the term “termination element” is a moiety, such as a nucleic acid sequence, that terminates translation of the expression sequence in the circular or linear polyribonucleotide.

As used herein, the term “translation efficiency” is a rate or amount of protein or peptide production from a ribonucleotide transcript. In some embodiments, translation efficiency can be expressed as amount of protein or peptide produced per given amount of transcript that codes for the protein or peptide, e.g., in a given period of time, e.g., in a given translation system, e.g., a cell-free translation system like rabbit reticulocyte lysate.

As used herein, the term “translation initiation sequence” is a nucleic acid sequence that initiates translation of an expression sequence in the circular or linear polyribonucleotide.

As used herein, the term “therapeutic polypeptide” refers to a polypeptide that when administered to or expressed in a subject provides some therapeutic benefit. In embodiments, a therapeutic polypeptide is used to treat or prevent a disease, disorder, or condition in a subject by administration of the therapeutic peptide to a subject or by expression in a subject of the therapeutic polypeptide. In alternative embodiments, a therapeutic polypeptide is expressed in a cell and the cell is administered to a subject to provide a therapeutic benefit.

As used herein, a “vector” means a piece of DNA, that is synthesized (e.g., using PCR), or that is taken from a virus, plasmid, or cell of a higher organism into which a foreign DNA fragment can be or has been inserted for cloning and/or expression purposes. In some embodiments, a vector can be stably maintained in an organism. A vector can include, for example, an origin of replication, a selectable marker or reporter gene, such as antibiotic resistance or GFP, and/or a multiple cloning site (MCS). The term includes linear DNA fragments (e.g., PCR products, linearized plasmid fragments), plasmid vectors, viral vectors, cosmids, bacterial artificial chromosomes (BACs), yeast artificial chromosomes (YACs), and the like. In one embodiment, the vectors provided herein include a multiple cloning site (MCS). In another embodiment, the vectors provided herein do not include an MCS.

Other features and advantages of the invention will be apparent from the following Detailed Description and the Claims.

### BRIEF DESCRIPTION OF THE DRAWINGS

The figures are meant to be illustrative of one or more features, aspects, or embodiments of the disclosure and are not intended to be limiting.

FIG. 1 is schematic depicting the design of an exemplary DNA construct of the disclosure.

FIG. 2 is a schematic depicting transcription of a DNA construct to produce a ligase-compatible linear RNA and subsequent circularization by contacting the ligase-compatible linear RNA with an RNA ligase.

FIG. 3 is an image depicting a denaturing polyacrylamide gel electrophoresis (PAGE) gel shift of circular RNA. Lane 1: Ladder with 1 kb, 500 nt RNA. Lane 2: IVT product, linear RNA. Lane 3: Post ligation aliquot, with high molecular weight circular RNA.

FIG. 4 is graph showing 1 pmol HCRSV RNA and ZmHSP RNA drive Nanoluc luciferase expression in insect cell extract (ICE) and wheat germ extract (WGE).

FIG. 5 is a graph showing 2 pmol of RNAs drive Nanoluc luciferase expression in Rabbit Reticulocyte Lysate.

FIG. 6 is an image showing a denaturing PAGE gel shift of circular RNA. Lane 1: Ladder with 1 kb, 500 nt RNA. Lane 2: IVT product, linear RNA. Lane 3: Post ligation aliquot, with high molecular weight circular RNA.

FIG. 7 shows a circularized RNA containing a Pepper aptamer was detected using fluorescence imaging of the aptamer. The gel was incubated in aptamer buffer containing 100 mM potassium chloride for 30 min and then stained with 10 micromolar ethidium bromide and 10 micromolar HBC525. Ethidium bromide signal false colored red, HBC525 signal false colored cyan. Lane 1: molecular weight ladder with relative size indicated. Lane 2: In vitro transcribed RNA construct. Lane 3: In vitro transcribed RNA construct contacted with RtcB RNA ligase; the higher molecular weight band in lane 3 corresponds to the circularized RNA.

### DETAILED DESCRIPTION

In general, the disclosure provides compositions and methods for producing, purifying, and using circular RNA.

### Polynucleotides

The disclosure features circular polyribonucleotide compositions, and methods of making circular polyribonucleotides.

In embodiments, a circular polyribonucleotide is produced from a linear polyribonucleotide (e.g., by ligation of ligase-compatible ends of the linear polyribonucleotide). In embodiments, a linear polyribonucleotide is transcribed from a deoxyribonucleotide template (e.g., a vector, a linearized vector, or a cDNA). Accordingly, the disclosure features deoxyribonucleotide, linear polyribonucleotide, and circular polyribonucleotide compositions useful in the production of circular polyribonucleotides.

### Template Deoxyribonucleotides

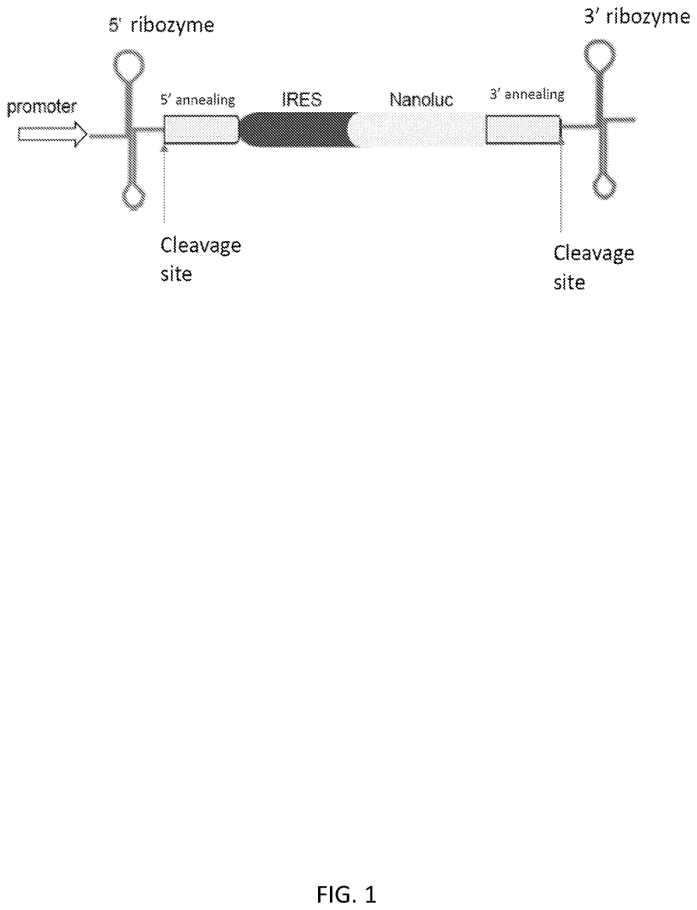

The disclosure features a deoxyribonucleotide for making circular RNA. The deoxyribonucleotide includes the following, operably linked in a 5′-to-3′ orientation: (A) a 5′ self-cleaving ribozyme; (B) a 5′ annealing region; (C) a polyribonucleotide cargo; (D) a 3′ annealing region; and (E) a 3′ self-cleaving ribozyme. In embodiments, the deoxyribonucleotide includes further elements, e.g., outside of or between any of elements (A), (B), (C), (D), and (E). In embodiments, any of the elements (A), (B), (C), (D), and/or (E) is separated from each other by a spacer sequence, as described herein. The design of an exemplary template deoxyribonucleotide is provided in FIG. 1.

In embodiments, the deoxyribonucleotide is, for example, a circular DNA vector, a linearized DNA vector, or a linear DNA (e.g., a cDNA, e.g., produced from a DNA vector).

In some embodiments, the deoxyribonucleotide further includes an RNA polymerase promoter operably linked to a sequence encoding a linear RNA described herein. In embodiments, the RNA polymerase promoter is heterologous to the sequence encoding the linear RNA. In some embodiments, the RNA polymerase promoter is a T7 promoter, a T6 promoter, a T4 promoter, a T3 promoter, an SP6 virus promoter, or an SP3 promoter.

In some embodiments, the deoxyribonucleotide includes a multiple-cloning site (MCS).

In some embodiments, the deoxyribonucleotide is used to produce circular RNA with the size range of about 100 to about 20,000 nucleotides. In some embodiments, the circular RNA is at least 100, 500, 550, 600, 650, 700, 750, 800, 850, 900, 950, 1,000, 1,100, 1,200, 1,300, 1,400, 1,500, 1,600 1,700, 1,800, 1,900, 2,000, 2,500, 3,000, 3,500, 4,000, 4,500 or 5,000 nucleotides in size. In some embodiments, the circular RNA is no more than 20,000, 15,000 10,000, 9,000, 8,000, 7,000, 6,000, 5,000 or 4,000 nucleotides in size.

### Precursor Linear Polyribonucleotides

The disclosure also features linear polyribonucleotides (e.g., precursor linear polyribonucleotides) including the following, operably linked in a 5′-to-3′ orientation: (A) a 5′ self-cleaving ribozyme; (B) a 5′ annealing region; (C) a polyribonucleotide cargo; (D) a 3′ annealing region; and (E) a 3′ self-cleaving ribozyme. The linear polyribonucleotide can include further elements, e.g., outside of or between any of elements (A), (B), (C), (D), and (E). For example, any of elements (A), (B), (C), (D), and/or (E) can be separated by a spacer sequence, as described herein.

In certain embodiments, provided herein is a method of generating precursor linear RNA by performing transcription in a cell-free system (e.g., in vitro transcription) using a deoxyribonucleotide (e.g., a vector, linearized vector, or cDNA) provided herein as a template (e.g., a vector, linearized vector, or cDNA provided herein with an RNA polymerase promoter positioned upstream of the region that codes for the linear RNA).

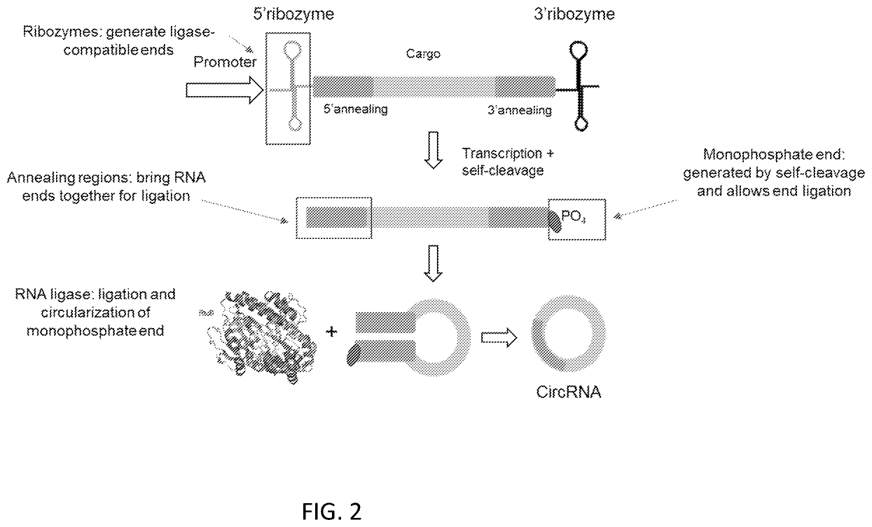

FIG. 2 is a schematic that depicts an exemplary process for producing a circular RNA from a precursor linear RNA. For example, a deoxyribonucleotide template can be transcribed to a produce a precursor linear RNA. Upon expression, under suitable conditions, and in no particular order, the 5′ and 3′ self-cleaving ribozymes each undergo a cleavage reaction thereby producing ligase-compatible ends (e.g., a 5′-hydroxyl and a 2′,3′-cyclic phosphate) and the 5′ and 3′ annealing regions bring the free ends into proximity. Accordingly, the precursor linear polyribonucleotide produces a ligase-compatible polyribonucleotide, which can be ligated (e.g., in the presence of a ligase) in order to produce a circular polyribonucleotide.

### Ligase-Compatible Linear Polyribonucleotides

The disclosure also features linear polyribonucleotides (e.g., ligase-compatible linear polyribonucleotides) including the following, operably linked in a 5′-to-3′ orientation: (B) a 5′ annealing region; (C) a polyribonucleotide cargo; and (D) a 3′ annealing region. The linear polyribonucleotide can include further elements, e.g., outside of or between any of elements (B), (C), and (D). For example, any elements (B), (C), and/or (D) can be separated by a spacer sequence, as described herein.

In some embodiments, the ligase-compatible linear polyribonucleotide includes a free 5′-hydroxyl group. In some embodiments, the ligase-compatible linear polyribonucleotide includes a free 2′,3′-cyclic phosphate.

In some embodiments, and under suitable conditions, the 3′ annealing region and the 5′ annealing region promote association of the free 3′ and 5′ ends (e.g., through partial or complete complementarity resulting thermodynamically favored association, e.g., hybridization).

In some embodiments, the proximity of the free hydroxyl and the 5′ end and a free 2′,3′-cyclic phosphate at the 3′ end favors recognition by ligase recognition, thereby improving the efficiency of circularization.

### Circular Polyribonucleotides

In some embodiments, the disclosure provides a circular RNA.

In some embodiments, the circular RNA includes a first annealing region, a polynucleotide cargo, and a second annealing region. In some embodiments, the first annealing region and the second annealing region are joined, thereby forming a circular polyribonucleotide.

In some embodiments, the circular RNA is a produced by a deoxyribonucleotide template, a precursor linear RNA, and/or a ligase-compatible linear RNA described herein (see, e.g., FIG. 2). In some embodiments, the circular RNA is produced by any of the methods described herein.

In some embodiments, the circular polyribonucleotide is at least about 20 nucleotides, at least about 30 nucleotides, at least about 40 nucleotides, at least about 50 nucleotides, at least about 75 nucleotides, at least about 100 nucleotides, at least about 200 nucleotides, at least about 300 nucleotides, at least about 400 nucleotides, at least about 500 nucleotides, at least about 1,000 nucleotides, at least about 2,000 nucleotides, at least about 5,000 nucleotides, at least about 6,000 nucleotides, at least about 7,000 nucleotides, at least about 8,000 nucleotides, at least about 9,000 nucleotides, at least about 10,000 nucleotides, at least about 12,000 nucleotides, at least about 14,000 nucleotides, at least about 15,000 nucleotides, at least about 16,000 nucleotides, at least about 17,000 nucleotides, at least about 18,000 nucleotides, at least about 19,000 nucleotides, or at least about 20,000 nucleotides.

In some embodiments, the circular polyribonucleotide is of a sufficient size to accommodate a binding site for a ribosome. In some embodiments, the size of a circular polyribonucleotide is a length sufficient to encode useful polypeptides, e.g., at least 20,000 nucleotides, at least 15,000 nucleotides, at least 10,000 nucleotides, at least 7,500 nucleotides, at least 5,000 nucleotides, at least 4,000 nucleotides, at least 3,000 nucleotides, at least 2,000 nucleotides, at least 1,000 nucleotides, at least 500 nucleotides, at least 1400 nucleotides, at least 300 nucleotides, at least 200 nucleotides, or at least 100 nucleotides.

In some embodiments, the circular polyribonucleotide includes one or more elements described elsewhere herein. In some embodiments, the elements can be separated from one another by a spacer sequence. In some embodiments, the elements can be separated from one another by 1 ribonucleotide, 2 nucleotides, about 5 nucleotides, about 10 nucleotides, about 15 nucleotides, about 20 nucleotides, about 30 nucleotides, about 40 nucleotides, about 50 nucleotides, about 60 nucleotides, about 80 nucleotides, about 100 nucleotides, about 150 nucleotides, about 200 nucleotides, about 250 nucleotides, about 300 nucleotides, about 400 nucleotides, about 500 nucleotides, about 600 nucleotides, about 700 nucleotides, about 800 nucleotides, about 900 nucleotides, about 1000 nucleotides, up to about 1 kb, at least about 1000 nucleotides, or any amount of nucleotides therebetween. In some embodiments, one or more elements are contiguous with one another, e.g., lacking a spacer element.

In some embodiments, the circular polyribonucleotide can include one or more repetitive elements described elsewhere herein. In some embodiments, the circular polyribonucleotide includes one or more modifications described elsewhere herein. In one embodiment, the circular RNA contains at least one nucleoside modification. In one embodiment, up to 100% of the nucleosides of the circular RNA are modified. In one embodiment, at least one nucleoside modification is a uridine modification or an adenosine modification.

As a result of its circularization, the circular polyribonucleotide can include certain characteristics that distinguish it from linear RNA. For example, the circular polyribonucleotide is less susceptible to degradation by exonuclease as compared to linear RNA. As such, the circular polyribonucleotide is more stable than a linear RNA, especially when incubated in the presence of an exonuclease. The increased stability of the circular polyribonucleotide compared with linear RNA makes circular polyribonucleotide more useful as a cell transforming reagent to produce polypeptides and can be stored more easily and for longer than linear RNA. The stability of the circular polyribonucleotide treated with exonuclease can be tested using methods standard in art which determine whether RNA degradation has occurred (e.g., by gel electrophoresis). Moreover, unlike linear RNA, the circular polyribonucleotide is less susceptible to dephosphorylation when the circular polyribonucleotide is incubated with phosphatase, such as calf intestine phosphatase.

### Ribozymes

Polynucleotide compositions described herein can include one or more self-cleaving ribozymes, e.g., one or more self-cleaving ribozymes described herein. A ribozyme is a catalytic RNA or catalytic region of RNA. A self-cleaving ribozyme is a ribozyme that is capable of catalyzing a cleavage reaction that occurs a nucleotide site within or at the terminus of the ribozyme sequence itself.

Exemplary self-cleaving ribozymes are known in the art and/or are provided herein. Exemplary self-cleaving ribozymes include Hammerhead, Hairpin, Hepatitis Delta Virus ribozyme (HDV), Varkud Satellite (VS), glmS ribozyme, Twister, Twister sister, Hatchet, and Pistol. Further exemplary self-cleaving ribozymes are described below and in Table 1.

In some embodiments, a polyribonucleotide of the disclosure includes a first (e.g., a 5′) self-cleaving ribozyme. In some embodiments, the ribozyme is selected from any of the ribozymes described herein. In some embodiments, a polyribonucleotide of the disclosure includes a second (e.g., a 3′) self-cleaving ribozyme. In some embodiments, the ribozyme is selected from any of the ribozymes described herein.

In some embodiments, the 5′ and 3′ self-cleaving ribozymes share at least 80%, 85%, 90%, 95%, 98%, or 99% sequence identity. In some embodiments, the 5′ and 3′ self-cleaving ribozymes are from the same family of self-cleaving ribozymes. In some embodiments, the 5′ and 3′ self-cleaving ribozymes share 100% sequence identity.

In some embodiments, the 5′ and 3′ self-cleaving ribozymes share less than 100%, 99%, 95%, 90%, 85%, or 80% sequence identity. In some embodiments, the 5′ and 3′ self-cleaving ribozymes are not from the same family of self-cleaving ribozymes.

In some embodiments, cleavage of the 5′ self-cleaving ribozyme produces a free 5′-hydroxyl residue on the corresponding linear polyribonucleotide. In some embodiments, the 5′ self-cleaving ribozyme is capable of self-cleavage at a site that is located within 10 ribonucleotides of the 3′ end of the 5′ self-cleaving ribozyme or that is located at the 3′ end of the 5′ self-cleaving ribozyme.

In some embodiments, cleavage of the 3′ self-cleaving ribozyme produces a free 3′-hydroxyl residue on the corresponding linear polyribonucleotide. In some embodiments, the 3′ self-cleaving ribozyme is capable of self-cleavage at a site that is located within 10 ribonucleotides of the 5′ end of the 3′ self-cleaving ribozyme or that is located at the 5′ end of the 3′ self-cleaving ribozyme.

The following are exemplary self-cleaving ribozymes contemplated by the disclosure. This list should not be considered to limit the scope of the disclosure.

RFam was used to identify the following self-cleaving ribozymes families. RFam is a public database containing extensive annotations of non-coding RNA elements and sequences, and in principle is the RNA analog of the PFam database that curates protein family membership. The RFam database's distinguishing characteristic is that RNA secondary structure is the primary predictor of family membership, in combination with primary sequence information. Non-coding RNAs are divided into families based on evolution from a common ancestor. These evolutionary relationships are determined by building a consensus secondary structure for a putative RNA family and then performing a specialized version of a multiple sequence alignment.

Twister: The twister ribozymes (e.g., Twister P1, P5, P3) are considered to be members of the small self-cleaving ribozyme family which includes the hammerhead, hairpin, hepatitis delta virus (HDV), Varkud satellite (VS), and glmS ribozymes. Twister ribozymes produce a 2′,3′-cyclic phosphate and 5′ hydroxyl product. See rfam.xfam.org/family/RF03160 for examples of Twister P1 ribozymes; rfam.xfam.org/family/RF03154 for examples of Twister P3 ribozymes; and rfam.xfam.org/family/RF02684 for examples of Twister P5 ribozymes.

Twister-sister: The twister sister ribozyme (TS) is a self-cleaving ribozyme with structural similarities to the Twister family of ribozymes. The catalytic products are a cyclic 2′,3′ phosphate and a 5′-hydroxyl group. See rfam.xfam.org/family/RF02681 for examples of Twister-sister ribozymes.

Hatchet: The hatchet ribozymes are self-cleaving ribozymes discovered by a bioinformatic analysis. See rfam.xfam.org/family/RF02678 for examples of Hatchet ribozymes.

HDV: The hepatitis delta virus (HDV) ribozyme is a self-cleaving ribozyme in the hepatitis delta virus. See rfam.xfam.org/family/RF00094 for examples of HDV ribozymes.

Pistol ribozyme: The pistol ribozyme is a self-cleaving ribozyme. The pistol ribozyme was discovered through comparative genomic analysis. Through mass spectrometry, it was found that the products contain 5′-hydroxyl and 2′,3′-cyclic phosphate functional groups. See rfam.xfam.org/family/RF02679 for examples of Pistol ribozymes.

HHR Type 1: The hammerhead ribozyme is a self-cleaving ribozyme that catalyzes reversible cleavage and ligation reactions at a specific site within an RNA molecule. See rfam.xfam.org/family/RF00163 for examples of HHR Type 1 ribozymes.

HHR Type 2: The hammerhead ribozyme is a self-cleaving ribozyme that catalyzes reversible cleavage and ligation reactions at a specific site within an RNA molecule. See rfam.xfam.org/family/RF02276 for examples of HHR Type 2 ribozymes.

HHR Type 3: The hammerhead ribozyme is a self-cleaving ribozyme that catalyzes reversible cleavage and ligation reactions at a specific site within an RNA molecule. These RNA structural motifs are found throughout nature. See rfam.xfam.org/family/RF00008 for examples of HHR Type 3 ribozymes.

HH9: The hammerhead ribozyme is a self-cleaving ribozyme that catalyzes reversible cleavage and ligation reactions at a specific site within an RNA molecule. See rfam.xfam.org/family/RF02275 for examples of HH9 ribozymes.

HH10: The hammerhead ribozyme is a self-cleaving ribozyme that catalyzes reversible cleavage and ligation reactions at a specific site within an RNA molecule. See rfam.xfam.org/family/RF02277 for examples of HH10 ribozymes.

glmS: The glucosamine-6-phosphate riboswitch ribozyme (glmS ribozyme) is an RNA structure that resides in the 5′ untranslated region (UTR) of the mRNA transcript of the glmS gene. See rfam.xfam.org/family/RF00234 for examples of glmS ribozymes.

GIR1: The Lariat capping ribozyme (formerly called GIR1 branching ribozyme) is an about 180 nt ribozyme with an apparent resemblance to a group I ribozyme. See rfam.xfam.org/family/RF01807 for examples of GIR1 ribozymes.

CPEB3: The mammalian CPEB3 ribozyme is a self-cleaving non-coding RNA located in the second intron of the CPEB3 gene. See rfam.xfam.org/family/RF00622 for examples of CPEB ribozymes.

drz-Agam 1 and drz-Agam 2: The drz-Agam-1 and drz-Agam 2 ribozymes were found by using a restrictive structure descriptor and closely resemble HDV and CPEB3 ribozymes. See rfam.xfam.org/family/RF01787 for examples of drz-Agam 1 ribozymes and rfam.xfam.org/family/RF01788 for examples of drz-Agam 2 ribozymes.

Hairpin: The hairpin ribozyme is a small section of RNA that can act as a ribozyme. Like the hammerhead ribozyme it is found in RNA satellites of plant viruses. See rfam.xfam.org/family/RF00173 for examples of hairpin ribozymes.

RAGATH-1: RNA structural motifs that were discovered using bioinformatics algorithms. These RNAs contained strong similarities to known ribozymes such as, but not limited to, hammerhead and HDV ribozymes. See rfam.xfam.org/family/RF03152 for examples of RAGATH-1 ribozymes.

RAGATH-5: RNA structural motifs that were discovered using bioinformatics algorithms. These RNAs contained strong similarities to known ribozymes such as, but not limited to, hammerhead and HDV ribozymes. See rfam.xfam.org/family/RF02685 for examples of RAGATH-5 ribozymes.

RAGATH-6: RNA structural motifs that were discovered using bioinformatics algorithms. These RNAs contained strong similarities to known ribozymes such as, but not limited to, hammerhead and HDV ribozymes. See rfam.xfam.org/family/RF02686 for examples of RAGATH-6 ribozymes.

RAGATH-13: RNA structural motifs that were discovered using bioinformatics algorithms. These RNAs contained strong similarities to known ribozymes such as, but not limited to, hammerhead and HDV ribozymes. See rfam.xfam.org/family/RF02688 for examples of RAGATH-13 ribozymes.

In some embodiments, a self-cleaving ribozyme is a ribozyme described herein, e.g., from a class described herein, or a ribozyme of Table 1, or a catalytically active fragment or portion thereof. In some embodiments, a ribozyme includes a sequence that is at least 85%, 90%, 95%, 96%, 97%, 98%, or 99% identical to any one of SEQ ID NOs: 24-571. In some embodiments, a ribozyme includes the sequence of any one of SEQ ID NOs: 24-571. In embodiments, the self-cleaving ribozyme is a fragment of a ribozyme disclosed in Table 1, e.g., a fragment that contains at least 20 contiguous nucleotides (e.g., at least 20, 25, 30, 35, 40, 45, 50, 55, or 60 contiguous nucleotides) of an intact ribozyme sequence and that has at least 30% (e.g., at least about 30, 40, 50, 60, 70, 75, 80, 85, 90, or 95%) catalytic activity of the intact ribozyme. In some embodiments, a ribozyme includes a catalytic region (e.g., a region capable of self-cleavage) of any one of SEQ ID NOs: 24-571, wherein the region is at least 10 nucleotides, 20 nucleotides, 30 nucleotide, 40 nucleotide, or 50 nucleotides in length or the region is between 10-200 nucleotides, 10-100 nucleotides, 10-50 nucleotides, 10-30 nucleotides, 10-200 nucleotides, 20-100 nucleotides, 20-50 nucleotides, 20-30 nucleotides. The disclosure also specifically contemplates the DNA sequences corresponding to each of the RNA sequences provided in Table 1.

TABLE 1
Exemplary self-cleaving ribozymes

| SEQ ID NO: | Accession Number | Descriptor |
|---|---|---|
| 24 | URS0000D66A6B_12908 | unclassified sequences type-P3 twister ribozyme |
| 25 | URS0000D6AAF0_12908 | unclassified sequences type-P3 twister ribozyme |
| 26 | URS0000D6663E_12908 | unclassified sequences type-P3 twister ribozyme |
| 27 | URS0000D6C266_12908 | unclassified sequences type-P3 twister ribozyme |
| 28 | URS0000D6AF2A_12908 | unclassified sequences type-P3 twister ribozyme |
| 29 | URS0000D6A2C3_12908 | unclassified sequences type-P3 twister ribozyme |
| 30 | URS0000D6726E_12908 | unclassified sequences type-P3 twister ribozyme |
| 31 | URS0000D66C2E_12908 | unclassified sequences type-P3 twister ribozyme |
| 32 | URS0000D659B0_12908 | unclassified sequences type-P1 twister ribozyme |
| 33 | URS0000D6DICA_12908 | unclassified sequences type-P1 twister ribozyme |
| 34 | URS0000D67E2B_12908 | unclassified sequences type-P1 twister ribozyme |
| 35 | URS0000D68054_12908 | unclassified sequences type-P1 twister ribozyme |
| 36 | URS0000D6D330_12908 | unclassified sequences type-P1 twister ribozyme |
| 37 | URS0000D6A800_12908 | unclassified sequences type-P1 twister ribozyme |
| 38 | URS0000D68297_12908 | unclassified sequences type-P1 twister ribozyme |
| 39 | URS0000D68DD8_12908 | unclassified sequences type-P1 twister ribozyme |
| 40 | URS0000D66D37_12908 | unclassified sequences type-P1 twister ribozyme |
| 41 | URS0000D68577_12908 | unclassified sequences type-P1 twister ribozyme |
| 42 | URS0000D68F79_12908 | unclassified sequences type-P1 twister ribozyme |
| 43 | URS0000D68EE0_12908 | unclassified sequences type-P1 twister ribozyme |
| 44 | URS0000D67CC2_12908 | unclassified sequences type-P1 twister ribozyme |
| 45 | URS0000D65864_12908 | unclassified sequences type-P1 twister ribozyme |
| 46 | URS0000D68DB5_12908 | unclassified sequences type-P1 twister ribozyme |
| 47 | URS0000D6B540_12908 | unclassified sequences type-P1 twister ribozyme |
| 48 | URS0000D6A03C_12908 | unclassified sequences type-P1 twister ribozyme |
| 49 | URS0000D6C02F_12908 | unclassified sequences type-P1 twister ribozyme |
| 50 | URS0000D6AF09_12908 | unclassified sequences type-P1 twister ribozyme |
| 51 | URS0000D67A5B_12908 | unclassified sequences type-P1 twister ribozyme |
| 52 | URS0000D66DD2_12908 | unclassified sequences type-P1 twister ribozyme |
| 53 | URS0000D667E4_12908 | unclassified sequences type-P1 twister ribozyme |
| 54 | URS0000D6A251_12908 | unclassified sequences type-P1 twister ribozyme |
| 55 | URS0000D6A995_12908 | unclassified sequences type-P1 twister ribozyme |
| 56 | URS0000D6A5FC_12908 | unclassified sequences type-P1 twister ribozyme |
| 57 | URS0000D67156_12908 | unclassified sequences type-P1 twister ribozyme |
| 58 | URS0000D6CC8F_12908 | unclassified sequences type-P1 twister ribozyme |
| 59 | URS0000D65A05_12908 | unclassified sequences type-P1 twister ribozyme |
| 60 | URS0000D6967F_12908 | unclassified sequences type-P1 twister ribozyme |
| 61 | URS0000D6755D_12908 | unclassified sequences type-P1 twister ribozyme |
| 62 | URS0000D68D61_12908 | unclassified sequences type-P1 twister ribozyme |
| 63 | URS0000D67BA2_12908 | unclassified sequences type-P1 twister ribozyme |
| 64 | URS0000D6B09E_12908 | unclassified sequences type-P1 twister ribozyme |
| 65 | URS0000D65D7A_12908 | unclassified sequences type-P1 twister ribozyme |
| 66 | URS0000D694CE_12908 | unclassified sequences type-P1 twister ribozyme |
| 67 | URS0000D68632_7029 | Acyrthosiphon pisum (pea aphid) type-P1 twister |
| | | ribozyme |
| 68 | URS0000D67356_12908 | unclassified sequences RAGATH-1 hammerhead |
| | | ribozyme |
| 69 | URS0000D6976A_12908 | unclassified sequences RAGATH-1 hammerhead |
| | | ribozyme |
| 70 | URS0000D6B94F_12908 | unclassified sequences RAGATH-1 hammerhead |
| | | ribozyme |
| 71 | URS0000D698D3_12908 | unclassified sequences RAGATH-1 hammerhead |
| | | ribozyme |
| 72 | URS0000D68882_12908 | unclassified sequences RAGATH-1 hammerhead |
| | | ribozyme |
| 73 | URS0000D6A535_12908 | unclassified sequences RAGATH-1 hammerhead |
| | | ribozyme |
| 74 | URS0000D6B98C_12908 | unclassified sequences RAGATH-1 hammerhead |
| | | ribozyme |
| 75 | URS0000D68B88_12908 | unclassified sequences RAGATH-1 hammerhead |
| | | ribozyme |
| 76 | HF986131.1 | Veillonella sp. CAG: 933 genomic scaffold, scf58 |
| 77 | BAAZ01000328.1 | Human gut metagenome DNA, contig sequence: F2- |
| | | X_000328. |
| 78 | BAAV01010313.1 | Human gut metagenome DNA, contig sequence: F1- |
| | | T_010313. |
| 79 | AACY021400709.1 | Marine metagenome 1091142135580, whole genome |
| | | shotgun sequence. |
| 80 | BABB01012728.1 | Human gut metagenome DNA, contig sequence: In- |
| | | A_012728. |
| 81 | BAAZ01000328.1 | Human gut metagenome DNA, contig sequence: F2- |
| | | X_000328. |
| 82 | AYUG01106618.1 | Fukomys damarensis contig106618, whole genome |
| | | shotgun sequence. |
| 83 | CM000825.5 | Sus scrofa isolate TJ Tabasco breed Duroc |
| | | chromosome 14, whole genome shotgun sequence. |
| 84 | AKHW03000178.1 | Alligator mississippiensis ScZkoYb_60, whole |
| | | genome shotgun sequence. |
| 85 | AFYH01145668.1 | Latimeria chalumnae contig145668, whole genome |
| | | shotgun sequence. |
| 86 | AKHW03006769.1 | Alligator mississippiensis ScZkoYb_55, whole |
| | | genome shotgun sequence. |
| 87 | AFYH01100904.1 | Latimeria chalumnae contig100904, whole genome |
| | | shotgun sequence. |
| 88 | AFYH01227694.1 | Latimeria chalumnae contig227694, whole genome |
| | | shotgun sequence. |
| 89 | GG666606.1 | Branchiostoma floridae genomic scaffold |
| | | BRAFLscaffold_190, whole genome shotgun |
| | | sequence. |
| 90 | KE695878.1 | Alligator sinensis unplaced genomic scaffold |
| | | scaffold150_1, whole genome shotgun sequence |
| 91 | AKHW03001485.1 | Alligator mississippiensis ScZkoYb_1.1, whole |
| | | genome shotgun sequence. |
| 92 | AKHW03000416.1 | Alligator mississippiensis ScZkoYb_58, whole |
| | | genome shotgun sequence. |
| 93 | AKHW03004037.1 | Alligator mississippiensis ScZkoYb_121, whole |
| | | genome shotgun sequence. |
| 94 | AFYH01110885.1 | Latimeria chalumnae contig110885, whole genome |
| | | shotgun sequence. |
| 95 | KE695937.1 | Alligator sinensis unplaced genomic scaffold |
| | | scaffold277_1, whole genome shotgun sequence |
| 96 | AAGJ05100549.1 | Strongylocentrotus purpuratus Contig100549_fixed, |
| | | whole genome shotgun sequence. |
| 97 | AKHW03003332.1 | Alligator mississippiensis ScZkoYb_244, whole |
| | | genome shotgun sequence. |
| 98 | AKHW03000533.1 | Alligator mississippiensis ScZkoYb_72, whole |
| | | genome shotgun sequence. |
| 99 | AFYH01070068.1 | Latimeria chalumnae contig070068, whole genome |
| | | shotgun sequence. |
| 100 | AAGV020425402.1 | Dasypus novemcinctus cont2.425401, whole genome |
| | | shotgun sequence. |
| 101 | CH477291.1 | Aedes aegypti strain Liverpool supercont1.106 |
| | | genomic scaffold, whole genome shotgun sequence. |
| 102 | KB663677.1 | Anopheles minimus strain MINIMUS1 unplaced |
| | | genomic scaffold supercont1.16, whole genome |
| | | shotgun sequence. |
| 103 | JXUM01096443.1 | Aedes albopictus isolate Foshan contig96443, whole |
| | | genome shotgun sequence. |
| 104 | CH477218.1 | Aedes aegypti strain Liverpool supercont1.33 |
| | | genomic scaffold, whole genome shotgun sequence. |
| 105 | CH479147.1 | Aedes aegypti strain Liverpool supercont1.2284 |
| | | genomic scaffold, whole genome shotgun sequence. |
| 106 | JXUM01057437.1 | Aedes albopictus isolate Foshan contig57437, whole |
| | | genome shotgun sequence. |
| 107 | JXUM01160006.1 | Aedes albopictus isolate Foshan contig160006, whole |
| | | genome shotgun sequence. |
| 108 | CH477452.1 | Aedes aegypti strain Liverpool supercont1.267 |
| | | genomic scaffold, whole genome shotgun sequence. |
| 109 | KE524294.1 | Anopheles sinensis unplaced genomic scaffold |
| | | AS2_scf7180000690996, whole genome shotgun |
| | | sequence. |
| 110 | CH477448.1 | Aedes aegypti strain Liverpool supercont1.263 |
| | | genomic scaffold, whole genome shotgun sequence. |
| 111 | JXUM01149242.1 | Aedes albopictus isolate Foshan contig149242, whole |
| | | genome shotgun sequence. |
| 112 | AJWK01002842.1 | Lutzomyia longipalpis Contig2844, whole genome |
| | | shotgun sequence. |
| 113 | CH477538.1 | Aedes aegypti strain Liverpool supercont1.353 |
| | | genomic scaffold, whole genome shotgun sequence. |
| 114 | NNAY01025263.1 | Trichomalopsis sarcophage strain Alberta |
| | | scaffold25490, whole genome shotgun sequence. |
| 115 | ABLF02028779.1 | Acyrthosiphon pisum strain LSR1 Contig29506, |
| | | whole genome shotgun sequence. |
| 116 | JXUM01110469.1 | Aedes albopictus isolate Foshan contig110469, whole |
| | | genome shotgun sequence. |
| 117 | KB663633.1 | Anopheles minimus strain MINIMUS1 unplaced |
| | | genomic scaffold supercont1.12, whole genome |
| | | shotgun sequence. |
| 118 | CM001417.1 | Lepisosteus oculatus linkage group LG14, whole |
| | | genome shotgun sequence. |
| 119 | CH477871.1 | Aedes aegypti strain Liverpool supercont1.686 |
| | | genomic scaffold, whole genome shotgun sequence. |
| 120 | CH477779.1 | Aedes aegypti strain Liverpool supercont1.594 |
| | | genomic scaffold, whole genome shotgun sequence. |
| 121 | JXUM01077081.1 | Aedes albopictus isolate Foshan contig77081, whole |
| | | genome shotgun sequence. |
| 122 | KI915051.1 | Anopheles farauti strain FARI unplaced genomic |
| | | scaffold supercont2.12, whole genome shotgun |
| | | sequence. |
| 123 | CH478303.1 | Aedes aegypti strain Liverpool supercont1.1120 |
| | | genomic scaffold, whole genome shotgun sequence. |
| 124 | JXUM01008119.1 | Aedes albopictus isolate Foshan contig8119, whole |
| | | genome shotgun sequence. |
| 125 | CH478279.1 | Aedes aegypti strain Liverpool supercont1.1096 |
| | | genomic scaffold, whole genome shotgun sequence. |
| 126 | ACPB03013890.1 | Rhodnius prolixus Rhodnius_prolixus-3.0.3-200.47, |
| | | whole genome shotgun sequence. |
| 127 | JXUM01176146.1 | Aedes albopictus isolate Foshan contig176146, whole |
| | | genome shotgun sequence. |
| 128 | JXUM01103962.1 | Aedes albopictus isolate Foshan contig103962, whole |
| | | genome shotgun sequence. |
| 129 | APCK01002835.1 | Anopheles albimanus strain ALBI9_A cont1.2834, |
| | | whole genome shotgun sequence. |
| 130 | JXUM01045626.1 | Aedes albopictus isolate Foshan contig45626, whole |
| | | genome shotgun sequence. |
| 131 | CM008154.1 | Anopheles albimanus strain ALBI9_A chromosome |
| | | 3L, whole genome shotgun sequence. |
| 132 | CH478188.1 | Aedes aegypti strain Liverpool supercont1.1004 |
| | | genomic scaffold, whole genome shotgun sequence. |
| 133 | KB664972.1 | Anopheles stephensi strain SDA-500 unplaced |
| | | genomic scaffold supercont1.615, whole genome |
| | | shotgun sequence. |
| 134 | CH477623.1 | Aedes aegypti strain Liverpool supercont1.438 |
| | | genomic scaffold, whole genome shotgun sequence. |
| 135 | CH477646.1 | Aedes aegypti strain Liverpool supercont1.461 |
| | | genomic scaffold, whole genome shotgun sequence. |
| 136 | CH477346.1 | Aedes aegypti strain Liverpool supercont1.161 |
| | | genomic scaffold, whole genome shotgun sequence. |
| 137 | CM000276.3 | Tribolium castaneum strain Georgia GA2 linkage |
| | | group LGX, whole genome shotgun sequence. |
| 138 | KI915054.1 | Anopheles farauti strain FAR1 unplaced genomic |
| | | scaffold supercont2.15, whole genome shotgun |
| | | sequence. |
| 139 | CH477466.1 | Aedes aegypti strain Liverpool supercont1.281 |
| | | genomic scaffold, whole genome shotgun sequence. |
| 140 | JXUM01134552.1 | Aedes albopictus isolate Foshan contig134552, whole |
| | | genome shotgun sequence. |
| 141 | DS233147.1 | Culex pipiens quinquefasciatus supercont3.1335 |
| | | genomic scaffold, whole genome shotgun sequence. |
| 142 | KB669981.1 | Anopheles epiroticus strain epiroticus2 unplaced |
| | | genomic scaffold supercont1.133, whole genome |
| | | shotgun sequence. |
| 143 | CM008155.1 | Anopheles albimanus strain ALBI9_A chromosome |
| | | 3R, whole genome shotgun sequence. |
| 144 | CH477479.1 | Aedes aegypti strain Liverpool supercont1.294 |
| | | genomic scaffold, whole genome shotgun sequence. |
| 145 | LNIX01000032.1 | Folsomia candida strain VU population |
| | | Fcan01_Sc032, whole genome shotgun sequence. |
| 146 | JQCR01000003.1 | Paenibacillus wynnii strain DSM 18334 unitig_3_1r, |
| | | whole genome shotgun sequence. |
| 147 | JH971417.1 | Agaricus bisporus var. burnettii JB137-S8 unplaced |
| | | genomic scaffold AGABI1scaffold_33, whole |
| | | genome shotgun sequence. |
| 148 | KK198763.1 | Eucalyptus grandis cultivar BRASUZ1 unplaced |
| | | genomic scaffold scaffold_11, whole genome shotgun |
| | | sequence. |
| 149 | KK198754.1 | Eucalyptus grandis cultivar BRASUZ1 unplaced |
| | | genomic scaffold scaffold_2, whole genome shotgun |
| | | sequence. |
| 150 | LHQN01020310.1 | Habropoda laboriosa contig20310, whole genome |
| | | shotgun sequence. |
| 151 | JROS01000118.1 | Desulfobulbus sp. Tol-SR contig_572, whole genome |
| | | shotgun sequence. |
| 152 | LJIJ01003888.1 | Orchesella cincta Ocin01_Sc3888, whole genome |
| | | shotgun sequence. |
| 153 | CM007892.1 | Helianthus annuus linkage group 3, whole genome |
| | | shotgun sequence. |
| 154 | AJKJ01000094.1 | Citreicella sp. 357 C357_106, whole genome shotgun |
| | | sequence. |
| 155 | CM001944.2 | Chlorocebus sabaeus isolate 1994-021 chromosome 4, |
| | | whole genome shotgun sequence. |
| 156 | KE504202.1 | Fomitopsis pinicola FP-58527 SS1 unplaced genomic |
| | | scaffold FOMPIscaffold_81, whole genome shotgun |
| | | sequence. |
| 157 | NNAY01010628.1 | Trichomalopsis sarcophagae strain Alberta |
| | | scaffold10693, whole genome shotgun sequence. |
| 158 | FN597036.1 | Vitis vinifera, whole genome shotgun sequence of line |
| | | PN40024, unoriented chromosome 13, chr13 |
| 159 | MGFD01000034.1 | Candidatus Uhrbacteria bacterium |
| | | RIFOXYB2 FULL_45_11 |
| | | rifoxyb2_full_scaffold_3973, whole genome shotgun |
| | | sequence. |
| 160 | AWGM01152003.1 | Asian citrus Psyllid, Diaphorina citri - Florida Strain, |
| | | whole genome shotgun sequence. |
| 161 | LGHO01003158.1 | Dufourea novaeangliae contig3158, whole genome |
| | | shotgun sequence. |
| 162 | KK198753.1 | Eucalyptus grandis cultivar BRASUZ1 unplaced |
| | | genomic scaffold scaffold_1, whole genome shotgun |
| | | sequence. |
| 163 | KI536799.1 | Citrus clementina cultivar Clemenules unplaced |
| | | genomic scaffold scaffold_5, whole genome shotgun |
| | | sequence. |
| 164 | LL256423.1 | Echinostoma caproni strain Egypt genome assembly, |
| | | scaffold: ECPE_scaffold0022838 |
| 165 | FN597024.1 | Vitis vinifera, whole genome shotgun sequence of line |
| | | PN40024, chromosome 6, chr6 |
| 166 | MGFG01000021.1 | Candidatus Uhrbacteria bacterium |
| | | RIFOXYC2_FULL_47_19 |
| | | rifoxyc2_full_scaffold_469, whole genome shotgun |
| | | sequence. |
| 167 | MHSH01000051.1 | Candidatus Taylorbacteria bacterium |
| | | RIFCSPLOWO2_02_FULL_46_40 |
| | | rifcsplowo2_02_scaffold_68864, whole genome |
| | | shotgun sequence. |
| 168 | MNVS01000076.1 | Candidatus Omnitrophica bacterium CG1_02_46_14 |
| | | cg1_0.2_scaffold_5404_c, whole genome shotgun |
| | | sequence. |
| 169 | JX483873.1 | Rhizobium phage RHEph01, complete genome. |
| 170 | KK198763.1 | Eucalyptus grandis cultivar BRASUZ1 unplaced |
| | | genomic scaffold scaffold_11, whole genome shotgun |
| | | sequence. |
| 171 | LGKD01404090.1 | Octopus bimaculoides Scaffold62703_contig_4, |
| | | whole genome shotgun sequence. |
| 172 | AAXJ01016906.1 | Perkinsus marinus ATCC 50983 |
| | | gcontig_1104296167808, whole genome shotgun |
| | | sequence. |
| 173 | LKEY01048241.1 | Trachymyrmex cornetzi contig48241, whole genome |
| | | shotgun sequence. |
| 174 | LCDF01000020.1 | Parcubacteria (Giovannonibacteria) bacterium |
| | | GW2011_GWF2_42_19 UV11_C0020, whole |
| | | genome shotgun sequence. |
| 175 | LGKD01378372.1 | Octopus bimaculoides Scaffold54493_contig_334, |
| | | whole genome shotgun sequence. |
| 176 | LGHO01000944.1 | Dufourea novaeangliae contig944, whole genome |
| | | shotgun sequence. |
| 177 | LL965256.1 | Schistosoma rodhaini strain Burundi genome |
| | | assembly, scaffold: SROB_scaffold0008277 |
| 178 | KL198013.1 | Pleurotus ostreatus PC15 unplaced genomic scaffold |
| | | scaffold_10, whole genome shotgun sequence. |
| 179 | KQ435798.1 | Melipona quadrifasciata isolate 111107301 unplaced |
| | | genomic scaffold scaffold95, whole genome shotgun |
| | | sequence. |
| 180 | BDFN01001407.1 | Ipomoea nil DNA, scaffold: scaffold1407, cultivar: |
| | | Tokyo-kokei standard. |
| 181 | JH687556.1 | Punctularia strigosozonata HHB-11173 SS5 unplaced |
| | | genomic scaffold PUNSTscaffold_19, whole genome |
| | | shotgun sequence. |
| 182 | KQ435803.1 | Melipona quadrifasciata isolate 111107301 unplaced |
| | | genomic scaffold scaffold98, whole genome shotgun |
| | | sequence. |
| 183 | LAUZ02000008.1 | Mycobacterium obuense strain UC1 |
| | | Mobu_contig000008, whole genome shotgun |
| | | sequence. |
| 184 | MFIE01000019.1 | Candidatus Giovannonibacteria bacterium |
| | | RIFCSPLOWO2_01_FULL_46_13 |
| | | rifcsplowo2_01_scaffold_439, whole genome shotgun |
| | | sequence. |
| 185 | LL113166.1 | Trichobilharzia regenti genome assembly, scaffold: |
| | | TRE_scaffold0102769 |
| 186 | LL959675.1 | Schistosoma rodhaini strain Burundi genome |
| | | assembly, scaffold: SROB_scaffold0002697 |
| 187 | JXUM01106395.1 | Aedes albopictus isolate Foshan contig106395, whole |
| | | genome shotgun sequence. |
| 188 | MHLO01000032.1 | Candidatus Lloydbacteria bacterium |
| | | RIFCSPHIGHO2_02_FULL_54_17 |
| | | rifcsphigho2_02_scaffold_4023, whole genome |
| | | shotgun sequence. |
| 189 | LJUD01000105.1 | Bacterium SM23_31 WORSMTZ_22961, whole |
| | | genome shotgun sequence. |
| 190 | CP000568.1 | Clostridium thermocellum ATCC 27405, complete |
| | | genome. |
| 191 | LDXR01000011.1 | Candidate division NC10 bacterium CSP1-5 |
| | | XU15_C0011, whole genome shotgun sequence. |
| 192 | JJRR01083242.1 | Balearica pavonina gibbericeps contig83242, whole |
| | | genome shotgun sequence. |
| 193 | MIAS01000104.1 | Spirochaetes bacterium GWB1_66_5 |
| | | gwb1_scaffold_16834, whole genome shotgun |
| | | sequence. |
| 194 | GG657595.1 | Clostridium asparagiforme DSM 15981 genomic |
| | | scaffold Scfld9, whole genome shotgun sequence. |
| 195 | MZGX01000001.1 | [Clostridium] hungatei strain DSM 14427 |
| | | CLHUN_contig000001, whole genome shotgun |
| | | sequence. |
| 196 | AAGJ05102072.1 | Strongy locentrotus purpuratus Contig102072_fixed, |
| | | whole genome shotgun sequence. |
| 197 | JYNH01000035.1 | Desulfosporosinus sp. 12 contig00035, whole genome |
| | | shotgun sequence. |
| 198 | MBSV01000063.1 | Clostridium sp. W14A NODE_41, whole genome |
| | | shotgun sequence. |
| 199 | AGTN01047810.1 | Bioreactor metagenome |
| | | PBDCA2_GBB5CE401D1Q9V_left, whole genome |
| | | shotgun sequence. |
| 200 | ABMG01007509.1 | Stromatolite metagenome 35133330, whole genome |
| | | shotgun sequence. |
| 201 | ADJT01008886.1 | Uncultured Ruminococcaceae bacterium |
| | | TS29_contig142355, whole genome shotgun |
| | | sequence. |
| 202 | ABSN01019877.1 | Freshwater sediment metagenome |
| | | 1wFormaldehyde_BCIB5337_x1, whole genome |
| | | shotgun sequence. |
| 203 | AACY023459782.1 | Marine metagenome ctg_1101668267133, whole |
| | | genome shotgun sequence. |
| 204 | ADGO01024387.1 | Compost metagenome contig24470, whole genome |
| | | shotgun sequence. |
| 205 | ADGO01161384.1 | Compost metagenome FHNL2OP04YM6SP, whole |
| | | genome shotgun sequence. |
| 206 | ADGO01160766.1 | Compost metagenome FHNL2OP04YQ5F0, whole |
| | | genome shotgun sequence. |
| 207 | AGTN01403367.1 | Bioreactor metagenome |
| | | PBDCA2_FISUTAU01BA9VK, whole genome |
| | | shotgun sequence. |
| 208 | AGTN01271243.1 | Bioreactor metagenome PBDCA2_contig37489, |
| | | whole genome shotgun sequence. |
| 209 | AJ001399.1 | Naegleria sp. NG872 SSU rRNA gene group I intron, |
| | | strain NG872 |
| 210 | AJ938153.1 | Didymium iridis partial IGS, 18S rRNA gene, I-DirI |
| | | gene and partial ITS1, isolate Pan2-16 |
| 211 | AM497931.1 | Naegleria sp. NG458 group I like ribozyme GIR1, |
| | | strain NG458 |
| 212 | DQ388519.1 | Heterolobosea sp. BA 16S small subunit ribosomal |
| | | RNA gene, partial sequence; and His-Cys box homing |
| | | endonuclease gene, complete cds. |
| 213 | FUXA01000016.1 | Eubacterium ruminantium strain ATCC 17233 |
| | | genome assembly, contig: |
| | | EI46DRAFT_scaffold00014.14 |
| 214 | AAAB01006002.1 | Anopheles gambiae str. PEST whole genome shotgun |
| | | sequencing project, whole genome shotgun sequence. |
| 215 | KR011063.1 | Tsukamurella phage TIN3, complete genome. |
| 216 | BBIW01000010.1 | Paenibacillus sp. TCA20 DNA, contig: |
| | | PspTCA2nb10. |
| 217 | CP013652.1 | Paenibacillus naphthalenovorans strain 32O-Y, |
| | | complete genome. |
| 218 | FP929053.1 | Ruminococcus sp. SR1 5 draft genome. |
| 219 | KB822441.1 | Clostridium sp. ASF502 genomic scaffold acMal- |
| | | supercont1.1, whole genome shotgun sequence. |
| 220 | JN035618.1 | Gordonia phage GTE7, complete genome. |
| 221 | KC821608.1 | Cellulophaga phage phi19: 3, complete genome. |
| 222 | CP009278.1 | Sphingobacterium sp. ML3W, complete genome. |
| 223 | CP015405.2 | Blautia sp. YL58, complete genome. |
| 224 | KE159636.1 | Lachnospiraceae bacterium A2 genomic scaffold |
| | | acPFL-supercont1.1, whole genome shotgun |
| | | sequence. |
| 225 | MNRF01000152.1 | Clostridiales bacterium 42_27 |
| | | Ley3_66761_scaffold_13135, whole genome shotgun |
| | | sequence. |
| 226 | KT151955.1 | Brevibacillus phage Jenst, complete genome. |
| 227 | KU998253.1 | Gordonia phage Orchid, complete genome. |
| 228 | LECW02000030.1 | Bacillus glycinifermentans strain GO-13 contig_36, |
| | | whole genome shotgun sequence. |
| 229 | LECW02000082.1 | Bacillus glycinifermentans strain GO-13 contig_9, |
| | | whole genome shotgun sequence. |
| 230 | CP002400.1 | Ethanoligenens harbinense YUAN-3, complete |
| | | genome. |
| 231 | MNRG01000094.1 | Clostridiales bacterium 44_9 |
| | | Ley3_66761_scaffold_7759, whole genome shotgun |
| | | sequence. |
| 232 | JN790865.1 | Bacillus phage B4, complete genome. |
| 233 | CP009278.1 | Sphingobacterium sp. ML3W, complete genome. |
| 234 | LECW02000023.1 | Bacillus glycinifermentans strain GO-13 contig_3, |
| | | whole genome shotgun sequence. |
| 235 | FP929062.1 | Clostridiales sp. SS3 4 draft genome. |
| 236 | FCNT01000042.1 | Alistipes sp. CHKCI003 isolate CHKC3 genome |
| | | assembly, contig: {contig42} |
| 237 | AGGO01000583.1 | Streptococcus sobrinus TCI-98 contig00583, whole |
| | | genome shotgun sequence. |
| 238 | AFHW01000093.1 | Paenibacillus elgii B69 Contig93, whole genome |
| | | shotgun sequence. |
| 239 | ABLZ01250225.1 | Marine metagenome 35801239, whole genome |
| | | shotgun sequence. |
| 240 | AACY023396520.1 | Marine metagenome ctg_1101668203871, whole |
| | | genome shotgun sequence. |
| 241 | ADCZ01000007.1 | Erysipelotrichaceae bacterium 2_2_44A cont1.7, |
| | | whole genome shotgun sequence. |
| 242 | ADCZ01000007.1 | Erysipelotrichaceae bacterium 2_2_44A cont1.7, |
| | | whole genome shotgun sequence. |
| 243 | ABPY01006745.1 | Microbial mat metagenome hsmat10_BHWZ5893_b1, |
| | | whole genome shotgun sequence. |
| 244 | AERA01001428.1 | Activated sludge metagenome contig01440, whole |
| | | genome shotgun sequence. |
| 245 | AACY020454254.1 | Marine metagenome 1096626606346, whole genome |
| | | shotgun sequence. |
| 246 | ABNK01016853.1 | Coral metagenome 39763165, whole genome shotgun |
| | | sequence. |
| 247 | AGFS01138167.1 | Atta colombica fungus garden Top 2030450980, |
| | | whole genome shotgun sequence. |
| 248 | CP000154.1 | Paenibacillus polymyxa E681, complete genome. |
| 249 | AACY022661277.1 | Marine metagenome ctg_1101667068628, whole |
| | | genome shotgun sequence. |
| 250 | AACY020496190.1 | Marine metagenome 1096626660187, whole genome |
| | | shotgun sequence. |
| 251 | AACY020454584.1 | Marine metagenome 1096626606768, whole genome |
| | | shotgun sequence. |
| 252 | AACY022753348.1 | Marine metagenome ctg_1101667160699, whole |
| | | genome shotgun sequence. |
| 253 | CP000817.1 | Lysinibacillus sphaericus C3-41, complete genome. |
| 254 | ABLX01143204.1 | Marine metagenome 32650920, whole genome |
| | | shotgun sequence. |
| 255 | AACY021048934.1 | Marine metagenome 2065701, whole genome shotgun |
| | | sequence. |
| 256 | ABPY01006745.1 | Microbial mat metagenome hsmat10_BHWZ5893_b1, |
| | | whole genome shotgun sequence. |
| 257 | CP001616.1 | Tolumonas auensis DSM 9187, complete genome. |
| 258 | CP000511.1 | Mycobacterium vanbaalenii PYR-1, complete |
| | | genome. |
| 259 | AE014299.2 | Shewanella oneidensis MR-1, complete genome. |
| 260 | NBLX01000010.1 | Desulfobacteraceae bacterium 4572_35.1 |
| | | ex4572_35.1_scaffold_634, whole genome shotgun |
| | | sequence. |
| 261 | ATHI01000003.1 | Desulfovibrio alkalitolerans DSM 16529 ctg12, whole |
| | | genome shotgun sequence. |
| 262 | JX182370.1 | Streptomyces phage R4, complete genome. |
| 263 | LSSF01000016.1 | Thermoplasmatales archaeon SG8-52-4 WOR_8- |
| | | 12_1532, whole genome shotgun sequence. |
| 264 | JNVM01000022.1 | Paenibacillus sp. MSt1 Contig_22, whole genome |
| | | shotgun sequence. |
| 265 | FTOE01000006.1 | Neptunomonas antarctica strain DSM 22306 genome |
| | | assembly, contig: Ga0111702_106 |
| 266 | CP002400.1 | Ethanoligenens harbinense YUAN-3, complete |
| | | genome. |
| 267 | MKWD01000005.1 | Rhodobacterales bacterium 65-51 |
| | | scnpilot_p_inoc_scaffold_125, whole genome shotgun |
| | | sequence. |
| 268 | CP011272.1 | Pirellula sp. SH-Sr6A, complete genome. |
| 269 | MKUZ01000009.1 | Devosia sp. 66-22 |
| | | SCNpilot_expt_1000_bf_scaffold_212, whole |
| | | genome shotgun sequence. |
| 270 | LT629781.1 | Verrucomicrobiaceae bacterium GAS474 genome |
| | | assembly, chromosome: I |
| 271 | MTQP01000067.1 | Saccharothrix sp. ALI-22-I Contig71, whole genome |
| | | shotgun sequence. |
| 272 | JXYC01000030.1 | Marinomonas sp. S3726 contig0030, whole genome |
| | | shotgun sequence. |
| 273 | JWLJ01000012.1 | Ruegeria sp. ANG-R contig_12, whole genome |
| | | shotgun sequence. |
| 274 | MTEL01000108.1 | Beggiatoa sp. IS2 Ga0073106_1108, whole genome |
| | | shotgun sequence. |
| 275 | JX182370.1 | Streptomyces phage R4, complete genome. |
| 276 | NFHL01000009.1 | Lachnoclostridium sp. An76 An76_contig_9, whole |
| | | genome shotgun sequence. |
| 277 | CP002039.1 | Herbaspirillum seropedicae SmR1, complete genome. |
| 278 | LN554852.1 | Moritella viscosa genome assembly, chromosome: 1 |
| 279 | AZQP01000024.1 | Fervidicella metallireducens AeB contig00024, whole |
| | | genome shotgun sequence. |
| 280 | MTQP01000067.1 | Saccharothrix sp. ALI-22-I Contig71, whole genome |
| | | shotgun sequence. |
| 281 | JX182370.1 | Streptomyces phage R4, complete genome. |
| 282 | CP002400.1 | Ethanoligenens harbinense YUAN-3, complete |
| | | genome. |
| 283 | KI271721.1 | Oscillibacter sp. KLE 1745 genomic scaffold |
| | | Scaffold306, whole genome shotgun sequence. |
| 284 | JH414702.1 | Subdoligranulum sp. 4_3_54A2FAA genomic |
| | | scaffold supercont1.5, whole genome shotgun |
| | | sequence. |
| 285 | CU468230.2 | Acinetobacter baumannii str. SDF, complete genome. |
| 286 | CP003275.1 | Streptomyces hygroscopicus subsp. jinggangensis |
| | | 5008, complete genome. |
| 287 | KI260480.1 | Ruminococcus callidus ATCC 27760 genomic |
| | | scaffold Scaffold724, whole genome shotgun |
| | | sequence. |
| 288 | MTQP01000067.1 | Saccharothrix sp. ALI-22-I Contig71, whole genome |
| | | shotgun sequence. |
| 289 | NFJL01000012.1 | Blautia sp. An249 An249_contig_12, whole genome |
| | | shotgun sequence. |
| 290 | CP015039.1 | Rhodovulum sp. P5, complete genome. |
| 291 | MGZL01000059.1 | Geobacteraceae bacterium GWC2_58_44 |
| | | gwc2_scaffold_235, whole genome shotgun sequence. |
| 292 | KQ948208.1 | Streptomyces yokosukanensis strain DSM 40224 |
| | | genomic scaffold PRJNA299221_s003, whole |
| | | genome shotgun sequence. |
| 293 | MDLD01000207.1 | Endozoicomonas sp. (ex Bugula neritina AB1) isolate |
| | | AB1-5 ACH42_contig000207, whole genome shotgun |
| | | sequence. |
| 294 | MGNC01000101.1 | Chloroflexi bacterium RBG_13_60_13 |
| | | RBG_13_scaffold_3543, whole genome shotgun |
| | | sequence. |
| 295 | AE014299.2 | Shewanella oneidensis MR-1, complete genome. |
| 296 | JX182370.1 | Streptomyces phage R4, complete genome. |
| 297 | FQXS01000001.1 | Desulfofustis glycolicus DSM 9705 genome |
| | | assembly, contig: EJ46DRAFT_scaffold00001.1 |
| 298 | JSEH01000038.1 | Desulfovibrio sp. TomC contig00038, whole genome |
| | | shotgun sequence. |
| 299 | CP010802.1 | Desulfuromonas soudanensis strain WTL |
| | | chromosome, complete genome. |
| 300 | JXYC01000020.1 | Marinomonas sp. S3726 contig0020, whole genome |
| | | shotgun sequence. |
| 301 | FP929045.1 | Faecalibacterium prausnitzii L2 6 draft genome. |
| 302 | FP929045.1 | Faecalibacterium prausnitzii L2 6 draft genome. |
| 303 | FP929045.1 | Faecalibacterium prausnitzii L2 6 draft genome. |
| 304 | FP929046.1 | Faecalibacterium prausnitzii SL3 3 draft genome. |
| 305 | ADJT01006171.1 | Uncultured Faecalibacterium sp. TS29_contig14193, |
| | | whole genome shotgun sequence. |
| 306 | BABD01005494.1 | Human gut metagenome DNA, contig sequence: In- |
| | | D_005494. |
| 307 | ADJT01006524.1 | Uncultured Faecalibacterium sp. TS29_contig122416, |
| | | whole genome shotgun sequence. |
| 308 | BAAU01028045.1 | Human gut metagenome DNA, contig sequence: F1- |
| | | S_028045. |
| 309 | BABG01005008.1 | Human gut metagenome DNA, contig sequence: In- |
| | | R_005008. |
| 310 | CCXP01000063.1 | Parasitella parasitica strain CBS 412.66 genome |
| | | assembly, contig: contig_63 |
| 311 | MHSK01000028.1 | Candidatus Taylorbacteria bacterium |
| | | RIFCSPLOWO2_12_FULL 43 20 |
| | | rifcsplowo2_12_scaffold_4872, whole genome |
| | | shotgun sequence. |
| 312 | MGJT01000029.1 | Candidatus Yanofskybacteria bacterium |
| | | RIFCSPHIGHO2_02_FULL_43_15c |
| | | rifcsphigho2_02_scaffold_6549, whole genome |
| | | shotgun sequence. |
| 313 | DQ112541.1 | Trichoplax adhaerens isolate Grell Red Sea |
| | | mitochondrion, complete genome. |
| 314 | AYUM01001090.1 | Galerina marginata CBS 339.88 |
| | | GALMAscaffold_102_Cont1090, whole genome |
| | | shotgun sequence. |
| 315 | KT283062.1 | Sclerotinia sclerotiorum 1980 UF-70 mitochondrion, |
| | | complete genome. |
| 316 | JRRC01306379.1 | Gossypium arboreum cultivar AKA8401 |
| | | contig_3227_1, whole genome shotgun sequence. |
| 317 | JX962719.1 | Acanthamoeba polyphaga moumouvirus, complete |
| | | genome. |
| 318 | LILC01000037.1 | Bacillus koreensis strain DSM 16467 scaffold4, whole |
| | | genome shotgun sequence. |
| 319 | AWUE01018231.1 | Corchorus olitorius cultivar O-4 contig18264, whole |
| | | genome shotgun sequence. |
| 320 | LJUB01000113.1 | Omnitrophica WOR_2 bacterium SM23_29 |
| | | WORSMTZ_35813, whole genome shotgun |
| | | sequence. |
| 321 | GG669565.1 | Rhizopus oryzae RA 99-880 supercont3.83 |
| | | mitochondrial scaffold, whole genome shotgun |
| | | sequence. |
| 322 | AOTI010097470.1 | Triticum urartu cultivar G1812 contig97470, whole |
| | | genome shotgun sequence. |
| 323 | GL541731.1 | Microbotryum lychnidis-dioicae p1A1 Lamole |
| | | unplaced genomic scaffold supercont1.89, whole |
| | | genome shotgun sequence. |
| 324 | CP002371.1 | Candidatus Liberibacter solanacearum CLso-ZC1, |
| | | complete genome. |
| 325 | KV453845.1 | Tortispora caseinolytica NRRL Y-17796 unplaced |
| | | genomic scaffold CANCAscaffold_5, whole genome |
| | | shotgun sequence. |
| 326 | CVQH01016224.1 | Verticillium longisporum isolate VL1 genome |
| | | assembly, contig: scaffold 246 |
| 327 | AUPC01004827.1 | Rhizophagus irregularis DAOM 181602 strain |
| | | DAOM 197198 GLOINscaffold_4832_Cont4827 |
| | | mitochondrial, whole genome shotgun sequence. |
| 328 | CP019082.1 | Paludisphaera borealis strain PX4, complete genome. |
| 329 | FAOM01435076.1 | Triticum aestivum genome assembly, contig: |
| | | Triticum_aestivum_CS42_TGACv1_scaffold_435076_5DL |
| 330 | CP001022.1 | Exiguobacterium sibiricum 255-15, complete genome. |
| 331 | CCXP01001784.1 | Parasitella parasitica strain CBS 412.66 genome |
| | | assembly, contig: contig_1784 |
| 332 | KQ257479.1 | Spizellomyces punctatus DAOM BR117 chromosome |
| | | Unknown supercont1.30, whole genome shotgun |
| | | sequence. |
| 333 | AAVU01000005.1 | Lyngbya sp. PCC 8106 1099428180522, whole |
| | | genome shotgun sequence. |
| 334 | DS267914.1 | Sclerotinia sclerotiorum 1980 scaffold_35 genomic |
| | | scaffold, whole genome shotgun sequence. |
| 335 | JN204424.1 | Marssonina brunnea f. sp. |
| | | 'multigermtubi' mitochondrion, complete |
| | | genome. |
| 336 | LCJR01000037.1 | Parcubacteria (Yanofskybacteria) bacterium |
| | | GW2011_GWA2_44_9 UW79_C0037, whole |
| | | genome shotgun sequence. |
| 337 | CP003614.1 | Oscillatoria nigro-viridis PCC 7112, complete |
| | | genome. |
| 338 | KF740664.1 | Pithovirus sibericum isolate P1084-T, complete |
| | | genome. |
| 339 | LAQI01000013.1 | Diplodia seriata DS_831_scaffold_v01_13, whole |
| | | genome shotgun sequence. |
| 340 | HF546977.1 | Rhizoctonia solani strain AG-1 IB complete |
| | | mitochondrial genome, isolate 7 3 14 |
| 341 | JN007486.1 | Chaetomium thermophilum var. thermophilum strain |
| | | DSM 1495 mitochondrion, complete genome. |
| 342 | CP011834.1 | Limnohabitans sp. 103DPR2, complete genome. |
| 343 | KE150417.1 | Staphylococcus sp. HGB0015 genomic scaffold aczIz- |
| | | supercont1.1, whole genome shotgun sequence. |
| 344 | LCRN01000027.1 | Parcubacteria (Uhrbacteria) bacterium |
| | | GW2011_GWC2_53_7 UY82_C0027, whole genome |
| | | shotgun sequence. |
| 345 | AWNH01000034.1 | Leptolyngbya sp. Heron Island J, whole genome |
| | | shotgun sequence. |
| 346 | KV442285.1 | Mortierella elongata AG-77 unplaced genomic |
| | | scaffold K457scaffold_276, whole genome shotgun |
| | | sequence. |
| 347 | MFJZ01000013.1 | Candidatus Gottesmanbacteria bacterium |
| | | RIFCSPLOWO2_01_FULL_49_10 |
| | | rifcsplowo2_01_scaffold_16705, whole genome |
| | | shotgun sequence. |
| 348 | KE136354.1 | Enterococcus dispar ATCC 51266 genomic scaffold |
| | | acpMG-supercont1.1, whole genome shotgun |
| | | sequence. |
| 349 | GL833121.1 | Aureococcus anophagefferens unplaced genomic |
| | | scaffold AURANscaffold_2, whole genome shotgun |
| | | sequence. |
| 350 | FN430284.1 | Tuber melanosporum whole genome shotgun |
| | | sequence assembly, scaffold_368, strain Mel28 |
| 351 | MNXD01000034.1 | Candidatus Gracilibacteria bacterium |
| | | CG1_02_38_174 cg_0.2_sub10_scaffold_1404_c, |
| | | whole genome shotgun sequence. |
| 352 | BA000022.2 | Synechocystis sp. PCC 6803 DNA, complete genome. |
| 353 | LCND01000001.1 | Parcubacteria bacterium GW2011_GWA2_46_9 |
| | | UX68_C0001, whole genome shotgun sequence. |
| 354 | LNYB01000085.1 | Legionella feeleii strain WO-44C Lfee_ctg085, whole |
| | | genome shotgun sequence. |
| 355 | LNYW01000016.1 | Legionella shakespearei DSM 23087 strain ATCC |
| | | 49655 Lsha_ctg016, whole genome shotgun sequence. |
| 356 | LNZB01000060.1 | Legionella waltersii strain ATCC 51914 Lwal_ctg060, |
| | | whole genome shotgun sequence. |
| 357 | LNYG01000012.1 | Legionella jamestowniensis strain JA-26-G1-E2 |
| | | Ljam_ctg012, whole genome shotgun sequence. |
| 358 | LN614829.1 | Legionella fallonii LLAP-10 genome assembly, |
| | | plasmid: III |
| 359 | ALWS01092670.1 | Pteropus alecto contig92670, whole genome shotgun |
| | | sequence. |
| 360 | JMFR01091464.1 | Pterocles gutturalis contig91464, whole genome |
| | | shotgun sequence. |
| 361 | KB673645.1 | Anopheles dirus strain WRAIR2 unplaced genomic |
| | | scaffold supercont1.9, whole genome shotgun |
| | | sequence. |
| 362 | KB663666.1 | Anopheles minimus strain MINIMUS1 unplaced |
| | | genomic scaffold supercont1.15, whole genome |
| | | shotgun sequence. |
| 363 | AXCM01007520.1 | Anopheles culicifacies strain species A-37_1 |
| | | cont1.7520, whole genome shotgun sequence. |
| 364 | KB668664.1 | Anopheles funestus strain FUMOZ unplaced genomic |
| | | scaffold supercont1.144, whole genome shotgun |
| | | sequence. |
| 365 | KE525305.1 | Anopheles sinensis unplaced genomic scaffold |
| | | AS2_scf7180000696013, whole genome shotgun |
| | | sequence. |
| 366 | KI421903.1 | Anopheles atroparvus strain EBRO unplaced genomic |
| | | scaffold supercont1.22, whole genome shotgun |
| | | sequence. |
| 367 | APCM01004036.1 | Anopheles christyi strain ACHKN1017 cont1.4036, |
| | | whole genome shotgun sequence. |
| 368 | KB672913.1 | Anopheles dirus strain WRAIR2 unplaced genomic |
| | | scaffold supercont1.24, whole genome shotgun |
| | | sequence. |
| 369 | EQ090202.1 | Anopheles gambiae M scf_1925491374 genomic |
| | | scaffold, whole genome shotgun sequence. |
| 370 | KB704418.1 | Anopheles arabiensis strain DONG5_A unplaced |
| | | genomic scaffold supercont1.17, whole genome |
| | | shotgun sequence. |
| 371 | KB663706.1 | Anopheles minimus strain MINIMUS1 unplaced |
| | | genomic scaffold supercont1.186, whole genome |
| | | shotgun sequence. |
| 372 | AXCO02023244.1 | Anopheles melas strain CM1001059_A cont2.23244, |
| | | whole genome shotgun sequence. |
| 373 | APCM01005619.1 | Anopheles christyi strain ACHKN1017 cont1.5619, |
| | | whole genome shotgun sequence. |
| 374 | AXCL01009283.1 | Anopheles maculatus strain maculatus3 cont1.9278, |
| | | whole genome shotgun sequence. |
| 375 | EQ090214.1 | Anopheles gambiae M scf_1925491386 genomic |
| | | scaffold, whole genome shotgun sequence. |
| 376 | KE524837.1 | Anopheles sinensis unplaced genomic scaffold |
| | | AS2_scf7180000695538, whole genome shotgun |
| | | sequence. |
| 377 | KB670480.1 | Anopheles epiroticus strain epiroticus2 unplaced |
| | | genomic scaffold supercont1.178, whole genome |
| | | shotgun sequence. |
| 378 | CM000356.1 | Anopheles gambiae str. PEST chromosome 2L, whole |
| | | genome shotgun sequence. |
| 379 | APCM01003711.1 | Anopheles christyi strain ACHKN1017 cont1.3711, |
| | | whole genome shotgun sequence. |
| 380 | AXCL01028988.1 | Anopheles maculatus strain maculatus3 cont1.28980, |
| | | whole genome shotgun sequence. |
| 381 | AXCO02008943.1 | Anopheles melas strain CM1001059_A cont2.8943, |
| | | whole genome shotgun sequence. |
| 382 | KB664714.1 | Anopheles stephensi strain SDA-500 unplaced |
| | | genomic scaffold supercont1.383, whole genome |
| | | shotgun sequence. |
| 383 | APCM01002748.1 | Anopheles christyi strain ACHKN1017 cont1.2748, |
| | | whole genome shotgun sequence. |
| 384 | KB663721.1 | Anopheles minimus strain MINIMUS1 unplaced |
| | | genomic scaffold supercont1.2, whole genome |
| | | shotgun sequence. |
| 385 | KB664850.1 | Anopheles stephensi strain SDA-500 unplaced |
| | | genomic scaffold supercont1.505, whole genome |
| | | shotgun sequence. |
| 386 | KB672980.1 | Anopheles dirus strain WRAIR2 unplaced genomic |
| | | scaffold supercont1.30, whole genome shotgun |
| | | sequence. |
| 387 | KB663633.1 | Anopheles minimus strain MINIMUS1 unplaced |
| | | genomic scaffold supercont1.12, whole genome |
| | | shotgun sequence. |
| 388 | EQ087528.1 | Anopheles gambiae M scf_1925488698 genomic |
| | | scaffold, whole genome shotgun sequence. |
| 389 | KE525351.1 | Anopheles sinensis unplaced genomic scaffold |
| | | AS2_scf7180000696059, whole genome shotgun |
| | | sequence. |
| 390 | CM000357.1 | Anopheles gambiae str. PEST chromosome 2R, whole |
| | | genome shotgun sequence. |
| 391 | KB704784.1 | Anopheles arabiensis strain DONG5_A unplaced |
| | | genomic scaffold supercont1.5, whole genome |
| | | shotgun sequence. |
| 392 | KE525305.1 | Anopheles sinensis unplaced genomic scaffold |
| | | AS2_scf7180000696013, whole genome shotgun |
| | | sequence. |
| 393 | KI915351.1 | Anopheles merus strain MAF unplaced genomic |
| | | scaffold supercont2.196, whole genome shotgun |
| | | sequence. |
| 394 | KB670814.1 | Anopheles epiroticus strain epiroticus2 unplaced |
| | | genomic scaffold supercont1.208, whole genome |
| | | shotgun sequence. |
| 395 | ADMH02001348.1 | Anopheles darlingi Cont6653, whole genome shotgun |
| | | sequence. |
| 396 | KB664491.1 | Anopheles stephensi strain SDA-500 unplaced |
| | | genomic scaffold supercont1.182, whole genome |
| | | shotgun sequence. |
| 397 | AAAB01008842.1 | Anopheles gambiae str. PEST whole genome shotgun |
| | | sequencing project, whole genome shotgun sequence. |
| 398 | KB672869.1 | Anopheles dirus strain WRAIR2 unplaced genomic |
| | | scaffold supercont1.20, whole genome shotgun |
| | | sequence. |
| 399 | AXCM01007295.1 | Anopheles culicifacies strain species A-37_1 |
| | | cont1.7295, whole genome shotgun sequence. |
| 400 | KB672924.1 | Anopheles dirus strain WRAIR2 unplaced genomic |
| | | scaffold supercont1.25, whole genome shotgun |
| | | sequence. |
| 401 | AXCM01008016.1 | Anopheles culicifacies strain species A-37_1 |
| | | cont1.8016, whole genome shotgun sequence. |
| 402 | KB665043.1 | Anopheles stephensi strain SDA-500 unplaced |
| | | genomic scaffold supercont1.68, whole genome |
| | | shotgun sequence. |
| 403 | KB663622.1 | Anopheles minimus strain MINIMUS1 unplaced |
| | | genomic scaffold supercont1.11, whole genome |
| | | shotgun sequence. |
| 404 | KI915188.1 | Anopheles merus strain MAF unplaced genomic |
| | | scaffold supercont2.33, whole genome shotgun |
| | | sequence. |
| 405 | FR883402.1 | Clostridium sp. CAG: 221 genomic scaffold, scf67 |
| 406 | LRVM01000018.1 | [Clostridium] neopropionicum strain DSM-3847 |
| | | CLNEO_contig000018, whole genome shotgun |
| | | sequence. |
| 407 | FR891245.1 | Clostridium sp. CAG: 465 genomic scaffold, scf33 |
| 408 | LGGA01000028.1 | Atribacteria bacterium 34_128 MPI_scaffold_1295, |
| | | whole genome shotgun sequence. |
| 409 | HF993644.1 | Clostridium sp. CAG: 793 genomic scaffold, scf49 |
| 410 | CP013217.1 | Kurthia sp. 11kri321, complete genome. |
| 411 | CSXB01000014.1 | Mycobacterium abscessus strain PAP053 genome |
| | | assembly, contig: ERS075544SCcontig000014 |
| 412 | GG665866.1 | Shuttleworthia satelles DSM 14600 genomic scaffold |
| | | Scfld0, whole genome shotgun sequence. |
| 413 | CP000721.1 | Clostridium beijerinckii NCIMB 8052, complete |
| | | genome. |
| 414 | FR897768.1 | Bacillus sp. CAG: 988 genomic scaffold, scf27 |
| 415 | CP000612.1 | Desulfotomaculum reducens MI-1, complete genome. |
| 416 | CP002360.1 | Mahella australiensis 50-1 BON, complete genome. |
| 417 | HF990741.1 | Clostridium sp. CAG: 7 genomic scaffold, scf260 |
| 418 | CP001983.1 | Bacillus megaterium QM B1551, complete genome. |
| 419 | CP000679.1 | Caldicellulosiruptor saccharolyticus DSM 8903, |
| | | complete genome. |
| 420 | LM995447.1 | [Clostridium] cellulosi genome assembly, |
| | | chromosome: I |
| 421 | FR880072.1 | Clostridium sp. CAG: 245 genomic scaffold, scf154 |
| 422 | CP000764.1 | Bacillus cereus subsp. cytotoxis NVH 391-98, |
| | | complete genome. |
| 423 | KK222758.1 | Staphylococcus aureus C0673 genomic scaffold |
| | | aedLz-supercont1.14, whole genome shotgun |
| | | sequence. |
| 424 | FQXM01000006.1 | Clostridium grantii DSM 8605 genome assembly, |
| | | contig: EJ34DRAFT_scaffold00005.5 |
| 425 | HF999313.1 | Clostridium bartlettii CAG: 1329 genomic scaffold, |
| | | scf11 |
| 426 | MEFT01000138.1 | Clostridium sp. SCN 57-10 ABT01_C0138, whole |
| | | genome shotgun sequence. |
| 427 | AE000513.1 | Deinococcus radiodurans R1 chromosome 1, complete |
| | | sequence. |
| 428 | AKKV01000005.1 | Fictibacillus macauensis ZFHKF-1 Contig05, whole |
| | | genome shotgun sequence. |
| 429 | EQ973344.1 | Clostridium methylpentosum DSM 5476 Scfld6 |
| | | genomic scaffold, whole genome shotgun sequence. |
| 430 | FR898135.1 | Clostridium sp. CAG: 470 genomic scaffold, scf38 |
| 431 | FQXV01000008.1 | Sporobacter termitidis DSM 10068 genome assembly, |
| | | contig: EK05DRAFT_scaffold00008.8 |
| 432 | GL538352.1 | Listeria grayi DSM 20601 genomic scaffold |
| | | SCAFFOLD1, whole genome shotgun sequence. |
| 433 | KI271673.1 | Oscillibacter sp. KLE 1745 genomic scaffold |
| | | Scaffold170, whole genome shotgun sequence. |
| 434 | CP003184.1 | Thermoanaerobacterium saccharolyticum JW SL- |
| | | YS485, complete genome. |
| 435 | APML01000007.1 | Gracilibacillus halophilus YIM-C55.5 contig_7, |
| | | whole genome shotgun sequence. |
| 436 | FRCF01000004.1 | Salinicoccus alkaliphilus DSM 16010 genome |
| | | assembly, contig: EJ97DRAFT_scaffold00003.3 |
| 437 | KB976103.1 | Butyricicoccus pullicaecorum 1.2 genomic scaffold |
| | | acBRa-supercont1.1, whole genome shotgun |
| | | sequence. |
| 438 | MNSY01000086.1 | Firmicutes bacterium CAG: 176_63_11 |
| | | Ley3_66761_scaffold_4747, whole genome shotgun |
| | | sequence. |
| 439 | LMZU01000039.1 | Microgenomates bacterium OLB23 |
| | | UZ22_OP11002CONTIG000039, whole genome |
| | | shotgun sequence. |
| 440 | ADFP01000071.1 | Pyramidobacter piscolens W5455 contig00008, whole |
| | | genome shotgun sequence. |
| 441 | LBMD01000017.1 | Bacillus sp. CHD6a contig17, whole genome shotgun |
| | | sequence. |
| 442 | FR899424.1 | Mycoplasma sp. CAG: 472 genomic scaffold, scf184 |
| 443 | CP000382.1 | Clostridium novyi NT, complete genome. |
| 444 | LVJI01000034.1 | Paenibacillus antarcticus strain CECT 5836 PBAT34, |
| | | whole genome shotgun sequence. |
| 445 | MFJY01000009.1 | Candidatus Gottesmanbacteria bacterium |
| | | RIFCSPLOWO2_01_FULL_48_11 |
| | | rifcsplowo2_01_scaffold_16357, whole genome |
| | | shotgun sequence. |
| 446 | GG666055.1 | Anaerococcus lactolyticus ATCC 51172 genomic |
| | | scaffold SCAFFOLD12, whole genome shotgun |
| | | sequence. |
| 447 | NIBQ01000002.1 | Enterococcus sp. 9D6_DIV0238 scaffold00002, |
| | | whole genome shotgun sequence. |
| 448 | FQZO01000003.1 | Clostridium amylolyticum strain DSM 21864 genome |
| | | assembly, contig: Ga0131114_103 |
| 449 | AWUE01022526.1 | Corchorus olitorius cultivar O-4 contig22559, whole |
| | | genome shotgun sequence. |
| 450 | CM003264.1 | Gossypium hirsutum cultivar TM-1 chromosome 15, |
| | | whole genome shotgun sequence |
| 451 | CM008305.1 | Astyanax mexicanus chromosome 6, whole genome |
| | | shotgun sequence. |
| 452 | BDDD01003557.1 | Cephalotus follicularis DNA, scaffold: scaffold3557, |
| | | isolate: St1. |
| 453 | MVGT01000217.1 | Macleaya cordata isolate BLH2017 scaffold525, |
| | | whole genome shotgun sequence. |
| 454 | MVGT01000535.1 | Macleaya cordata isolate BLH2017 scaffold7799, |
| | | whole genome shotgun sequence. |
| 455 | URS0000D6C49D_12908 | unclassified sequences type-P1 twister ribozyme |
| 456 | URS0000D669BF_12908 | unclassified sequences type-P1 twister ribozyme |
| 457 | BABG01005008.1 | Human gut metagenome DNA, contig sequence: In- |
| | | R_005008. |
| 458 | JMFP01107431.1 | Pygoscelis adeliae contig107431, whole genome |
| | | shotgun sequence. |
| 459 | JJRS01104940.1 | Acanthisitta chloris contig104940, whole genome |
| | | shotgun sequence. |
| 460 | AJIM01057739.1 | Chelonia mydas contig57739, whole genome shotgun |
| | | sequence. |
| 461 | JJRT01033602.1 | Struthio camelus australis contig33602, whole |
| | | genome shotgun sequence. |
| 462 | JMFM02047454.1 | Manacus vitellinus contig47454, whole genome |
| | | shotgun sequence. |
| 463 | AJIM01198956.1 | Chelonia mydas contig198956, whole genome |
| | | shotgun sequence. |
| 464 | AJIM01141094.1 | Chelonia mydas contig141094, whole genome |
| | | shotgun sequence. |
| 465 | AFYH01061484.1 | Latimeria chalumnae contig061484, whole genome |
| | | shotgun sequence. |
| 466 | JJRU01042547.1 | Picoides pubescens contig42547, whole genome |
| | | shotgun sequence. |
| 467 | ADON01108924.1 | Anas platyrhynchos breed Pekin duck contig108924, |
| | | whole genome shotgun sequence. |
| 468 | JMFV01091687.1 | Apaloderma vittatum contig91687, whole genome |
| | | shotgun sequence. |
| 469 | JMFR01086319.1 | Pterocles gutturalis contig86319, whole genome |
| | | shotgun sequence. |
| 470 | CM000102.4 | Gallus gallus isolate RJF #256 breed Red Jungle fowl, |
| | | inbred line UCD001 chromosome 10, whole genome |
| | | shotgun sequence. |
| 471 | CM001999.1 | Ficedula albicollis isolate OC2 chromosome 10, |
| | | whole genome shotgun sequence. |
| 472 | JJRP01035117.1 | Colius striatus contig35117, whole genome shotgun |
| | | sequence. |
| 473 | JJRV01032988.1 | Calypte anna contig32988, whole genome shotgun |
| | | sequence. |
| 474 | AFYH01209269.1 | Latimeria chalumnae contig209269, whole genome |
| | | shotgun sequence. |
| 475 | JJRJ01071858.1 | Merops nubicus contig71858, whole genome shotgun |
| | | sequence. |
| 476 | AFYH01106573.1 | Latimeria chalumnae contig106573, whole genome |
| | | shotgun sequence. |
| 477 | JJRC01072922.1 | Egretta garzetta contig72922, whole genome shotgun |
| | | sequence. |
| 478 | AKHW03006215.1 | Alligator mississippiensis ScZkoYb_152, whole |
| | | genome shotgun sequence. |
| 479 | AAIY01498693.1 | Echinops telfairi cont1.498693, whole genome |
| | | shotgun sequence. |
| 480 | D00721.1 | Chicory yellow mottle virus satellite RNA gene for |
| | | hypothetical protein, complete cds. |
| 481 | M21212.1 | Arabis mosaic virus small satellite RNA, complete |
| | | genome. |
| 482 | M14879.1 | Tobacco ringspot virus satellite RNA. |
| 483 | LKEX01021873.1 | Cyphomyrmex costatus contig21873, whole genome |
| | | shotgun sequence. |
| 484 | AFTI01028208.1 | Crassostrea gigas strain 05x7-T-G4-1.051#20 |
| | | contig28208, whole genome shotgun sequence. |
| 485 | FP929037.1 | Clostridium saccharolyticum-like K10 draft genome. |
| 486 | JEMT01023831.1 | Rhizophagus irregularis DAOM 197198w |
| | | jcf7180003189428, whole genome shotgun sequence. |
| 487 | AEAB01026452.1 | Camponotus floridanus CamFlo_1.0_4.contig2489, |
| | | whole genome shotgun sequence. |
| 488 | KN823065.1 | Tulasnella calospora MUT 4182 unplaced genomic |
| | | scaffold scaffold_124, whole genome shotgun |
| | | sequence. |
| 489 | NNAY01026514.1 | Trichomalopsis sarcophage strain Alberta |
| | | scaffold26742, whole genome shotgun sequence. |
| 490 | AECU01000025.1 | Faecalibacterium cf. prausnitzii KLE1255 |
| | | F_prausnitziiKLE1255.K95-1.0_Cont34.1, whole |
| | | genome shotgun sequence. |
| 491 | LM398097.1 | Hymenolepis nana genome assembly, scaffold: |
| | | HNAJ_contig0000132 |
| 492 | MNRE01000164.1 | Clostridiales bacterium 41_21_two_genomes |
| | | Ley3_66761_scaffold_672, whole genome shotgun |
| | | sequence. |
| 493 | KN823040.1 | Tulasnella calospora MUT 4182 unplaced genomic |
| | | scaffold scaffold_99, whole genome shotgun |
| | | sequence. |
| 494 | KK107279.1 | Cerapachys biroi unplaced genomic scaffold |
| | | scaffold278, whole genome shotgun sequence. |
| 495 | FR886101.1 | Clostridium clostridioforme CAG: 132 genomic |
| | | scaffold, scf345 |
| 496 | LL216641.1 | Heligmosomoides polygyrus genome assembly, |
| | | scaffold: HPBE_contig0009563 |
| 497 | GL341474.1 | Nasonia vitripennis unplaced genomic scaffold |
| | | ChrUn_0243, whole genome shotgun sequence. |
| 498 | HF994873.1 | Ruminococcus sp. CAG: 724 genomic scaffold, scf297 |
| 499 | LKEX01015289.1 | Cyphomyrmex costatus contig15289, whole genome |
| | | shotgun sequence. |
| 500 | FP929052.1 | Ruminococcus sp. 18P13 draft genome. |
| 501 | GL637601.1 | Caenorhabditis tropicalis strain JU1373 unplaced |
| | | genomic scaffold Scaffold629, whole genome shotgun |
| | | sequence. |
| 502 | LM407409.1 | Hymenolepis nana genome assembly, scaffold: |
| | | HNAJ_contig0006064 |
| 503 | FR897605.1 | Anaerotruncus sp. CAG: 390 genomic scaffold, scf127 |
| 504 | FP929045.1 | Faecalibacterium prausnitzii L2 6 draft genome. |
| 505 | NNAY01000035.1 | Trichomalopsis sarcophagae strain Alberta scaffold35, |
| | | whole genome shotgun sequence. |
| 506 | NNAY01018372.1 | Trichomalopsis sarcophagae strain Alberta |
| | | scaffold18563, whole genome shotgun sequence. |
| 507 | KN169778.1 | Steinernema glaseri strain NC unplaced genomic |
| | | scaffold GLAS_3282, whole genome shotgun |
| | | sequence. |
| 508 | JOOK01112482.1 | Oesophagostomum dentatum strain OD-Hann |
| | | O_dentatum-1.0_Cont728411.1, whole genome |
| | | shotgun sequence. |
| 509 | KQ965786.1 | Gonapodya prolifera JEL478 unplaced genomic |
| | | scaffold M427scaffold_56, whole genome shotgun |
| | | sequence. |
| 510 | KQ965870.1 | Gonapodya prolifera JEL478 unplaced genomic |
| | | scaffold M427scaffold_140, whole genome shotgun |
| | | sequence. |
| 511 | NNAY01015791.1 | Trichomalopsis sarcophagae strain Alberta |
| | | scaffold15944, whole genome shotgun sequence. |
| 512 | FBWL01000170.1 | Clostridium sp. C105KSO13 isolate C105KSO131 |
| | | genome assembly, contig: {contig170} |
| 513 | MNRE01000064.1 | Clostridiales bacterium 41_21_two_genomes |
| | | Ley3_66761_scaffold_1913, whole genome shotgun |
| | | sequence. |
| 514 | LKEX01010795.1 | Cyphomyrmex costatus contig10795, whole genome |
| | | shotgun sequence. |
| 515 | FR901357.1 | Ruminococcus sp. CAG: 353 genomic scaffold, scf176 |
| 516 | LM398231.1 | Hymenolepis nana genome assembly, scaffold: |
| | | HNAJ_scaffold0000733 |
| 517 | ABEG02002846.1 | Caenorhabditis brenneri strain PB2801 C_brenneri- |
| | | 6.0.1_Cont82.14, whole genome shotgun sequence. |
| 518 | BAAZ01007529.1 | Human gut metagenome DNA, contig sequence: F2- |
| | | X_007529. |
| 519 | ADJT01005907.1 | Uncultured Faecalibacterium sp. TS29_contig04278, |
| | | whole genome shotgun sequence. |
| 520 | ACII01000060.1 | Ruminococcus sp. 5_1_39B_FAA cont1.60, whole |
| | | genome shotgun sequence. |
| 521 | AACY021109846.1 | Marine metagenome 1577600, whole genome shotgun |
| | | sequence. |
| 522 | BAAX01032439.1 | Human gut metagenome DNA, contig sequence: F2- |
| | | V_032439. |
| 523 | BAAZ01004974.1 | Human gut metagenome DNA, contig sequence: F2- |
| | | X 004974. |
| 524 | BAAY01003903.1 | Human gut metagenome DNA, contig sequence: F2- |
| | | W_003903. |
| 525 | ABVR01000037.1 | Coprococcus comes ATCC 27758 C_comes- |
| | | 1.0.1_Cont1600, whole genome shotgun sequence. |
| 526 | AACY021449234.1 | Marine metagenome 1095527145240, whole genome |
| | | shotgun sequence. |
| 527 | AMPZ01025371.1 | Schistosoma haematobium scaffold1749_15, whole |
| | | genome shotgun sequence. |
| 528 | LM184686.1 | Schistosoma mattheei strain Denwood, Zambia |
| | | genome assembly, scaffold: SMTD_contig0008514 |
| 529 | LL877594.1 | Schistosoma margrebowiei strain Zambia genome |
| | | assembly, scaffold: SMRZ_scaffold0000569 |
| 530 | LM066427.1 | Schistosoma curassoni strain Dakar, Senegal genome |
| | | assembly, scaffold: SCUD_scaffold0001340 |
| 531 | AMPZ01016641.1 | Schistosoma haematobium scaffold839_8, whole |
| | | genome shotgun sequence. |
| 532 | LL960995.1 | Schistosoma rodhaini strain Burundi genome |
| | | assembly, scaffold: SROB_scaffold0004017 |
| 533 | LL962685.1 | Schistosoma rodhaini strain Burundi genome |
| | | assembly, scaffold: SROB_scaffold0005707 |
| 534 | LL959719.1 | Schistosoma rodhaini strain Burundi genome |
| | | assembly, scaffold: SROB_scaffold0002741 |
| 535 | LL001662.1 | Trichobilharzia regenti genome assembly, scaffold: |
| | | TRE_scaffold0001662 |
| 536 | AMPZ01012007.1 | Schistosoma haematobium scaffold572_14, whole |
| | | genome shotgun sequence. |
| 537 | LL038740.1 | Trichobilharzia regenti genome assembly, scaffold: |
| | | TRE_scaffold0038465 |
| 538 | AMPZ01005699.1 | Schistosoma haematobium scaffold265_6, whole |
| | | genome shotgun sequence. |
| 539 | LL960174.1 | Schistosoma rodhaini strain Burundi genome |
| | | assembly, scaffold: SROB_scaffold0003196 |
| 540 | HE601624.1 | Schistosoma mansoni strain Puerto Rico chromosome |
| | | 1, complete genome |
| 541 | HE601627.1 | Schistosoma mansoni strain Puerto Rico chromosome |
| | | 4, complete genome |
| 542 | LL878569.1 | Schistosoma margrebowiei strain Zambia genome |
| | | assembly, scaffold: SMRZ_scaffold0001143 |
| 543 | LL877199.1 | Schistosoma margrebowiei strain Zambia genome |
| | | assembly, scaffold: SMRZ_contig0000066 |
| 544 | LL964478.1 | Schistosoma rodhaini strain Burundi genome |
| | | assembly, scaffold: SROB_scaffold0007499 |
| 545 | LM149431.1 | Schistosoma mattheei strain Denwood, Zambia |
| | | genome assembly, scaffold: SMTD_scaffold0000113 |
| 546 | LL959395.1 | Schistosoma rodhaini strain Burundi genome |
| | | assembly, scaffold: SROB_scaffold0002417 |
| 547 | LL876856.1 | Schistosoma margrebowiei strain Zambia genome |
| | | assembly, scaffold: SMRZ_scaffold0000011 |
| 548 | LL238470.1 | Echinostoma caproni strain Egypt genome assembly, |
| | | scaffold: ECPE_scaffold0005374 |
| 549 | LM120165.1 | Schistosoma curassoni strain Dakar, Senegal genome |
| | | assembly, scaffold: SCUD_contig0027497 |
| 550 | LL039251.1 | Trichobilharzia regenti genome assembly, scaffold: |
| | | TRE_scaffold0038963 |
| 551 | LL957289.1 | Schistosoma rodhaini strain Burundi genome |
| | | assembly, scaffold: SROB_scaffold0000311 |
| 552 | LM169888.1 | Schistosoma mattheei strain Denwood, Zambia |
| | | genome assembly, scaffold: SMTD_scaffold0017800 |
| 553 | LL878022.1 | Schistosoma margrebowiei strain Zambia genome |
| | | assembly, scaffold: SMRZ_contig0000349 |
| 554 | LL003993.1 | Trichobilharzia regenti genome assembly, scaffold: |
| | | TRE_scaffold0003993 |
| 555 | LM067904.1 | Schistosoma curassoni strain Dakar, Senegal genome |
| | | assembly, scaffold: SCUD_scaffold0002666 |
| 556 | LGKD01170204.1 | Octopus bimaculoides Scaffold16004_contig_23, |
| | | whole genome shotgun sequence. |
| 557 | HE601630.1 | Schistosoma mansoni strain Puerto Rico chromosome |
| | | 7, complete genome |
| 558 | JACJ01014299.1 | Opisthorchis viverrini opera_v5_148.27, whole |
| | | genome shotgun sequence. |
| 559 | AMPZ01005908.1 | Schistosoma haematobium scaffold104_9, whole |
| | | genome shotgun sequence. |
| 560 | AMPZ01001461.1 | Schistosoma haematobium scaffold15_47, whole |
| | | genome shotgun sequence. |
| 561 | AMPZ01011692.1 | Schistosoma haematobium scaffold555_12, whole |
| | | genome shotgun sequence. |
| 562 | LL877183.1 | Schistosoma margrebowiei strain Zambia genome |
| | | assembly, scaffold: SMRZ_scaffold0000277 |
| 563 | AMPZ01013432.1 | Schistosoma haematobium scaffold631_7, whole |
| | | genome shotgun sequence. |
| 564 | AMPZ01007250.1 | Schistosoma haematobium scaffold313_14, whole |
| | | genome shotgun sequence. |
| 565 | LL957011.1 | Schistosoma rodhaini strain Burundi genome |
| | | assembly, scaffold: SROB_scaffold0000033 |
| 566 | LM069637.1 | Schistosoma curassoni strain Dakar, Senegal genome |
| | | assembly, scaffold: SCUD_scaffold0004111 |
| 567 | LL877504.1 | Schistosoma margrebowiei strain Zambia genome |
| | | assembly, scaffold: SMRZ_contig0000159 |
| 568 | HE601631.1 | Schistosoma mansoni strain Puerto Rico chromosome |
| | | W, complete genome |
| 569 | LL030011.1 | Trichobilharzia regenti genome assembly, scaffold: |
| | | TRE_scaffold0029912 |
| 570 | LL036185.1 | Trichobilharzia regenti genome assembly, scaffold: |
| | | TRE_scaffold0035981 |
| 571 | BAAZ01000382.1 | Human gut metagenome DNA, contig sequence: F2- |
| | | X_000382. |

### Annealing Regions

Polynucleotide compositions described herein can include two or more annealing regions, e.g., two or more annealing regions described herein. An annealing region, or pair of annealing regions, are those that contain a portion with a high degree of complementarity that promotes hybridization under suitable conditions.

An annealing region includes at least a complementary region described below. The high degree of complementarity of the complementary region promotes the association of annealing region pairs. Where a first annealing region (e.g., a 5′ annealing region) is located at or near the 5′ end of a linear RNA and a second annealing region (e.g., a 3′ annealing region) is located at or near the 3′ end of a linear RNA, association of the annealing regions brings the 5′ and 3′ ends into proximity. In some embodiments, this favors circularization of the linear RNA by ligation of the 5′ and 3′ ends.

In embodiments, an annealing region further includes a non-complementary region as described below. A non-complementary region can be added to the complementary region to allow for the ends of the RNA to remain flexible, unstructured, or less structured than the complementarity region. The availability of flexible and/or single-stranded free 5′ and 3′ ends supports ligation and therefore circularization efficiency.

In some embodiments, each annealing region includes 5 to 100 ribonucleotides (e.g., 5 to 80, 5 to 50, 5 to 30, 5 to 20, 10 to 100, 10 to 80, 10 to 50, or 10 to 30 ribonucleotides). In some embodiments, a 5′ annealing region includes 5 to 100 ribonucleotides (e.g., 5 to 80, 5 to 50, 5 to 30, 5 to 20, 10 to 100, 10 to 80, 10 to 50, or 10 to 30 ribonucleotides). In some embodiments, a 3′ annealing region includes 5 to 100 ribonucleotides.

### Complementary Regions

A complementary region is a region that favors association with a corresponding complementary region, under suitable conditions. For example, a pair of complementary regions can share a high degree of sequence complementarity (e.g., a first complementary region is the reverse complement of a second complementary region, at least in part). When two complementary regions associate (e.g., hybridize), they can form a highly structured secondary structure, such as a stem or stem loop.

In some embodiments, the polyribonucleotide includes a 5′ complementary region and a 3′ complementary region. In some embodiments, the 5′ complementary region has between 5 and 50 ribonucleotides (e.g., 5-40, 5-30, 5-20, 5-10, 10-50, 10-40, 10-30, 10-20, or 20-50 ribonucleotides). In some embodiments, the 3′ complementary region has between 5 and 50 ribonucleotides (e.g., 5-40, 5-30, 5-20, 5-10, 10-50, 10-40, 10-30, 10-20, or 20-50 ribonucleotides).

In some embodiments, the 5′ complementary region and the 3′ complementary region have between 50% and 100% sequence complementarity (e.g., between 60%-100%, 70%-100%, 80%-100%, 90%-100%, or 100% sequence complementarity).

In some embodiments, the 5′ complementary region and the 3′ complementary region have a free energy of binding of less than −5 kcal/mol (e.g., less than −10 kcal/mol, less than −20 kcal/mol, or less than −30 kcal/mol).

In some embodiments, the 5′ complementary region and the 3′ complementary region have a Tm of binding of at least 10° C., at least 15° C., at least 20° C., at least 30° C., at least 40° C., at least 50° C., at least 60° C., at least 70° C., at least 80° C., or at least 90° C.

In some embodiments, the 5′ complementary region and the 3′ complementary region include no more than 10 mismatches, e.g., 10, 9, 8, 7, 6, 5, 4, 3, or 2 mismatches, or 1 mismatch (i.e., when the 5′ complementary region and the 3′ complementary region hybridize to each other). A mismatch can be, e.g., a nucleotide in the 5′ complementary region and a nucleotide in the 3′ complementary region that are opposite each other (i.e., when the 5′ complementary region and the 3′ complementary region are hybridized) but that do not form a Watson-Crick base-pair. A mismatch can be, e.g., an unpaired nucleotide that forms a kink or bulge in either the 5′ complementary region or the 3′ complementary region. In some embodiments, the 5′ complementary region and the 3′ complementary region do not include any mismatches.

### Non-Complementary Regions

A non-complementary region is a region that disfavors association with a corresponding non-complementary region, under suitable conditions. For example, a pair of non-complementary regions can share a low degree of sequence complementarity (e.g., a first non-complementary region is not a reverse complement of a second non-complementary region). When two non-complementary regions are in proximity, they do not form a highly structured secondary structure, such as a stem or stem loop.

In some embodiments, the polyribonucleotide includes a 5′ non-complementary region and a 3′ non-complementary region. In some embodiments, the 5′ non-complementary region has between 5 and 50 ribonucleotides (e.g., 5-40, 5-30, 5-20, 5-10, 10-50, 10-40, 10-30, 10-20, or 20-50 ribonucleotides). In some embodiments, the 3′ non-complementary region has between 5 and 50 ribonucleotides (e.g., 5-40, 5-30, 5-20, 5-10, 10-50, 10-40, 10-30, 10-20, or 20-50 ribonucleotides).

In some embodiments the 5′ non-complementary region is located 5′ to the 5′ complementary region (e.g., between the 5′ self-cleaving ribozyme and the 5′ complementary region). In some embodiments, the 3′ non-complementary region is located 3′ to the 3′ complementary region (e.g., between the 3′ complementary region and the 3′ self-cleaving ribozyme).

In some embodiments, the 5′ non-complementary region and the 3′ non-complementary region have between 0% and 50% sequence complementarity (e.g., between 0%-40%, 0%-30%, 0%-20%, 0%-10%, or 0% sequence complementarity).

In some embodiments, the 5′ non-complementary region and the 3′ non-complementary region have a free energy of binding of greater than −5 kcal/mol.

In some embodiments, the 5′ complementary region and the 3′ complementary region have a Tm of binding of less than 10° C.

In some embodiments, the 5′ non-complementary region and the 3′ non-complementary region include at least 1, 2, 3, 4, 5, 6, 7, 8, 9, or 10 mismatches.

### Polyribonucleotide Cargo

A polyribonucleotide cargo described herein includes any sequence including at least one polyribonucleotide.

A polyribonucleotide cargo may, for example, include at least about 40 nucleotides, at least about 50 nucleotides, at least about 75 nucleotides, at least about 100 nucleotides, at least about 200 nucleotides, at least about 300 nucleotides, at least about 400 nucleotides, at least about 500 nucleotides, at least about 1,000 nucleotides, at least about 2,000 nucleotides, at least about 5,000 nucleotides, at least about 6,000 nucleotides, at least about 7,000 nucleotides, at least about 8,000 nucleotides, at least about 9,000 nucleotides, at least about 10,000 nucleotides, at least about 12,000 nucleotides, at least about 14,000 nucleotides, at least about 15,000 nucleotides, at least about 16,000 nucleotides, at least about 17,000 nucleotides, at least about 18,000 nucleotides, at least about 19,000 nucleotides, or at least about 20,000 nucleotides. In some embodiments, the polyribonucleotides cargo includes between 1-20,000 nucleotides, 1-10,000 nucleotides, 1-5,000 nucleotides, 100-20,000 nucleotide, 100-10,000 nucleotides, 100-5,000 nucleotides, 500-20,000 nucleotides, 500-10,000 nucleotides, 500-5,000 nucleotides, 1,000-20,000 nucleotides, 1,000-10,000 nucleotides, or 1,000-5,000 nucleotides.

In embodiments, the polyribonucleotide cargo includes one or multiple coding (or expression) sequences, wherein each coding sequence encodes a polypeptide. In embodiments, the polyribonucleotide cargo includes one or multiple noncoding sequences. In embodiments, the polynucleotide cargo consists entirely of non-coding sequence(s). In embodiments, the polyribonucleotide cargo includes a combination of coding (or expression) and noncoding sequences.

In embodiments, the polyribonucleotide cargo includes multiple copies (e.g., 2, 3, 4, 5, 6, 7, 8, 9, 10, or even more than 10) of a single coding sequence. For example, the polyribonucleotide can include multiple copies of a sequence encoding a single protein. In other embodiments, the polyribonucleotide cargo includes at least one copy (e.g., 1, 2, 3, 4, 5, 6, 7, 8, 9, 10, or even more than 10 copies) each of two or more (e.g., 2, 3, 4, 5, 6, 7, 8, 9, 10, or more than 10) different coding sequences. For example, the polynucleotide cargo can include two copies of a first coding sequence and three copies of a second coding sequence.

In embodiments, the polyribonucleotide cargo includes one or more copies of at least one non-coding sequence. In embodiments, the at least one non-coding RNA sequence includes at least one RNA selected from the group consisting of: an RNA aptamer, a long non-coding RNA (lncRNA), a transfer RNA-derived fragment (tRF), a transfer RNA (tRNA), a ribosomal RNA (rRNA), a small nuclear RNA (snRNA), a small nucleolar RNA (snoRNA), and a Piwi-interacting RNA (piRNA); or a fragment of any one of these RNAs. In embodiments, the at least one non-coding RNA sequence includes at least one regulatory RNA, e.g., at least one RNA selected from the group consisting of a microRNA (miRNA) or miRNA precursor (see, e.g., U.S. Pat. Nos. 8,395,023, 8,946,511, 8,410,334 or 10,570,414), a microRNA recognition site (see, e.g., U.S. Pat. Nos. 8,334,430 or 10,876,126), a small interfering RNA (siRNA) or siRNA precursor (such as, but not limited to, an RNA sequence that forms an RNA hairpin or RNA stem-loop or RNA stem) (see, e.g., U.S. Pat. Nos. 8,404,927 or 10,378,012), a small RNA recognition site (see, e.g., U.S. Pat. No. 9,139,838), a trans-acting siRNA (ta-siRNA) or ta-siRNA precursor (see, e.g., U.S. Pat. No. 8,030,473), a phased sRNA or phased RNA precursor (see, e.g., U.S. Pat. No. 8,404,928), a phased sRNA recognition site (see, e.g., U.S. Pat. No. 9,309,512), a miRNA decoy (see, e.g., U.S. Pat. Nos. 8,946,511 or 10,435,686), a miRNA cleavage blocker (see, e.g., U.S. Pat. No. 9,040,774), a cis-acting riboswitch, a trans-acting riboswitch, and a ribozyme; all of these cited US patents are incorporated in their entirety herein. In embodiments, the at least one non-coding RNA sequence includes an RNA sequence that is complementary or anti-sense to a target sequence, for example, a target sequence encoded by a messenger RNA or encoded by DNA of a subject genome; such an RNA sequence is useful, e.g., for recognizing and binding to a target sequence through Watson-Crick base-pairing. In embodiments, the polyribonucleotide cargo includes multiple copies (e.g., 2, 3, 4, 5, 6, 7, 8, 9, 10, or even more than 10) of a single noncoding sequence. For example, the polyribonucleotide can include multiple copies of a sequence encoding a single microRNA precursor or multiple copies of a guide RNA sequence. In other embodiments, the polyribonucleotide cargo includes at least one copy (e.g., 1, 2, 3, 4, 5, 6, 7, 8, 9, 10, or even more than 10 copies) each of two or more (e.g., 2, 3, 4, 5, 6, 7, 8, 9, 10, or more than 10) different noncoding sequences. In one example, the polynucleotide cargo includes two copies of a first noncoding sequence and three copies of a second noncoding sequence. In another example, the polyribonucleotide cargo includes at least one copy each of two or more different miRNA precursors. In another example, the polyribonucleotide cargo includes (a) an RNA sequence that is complementary or anti-sense to a target sequence, and (b) a ribozyme or aptamer.

In some embodiments, circular polyribonucleotides made as described herein are used as effectors in therapy and/or agriculture. For example, a circular polyribonucleotide made by the methods described herein (e.g., the cell-free methods described herein) can be administered to a subject (e.g., in a pharmaceutical, veterinary, or agricultural composition). In another example, a circular polyribonucleotide made by the methods described herein (e.g., the cell-free methods described herein) can be delivered to a cell.

In some embodiments, the circular polyribonucleotide includes any feature or any combination of features as disclosed in International Patent Publication No. WO2019/118919, which is hereby incorporated by reference in its entirety.

### Polypeptide Expression Sequences

In some embodiments, the circular polyribonucleotide described herein (e.g., the polyribonucleotide cargo of the circular polyribonucleotide) includes one or more expression sequences (i.e., coding sequences), wherein each expression sequence encodes a polypeptide. In some embodiments, the circular polyribonucleotide includes two, three, four, five, six, seven, eight, nine, ten or more expression sequences.

Each encoded polypeptide can be linear or branched. The polypeptide can have a length from about 5 to about 40,000 amino acids, about 15 to about 35,000 amino acids, about 20 to about 30,000 amino acids, about 25 to about 25,000 amino acids, about 50 to about 20,000 amino acids, about 100 to about 15,000 amino acids, about 200 to about 10,000 amino acids, about 500 to about 5,000 amino acids, about 1,000 to about 2,500 amino acids, or any range therebetween. In some embodiments, the polypeptide has a length of less than about 40,000 amino acids, less than about 35,000 amino acids, less than about 30,000 amino acids, less than about 25,000 amino acids, less than about 20,000 amino acids, less than about 15,000 amino acids, less than about 10,000 amino acids, less than about 9,000 amino acids, less than about 8,000 amino acids, less than about 7,000 amino acids, less than about 6,000 amino acids, less than about 5,000 amino acids, less than about 4,000 amino acids, less than about 3,000 amino acids, less than about 2,500 amino acids, less than about 2,000 amino acids, less than about 1,500 amino acids, less than about 1,000 amino acids, less than about 900 amino acids, less than about 800 amino acids, less than about 700 amino acids, less than about 600 amino acids, less than about 500 amino acids, less than about 400 amino acids, less than about 300 amino acids, or less can be useful.

Polypeptides included herein can include naturally occurring polypeptides or non-naturally occurring polypeptides. In some instances, the polypeptide can be a functional fragment or variant of a reference polypeptide (e.g., an enzymatically active fragment or variant of an enzyme). For example, the polypeptide can be a functionally active variant of any of the polypeptides described herein with at least 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, or 99% identity, e.g., over a specified region or over the entire sequence, to a sequence of a polypeptide described herein or a naturally occurring polypeptide. In some instances, the polypeptide can have at least 50% (e.g., at least 50%, 60%, 70%, 80%, 90%, 95%, 97%, 99%, or greater) identity to a protein of interest.

Some examples of a polypeptide include, but are not limited to, a fluorescent tag or marker, an antigen, a therapeutic polypeptide, or a polypeptide for agricultural applications.

A therapeutic polypeptide can be a hormone, a neurotransmitter, a growth factor, an enzyme (e.g., oxidoreductase, metabolic enzyme, mitochondrial enzyme, oxygenase, dehydrogenase, ATP-independent enzyme, lysosomal enzyme, desaturase), a cytokine, an antigen binding polypeptide (e.g., antigen binding antibody or antibody-like fragments, such as single chain antibodies, nanobodies or other Ig heavy chain and/or light chain containing polypeptides), an Fc fusion protein, an anticoagulant, a blood factor, a bone morphogenetic protein, an interferon, an interleukin, and a thrombolytic.

In some cases, the circular polyribonucleotide expresses a non-human protein.

A polypeptide for agricultural applications can be a bacteriocin, a lysin, an antimicrobial polypeptide, an antifungal polypeptide, a nodule C-rich peptide, a bacteriocyte regulatory peptide, a peptide toxin, a pesticidal polypeptide (e.g., insecticidal polypeptide and/or nematocidal polypeptide), an antigen binding polypeptide (e.g., antigen binding antibody or antibody-like fragments, such as single chain antibodies, nanobodies or other Ig heavy chain and/or light chain containing polypeptides), an enzyme (e.g., nuclease, amylase, cellulase, peptidase, lipase, chitinase), a peptide pheromone, and a transcription factor.

In some embodiments, the circular polyribonucleotide expresses an antibody, e.g., an antibody fragment, or a portion thereof. In some embodiments, the antibody expressed by the circular polyribonucleotide can be of any isotype, such as IgA, IgD, IgE, IgG, IgM. In some embodiments, the circular polyribonucleotide expresses a portion of an antibody, such as a light chain, a heavy chain, a Fc fragment, a CDR (complementary determining region), a Fv fragment, or a Fab fragment, a further portion thereof. In some embodiments, the circular polyribonucleotide expresses one or more portions of an antibody. For instance, the circular polyribonucleotide can include more than one expression sequence, each of which expresses a portion of an antibody, and the sum of which can constitute the antibody. In some cases, the circular polyribonucleotide includes one expression sequence coding for the heavy chain of an antibody, and another expression sequence coding for the light chain of the antibody. In some cases, when the circular polyribonucleotide is expressed in a cell or a cell-free environment, the light chain and heavy chain can be subject to appropriate modification, folding, or other post-translation modification to form a functional antibody.

In embodiments, polypeptides include multiple polypeptides, e.g., multiple copies of one polypeptide sequence, or multiple different polypeptide sequences. In embodiments, multiple polypeptides are connected by linker amino acids or spacer amino acids.

In embodiments, the polynucleotide cargo includes sequence encoding a signal peptide. Many signal peptide sequences have been described, for example, the Tat (Twin-arginine translocation) signal sequence is typically an N-terminal peptide sequence containing a consensus SRRxFLK “twin-arginine” motif, which serves to translocate a folded protein containing such a Tat signal peptide across a lipid bilayer. See also, e.g., the Signal Peptide Database publicly available at www[dot]signalpeptide[dot]de. Signal peptides are also useful for directing a protein to specific organelles; see, e.g., the experimentally determined and computationally predicted signal peptides disclosed in the Spdb signal peptide database, publicly available at proline[dot]bic[dot]nus[dot]edu[dot]sg/spdb.

In embodiments, the polynucleotide cargo includes sequence encoding a cell-penetrating peptide (CPP). Hundreds of CPP sequences have been described; see, e.g., the database of cell-penetrating peptides, CPPsite, publicly available at crdd[dot]osdd[dot]net/raghava/cppsite/. An example of a commonly used CPP sequence is a poly-arginine sequence, e.g., octoarginine or nonoarginine, which can be fused to the C-terminus of the CGI peptide.

In embodiments, the polynucleotide cargo includes sequence encoding a self-assembling peptide; see, e.g., Miki et al. (2021) Nature Communications, 21:3412, DOI: 10.1038/s41467-021-23794-6.

### Therapeutic Polypeptides

In some embodiments, the circular polyribonucleotide described herein (e.g., the polyribonucleotide cargo of the circular polyribonucleotide) includes at least one expression sequence encoding a therapeutic polypeptide. A therapeutic polypeptide is a polypeptide that when administered to or expressed in a subject provides some therapeutic benefit. Administration to a subject or expression in a subject of a therapeutic polypeptide can be used to treat or prevent a disease, disorder, or condition or a symptom thereof. In some embodiments, the circular polyribonucleotide encodes two, three, four, five, six, seven, eight, nine, ten or more therapeutic polypeptides.

In some embodiments, the circular polyribonucleotide includes an expression sequence encoding a therapeutic protein. The protein can treat the disease in the subject in need thereof. In some embodiments, the therapeutic protein can compensate for a mutated, under-expressed, or absent protein in the subject in need thereof. In some embodiments, the therapeutic protein can target, interact with, or bind to a cell, tissue, or virus in the subject in need thereof.

A therapeutic polypeptide can be a polypeptide that can be secreted from a cell, or localized to the cytoplasm, nucleus, or membrane compartment of a cell.

A therapeutic polypeptide can be a hormone, a neurotransmitter, a growth factor, an enzyme (e.g., oxidoreductase, metabolic enzyme, mitochondrial enzyme, oxygenase, dehydrogenase, ATP-independent enzyme, lysosomal enzyme, desaturase), a cytokine, a transcription factor, an antigen binding polypeptide (e.g., antigen binding antibody or antibody-like fragments, such as single chain antibodies, nanobodies or other Ig heavy chain and/or light chain containing polypeptides), an Fc fusion protein, an anticoagulant, a blood factor, a bone morphogenetic protein, an interferon, an interleukin, a thrombolytic, an antigen (e.g., a tumor, viral, or bacterial antigen), a nuclease (e.g., an endonuclease such as a Cas protein, e.g., Cas9), a membrane protein (e.g., a chimeric antigen receptor (CAR), a transmembrane receptor, a G-protein-coupled receptor (GPCR), a receptor tyrosine kinase (RTK), an antigen receptor, an ion channel, or a membrane transporter), a secreted protein, a gene editing protein (e.g., a CRISPR-Cas, TALEN, or zinc finger), or a gene writing protein (see, e.g., International Patent Application Publication WO/2020/047124, incorporated in its entirety herein by reference).

In some embodiments, the therapeutic polypeptide is an antibody, e.g., a full-length antibody, an antibody fragment, or a portion thereof. In some embodiments, the antibody expressed by the circular polyribonucleotide can be of any isotype, such as IgA, IgD, IgE, IgG, IgM. In some embodiments, the circular polyribonucleotide expresses a portion of an antibody, such as a light chain, a heavy chain, a Fc fragment, a CDR (complementary determining region), a Fv fragment, or a Fab fragment, a further portion thereof. In some embodiments, the circular polyribonucleotide expresses one or more portions of an antibody. For instance, the circular polyribonucleotide can include more than one expression sequence, each of which expresses a portion of an antibody, and the sum of which can constitute the antibody. In some cases, the circular polyribonucleotide includes one expression sequence coding for the heavy chain of an antibody, and another expression sequence coding for the light chain of the antibody. When the circular polyribonucleotide is expressed in a cell, the light chain and heavy chain can be subject to appropriate modification, folding, or other post-translation modification to form a functional antibody.

In some embodiments, circular polyribonucleotides made as described herein are used as effectors in therapy and/or agriculture. For example, a circular polyribonucleotide made by the methods described herein (e.g., the cell-free methods described herein) can be administered to a subject (e.g., in a pharmaceutical, veterinary, or agricultural composition). In embodiments, the subject is a vertebrate animal (e.g., mammal, bird, fish, reptile, or amphibian). In embodiments, the subject is a human. In embodiments, the method subject is a non-human mammal. In embodiments, the subject is a non-human mammal such as a non-human primate (e.g., monkeys, apes), ungulate (e.g., cattle, buffalo, sheep, goat, pig, camel, llama, alpaca, deer, horses, donkeys), carnivore (e.g., dog, cat), rodent (e.g., rat, mouse), or lagomorph (e.g., rabbit). In embodiments, the subject is a bird, such as a member of the avian taxa Galliformes (e.g., chickens, turkeys, pheasants, quail), Anseriformes (e.g., ducks, geese), Paleaognathae (e.g., ostriches, emus), Columbiformes (e.g., pigeons, doves), or Psittaciformes (e.g., parrots). In embodiments, the subject is an invertebrate such as an arthropod (e.g., insects, arachnids, crustaceans), a nematode, an annelid, a helminth, or a mollusc. In embodiments, the subject is an invertebrate agricultural pest or an invertebrate that is parasitic on an invertebrate or vertebrate host. In embodiments, the subject is a plant, such as an angiosperm plant (which can be a dicot or a monocot) or a gymnosperm plant (e.g., a conifer, a cycad, a gnetophyte, a Ginkgo), a fern, horsetail, clubmoss, or a bryophyte. In embodiments, the subject is a eukaryotic alga (unicellular or multicellular). In embodiments, the subject is a plant of agricultural or horticultural importance, such as row crop plants, fruit-producing plants and trees, vegetables, trees, and ornamental plants including ornamental flowers, shrubs, trees, groundcovers, and turf grasses.

### Plant-Modifying Polypeptides

In some embodiments, the circular polyribonucleotide described herein (e.g., the polyribonucleotide cargo of the circular polyribonucleotide) includes at least one expression sequence encoding a plant-modifying polypeptide. A plant-modifying polypeptide refers to a polypeptide that can alter the genetic properties (e.g., increase gene expression, decrease gene expression, or otherwise alter the nucleotide sequence of DNA or RNA), epigenetic properties, or physiological or biochemical properties of a plant in a manner that results in an increase or decrease in plant fitness. In some embodiments, the circular polyribonucleotide encodes two, three, four, five, six, seven, eight, nine, ten or more different plant-modifying polypeptides, or multiple copies of one or more plant-modifying polypeptides. A plant-modifying polypeptide can increase the fitness of a variety of plants or can be one that targets one or more specific plants (e.g., a specific species or genera of plants).

Examples of polypeptides that can be used herein can include an enzyme (e.g., a metabolic recombinase, a helicase, an integrase, a RNAse, a DNAse, or a ubiquitination protein), a pore-forming protein, a signaling ligand, a cell penetrating peptide, a transcription factor, a receptor, an antibody, a nanobody, a gene editing protein (e.g., CRISPR-Cas endonuclease, TALEN, or zinc finger), a gene writing protein (see, e.g., International Patent Application Publication WO/2020/047124, incorporated in its entirety herein by reference), a riboprotein, a protein aptamer, or a chaperone.

### Agricultural Polypeptides

In some embodiments, the circular polyribonucleotide described herein (e.g., the polyribonucleotide cargo of the circular polyribonucleotide) includes at least one expression sequence encoding an agricultural polypeptide. An agricultural polypeptide is a polypeptide that is suitable for an agricultural use. In embodiments, an agricultural polypeptide is applied to a plant or seed (e.g., by foliar spray, dusting, injection, or seed coating) or to the plant's environment (e.g., by soil drench or granular soil application), resulting in an alteration of the plant's fitness. Embodiments of an agricultural polypeptide include polypeptides that alter a level, activity, or metabolism of one or more microorganisms resident in or on a plant or non-human animal host, the alteration resulting in an increase in the host's fitness. In some embodiments the agricultural polypeptide is a plant polypeptide. In some embodiments, the agricultural polypeptide is an insect polypeptide. In some embodiments, the agricultural polypeptide has a biological effect when contacted with a non-human vertebrate animal, invertebrate animal, microbial, or plant cell.

In some embodiments, the circular polyribonucleotide encodes two, three, four, five, six, seven, eight, nine, ten or more agricultural polypeptides, or multiple copies of one or more agricultural polypeptides.

Embodiments of polypeptides useful in agricultural applications include, for example, bacteriocins, lysins, antimicrobial peptides, nodule C-rich peptides, and bacteriocyte regulatory peptides. Such polypeptides can be used to alter the level, activity, or metabolism of target microorganisms for increasing the fitness of insects, such as honeybees and silkworms. Embodiments of agriculturally useful polypeptides include peptide toxins, such as those naturally produced by entomopathogenic bacteria (e.g., Bacillus thuringiensis, Photorhabdus luminescens, Serratia entomophila, or Xenorhabdus nematophila), as is known in the art. Embodiments of agriculturally useful polypeptides include polypeptides (including small peptides such as cyclodipeptides or diketopiperazines) for controlling agriculturally important pests or pathogens, e.g., antimicrobial polypeptides or antifungal polypeptides for controlling diseases in plants, or pesticidal polypeptides (e.g., insecticidal polypeptides and/or nematicidal polypeptides) for controlling invertebrate pests such as insects or nematodes. Embodiments of agriculturally useful polypeptides include antibodies, nanobodies, and fragments thereof, e.g., antibody or nanobody fragments that retain at least some (e.g., at least 10%) of the specific binding activity of the intact antibody or nanobody. Embodiments of agriculturally useful polypeptides include transcription factors, e.g., plant transcription factors; see, e.g., the “AtTFDB” database listing the transcription factor families identified in the model plant Arabidopsis thaliana), publicly available at agris-knowledgebase[dot]org/AtTFDB/. Embodiments of agriculturally useful polypeptides include nucleases, for example, exonucleases or endonucleases (e.g., Cas nucleases such as Cas9 or Cas12a). Embodiments of agriculturally useful polypeptides further include cell-penetrating peptides, enzymes (e.g., amylases, cellulases, peptidases, lipases, chitinases), peptide pheromones (for example, yeast mating pheromones, invertebrate reproductive and larval signaling pheromones, see, e.g., Altstein (2004) Peptides, 25:1373-1376).

Embodiments of agriculturally useful polypeptides confer a beneficial agronomic trait, e.g., herbicide tolerance, insect control, modified yield, increased fungal or oomycte disease resistance, increased virus resistance, increased nematode resistance, increased bacterial disease resistance, plant growth and development, modified starch production, modified oils production, high oil production, modified fatty acid content, high protein production, fruit ripening, enhanced animal and human nutrition, production of biopolymers, environmental stress resistance, pharmaceutical peptides and secretable peptides, improved processing traits, improved digestibility (e.g., reduced levels of toxins or reduced levels of compounds with “anti-nutritive” qualities such as lignins, lectins, and phytates), enzyme production, flavor, nitrogen fixation, hybrid seed production, fiber production, and biofuel production. Non-limiting examples of agriculturally useful polypeptides include polypeptides that confer herbicide resistance (U.S. Pat. Nos. 6,803,501; 6,448,476; 6,248,876; 6,225,114; 6,107,549; 5,866,775; 5,804,425; 5,633,435; and 5,463,175), increased yield (U.S. Pat. Nos. RE38,446; 6,716,474; 6,663,906; 6,476,295; 6,441,277; 6,423,828; 6,399,330; 6,372,211; 6,235,971; 6,222,098; and 5,716,837), insect control (U.S. Pat. Nos. 6,809,078; 6,713,063; 6,686,452; 6,657,046; 6,645,497; 6,642,030; 6,639,054; 6,620,988; 6,593,293; 6,555,655; 6,538,109; 6,537,756; 6,521,442; 6,501,009; 6,468,523; 6,326,351; 6,313,378; 6,284,949; 6,281,016; 6,248,536; 6,242,241; 6,221,649; 6,177,615; 6,156,573; 6,153,814; 6,110,464; 6,093,695; 6,063,756; 6,063,597; 6,023,013; 5,959,091; 5,942,664; 5,942,658, 5,880,275; 5,763,245; 5,763,241; 10,017,549; 10,233,217; 10,487,123; 10,494,408; 10,494,409; 10,611,806; 10,612,037; 10,669,317; 10,827,755; 11,254,950; 11,267,849; 11,130,965; 11,136,593; and 11,180,774), fungal disease resistance (U.S. Pat. Nos. 6,653,280; 6,573,361; 6,506,962; 6,316,407; 6,215,048; 5,516,671; 5,773,696; 6,121,436; 6,316,407; and 6,506,962), virus resistance (U.S. Pat. Nos. 6,617,496; 6,608,241; 6,015,940; 6,013,864; 5,850,023; and 5,304,730), nematode resistance (U.S. Pat. No. 6,228,992), bacterial disease resistance (U.S. Pat. No. 5,516,671), plant growth and development (U.S. Pat. Nos. 6,723,897 and 6,518,488), starch production (U.S. Pat. Nos. 6,538,181; 6,538,179; 6,538,178; 5,750,876; 6,476,295), modified oils production (U.S. Pat. Nos. 6,444,876; 6,426,447; and 6,380,462), high oil production (U.S. Pat. Nos. 6,495,739; 5,608,149; 6,483,008; and 6,476,295), modified fatty acid content (U.S. Pat. Nos. 6,828,475; 6,822,141; 6,770,465; 6,706,950; 6,660,849; 6,596,538; 6,589,767; 6,537,750; 6,489,461; and 6,459,018), high protein production (U.S. Pat. No. 6,380,466), fruit ripening (U.S. Pat. No. 5,512,466), enhanced animal and human nutrition (U.S. Pat. Nos. 6,723,837; 6,653,530; 6,5412,59; 5,985,605; and 6,171,640), biopolymers (U.S. Pat. Nos. RE37,543; 6,228,623; and U.S. Pat. Nos. 5,958,745, and 6,946,588), environmental stress resistance (U.S. Pat. No. 6,072,103), pharmaceutical peptides and secretable peptides (U.S. Pat. Nos. 6,812,379; 6,774,283; 6,140,075; and 6,080,560), improved processing traits (U.S. Pat. No. 6,476,295), improved digestibility (U.S. Pat. No. 6,531,648) low raffinose (U.S. Pat. No. 6,166,292), industrial enzyme production (U.S. Pat. No. 5,543,576), improved flavor (U.S. Pat. No. 6,011,199), nitrogen fixation (U.S. Pat. No. 5,229,114), hybrid seed production (U.S. Pat. No. 5,689,041), fiber production (U.S. Pat. Nos. 6,576,818; 6,271,443; 5,981,834; and 5,869,720) and biofuel production (U.S. Pat. No. 5,998,700).

### Secreted Polypeptide Effectors

In some embodiments, the circular polyribonucleotide described herein (e.g., the polyribonucleotide cargo of the circular polyribonucleotide) includes at least one coding sequence encoding a secreted polypeptide effector. Exemplary secreted polypeptide effectors or proteins that can be expressed include, e.g., cytokines and cytokine receptors, polypeptide hormones and receptors, growth factors, clotting factors, therapeutic replacement enzymes and therapeutic non-enzymatic effectors, regeneration, repair, and fibrosis factors, transformation factors, and proteins that stimulate cellular regeneration, non-limiting examples of which are described herein, e.g., in the tables below.

### Cytokines and Cytokine Receptors:

In some embodiments, an effector described herein comprises a cytokine of Table 3, or a functional variant or fragment thereof, e.g., a protein having at least 80%, 85%, 90%, 95%, 967%, 98%, 99% identity to a protein sequence disclosed in Table 3 by reference to its UniProt ID. In some embodiments, the functional variant binds to the corresponding cytokine receptor with a Kd of no more than 10%, 20%, 30%, 40%, or 50% higher or lower than the Kd of the corresponding wild-type cytokine for the same receptor under the same conditions. In some embodiments, the effector comprises a fusion protein comprising a first region (e.g., a cytokine polypeptide of Table 3 or a functional variant or fragment thereof) and a second, heterologous region. In some embodiments, the first region is a first cytokine polypeptide of Table 3. In some embodiments, the second region is a second cytokine polypeptide of Table 3, wherein the first and second cytokine polypeptides form a cytokine heterodimer with each other in a wild-type cell. In some embodiments, the polypeptide of Table 3 or functional variant thereof comprises a signal sequence, e.g., a signal sequence that is endogenous to the effector, or a heterologous signal sequence.

In some embodiments, an effector described herein comprises an antibody or fragment thereof that binds a cytokine of Table 3. In some embodiments, the antibody molecule comprises a signal sequence.

TABLE 3
Exemplary cytokines and cytokine receptors

| Cytokine | Cytokine receptor(s) | Entrez Gene ID¹ | UniProt ID² |
|---|---|---|---|
| IL-1α, IL-1β, | IL-1 type 1 receptor, | 3552, 3553 | P01583, P01584 |
| or a heterodimer thereof | IL-1 type 2 receptor | | |
| IL-1Ra | IL-1 type 1 receptor, | 3454, 3455 | P17181, P48551 |
| | IL-1 type 2 receptor | | |
| IL-2 | IL-2R | 3558 | P60568 |
| IL-3 | IL-3 receptor α + | 3562 | P08700 |
| | β c (CD131) | | |
| IL-4 | IL-4R type I, IL-4R | 3565 | P05112 |
| | type II | | |
| IL-5 | IL-5R | 3567 | P05113 |
| IL-6 | IL-6R (sIL-6R) gp130 | 3569 | P05231 |
| IL-7 | IL-7R and sIL-7R | 3574 | P13232 |
| IL-8 | CXCR1 and CXCR2 | 3576 | P10145 |
| IL-9 | IL-9R | 3578 | P15248 |
| IL-10 | IL-10R1/IL-10R2 complex | 3586 | P22301 |
| IL-11 | IL-11Rα 1 gp130 | 3589 | P20809 |
| IL-12 (e.g., p35, p40, | IL-12Rβ1 and | 3593, 3592 | P29459, P29460 |
| or a heterodimer thereof) | IL-12Rβ2 | | |
| IL-13 | IL-13R1α1 and | 3596 | P35225 |
| | IL-13R1α2 | | |
| IL-14 | IL-14R | 30685 | P40222 |
| IL-15 | IL-15R | 3600 | P40933 |
| IL-16 | CD4 | 3603 | Q14005 |
| IL-17A | IL-17RA | 3605 | Q16552 |
| IL-17B | IL-17RB | 27190 | Q9UHF5 |
| IL-17C | IL-17RA to IL-17RE | 27189 | Q9P0M4 |
| IL-17D | SEF | 53342 | Q8TAD2 |
| IL-17F | IL-17RA, IL-17RC | 112744 | Q96PD4 |
| IL-18 | IL-18 receptor | 3606 | Q14116 |
| IL-19 | IL-20R1/IL-20R2 | 29949 | Q9UHD0 |
| IL-20 | L-20R1/IL-20R2 and | 50604 | Q9NYY1 |
| | IL-22R1/IL-20R2 | | |
| IL-21 | IL-21R | 59067 | Q9HBE4 |
| IL-22 | IL-22R | 50616 | Q9GZX6 |
| IL-23 (e.g., p19, p40, | IL-23R | 51561 | Q9NPF7 |
| or a heterodimer thereof) | | | |
| IL-24 | IL-20R1/IL-20R2 and | 11009 | Q13007 |
| | IL-22R1/IL-20R2 | | |
| IL-25 | IL-17RA and IL-17RB | 64806 | Q9H293 |
| IL-26 | IL-10R2 chain and | 55801 | Q9NPH9 |
| | IL-20R1 chain | | |
| IL-27 (e.g., p28, EBI3, | WSX-1 and gp130 | 246778 | Q8NEV9 |
| or a heterodimer thereof) | | | |
| IL-28A, IL-28B, and IL29 | IL-28R1/IL-10R2 | 282617, 282618 | Q8IZI9, Q8IU54 |
| IL-30 | IL6R/gp130 | 246778 | Q8NEV9 |
| IL-31 | IL-31RA/OSMRβ | 386653 | Q6EBC2 |
| IL-32 | | 9235 | P24001 |
| IL-33 | ST2 | 90865 | O95760 |
| IL-34 | Colony-stimulating factor 1 | 146433 | Q6ZMJ4 |
| | receptor | | |
| IL-35 (e.g., p35, EBI3, | IL-12Rβ2/gp130; | 10148 | Q14213 |
| or a heterodimer thereof) | IL-12Rβ2/IL-12Rβ2; | | |
| | gp130/gp130 | | |
| IL-36 | IL-36Ra | 27179 | Q9UHA7 |
| IL-37 | IL-18Rα and IL-18BP | 27178 | Q9NZH6 |
| IL-38 | IL-1R1, IL-36R | 84639 | Q8WWZ1 |
| IFN-α | IFNAR | 3454 | P17181 |
| IFN-β | IFNAR | 3454 | P17181 |
| IFN-γ | IFNGR1/IFNGR2 | 3459 | P15260 |
| TGF-β | TβR-I and TβR-II | 7046, 7048 | P36897, P37173 |
| TNF-α | TNFR1, TNFR2 | 7132, 7133 | P19438, P20333 |

¹Sequence available on the NCBI database on the world wide web internet site “ncbi.nlm.nih.gov/gene”, Maglott D, et al. Gene: a gene-centered information resource at NCBI. Nucleic Acids Res. 2014. pii: gku1055.
²Sequence available on the Uniprot database on the world wide web internet site “uniprot.org/uniprot/”; UniProt: the universal protein knowledgebase in 2021. Nucleic Acids Res. 49: D1 (2021).

### Polypeptide Hormones and Receptors

In some embodiments, an effector described herein comprises a hormone of Table 4, or a functional variant thereof, e.g., a protein having at least 80%, 85%, 90%, 95%, 967%, 98%, 99% identity to a protein sequence disclosed in Table 4 by reference to its UniProt ID. In some embodiments, the functional variant binds to the corresponding receptor with a Kd of no more than 10%, 20%, 30%, 40%, or 50% higher than the Kd of the corresponding wild-type hormone for the same receptor under the same conditions. In some embodiments, the polypeptide of Table 4 or functional variant thereof comprises a signal sequence, e.g., a signal sequence that is endogenous to the effector, or a heterologous signal sequence.

In some embodiments, an effector described herein comprises an antibody molecule (e.g., an scFv) that binds a hormone of Table 4. In some embodiments, an effector described herein comprises an antibody molecule (e.g., an scFv) that binds a hormone receptor of Table 4. In some embodiments, the antibody molecule comprises a signal sequence.

TABLE 4
Exemplary polypeptide hormones and receptors

| Hormone | Receptor | Entrez Gene ID¹ | UniProt ID² |
|---|---|---|---|
| Natriuretic Peptide, e.g., | NPRA, NPRB, NPRC | 4878 | P01160 |
| Atrial Natriuretic Peptide | | | |
| (ANP) | | | |
| Brain Natriuretic Peptide | NPRA, NPRB | 4879 | P16860 |
| (BNP) | | | |
| C-type natriuretic peptide | NPRB | 4880 | P23582 |
| (CNP) | | | |
| Growth hormone (GH) | GHR | 2690 | P10912 |
| Prolactin (PRL) | PRLR | 5617 | P01236 |
| Thyroid-stimulating hormone | TSH receptor | 7253 | P16473 |
| (TSH) | | | |
| Adrenocorticotropic hormone | ACTH receptor | 5443 | P01189 |
| (ACTH) | | | |
| Follicle-stimulating hormone | FSHR | 2492 | P23945 |
| (FSH) | | | |
| Luteinizing hormone (LH) | LHR | 3973 | P22888 |
| Antidiuretic hormone (ADH) | Vasopressin receptors, | 554 | P30518 |
| | e.g., V2; AVPR1A; AVPR1B; | | |
| | AVPR3; AVPR2 | | |
| Oxytocin | OXTR | 5020 | P01178 |
| Calcitonin | Calcitonin receptor (CT) | 796 | P01258 |
| Parathyroid hormone (PTH) | PTH1R and PTH2R | 5741 | P01270 |
| Insulin | Insulin receptor (IR) | 3630 | P01308 |
| Glucagon | Glucagon receptor | 2641 | P01275 |
| GIP | GIPR | 2695 | P09681 |
| Fibroblast growth factor 19 | FGFR4 | 9965 | O95750 |
| (FGF19) | | | |
| Fibroblast growth factor 21 | FGFR1c, 2c, 3c | 26291 | Q9NSA1 |
| (FGF21) | | | |
| Fibroblast growth factor 23 | FGFR1, 2, 4 | 8074 | Q9GZV9 |
| (FGF23) | | | |
| Melanocyte-stimulating | MC1R, MC4R, MC5R | | |
| hormone (alpha- MSH) | | | |
| Melanocyte-stimulating | MC4R | | |
| hormone (beta- MSH) | | | |
| Melanocyte-stimulating | MC1R, MC3R, MC4R, | | |
| hormone (gamma- MSH) | MC5R | | |
| Proopiomelanocortin POMC | MC1R, MC3R, MC4R, | 5443 | P01189 |
| (alpha- beta-, gamma-, MSH | MC5R | | |
| precursor) | | | |
| Glycoprotein hormones alpha | | 1081 | P01215 |
| chain (CGA) | | | |
| Follicle-stimulating hormone | FSHR | 2488 | P01225 |
| beta (FSHB) | | | |
| Leptin | LEPR | 3952 | P41159 |
| Ghrelin | GHSR | 51738 | Q9UBU3 |

¹Sequence available on the NCBI database on the world wide web internet site “ncbi.nlm.nih.gov/gene”, Maglott D, et al. Gene: a gene-centered information resource at NCBI. Nucleic Acids Res. 2014. pii: gku1055.
²Sequence available on the Uniprot database on the world wide web internet site “uniprot.org/uniprot/”; UniProt: the universal protein knowledgebase in 2021. Nucleic Acids Res. 49: D1 (2021).

### Growth Factors:

In some embodiments, an effector described herein comprises a growth factor of Table 5, or a functional variant thereof, e.g., a protein having at least 80%, 85%, 90%, 95%, 967%, 98%, 99% identity to a protein sequence disclosed in Table 5 by reference to its UniProt ID. In some embodiments, the functional variant binds to the corresponding receptor with a Kd of no more than 10%, 20%, 30%, 40%, or 50% higher than the Kd of the corresponding wild-type growth factor for the same receptor under the same conditions. In some embodiments, the polypeptide of Table 5 or functional variant thereof comprises a signal sequence, e.g., a signal sequence that is endogenous to the effector, or a heterologous signal sequence.

In some embodiments, an effector described herein comprises an antibody or fragment thereof that binds a growth factor of Table 5. In some embodiments, an effector described herein comprises an antibody molecule (e.g., an scFv) that binds a growth factor receptor of Table 5. In some embodiments, the antibody molecule comprises a signal sequence.

TABLE 5
Exemplary growth factors

| | | Entrez Gene ID¹ | UniProt ID² |
|---|---|---|---|
| PDGF family | | | |
| PDGF (e.g., PDGF-1, | PDGF receptor, | 5156 | P16234 |
| PDGF-2, or a | e.g., PDGFRα, | | |
| heterodimer thereof) | PDGFRβ | | |
| CSF-1 | CSF1R | 1435 | P09603 |
| SCF | CD117 | 3815 | P10721 |
| VEGF family | | | |
| VEGF (e.g., isoforms | VEGFR-1, | 2321 | P17948 |
| VEGF 121, VEGF 165, | VEGFR-2 | | |
| VEGF 189, and VEGF | | | |
| 206) | | | |
| VEGF-B | VEGFR-1 | 2321 | P17949 |
| VEGF-C | VEGFR-2 and | 2324 | P35916 |
| | VEGFR -3 | | |
| PlGF | VEGFR-1 | 5281 | Q07326 |
| EGF family | | | |
| EGF | EGFR | 1950 | P01133 |
| TGF-α | EGFR | 7039 | P01135 |
| amphiregulin | EGFR | 374 | P15514 |
| HB-EGF | EGFR | 1839 | Q99075 |
| betacellulin | EGFR, ErbB-4 | 685 | P35070 |
| epiregulin | EGFR, ErbB-4 | 2069 | O14944 |
| Heregulin | EGFR, ErbB-4 | 3084 | Q02297 |
| FGF family | | | |
| FGF-1, FGF-2, FGF-3, | FGFR1, FGFR2, | 2246, 2247, 2248, 2249, | P05230, P09038, |
| FGF-4, FGF-5, FGF-6, | FGFR3, and FGFR4 | 2250, 2251, 2252, 2253, | P11487, P08620, |
| FGF-7, FGF-8, FGF-9 | | 2254 | P12034, P10767, |
| | | | P21781, P55075, P31371 |
| Insulin family | | | |
| Insulin | IR | 3630 | P01308 |
| IGF-I | IGF-I receptor, | 3479 | P05019 |
| | IGF-II receptor | | |
| IGF-II | IGF-II receptor | 3481 | P01344 |
| HGF family | | | |
| HGF | MET receptor | 3082 | P14210 |
| MSP | RON | 4485 | P26927 |
| Neurotrophin family | | | |
| NGF | LNGFR, trkA | 4803 | P01138 |
| BDNF | trkB | 627 | P23560 |
| NT-3 | trkA, trkB, trkC | 4908 | P20783 |
| NT-4 | trkA, trkB | 4909 | P34130 |
| NT-5 | trkA, trkB | 4909 | P34130 |
| Angiopoietin family | | | |
| ANGPT1 | HPK-6/TEK | 284 | Q15389 |
| ANGPT2 | HPK-6/TEK | 285 | O15123 |
| ANGPT3 | HPK-6/TEK | 9068 | O95841 |
| ANGPT4 | HPK-6/TEK | 51378 | Q9Y264 |
| ANGPTL2 | LILRB2 & integrin | 23452 | Q9UKU9 |
| | α5β1 | | |
| ANGPTL3 | LPL | 27329 | Q9Y5C1 |
| ANGPTL4 | | 51129 | Q9BY76 |
| ANGPTL8 | PirB | 55908 | Q6UXH0 |

¹Sequence available on the NCBI database on the world wide web internet site “ncbi.nlm.nih.gov/gene”, Maglott D, et al. Gene: a gene-centered information resource at NCBI. Nucleic Acids Res. 2014. pii: gku1055.
²Sequence available on the Uniprot database on the world wide web internet site “uniprot.org/uniprot/”; UniProt: the universal protein knowledgebase in 2021. Nucleic Acids Res. 49: D1 (2021).

### Clotting Factors:

In some embodiments, an effector described herein comprises a polypeptide of Table 6, or a functional variant thereof, e.g., a protein having at least 80%, 85%, 90%, 95%, 967%, 98%, 99% identity to a protein sequence disclosed in Table 6 by reference to its UniProt ID. In some embodiments, the functional variant catalyzes the same reaction as the corresponding wild-type protein, e.g., at a rate no less than 10%, 20%, 30%, 40%, or 50% lower or higher than the wild-type protein. In some embodiments, the polypeptide of Table 6 or functional variant thereof comprises a signal sequence, e.g., a signal sequence that is endogenous to the effector, or a heterologous signal sequence.

TABLE 6
Clotting-associated factors

| Effector | Indication | Entrez Gene ID¹ | UniProt ID² |
|---|---|---|---|
| Factor I | Afibrinogenomia | 2243, 2266, 2244 | P02671, P02679, P02675 |
| (fibrinogen) | | | |
| Factor II | Factor II Deficiency | 2147 | P00734 |
| Factor IX | Hemophilia B | 2158 | P00740 |
| Factor V | Owren's disease | 2153 | P12259 |
| Factor VIII | Hemophilia A | 2157 | P00451 |
| Factor X | Stuart-Prower Factor | 2159 | P00742 |
| | Deficiency | | |
| Factor XI | Hemophilia C | 2160 | P03951 |
| Factor XIII | Fibrin Stabilizing factor | 2162, 2165 | P00488, P05160 |
| | deficiency | | |
| vWF | von Willebrand disease | 7450 | P04275 |

¹Sequence available on the NCBI database on the world wide web internet site “ncbi.nlm.nih.gov/gene”, Maglott D, et al. Gene: a gene-centered information resource at NCBI. Nucleic Acids Res. 2014. pii: gku1055.
²Sequence available on the Uniprot database on the world wide web internet site “uniprot.org/uniprot/”; UniProt: the universal protein knowledgebase in 2021. Nucleic Acids Res. 49: D1 (2021).

### Therapeutic Replacement Enzymes:

In some embodiments, an effector described herein comprises an enzyme of Table 7, or functional variant thereof, e.g., a protein having at least 80%, 85%, 90%, 95%, 967%, 98%, 99% identity to a protein sequence disclosed in Table 7 by reference to its UniProt ID. In some embodiments, the functional variant catalyzes the same reaction as the corresponding wild-type protein, e.g., at a rate no less or no more than 10%, 20%, 30%, 40%, or 50% lower than the wild-type protein.

TABLE 7
Exemplary enzymatic effectors for enzyme deficiency

| Effector | Deficiency | Entrez Gene ID¹ | UniProt ID² |
|---|---|---|---|
| 3-methylcrotonyl-CoA | 3-methylcrotonyl-CoA | 56922, 64087 | Q96RQ3, Q9HCC0 |
| carboxylase | carboxylase deficiency | | |
| Acetyl-CoA- | Mucopolysaccharidosis MPS | 138050 | Q68CP4 |
| glucosaminide N- | III (Sanfilippo's syndrome) | | |
| acetyltransferase | Type III-C | | |
| ADAMTS13 | Thrombotic | 11093 | Q76LX8 |
| | Thrombocytopenic Purpura | | |
| adenine | Adenine | 353 | P07741 |
| phosphoribosyltransferase | phosphoribosyltransferase | | |
| | deficiency | | |
| Adenosine deaminase | Adenosine deaminase | 100 | P00813 |
| | deficiency | | |
| ADP-ribose protein | Glutamyl ribose-5-phosphate | 26119, 54936 | Q5SW96, Q9NX46 |
| hydrolase | storage disease | | |
| alpha glucosidase | Glycogen storage disease | 2548 | P10253 |
| | type 2 (Pompe's disease) | | |
| Arginase | Familial hyperarginemia | 383, 384 | P05089, P78540 |
| Arylsulfatase A | Metachromatic | 410 | P15289 |
| | leukodystrophy | | |
| Cathepsin K | Pycnodysostosis | 1513 | P43235 |
| Ceramidase | Farber's disease | 125981, 340485, 55331 | Q8TDN7, |
| | (lipogranulomatosis) | | Q5QJU3, Q9NUN7 |
| Cystathionine B | Homocystinuria | 875 | P35520 |
| synthase | | | |
| Dolichol-P-mannose | Congenital disorders of N- | 8813, 54344 | O60762, Q9P2X0 |
| synthase | glycosylation CDG Ie | | |
| Dolicho-P- | Congenital disorders of N- | 84920 | Q5BKT4 |
| Glc: Man9GlcNAc2-PP- | glycosylation CDG Ic | | |
| dolichol | | | |
| glucosyltransferase | | | |
| Dolicho-P- | Congenital disorders of N- | 10195 | Q92685 |
| Man: Man5GlcNAc2- | glycosylation CDG Id | | |
| PP-dolichol | | | |
| mannosyltransferase | | | |
| Dolichyl-P-glucose: Glc- | Congenital disorders of N- | 79053 | Q9BVK2 |
| 1-Man-9-GlcNAc-2-PP- | glycosylation CDG Ih | | |
| dolichyl-α-3- | | | |
| glucosyltransferase | | | |
| Dolichyl-P- | Congenital disorders of N- | 79087 | Q9BV10 |
| mannose: Man-7- | glycosylation CDG Ig | | |
| GlcNAc-2-PP-dolichyl- | | | |
| α-6-mannosyltransferase | | | |
| Factor II | Factor II Deficiency | 2147 | P00734 |
| Factor IX | Hemophilia B | 2158 | P00740 |
| Factor V | Owren's disease | 2153 | P12259 |
| Factor VIII | Hemophilia A | 2157 | P00451 |
| Factor X | Stuart-Prower Factor | 2159 | P00742 |
| | Deficiency | | |
| Factor XI | Hemophilia C | 2160 | P03951 |
| Factor XIII | Fibrin Stabilizing factor | 2162, 2165 | P00488, P05160 |
| | deficiency | | |
| Galactosamine-6-sulfate | Mucopolysaccharidosis MPS | 2588 | P34059 |
| sulfatase | IV (Morquio's syndrome) | | |
| | Type IV-A | | |
| Galactosylceramide β- | Krabbe's disease | 2581 | P54803 |
| galactosidase | | | |
| Ganglioside β- | GM1 gangliosidosis, | 2720 | P16278 |
| galactosidase | generalized | | |
| Ganglioside β- | GM2 gangliosidosis | 2720 | P16278 |
| galactosidase | | | |
| Ganglioside β- | Sphingolipidosis Type I | 2720 | P16278 |
| galactosidase | | | |
| Ganglioside β- | Sphingolipidosis Type II | 2720 | P16278 |
| galactosidase | (juvenile type) | | |
| Ganglioside β- | Sphingolipidosis Type III | 2720 | P16278 |
| galactosidase | (adult type) | | |
| Glucosidase I | Congenital disorders of N- | 2548 | P10253 |
| | glycosylation CDG IIb | | |
| Glucosylceramide β- | Gaucher's disease | 2629 | P04062 |
| glucosidase | | | |
| Heparan-S-sulfate | Mucopolysaccharidosis MPS | 6448 | P51688 |
| sulfamidase | III (Sanfilippo's syndrome) | | |
| | Type III-A | | |
| homogentisate oxidase | Alkaptonuria | 3081 | Q93099 |
| Hyaluronidase | Mucopolysaccharidosis MPS | 3373, 8692, | Q12794, Q12891, |
| | IX (hyaluronidase deficiency) | 8372, 23553 | O43820, Q2M3T9 |
| Iduronate sulfate | Mucopolysaccharidosis MPS | 3423 | P22304 |
| sulfatase | II (Hunter's syndrome) | | |
| Lecithin-cholesterol | Complete LCAT deficiency, | 3931 | 606967 |
| acyltransferase (LCAT) | Fish-eye disease, | | |
| | atherosclerosis, | | |
| | hypercholesterolemia | | |
| Lysine oxidase | Glutaric acidemia type I | 4015 | P28300 |
| Lysosomal acid lipase | Cholesteryl ester storage | 3988 | P38571 |
| | disease (CESD) | | |
| Lysosomal acid lipase | Lysosomal acid lipase | 3988 | P38571 |
| | deficiency | | |
| lysosomal acid lipase | Wolman's disease | 3988 | P38571 |
| Lysosomal pepstatin- | Ceroid lipofuscinosis Late | 1200 | O14773 |
| insensitive peptidase | infantile form (CLN2, | | |
| | Jansky-Bielschowsky | | |
| | disease) | | |
| Mannose (Man) | Congenital disorders of N- | 4351 | P34949 |
| phosphate (P) isomerase | glycosylation CDG Ib | | |
| Mannosyl-α-1,6- | Congenital disorders of N- | 4247 | Q10469 |
| glycoprotein-β-1,2-N- | glycosylation CDG IIa | | |
| acetylglucosminyltransferase | | | |
| Metalloproteinase-2 | Winchester syndrome | 4313 | P08253 |
| methylmalonyl-CoA | Methylmalonic acidemia | 4594 | P22033 |
| mutase | (vitamin b12 non-responsive) | | |
| N-Acetyl | Mucopolysaccharidosis MPS | 411 | P15848 |
| galactosamine α-4- | VI (Maroteaux-Lamy | | |
| sulfate sulfatase | syndrome) | | |
| (arylsulfatase B) | | | |
| N-acetyl-D- | Mucopolysaccharidosis MPS | 4669 | P54802 |
| glucosaminidase | III (Sanfilippo's syndrome) | | |
| | Type III-B | | |
| N-Acetyl- | Schindler's disease Type I | 4668 | P17050 |
| galactosaminidase | (infantile severe form) | | |
| N-Acetyl- | Schindler's disease Type II | 4668 | P17050 |
| galactosaminidase | (Kanzaki disease, adult-onset | | |
| | form) | | |
| N-Acetyl- | Schindler's disease Type III | 4668 | P17050 |
| galactosaminidase | (intermediate form) | | |
| N-acetyl-glucosaminine- | Mucopolysaccharidosis MPS | 2799 | P15586 |
| 6-sulfate sulfatase | III (Sanfilippo's syndrome) | | |
| | Type III-D | | |
| N-acetylglucosaminyl- | Mucolipidosis ML III | 79158 | Q3T906 |
| 1-phosphotransferase | (pseudo-Hurler's | | |
| | polydystrophy) | | |
| N-Acetylglucosaminyl- | Mucolipidosis ML II (I-cell | 79158 | Q3T906 |
| 1-phosphotransferase | disease) | | |
| catalytic subunit | | | |
| N-acetylglucosaminyl- | Mucolipidosis ML III | 84572 | Q9UJJ9 |
| 1-phosphotransferase, | (pseudo-Hurler's | | |
| substrate-recognition | polydystrophy) Type III-C | | |
| subunit | | | |
| N-Aspartylglucosaminidase | Aspartylglucosaminuria | 175 | P20933 |
| Neuraminidase 1 | Sialidosis | 4758 | Q99519 |
| (sialidase) | | | |
| Palmitoyl-protein | Ceroid lipofuscinosis Adult | 5538 | P50897 |
| thioesterase-1 | form (CLN4, Kufs' disease) | | |
| Palmitoyl-protein | Ceroid lipofuscinosis | 5538 | P50897 |
| thioesterase-1 | Infantile form (CLN1, | | |
| | Santavuori-Haltia disease) | | |
| Phenylalanine | Phenylketonuria | 5053 | P00439 |
| hydroxylase | | | |
| Phosphomannomutase-2 | Congenital disorders of N- | 5373 | O15305 |
| | glycosylation CDG Ia (solely | | |
| | neurologic and neurologic- | | |
| | multivisceral forms) | | |
| Porphobilinogen | Acute Intermittent Porphyria | 3145 | P08397 |
| deaminase | | | |
| Purine nucleoside | Purine nucleoside | 4860 | P00491 |
| phosphorylase | phosphorylase deficiency | | |
| pyrimidine 5′ | Hemolytic anemia and/or | 51251 | Q9H0P0 |
| nucleotidase | pyrimidine 5′ nucleotidase | | |
| | deficiency | | |
| Sphingomyelinase | Niemann-Pick disease type A | 6609 | P17405 |
| Sphingomyelinase | Niemann-Pick disease type B | 6609 | P17405 |
| Sterol 27-hydroxylase | Cerebrotendinous | 1593 | Q02318 |
| | xanthomatosis (cholestanol | | |
| | lipidosis) | | |
| Thymidine | Mitochondrial | 1890 | P19971 |
| phosphorylase | neurogastrointestinal | | |
| | encephalomyopathy | | |
| | (MNGIE) | | |
| Trihexosylceramide | Fabry's disease | 2717 | P06280 |
| α-galactosidase | | | |
| tyrosinase, e.g., OCA1 | albinism, e.g., ocular | 7299 | P14679 |
| | albinism | | |
| UDP-GlcNAc: dolichyl- | Congenital disorders of N- | 1798 | Q9H3H5 |
| P NAcGlc | glycosylation CDG Ij | | |
| phosphotransferase | | | |
| UDP-N- | Sialuria French type | 10020 | Q9Y223 |
| acetylglucosamine-2- | | | |
| epimerase/N- | | | |
| acetylmannosamine | | | |
| kinase, sialin | | | |
| Uricase | Lesch-Nyhan syndrome, gout | 391051 | No protein |
| uridine diphosphate | Crigler-Najjar syndrome | 54658 | P22309 |
| glucuronyl-transferase | | | |
| (e.g., UGT1A1) | | | |
| α-1,2- | Congenital disorders of N- | 79796 | Q9H6U8 |
| Mannosyltransferase | glycosylation CDG Il | | |
| | (608776) | | |
| α-1,2- | Congenital disorders of N- | 79796 | Q9H6U8 |
| Mannosyltransferase | glycosylation, type I (pre- | | |
| | Golgi glycosylation defects) | | |
| α-1,3- | Congenital disorders of N- | 440138 | Q2TAA5 |
| Mannosyltransferase | glycosylation CDG Ii | | |
| α-D-Mannosidase | α-Mannosidosis, type I | 10195 | Q92685 |
| | (severe) or II (mild) | | |
| α-L-Fucosidase | Fucosidosis | 4123 | Q9NTJ4 |
| α-l-Iduronidase | Mucopolysaccharidosis MPS | 2517 | P04066 |
| | I H/S (Hurler-Scheie | | |
| | syndrome) | | |
| α-l-Iduronidase | Mucopolysaccharidosis MPS | 3425 | P35475 |
| | I-H (Hurler's syndrome) | | |
| α-l-Iduronidase | Mucopolysaccharidosis MPS | 3425 | P35475 |
| | I-S (Scheie's syndrome) | | |
| β-1,4- | Congenital disorders of N- | 3425 | P35475 |
| Galactosyltransferase | glycosylation CDG IId | | |
| β-1,4- | Congenital disorders of N- | 2683 | P15291 |
| Mannosyltransferase | glycosylation CDG Ik | | |
| β-D-Mannosidase | β-Mannosidosis | 56052 | Q9BT22 |
| β-Galactosidase | Mucopolysaccharidosis MPS | 4126 | O00462 |
| | IV (Morquio's syndrome) | | |
| | Type IV-B | | |
| β-Glucuronidase | Mucopolysaccharidosis MPS | 2720 | P16278 |
| | VII (Sly's syndrome) | | |
| β-Hexosaminidase A | Tay-Sachs disease | 2990 | P08236 |
| β-Hexosaminidase B | Sandhoff's disease | 3073 | P06865 |

¹Sequence available on the NCBI database on the world wide web internet site “ncbi.nlm.nih.gov/gene”, Maglott D, et al. Gene: a gene-centered information resource at NCBI. Nucleic Acids Res. 2014. pii: gku1055.
²Sequence available on the Uniprot database on the world wide web internet site “uniprot.org/uniprot/”; UniProt: the universal protein knowledgebase in 2021. Nucleic Acids Res. 49: D1 (2021).

### Other Non-Enzymatic Effectors:

In some embodiments, a therapeutic polypeptide described herein comprises a polypeptide of Table 8, or a functional variant thereof, e.g., a protein having at least 80%, 85%, 90%, 95%, 967%, 98%, 99% identity to a protein sequence disclosed in Table 8 by reference to its UniProt ID.

TABLE 8
Exemplary non-enzymatic effectors and corresponding indications

| Effector | Indication | Entrez Gene ID¹ | UniProt ID² |
|---|---|---|---|
| Survival motor neuron | spinal muscular atrophy | 6606 | Q16637 |
| protein (SMN) | | | |
| Dystrophin | muscular dystrophy | 1756 | P11532 |
| | (e.g., Duchenne | | |
| | muscular dystrophy or | | |
| | Becker muscular | | |
| | dystrophy) | | |
| Complement protein, | Complement Factor I | 3426 | P05156 |
| e.g., Complement | deficiency | | |
| factor C1 | | | |
| Complement factor H | Atypical hemolytic | 3075 | P08603 |
| | uremic syndrome | | |
| Cystinosin (lysosomal | Cystinosis | 1497 | O60931 |
| cystine transporter) | | | |
| Epididymal secretory | Niemann-Pick disease | 10577 | P61916 |
| protein 1 (HE1; NPC2 | Type C2 | | |
| protein) | | | |
| GDP-fucose | Congenital disorders of | 55343 | Q96A29 |
| transporter-1 | N-glycosylation CDG | | |
| | IIc (Rambam-Hasharon | | |
| | syndrome) | | |
| GM2 activator protein | GM2 activator protein | 2760 | Q17900 |
| | deficiency (Tay-Sachs | | |
| | disease AB variant, | | |
| | GM2A) | | |
| Lysosomal | Ceroid lipofuscinosis | 1207 | Q13286 |
| transmembrane CLN3 | Juvenile form (CLN3, | | |
| protein | Batten disease, Vogt- | | |
| | Spielmeyer disease) | | |
| Lysosomal | Ceroid lipofuscinosis | 1203 | O75503 |
| transmembrane CLN5 | Variant late infantile | | |
| protein | form, Finnish type | | |
| | (CLN5) | | |
| Na phosphate | Infantile sialic acid | 26503 | Q9NRA2 |
| cotransporter, sialin | storage disorder | | |
| Na phosphate | Sialuria Finnish type | 26503 | Q9NRA2 |
| cotransporter, sialin | (Salla disease) | | |
| NPC1 protein | Niemann-Pick disease | 4864 | O15118 |
| | Type C1/Type D | | |
| Oligomeric Golgi | Congenital disorders of | 91949 | P83436 |
| complex-7 | N-glycosylation CDG | | |
| | IIe | | |
| Prosaposin | Prosaposin deficiency | 5660 | P07602 |
| Protective | Galactosialidosis | 5476 | P10619 |
| protein/cathepsin A | (Goldberg's syndrome, | | |
| (PPCA) | combined | | |
| | neuraminidase and β- | | |
| | galactosidase | | |
| | deficiency) | | |
| Protein involved in | Congenital disorders of | 9526 | O75352 |
| mannose-P-dolichol | N-glycosylation CDG If | | |
| utilization | | | |
| Saposin B | Saposin B deficiency | 5660 | P07602 |
| | (sulfatide activator | | |
| | deficiency) | | |
| Saposin C | Saposin C deficiency | 5660 | P07602 |
| | (Gaucher's activator | | |
| | deficiency) | | |
| Sulfatase-modifying | Mucosulfatidosis | 285362 | Q8NBK3 |
| factor-1 | (multiple sulfatase | | |
| | deficiency) | | |
| Transmembrane | Ceroid lipofuscinosis | 54982 | Q9NWW5 |
| CLN6 protein | Variant late infantile | | |
| | form (CLN6) | | |
| Transmembrane | Ceroid lipofuscinosis | 2055 | Q9UBY8 |
| CLN8 protein | Progressive epilepsy | | |
| | with intellectual | | |
| | disability | | |
| vWF | von Willebrand disease | 7450 | P04275 |
| Factor I (fibrinogen) | Afibrinogenomia | 2243, 2244, 2266 | P02671, P02675, |
| | | | P02679 |
| erythropoietin (hEPO) | | | |

¹Sequence available on the NCBI database on the world wide web internet site “ncbi.nlm.nih.gov/gene”, Maglott D, et al. Gene: a gene-centered information resource at NCBI. Nucleic Acids Res. 2014. pii: gku1055.
²Sequence available on the Uniprot database on the world wide web internet site “uniprot.org/uniprot/”; UniProt: the universal protein knowledgebase in 2021. Nucleic Acids Res. 49: D1 (2021).

### Regeneration, Repair and Fibrosis Factors

Therapeutic polypeptides described herein also include growth factors, e.g., as disclosed in Table 9, or functional variants thereof, e.g., a protein having at least 80%, 85%, 90%, 95%, 967%, 98%, 99% identity to a protein sequence disclosed in Table 9 by reference to its NCBI protein accession number. Also included are antibodies or fragments thereof against such growth factors, or miRNAs that promote regeneration and repair.

TABLE 9

| | NCBI Gene | |
|---|---|---|
| Target | accession # ¹ | NCBI Protein accession # ² |
| VEGF-A | NG_008732 | NP_001165094 |
| NRG-1 | NG_012005 | NP_001153471 |
| FGF2 | NG_029067 | NP_001348594 |
| FGF1 | Gene ID: 2246 | NP_001341882 |
| miR199-3p | MIMAT0000232 | n/a |
| miR590-3p | MIMAT0004801 | n/a |
| miR17-92 | MI0000071 | On the world wide web internet site |
| | | “ncbi.nlm.nih.gov/pmc/articles/PMC2732113/figure/F1/” |
| miR222 | MI0000299 | n/a |
| miR302-367 | MIR302A And | On the world wide web internet site |
| | | “ncbi.nlm.nih.gov/pmc/articles/PMC4400607/” |
| | MIR367 | |

¹ Sequence available on the world wide web internet site “ncbi.nlm.nih.gov/gene” (Maglott D, et al. Gene: a gene-centered information resource at NCBI. Nucleic Acids Res. 2014. Pii: gku1055.)
² Sequence available on the world wide web internet site “ncbi.nlm.nih.gov/protein/”

### Transformation Factors:

Therapeutic polypeptides described herein also include transformation factors, e.g., protein factors that transform fibroblasts into differentiated cell e.g., factors disclosed in Table 10 or functional variants thereof, e.g., a protein having at least 80%, 85%, 90%, 95%, 967%, 98%, 99% identity to a protein sequence disclosed in Table 10 by reference to its UniProt ID.

TABLE 10
Polypeptides indicated for organ repair by transforming fibroblasts

| | NCBI Gene | |
|---|---|---|
| Target | accession # ¹ | NCBI Protein accession # ² |
| MESP1 | Gene ID: 55897 | EAX02066 |
| ETS2 | GeneID: 2114 | NP_005230 |
| HAND2 | GeneID: 9464 | NP_068808 |
| MYOCARDIN | GeneID: 93649 | NP_001139784 |
| ESRRA | Gene ID: 2101 | AAH92470 |
| miR1 | MI0000651 | n/a |
| miR133 | MI000450 | n/a |
| TGFb | GeneID: 7040 | NP_000651.3 |
| WNT | Gene ID: 7471 | NP_005421 |
| JAK | Gene ID: 3716 | NP_001308784 |
| NOTCH | GeneID: 4851 | XP_011517019 |

¹ Sequence available on the world wide web internet site “ncbi.nlm.nih.gov/gene” (Maglott D, et al. Gene: a gene-centered information resource at NCBI. Nucleic Acids Res. 2014. Pii: gku1055.)
² Sequence available on the world wide web internet site “ncbi.nlm.nih.gov/protein/”

Proteins that Stimulate Cellular Regeneration:

Therapeutic polypeptides described herein also include proteins that stimulate cellular regeneration e.g., proteins disclosed in Table 11 or functional variants thereof, e.g., a protein having at least 80%, 85%, 90%, 95%, 967%, 98%, 99% identity to a protein sequence disclosed in Table 11 by reference to its UniProt ID.

TABLE 11

| Target | Gene accession # ¹ | Protein accession # ² |
|---|---|---|
| MST1 | NG_016454 | NP_066278 |
| STK30 | Gene ID: 26448 | NP_036103 |
| MST2 | Gene ID: 6788 | NP_006272 |
| SAV1 | Gene ID: 60485 | NP_068590 |
| LATS1 | Gene ID: 9113 | NP_004681 |
| LATS2 | Gene ID: 26524 | NP_055387 |
| YAP1 | NG_029530 | NP_001123617 |
| CDKN2b | NG_023297 | NP_004927 |
| CDKN2a | NG_007485 | NP_478102 |

¹ Sequence available on the world wide web internet site “ncbi.nlm.nih.gov/gene” (Maglott D, et al. Gene: a gene-centered information resource at NCBI. Nucleic Acids Res. 2014. Pii: gku1055.)
² Sequence available on the world wide web internet site “ncbi.nlm.nih.gov/protein/”

In some embodiments, the circular polyribonucleotide comprises one or more expression sequences (coding sequences) and is configured for persistent expression in a cell of a subject in vivo. In some embodiments, the circular polyribonucleotide is configured such that expression of the one or more expression sequences in the cell at a later time point is equal to or higher than an earlier time point. In such embodiments, the expression of the one or more expression sequences can be either maintained at a relatively stable level or can increase over time. The expression of the expression sequences can be relatively stable for an extended period of time. For instance, in some cases, the expression of the one or more expression sequences in the cell over a time period of at least 7, 8, 9, 10, 12, 14, 16, 18, 20, 22, 23 or more days does not decrease by 50%, 45%, 40%, 35%, 30%, 25%, 20%, 15%, 10%, or 5%. In some cases, in some cases, the expression of the one or more expression sequences in the cell is maintained at a level that does not vary by more than 50%, 45%, 40%, 35%, 30%, 25%, 20%, 15%, 10%, or 5% for at least 7, 8, 9, 10, 12, 14, 16, 18, 20, 22, 23 or more days.

### Internal Ribosomal Entry Sites (IRESs)

In some embodiments, the circular polyribonucleotide described herein (e.g., the polyribonucleotide cargo of the circular polyribonucleotide) includes one or more internal ribosome entry site (IRES) elements. In some embodiments, the IRES is operably linked to one or more expression sequences (e.g., each IRES is operably linked to one or more expression sequences). In embodiments, the IRES is located between a heterologous promoter and the 5′ end of a coding sequence.

A suitable IRES element to include in a circular polyribonucleotide includes an RNA sequence capable of engaging a eukaryotic ribosome. In some embodiments, the IRES element is at least about 5 nt, at least about 8 nt, at least about 9 nt, at least about 10 nt, at least about 15 nt, at least about 20 nt, at least about 25 nt, at least about 30 nt, at least about 40 nt, at least about 50 nt, at least about 100 nt, at least about 200 nt, at least about 250 nt, at least about 350 nt, or at least about 500 nt.

In some embodiments, the IRES element is derived from the DNA of an organism including, but not limited to, a virus, a mammal, and a Drosophila. Such viral DNA can be derived from, but is not limited to, picornavirus complementary DNA (cDNA), with encephalomyocarditis virus (EMCV) cDNA and poliovirus cDNA. In one embodiment, Drosophila DNA from which an IRES element is derived includes, but is not limited to, an Antennapedia gene from Drosophila melanogaster.

In some embodiments, if present, the IRES sequence is an IRES sequence of Taura syndrome virus, Triatoma virus, Theiler's encephalomyelitis virus, simian Virus 40, Solenopsis invicta virus 1, Rhopalosiphum padi virus, Reticuloendotheliosis virus, fuman poliovirus 1, Plautia stall intestine virus, Kashmir bee virus, Human rhinovirus 2, Homalodisca coagulata virus-1, Human Immunodeficiency Virus type 1, Homalodisca coagulata virus-1, Himetobi P virus, Hepatitis C virus, Hepatitis A virus, Hepatitis GB virus, foot and mouth disease virus, Human enterovirus 71, Equine rhinitis virus, Ectropis obliqua picorna-like virus, Encephalomyocarditis virus (EMCV), Drosophila C Virus, Crucifer tobamo virus, Cricket paralysis virus, Bovine viral diarrhea virus 1, Black Queen Cell Virus, Aphid lethal paralysis virus, Avian encephalomyelitis virus, Acute bee paralysis virus, Hibiscus chlorotic ringspot virus, Classical swine fever virus, Human FGF2, Human SFTPA1, Human AML1/RUNX1, Drosophila antennapedia, Human AQP4, Human AT1R, Human BAG-1, Human BCL2, Human BiP, Human c-IAP1, Human c-myc, Human eIF4G, Mouse NDST4L, Human LEF1, Mouse HIF1 alpha, Human n.myc, Mouse Gtx, Human p27kip1, Human PDGF2/c-sis, Human p53, Human Pim-1, Mouse Rbm3, Drosophila reaper, Canine Scamper, Drosophila Ubx, Human UNR, Mouse UtrA, Human VEGF-A, Human XIAP, Salivirus, Cosavirus, Parechovirus, Drosophila hairless, S. cerevisiae TFIID, S. cerevisiae YAP1, Human c-src, Human FGF-1, Simian picomavirus, Turnip crinkle virus, an aptamer to eIF4G, Coxsackievirus B3 (CVB3) or Coxsackievirus A (CVB1/2). In yet another embodiment, the IRES is an IRES sequence of Coxsackievirus B3 (CVB3). In a further embodiment, the IRES is an IRES sequence of Encephalomyocarditis virus.

In some embodiments, the circular polyribonucleotide includes at least one IRES flanking at least one (e.g., 2, 3, 4, 5 or more) expression sequence. In some embodiments, the IRES flanks both sides of at least one (e.g., 2, 3, 4, 5 or more) expression sequence. In some embodiments, the circular polyribonucleotide includes one or more IRES sequences on one or both sides of each expression sequence, leading to separation of the resulting peptide(s) and or polypeptide(s).

### Regulatory Elements

In some embodiments, the circular polyribonucleotide described herein (e.g., the polyribonucleotide cargo of the circular polyribonucleotide) includes one or more regulatory elements. In some embodiments, the circular polyribonucleotide includes a regulatory element, e.g., a sequence that modifies expression of an expression sequence within the circular polyribonucleotide.

A regulatory element can include a sequence that is located adjacent to an expression sequence that encodes an expression product. A regulatory element can be linked operatively to the adjacent sequence. A regulatory element can increase an amount of product expressed as compared to an amount of the expressed product when no regulatory element exists. In addition, one regulatory element can increase an amount of products expressed for multiple expression sequences attached in tandem. Hence, one regulatory element can enhance the expression of one or more expression sequences. Multiple regulatory elements are well-known to persons of ordinary skill in the art.

In some embodiments, the regulatory element is a translation modulator. A translation modulator can modulate translation of the expression sequence in the circular polyribonucleotide. A translation modulator can be a translation enhancer or suppressor. In some embodiments, the circular polyribonucleotide includes at least one translation modulator adjacent to at least one expression sequence. In some embodiments, the circular polyribonucleotide includes a translation modulator adjacent each expression sequence. In some embodiments, the translation modulator is present on one or both sides of each expression sequence, leading to separation of the expression products, e.g., peptide(s) and or polypeptide(s).

In some embodiments, the polyribonucleotide cargo includes at least one non-coding RNA sequence that includes a regulatory RNA. In some embodiments, the non-coding RNA sequence regulates a target sequence in trans. In some embodiments, the target sequence includes a nucleotide sequence of a gene of a subject genome, wherein the subject genome is a genome of a vertebrate animal, an invertebrate animal, a fungus, a plant, or a microbe. In embodiments, the subject genome is a genome of a human, a non-human mammal, a reptile, a bird, an amphibian, or a fish. In embodiments, the subject genome is a genome of an insect, an arachnid, a nematode, or a mollusk. In embodiments, the subject genome is a genome of a monocot, a dicot, a gymnosperm, or a eukaryotic alga. In embodiments, the subject genome is a genome of a bacterium, a fungus, or an archaeon. In embodiments, the target sequence comprises a nucleotide sequence of a gene found in multiple subject genomes (e.g., in the genome of multiple species within a given genus).

In some embodiments, the in trans regulation of the target sequence by the at least one non-coding RNA sequence is upregulation of expression of the target sequence. In some embodiments the in trans regulation of the target sequence by the at least one non-coding RNA sequence is downregulation of expression of the target sequence. In some embodiments, the trans regulation of the target sequence by the at least one non-coding RNA sequence is inducible expression of the target sequence. For example, the inducible expression can be inducible by an environmental condition (e.g., light, temperature, water, or nutrient availability), by circadian rhythm, by an endogenously or exogenously provided inducing agent (e.g., a small RNA, a ligand). In some embodiments, the at least one non-coding RNA sequence is inducible by the physiological state of the prokaryotic system (e.g., growth phase, transcriptional regulatory state, and intracellular metabolite concentration). For example, an exogenously provided ligand (e.g., arabinose, rhamnose, or IPTG) can be provided to induce expression using an inducible promoter (e.g., PBAD, Prha, and lacUV5).

In some embodiments, the at least one non-coding RNA sequence includes a regulatory RNA selected from the group consisting of: a small interfering RNA (siRNA) or a precursor thereof, a double-stranded RNA (dsRNA) or at least partially double-stranded RNA (e.g., RNA comprising one or more stem-loops); a hairpin RNA (hpRNA), a microRNA (miRNA) or precursor thereof (e.g., a pre-miRNA or a pri-miRNA); a phased small interfering RNA (phasiRNA) or precursor thereof; a heterochromatic small interfering RNA (hcsiRNA) or precursor thereof; and a natural antisense short interfering RNA (natsiRNA) or precursor thereof. In some embodiments, the at least one non-coding RNA sequence includes a guide RNA (gRNA) or precursor thereof, or a heterologous RNA sequence that is recognizable and can be bound by a guide RNA. In some embodiments, the regulatory element is a microRNA (miRNA) or a miRNA binding site, or a siRNA or siRNA binding site.

In some embodiments, the circular polyribonucleotide described herein (e.g., the polyribonucleotide cargo of the circular polyribonucleotide) includes at least one agriculturally useful non-coding RNA sequence that when provided to a particular plant tissue, cell, or cell type confers a desirable characteristic, such as a desirable characteristic associated with plant morphology, physiology, growth, development, yield, product, nutritional profile, disease or pest resistance, and/or environmental or chemical tolerance. In embodiments, the agriculturally useful non-coding RNA sequence causes the targeted modulation of gene expression of an endogenous gene, for example via antisense (see e.g., U.S. Pat. No. 5,107,065); inhibitory RNA (“RNAi”, including modulation of gene expression via miRNA-, siRNA-, trans-acting siRNA-, and phased sRNA-mediated mechanisms, e.g., as described in published applications US 2006/0200878 and US 2008/0066206, and in U.S. patent application Ser. No. 11/974,469); or cosuppression-mediated mechanisms. In embodiments, the agriculturally useful non-coding RNA sequence is a catalytic RNA molecule (e.g., a ribozyme or a riboswitch; see e.g., US 2006/0200878) engineered to cleave a desired endogenous mRNA product. Agriculturally useful non-coding RNA sequences are known in the art, e.g., an anti-sense oriented RNA that regulates gene expression in plant cells is disclosed in U.S. Pat. Nos. 5,107,065 and 5,759,829, and a sense-oriented RNA that regulates gene expression in plants is disclosed in U.S. Pat. Nos. 5,283,184 and 5,231,020. Providing an agriculturally useful non-coding RNA to a plant cell can also be used to regulate gene expression in an organism associated with a plant, e.g., an invertebrate pest of the plant or a microbial pathogen (e.g., a bacterium, fungus, oomycete, or virus) that infects the plant, or a microbe that is associated (e.g., in a symbiosis) with an invertebrate pest of the plant.

### Translation Initiation Sequences

In some embodiments, the circular polyribonucleotide described herein (e.g., the polyribonucleotide cargo of the circular polyribonucleotide) includes at least one translation initiation sequence. In some embodiments, the circular polyribonucleotide includes a translation initiation sequence operably linked to an expression sequence.

In some embodiments, the circular polyribonucleotide encodes a polypeptide and can include a translation initiation sequence, e.g., a start codon. In some embodiments, the translation initiation sequence includes a Kozak or Shine-Dalgarno sequence. In some embodiments, the circular polyribonucleotide includes the translation initiation sequence, e.g., Kozak sequence, adjacent to an expression sequence. In some embodiments, the translation initiation sequence is a non-coding start codon. In some embodiments, the translation initiation sequence, e.g., Kozak sequence, is present on one or both sides of each expression sequence, leading to separation of the expression products. In some embodiments, the circular polyribonucleotide includes at least one translation initiation sequence adjacent to an expression sequence. In some embodiments, the translation initiation sequence provides conformational flexibility to the circular polyribonucleotide. In some embodiments, the translation initiation sequence is within a substantially single stranded region of the circular polyribonucleotide.

The circular polyribonucleotide can include more than 1 start codon such as, but not limited to, at least 2, at least 3, at least 4, at least 5, at least 6, at least 7, at least 8, at least 9, at least 10, at least 11, at least 12, at least 13, at least 14, at least 15, at least 16, at least 17, at least 18, at least 19, at least 20, at least 25, at least 30, at least 35, at least 40, at least 50, at least 60 or more than 60 start codons. Translation can initiate on the first start codon or can initiate downstream of the first start codon.

In some embodiments, the circular polyribonucleotide can initiate at a codon which is not the first start codon, e.g., AUG. Translation of the circular polyribonucleotide can initiate at an alternative translation initiation sequence, such as, but not limited to, ACG, AGG, AAG, CTG/CUG, GTG/GUG, ATA/AUA, ATT/AUU, TTG/UUG. In some embodiments, translation begins at an alternative translation initiation sequence under selective conditions, e.g., stress induced conditions. As a non-limiting example, the translation of the circular polyribonucleotide can begin at alternative translation initiation sequence, such as ACG. As another non-limiting example, the circular polyribonucleotide translation can begin at alternative translation initiation sequence, CTG/CUG. As yet another non-limiting example, the circular polyribonucleotide translation can begin at alternative translation initiation sequence, GTG/GUG. As yet another non-limiting example, the circular polyribonucleotide can begin translation at a repeat-associated non-AUG (RAN) sequence, such as an alternative translation initiation sequence that includes short stretches of repetitive RNA e.g., CGG, GGGGCC, CAG, CTG.

### Termination Elements

In some embodiments, the circular polyribonucleotide described herein (e.g., the polyribonucleotide cargo of the circular polyribonucleotide) includes least one termination element. In some embodiments, the circular polyribonucleotide includes a termination element operably linked to an expression sequence.

In some embodiments, the circular polyribonucleotide includes one or more expression sequences, and each expression sequence can optionally have a termination element. In some embodiments, the circular polyribonucleotide includes one or more expression sequences, and the expression sequences lack a termination element, such that the circular polyribonucleotide is continuously translated. Exclusion of a termination element can result in rolling circle translation or continuous expression of expression product.

### Non-Coding Sequences

In some embodiments, the circular polyribonucleotide described herein (e.g., the polyribonucleotide cargo of the circular polyribonucleotide) includes one or more non-coding sequence, e.g., a sequence that does not encode the expression of polypeptide. In some embodiments, the circular polyribonucleotide includes two, three, four, five, six, seven, eight, nine, ten, or more than ten non-coding sequences. In some embodiments, the circular polyribonucleotide does not encode a polypeptide expression sequence.

Noncoding sequences can be natural or synthetic sequences. In some embodiments, a noncoding sequence can alter cellular behavior, such as e.g., lymphocyte behavior. In some embodiments, the noncoding sequences are antisense to cellular RNA sequences.

In some embodiments, the circular polyribonucleotide includes regulatory nucleic acids that are RNA or RNA-like structures typically between about 5-500 base pairs (bp), depending on the specific RNA structure (e.g., miRNA 5-30 bp, lncRNA 200-500 bp) and can have a nucleobase sequence identical (complementary) or nearly identical (substantially complementary) to a coding sequence in an expressed target gene within the cell. In embodiments, the circular polyribonucleotide includes regulatory nucleic acids that encode an RNA precursor that can be processed to a smaller RNA, e.g., a miRNA precursor, which can be from about 50 to about 1000 bp, that can be processed to a smaller miRNA intermediate or a mature miRNA.

Long non-coding RNAs (lncRNA) are defined as non-protein coding transcripts longer than 100 nucleotides. Many lncRNAs are characterized as tissue-specific. Divergent lncRNAs that are transcribed in the opposite direction to nearby protein-coding genes include a significant proportion (e.g., about 20% of total lncRNAs in mammalian genomes) and possibly regulate the transcription of the nearby gene. In one embodiment, the circular polyribonucleotide provided herein includes a sense strand of a lncRNA. In one embodiment, the circular polyribonucleotide provided herein includes an antisense strand of a lncRNA.

In embodiments, the circular polyribonucleotide encodes a regulatory nucleic acid that is substantially complementary, or fully complementary, to all or to at least one fragment of an endogenous gene or gene product (e.g., mRNA). In embodiments, the regulatory nucleic acids complement sequences at the boundary between introns and exons, in between exons, or adjacent to an exon, to prevent the maturation of newly generated nuclear RNA transcripts of specific genes into mRNA for transcription. The regulatory nucleic acids that are complementary to specific genes can hybridize with the mRNA for that gene and prevent its translation. The antisense regulatory nucleic acid can be DNA, RNA, or a derivative or hybrid thereof. In some embodiments, the regulatory nucleic acid includes a protein-binding site that can bind to a protein that participates in regulation of expression of an endogenous gene or an exogenous gene.

In embodiments, the circular polyribonucleotide encodes at least one regulatory RNA that hybridizes to a transcript of interest wherein the regulatory RNA has a length of between about 5 to 30 nucleotides, between about 10 to 30 nucleotides, or about 11, 12, 13, 14, 15, 16, 17, 18, 19, 20, 21, 22, 23, 24, 25, 26, 27, 28, 29, 30, or more than 30 nucleotides. In embodiments, the degree of sequence identity of the regulatory nucleic acid to the targeted transcript is at least 75%, at least 80%, at least 85%, at least 90%, or at least 95%.

In embodiments, the circular polyribonucleotide encodes a microRNA (miRNA) molecule identical to about 5 to about 25 contiguous nucleotides of a target gene, or encodes a precursor to that miRNA. In some embodiments, the miRNA has a sequence that allows the miRNA to recognize and bind to a specific target mRNA. In embodiments, the miRNA sequence commences with the dinucleotide AA, includes a GC-content of about 30-70% (about 30-60%, about 40-60%, or about 45%-55%), and does not have a high percentage identity to any nucleotide sequence other than the target in the genome of the subject (e.g., a mammal) in which it is to be introduced, for example as determined by standard BLAST search.

In some embodiments, the circular polyribonucleotide includes at least one miRNA (or miRNA precursor), e.g., 2, 3, 4, 5, 6, or more miRNAs or miRNA precursors. In some embodiments, the circular polyribonucleotide includes a sequence that encodes a miRNA (or its precursor) having at least about 75%, 80%, 85%, 90% 95%, 96%, 97%, 98%, 99% or 100% nucleotide complementarity to a target sequence.

siRNAs and shRNAs resemble intermediates in the processing pathway of the endogenous microRNA (miRNA) genes. In some embodiments, siRNAs can function as miRNAs and vice versa. MicroRNAs, like siRNAs, use RISC to downregulate target genes, but unlike siRNAs, most animal miRNAs do not cleave the mRNA. Instead, miRNAs reduce protein output through translational suppression or polyA removal and mRNA degradation. Known miRNA binding sites are within mRNA 3′ UTRs; miRNAs seem to target sites with near-perfect complementarity to nucleotides 2-8 from the miRNA's 5′ end. This region is known as the seed region. Because mature siRNAs and miRNAs are interchangeable, exogenous siRNAs downregulate mRNAs with seed complementarity to the siRNA.

Lists of known miRNA sequences can be found in databases maintained by research organizations, such as Wellcome Trust Sanger Institute, Penn Center for Bioinformatics, Memorial Sloan Kettering Cancer Center, and European Molecule Biology Laboratory, among others. Known effective siRNA sequences and cognate binding sites are also well represented in the relevant literature. RNAi molecules are readily designed and produced by technologies known in the art. In addition, there are computational tools that increase the chance of finding effective and specific sequence motifs.

Plant miRNAs, their precursors, and their target genes, are known in the art; see, e.g., U.S. Pat. Nos. 8,697,949, 8,946,511, and 9,040,774, and see also the publicly available microRNA database “miRbase” available at miRbase[dot]org. A naturally occurring miRNA or miRNA precursor sequence can be engineered or have its sequence modified in order for the resulting mature miRNA to recognize and bind to a target sequence of choice; examples of engineering both plant and animal miRNAs and miRNA precursors have been well demonstrated; see, e.g., U.S. Pat. Nos. 8,410,334, 8,536,405, and 9,708,620. All of the cited patents and the miRNA and miRNA precursors sequences disclosed therein are incorporated herein by reference.

### Spacer Sequences

In some embodiments, the circular polyribonucleotide described herein includes one or more spacer sequences. A spacer refers to any contiguous nucleotide sequence (e.g., of one or more nucleotides) that provides distance and/or flexibility between two adjacent polynucleotide regions. Spacers can be present in between any of the nucleic acid elements described herein. Spacers can also be present within a nucleic acid element described herein.

For example, wherein a nucleic acid includes any two or more of the following elements: (A) a 5′ self-cleaving ribozyme; (B) a 5′ annealing region; (C) a polyribonucleotide cargo; (D) a 3′ annealing region; and/or (E) a 3′ self-cleaving ribozyme; a spacer region can be present between any one or more of the elements. Any of elements (A), (B), (C), (D), and/or (E) can be separated by a spacer sequence, as described herein. For example, there can be a spacer between (A) and (B), between (B) and (C), between (C) and (D), and/or between (D) and (E).

Spacers can also be present within a nucleic acid region described herein. For example, a polynucleotide cargo region can include one or multiple spacers. Spacers can separate regions within the polynucleotide cargo.

In some embodiments, the spacer sequence can be, for example, at least 5 nucleotides in length, at least 10 nucleotides in length, at least 15 nucleotides in length, or at least 30 nucleotides in length. In some embodiments, the spacer sequence is at least 7, 8, 9, 10, 11, 12, 13, 14, 15, 16, 17, 18, 19, 20, 25 or 30 nucleotides in length. In some embodiments, the spacer sequence is no more than 100, 90, 80, 70, 60, 50, 45, 40, 35 or 30 nucleotides in length. In some embodiments the spacer sequence is between 20 and 50 nucleotides in length. In certain embodiments, the spacer sequence is 10, 11, 12, 13, 14, 15, 16, 17, 18, 19, 20, 21, 22, 23, 24, 25, 26, 27, 28, 29, 30, 31, 32, 33, 34,35, 36, 37, 38, 39,40,41, 42, 43, 44, 45, 46, 47, 48, 49 or 50 nucleotides in length.

In some embodiments, the spacer region can be between 5 and 1000, 5 and 900, 5 and 800, 5 and 700, 5 and 600, 5 and 500, 5 and 400, 5 and 300, 5 and 200, 5 and 100, 100 and 200, 100 and 300, 100 and 400, 100 and 500, 100 and 600, 100 and 700, 100 and 800, 100 and 900, or 100 and 1000 polyribonucleotides in length between the 5′ annealing region and the polyribonucleotide cargo. The spacer sequences can be polyA sequences, polyA-C sequences, polyC sequences, or poly-U sequences.

A spacer sequences can be used to separate an IRES from adjacent structural elements to maintain the structure and function of the IRES or the adjacent element. A spacer can be specifically engineered depending on the IRES. In some embodiments, an RNA folding computer software, such as RNAFold, can be utilized to guide designs of the various elements of the vector, including the spacers.

In some embodiments, the polyribonucleotide includes a 5′ spacer sequence (e.g., between the 5′ annealing region and the polyribonucleotide cargo). In some embodiments, the 5′ spacer sequence is at least 10 nucleotides in length. In another embodiment, the 5′ spacer sequence is at least 15 nucleotides in length. In a further embodiment, the 5′ spacer sequence is at least 30 nucleotides in length. In some embodiments, the 5′ spacer sequence is at least 7, 8, 9, 10, 11, 12, 13, 14, 15, 16, 17, 18, 19, 20, 25 or 30 nucleotides in length. In some embodiments, the 5′ spacer sequence is no more than 100, 90, 80, 70, 60, 50, 45, 40, 35 or 30 nucleotides in length. In some embodiments the 5′ spacer sequence is between 20 and 50 nucleotides in length. In certain embodiments, the 5′ spacer sequence is 10, 11, 12, 13, 14, 15, 16, 17, 18, 19, 20, 21, 22, 23, 24, 25, 26, 27, 28, 29, 30, 31, 32, 33, 34, 35, 36 37, 38, 39, 40, 41, 42, 43, 44, 45, 46, 47, 48, 49 or 50 nucleotides in length. In one embodiment, the 5′ spacer sequence is a polyA sequence. In another embodiment, the 5′ spacer sequence is a polyA-C sequence.

In some embodiments, the polyribonucleotide includes a 3′ spacer sequence (e.g., between the 3′ annealing region and the polyribonucleotide cargo). In some embodiments, the 3′ spacer sequence is at least 10 nucleotides in length. In another embodiment, the 3′ spacer sequence is at least 15 nucleotides in length. In a further embodiment, the 3′ spacer sequence is at least 30 nucleotides in length. In some embodiments, the 3′ spacer sequence is at least 7, 8, 9, 10, 11, 12, 13, 14, 15, 16, 17, 18, 19, 20, 25 or 30 nucleotides in length. In some embodiments, the 3′ spacer sequence is no more than 100, 90, 80, 70, 60, 50, 45, 40, 35 or 30 nucleotides in length. In some embodiments the 3′ spacer sequence is between 20 and 50 nucleotides in length. In certain embodiments, the 3′ spacer sequence is 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49 or 50 nucleotides in length. In one embodiment, the 3′ spacer sequence is a polyA sequence. In another embodiment, the 5′ spacer sequence is a polyA-C sequence.

In one embodiment, the polyribonucleotide includes a 5′ spacer sequence, but not a 3′ spacer sequence. In another embodiment, the polyribonucleotide includes a 3′ spacer sequence, but not a 5′ spacer sequence. In another embodiment, the polyribonucleotide includes neither a 5′ spacer sequence, nor a 3′ spacer sequence. In another embodiment, the polyribonucleotide does not include an IRES sequence. In a further embodiment, the polyribonucleotide does not include an IRES sequence, a 5′ spacer sequence or a 3′ spacer sequence.

In some embodiments, the spacer sequence includes at least 3 ribonucleotides, at least 4 ribonucleotides, at least 5 ribonucleotides, at least about 8 ribonucleotides, at least about 10 ribonucleotides, at least about 12 ribonucleotides, at least about 15 ribonucleotides, at least about 20 ribonucleotides, at least about 25 ribonucleotides, at least about 30 ribonucleotides, at least about 40 ribonucleotides, at least about 50 ribonucleotides, at least about 60 ribonucleotides, at least about 70 ribonucleotides, at least about 80 ribonucleotides, at least about 90 ribonucleotides, at least about 100 ribonucleotides, at least about 120 ribonucleotides, at least about 150 ribonucleotides, at least about 200 ribonucleotides, at least about 250 ribonucleotides, at least about 300 ribonucleotides, at least about 400 ribonucleotides, at least about 500 ribonucleotides, at least about 600 ribonucleotides, at least about 700 ribonucleotides, at least about 800 ribonucleotides, at least about 900 ribonucleotides, or at least about 100 ribonucleotides.

### Ligases

RNA ligases are a class of enzymes that utilize ATP to catalyze the formation of a phosphodiester bond between the ends of RNA molecules. Endogenous RNA ligases repair nucleotide breaks in single-stranded, duplexed RNA within plant, animal, human, bacterial, archaeal, and fungal cells—as well as viruses.

The present disclosure provides a method of producing circular RNA by contacting a linear RNA (e.g., a ligase-compatible linear RNA as described herein) with an RNA ligase.

In some embodiments, the RNA ligase in a tRNA ligase, or a variant thereof. In some embodiments the tRNA ligase is a T4 ligase, an RtcB ligase, a TRL-1 ligase, and Rnl1 ligase, an Rnl2 ligase, a LIG1 ligase, a LIG2 ligase a PNK/PNL ligase, a PF0027 ligase, a thpR ligT ligase, a ytlPor ligase, or a variant thereof (e.g., a mutational variant that retains ligase function).

In some embodiments, the RNA ligase is a plant RNA ligase or a variant thereof. In some embodiments, the RNA ligase is a chloroplast RNA ligase or a variant thereof. In embodiments, the RNA ligase is a eukaryotic algal RNA ligase or a variant thereof. In some embodiments, the RNA ligase is an RNA ligase from archaea or a variant thereof. In some embodiments, the RNA ligase is a bacterial RNA ligase or a variant thereof. In some embodiments, the RNA ligase is a eukaryotic RNA ligase or a variant thereof. In some embodiments, the RNA ligase is a viral RNA ligase or a variant thereof. In some embodiments, the RNA ligase is a mitochondrial RNA ligase or a variant thereof.

In some embodiments, the RNA ligase is a ligase described in Table 2, or a variant thereof.

TABLE 2
Exemplary tRNA ligases

| Organism | Domain | Gene | Protein | Uniprot ID |
|---|---|---|---|---|
| Pyrobaculum aerophilum | Archaea | rtcb | RNA-splicing | Q8ZY09 |
| | | | ligase RtcB | |
| Sulfolobus acidocaldarius | Archaea | rtcb | RNA-splicing | Q4J977 |
| (thermophile) | | | ligase RtcB | |
| Pyrococcus furiosus | Archaea | rtcb | RNA-splicing | Q8U0H4 |
| (thermophile) | | | ligase RtcB | |
| Bacillus cereus | Bacteria (Gram | rtcb | RNA-splicing | A0A2A8ZZV1 |
| | Positive) | | ligase RtcB | |
| Escherichia coli | Bacteria (Gram | rtcb | RNA-splicing | P46850 |
| (K12 strain) | Negative) | | ligase RtcB | |
| Caenorhabditis elegans | Eukarya | rtcb-1 | RNA-splicing | P90838 |
| | (Animalia) | | ligase RtcB | |
| | | | homolog | |
| Saccharomyces cerevisiae | Eukarya (Fungi) | TRL1 | tRNA ligase | P09880 |
| Arabidopsis thaliana | Eukarya (Plantae) | RNL | tRNA ligase 1 | Q0WL81 |
| Enterobacteria phage | Virus | Y10A | RNA ligase 2 | P32277 |
| T4 | | | | |
| Candida albicans | Eukarya (Fungi) | LIG1 | tRNA ligase | P43075 |
| Trypanosoma brucei | Eukarya | LIG1 | RNA-editing | P86926 |
| brucei | | | ligase 1, | |
| | | | mitochondrial | |
| Trypanosoma brucei | Eukarya | LIG2 | RNA-editing | P86924 |
| brucei | | | ligase 2, | |
| | | | mitochondrial | |
| Enterobacteria phage | Virus | Gene 63 | tRNA ligase 1 | P00971 |
| T4 | | | | |
| Autographa californica | Virus | PNK/PNL | Putative | P41476 |
| nuclear polyhedrosis | | | bifunctional | |
| virus (AcMNPV) | | | polynucleotide | |
| | | | kinase/RNA ligase | |
| Pyrococcus furiosus | Archaea | PF0027 | RNA 2′,3′-cyclic | Q8U4Q3 |
| (thermophile) | | | phosphodiesterase | |
| Escherichia coli | Bacteria (Gram | thpR ligT | RNA 2′,3′-cyclic | P37025 |
| (K12 strain) | Negative) | | phosphodiesterase | |
| Bacillus subtilis | Bacteria (Gram | ytlP | RNA 2′,3′-cyclic | O34570 |
| | Positive) | | phosphodiesterase | |

### Methods of Production

The disclosure also provides methods of producing a circular RNA in a cell-free system. FIG. 2 is a schematic that depicts an exemplary process for producing a circular RNA from a precursor linear RNA. For example, a deoxyribonucleotide template can be transcribed in a cell-free system (e.g., by in vitro transcription) to a produce a precursor linear RNA. Upon expression, under suitable conditions, and in no particular order, the 5′ and 3′ self-cleaving ribozymes each undergo a cleavage reaction thereby producing ligase-compatible ends (e.g., a 5′-hydroxyl and a 2′,3′-cyclic phosphate) and the 5′ and 3′ annealing regions bring the free ends into proximity. Accordingly, the precursor linear polyribonucleotide produces a ligase-compatible polyribonucleotide, which can be ligated (e.g., in the presence of a ligase) in order to produce a circular polyribonucleotide.

In some embodiments, the disclosure provides a method of producing a circular polyribonucleotide (e.g., in a cell-free system), the method including: providing a linear polyribonucleotide (e.g., a precursor linear polyribonucleotide described herein) wherein the linear polyribonucleotide is in solution under conditions suitable for cleavage of the 5′ self-cleaving ribozyme and the 3′ self-cleaving ribozyme thereby producing a ligase-compatible linear polyribonucleotide; and contacting the ligase-compatible linear polyribonucleotide with a ligase under conditions suitable for ligation of the 5′ and 3′ ends of the ligase-compatible linear polyribonucleotide; thereby producing a circular polyribonucleotide.

In some embodiments, the disclosure provides a method of producing a circular polyribonucleotide, the method including: providing a deoxyribonucleotide encoding the linear polyribonucleotide (e.g., a precursor linear polyribonucleotide described herein); transcribing the deoxyribonucleotide in a cell-free system to produce the linear polyribonucleotide; wherein the transcribing occurs under conditions suitable for cleavage of the 5′ self-cleaving ribozyme and 3′ self-cleaving ribozyme thereby producing a ligase-compatible linear polyribonucleotide; optionally purifying the ligase-compatible linear polyribonucleotide; and contacting the ligase-compatible linear polyribonucleotide with a ligase under conditions suitable for ligation of the 5′ and 3′ ends of the ligase-compatible linear polyribonucleotide, thereby producing a circular polyribonucleotide.

In some embodiments, the disclosure provides a method of producing a circular polyribonucleotide, the method including: providing a deoxyribonucleotide encoding a linear polyribonucleotide; transcribing the deoxyribonucleotide in a cell-free system to produce the linear polyribonucleotide, wherein the transcribing occurs in a solution comprising a ligase and under conditions suitable for ligation of the 5′ and 3′ ends of the linear polyribonucleotide, thereby producing a circular polyribonucleotide. In some embodiments, the linear polyribonucleotide comprises a 5′ self-cleaving ribozyme and a 3′ self-cleaving ribozyme. In some embodiments, the linear polyribonucleotide comprises a 5′ split-intron and a 3′ split-intron (e.g., a self-splicing construct for producing a circular polyribonucleotide). In some embodiments, the linear polyribonucleotide comprises a 5′ annealing region and a 3′ annealing region.

In some embodiments, this disclosure provides a method of producing a circular polyribonucleotide in a cell-free system, the method including the steps of: (a) subjecting a linear polyribonucleotide to conditions suitable for cleavage of self-cleaving ribozymes, wherein the linear polyribonucleotide comprises the following, operably linked in a 5′ to 3′ orientation: (i) a 5′ self-cleaving ribozyme; (ii) a 5′ annealing region comprising a 5′ complementary region; (iii) a polyribonucleotide cargo; (iv) a 3′ annealing region comprising a 3′ complementary region; and (v) a 3′ self-cleaving ribozyme; wherein the 5′ complementary region and the 3′ complementary region have a free energy of binding of less than −5 kcal/mol, and/or wherein the 5′ complementary region and the 3′ complementary region have a Tm of binding of at least 10° C.; and whereby the 5′ self-cleaving ribozyme and the 3′ self-cleaving ribozyme are cleaved to produce a ligase-compatible linear polyribonucleotide; (b) optionally purifying the ligase-compatible linear polyribonucleotide; and (c) in a cell-free system, contacting the ligase-compatible linear polyribonucleotide with an RNA ligase under conditions suitable for ligation of the 5′ and 3′ ends of the ligase-compatible linear polyribonucleotide, optionally wherein the RNA ligase is a tRNA ligase; thereby producing a circular polyribonucleotide. In embodiments, the linear polyribonucleotide is produced in a cell-free system from a DNA construct. In embodiments, the polyribonucleotide cargo includes coding sequence, non-coding sequence, or both coding and non-coding sequence. In embodiments, the polyribonucleotide cargo includes an IRES or a 5′ UTR sequence 5′ to and operably linked to the at least one coding sequence that encodes a polypeptide of interest, optionally with intervening ribonucleotide between the IRES or 5′ UTR sequence and the at least one coding sequence. In embodiments, the polyribonucleotide cargo includes a 3′ UTR sequence 3′ to and operably linked to the at least one coding sequence that encodes a polypeptide of interest, optionally with intervening ribonucleotides between the 3′ UTR sequence and the at least one coding sequence.

Suitable conditions can include any conditions (e.g., a solution or a buffer) that mimic physiological conditions in one or more respects. In some embodiments, suitable conditions include between 0.1-100 mM Mg²⁺ ions or a salt thereof (e.g., 1-100 mM, 1-50 mM, 1-20 mM, 5-50 mM, 5-20 mM, or 5-15 mM). In some embodiments, suitable conditions include between 1-1000 mM K⁺ ions or a salt thereof such as KCl (e.g., 1-1000 mM, 1-500 mM, 1-200 mM, 50-500 mM, 100-500 mM, or 100-300 mM). In some embodiments, suitable conditions include between 1-1000 mM Cl⁻ ions or a salt thereof such as KCl (e.g., 1-1000 mM, 1-500 mM, 1-200 mM, 50-500 mM, 100-500 mM, or 100-300 mM). In some embodiments, suitable conditions include a pH of 4 to 10 (e.g., pH of 5 to 9, pH of 6 to 9, or pH of 6.5 to 8.5). In some embodiments, suitable conditions include a temperature of 4° C. to 50° C. (e.g., 10° C. to 40° C., 15° C. to 40° C., 20° C. to 40° C., or 30° C. to 40° C.),

In some embodiments, suitable conditions include guanosine-5′-triphosphate (GTP) (e.g., 1-1000 μM, 1-500 μM, 1-200 μM, 50-500 μM, 100-500 μM, or 100-300 μM). In some embodiments, suitable conditions include between 0.1-100 mM Mn²⁺ ions or a salt thereof such as MnCl₂ (e.g., 0.1-100 mM, 0.1-50 mM, 0.1-20 mM, 0.1-10 mM, 0.1-5 mM, 0.1-2 mM, 0.5-50 mM, 0.5-20 mM, 0.5-15 mM, 0.5-5 mM, 0.5-2 mM, or 0.1-10 mM). In some embodiments, suitable conditions include dithiothreitol (DTT) (e.g., 1-1000 μM, 1-500 μM, 1-200 μM, 50-500 μM, 100-500 μM, 100-300 μM, 0.1-100 mM, 0.1-50 mM, 0.1-20 mM, 0.1-10 mM, 0.1-5 mM, 0.1-2 mM, 0.5-50 mM, 0.5-20 mM, 0.5-15 mM, 0.5-5 mM, 0.5-2 mM, or 0.1-10 mM).

In some embodiments the linear polyribonucleotide is produced from a deoxyribonucleic acid, e.g., a deoxyribonucleic acid described herein, such as a DNA vector, a linearized DNA vector, or a cDNA. In some embodiments, the linear polyribonucleotide is transcribed from the deoxyribonucleic acid by transcription in a cell-free system (e.g., in vitro transcription).

### One-Pot Method

In some embodiments, the ligase-compatible linear polyribonucleotide is not purified prior to contacting the ligase-compatible linear polyribonucleotide with a ligase. In some embodiments, the transcription in a cell-free system (e.g., in vitro transcription) of the linear RNA from the DNA template, the self-cleavage of the precursor linear RNA to form the ligase-compatible linear RNA, and ligation of the ligase-compatible linear RNA to produce a circular RNA are performed in a single reaction vessel, in the same reaction conditions, and/or without an intermediate purification step for any RNA component. In some embodiments, transcription in a cell-free system (e.g., in vitro transcription) of the linear polyribonucleotide is performed in a solution including the ligase.

In some embodiments, the disclosure provides a method of producing a circular polyribonucleotide, the method including: providing a deoxyribonucleotide encoding the linear polyribonucleotide (e.g., a precursor linear polyribonucleotide described herein); transcribing the deoxyribonucleotide to produce the linear polyribonucleotide; wherein the transcribing occurs under conditions suitable for cleavage of the 5′ self-cleaving ribozyme and 3′ self-cleaving ribozyme thereby producing a ligase-compatible linear polyribonucleotide; and wherein the transcribing occurs in a solution including a ligase and under conditions suitable for ligation of the 5′ and 3′ ends of the ligase-compatible linear polyribonucleotide, thereby producing a circular polyribonucleotide. Suitable conditions include conditions described previously herein.

### Methods of Purification

One or more purification step can be included in the methods described herein. For example, in some embodiments, the ligase-compatible linear polyribonucleotide is substantively enriched or pure (e.g., purified) prior to contacting the ligase-compatible linear polyribonucleotide with a ligase. In other embodiments, the ligase-compatible linear polyribonucleotide is not purified prior to contacting the ligase-compatible linear polyribonucleotide with a ligase. In some embodiments, the resulting circular RNA is purified.

Purification can include separating or enriching the desired reaction product from one or more undesired components, such as any unreacted stating material, byproducts, enzymes, or other reaction components. For example, purification of the ligase-compatible linear polyribonucleotide following transcription in a cell-free system (e.g., in vitro transcription) and cleavage can include separation and/or enrichment from the DNA template prior to contacting the ligase-compatible linear polyribonucleotide with an RNA ligase. Purification of the circular RNA product following ligation can be used to separate and/or enrich the circular RNA from its corresponding linear RNA. Methods of purification of RNA are known to those of skill in the art and include enzymatic purification or by chromatography.

### Bioreactors

In some embodiments, any method of producing a circular polyribonucleotide described herein can be performed in a bioreactor. A bioreactor refers to any vessel in which a chemical process is carried out which involves organisms or biochemically active substances derived from such organisms. In particular, bioreactors can be compatible with the cell-free methods for production of circular RNA described herein. A vessel for a bioreactor can include a culture flask, a dish, or a bag that can be single-use (disposable), autoclavable, or sterilizable. A bioreactor can be made of glass, or it can be polymer-based, or it can be made of other materials.

Examples of bioreactors include, without limitation, stirred tank (e.g., well mixed) bioreactors and tubular (e.g., plug flow) bioreactors, airlift bioreactors, membrane stirred tanks, spin filter stirred tanks, vibromixers, fluidized bed reactors, and membrane bioreactors. The mode of operating the bioreactor can be a batch or continuous processes. A bioreactor is continuous when the reagent and product streams are continuously being fed and withdrawn from the system. A batch bioreactor can have a continuous recirculating flow, but no continuous feeding of reagents or product harvest.

Some methods of this disclosure are directed to large-scale production of circular polyribonucleotides. For large-scale production methods, the method can be performed in a volume of 1 liter (L) to 50 L, or more (e.g., 5 L, 10 L, 15 L, 20 L, 25 L, 30 L, 35 L, 40 L, 45 L, 50 L, or more). In some embodiments, the method can be performed in a volume of 5 L to 10 L, 5 L to 15 L, 5 L to 20 L, 5 L to 25 L, 5 L to 30 L, 5 L to 35 L, 5 L to 40 L, 5 L to 45 L, 10 L to 15 L, 10 L to 20 L, 10 L to 25 L, 20 L to 30 L, 10 L to 35 L, 10 L to 40 L, 10 L to 45 L, 10 L to 50 L, 15 L to 20 L, 15 L to 25 L, 15 L to 30 L, 15 L to 35 L, 15 L to 40 L, 15 L to 45 L, or 15 to 50 L.

In some embodiments, a bioreactor can produce at least 1 g of circular RNA. In some embodiments, a bioreactor can produce 1-200 g of circular RNA (e.g., 1-10 g, 1-20 g, 1-50 g, 10-50 g, 10-100 g, 50-100 g, of 50-200 g of circular RNA). In some embodiments, the amount produced is measure per liter (e.g., 1-200 g per liter), per batch or reaction (e.g., 1-200 g per batch or reaction), or per unit time (e.g., 1-200 g per hour or per day).

In some embodiments, more than one bioreactor can be utilized in series to increase the production capacity (e.g., one, two, three, four, five, six, seven, eight, or nine bioreactors can be used in series).

### Methods of Use

In some embodiments, circular polyribonucleotides made as described herein are used as effectors in therapy and/or agriculture. For example, a circular polyribonucleotide made by the methods described herein (e.g., the cell-free methods described herein) can be administered to a subject (e.g., in a pharmaceutical, veterinary, or agricultural composition). In some embodiments, the subject is a vertebrate animal (e.g., mammal, bird, fish, reptile, or amphibian). In some embodiments, the subject is a human. In some embodiments, the subject is a non-human mammal. In some embodiments, the subject is a non-human mammal such as a non-human primate, ungulate, carnivore, rodent, or lagomorph. In some embodiments, the subject is a bird, reptile, or amphibian. In some embodiments, the subject is an invertebrate animal. In some embodiments, the subject is a plant or eukaryotic alga. In some embodiments, the subject is a plant, such as angiosperm plant (which can be a dicot or a monocot) or a gymnosperm plant (e.g., a conifer, a cycad, a gnetophyte, a Ginkgo), a fern, horsetail, clubmoss, or a bryophyte. In embodiments, the subject is a plant of agricultural or horticultural importance, such as a row crop, fruit, vegetable, tree, or ornamental plant. In some embodiments, a circular polyribonucleotide made by the methods described herein (e.g., the cell-free methods described herein) can be delivered to a cell.

### Formulations

In some embodiments of this disclosure a circular polyribonucleotide described herein (e.g., a circular polyribonucleotide made by the cell-free methods described herein) can be formulated in composition, e.g., a composition for delivery to a cell, a plant, an invertebrate animal, a non-human vertebrate animal, or a human subject, e.g., an agricultural, veterinary, or pharmaceutical composition.

Therefore, in some embodiments, the disclosure also relates to compositions including a circular polyribonucleotide (e.g., a circular polyribonucleotide made by the cell-free methods described herein) and a pharmaceutically acceptable carrier. In one aspect, this disclosure provides pharmaceutical compositions including an effective amount of a polyribonucleotide described herein and a pharmaceutically acceptable excipient. Pharmaceutical compositions of this disclosure can include a polyribonucleotide as described herein, in combination with one or more pharmaceutically or physiologically acceptable carriers, excipients or diluents.

In some embodiments, a pharmaceutically acceptable carrier can be an ingredient in a pharmaceutical composition, other than an active ingredient, which is nontoxic to the subject. A pharmaceutically acceptable carrier can include, but is not limited to, a buffer, excipient, stabilizer, or preservative. Examples of pharmaceutically acceptable carriers are solvents, dispersion media, coatings, antibacterial and antifungal agents, isotonic and absorption delaying agents, and the like that are physiologically compatible, such as salts, buffers, saccharides, antioxidants, aqueous or non-aqueous carriers, preservatives, wetting agents, surfactants or emulsifying agents, or combinations thereof. The amounts of pharmaceutically acceptable carrier(s) in the pharmaceutical compositions can be determined experimentally based on the activities of the carrier(s) and the desired characteristics of the formulation, such as stability and/or minimal oxidation.

In some embodiments, such compositions can include buffers such as acetic acid, citric acid, histidine, boric acid, formic acid, succinic acid, phosphoric acid, carbonic acid, malic acid, aspartic acid, Tris buffers, HEPPSO, HEPES, neutral buffered saline, phosphate buffered saline and the like; carbohydrates such as glucose, sucrose, mannose, or dextrans, mannitol; proteins; polypeptides or amino acids such as glycine; antioxidants; chelating agents such as EDTA or glutathione; adjuvants (e.g., aluminum hydroxide); antibacterial and antifungal agents; and preservatives.

In certain embodiments, compositions of this disclosure can be formulated for a variety of means of parenteral or non-parenteral administration. In one embodiment, the compositions can be formulated for infusion or intravenous administration. Compositions disclosed herein can be provided, for example, as sterile liquid preparations, e.g., isotonic aqueous solutions, emulsions, suspensions, dispersions, or viscous compositions, which can be buffered to a desirable pH. Formulations suitable for oral administration can include liquid solutions, capsules, sachets, tablets, lozenges, and troches, powders liquid suspensions in an appropriate liquid and emulsions.

Pharmaceutical compositions of this disclosure can be administered in a manner appropriate to the disease to be treated or prevented. The quantity and frequency of administration will be determined by such factors as the condition of the subject, and the type and severity of the subject's disease, although appropriate dosages can be determined by clinical trials.

In embodiments, a circular polyribonucleotide as described in this disclosure is provided in a formulation suited to agricultural applications, e.g., as a liquid solution or emulsion, concentrate (liquid, emulsion, gel, or solid), powder, granules, pastes, gels, bait, or seed coating or seed treatment. Embodiments of such agricultural formulations are applied to a plant or to a plant's environment, e.g., as a foliar spray, dust application, granular application, root or soil drench, in-furrow treatment, granular soil treatments, baits, hydroponic solution, or injectable formulation. Some embodiments of such agricultural formulations include additional components, such as excipients, diluents, surfactants, spreaders, stickers, safeners, stabilizers, buffers, drift control agents, retention agents, oil concentrates, defoamers, foam markers, scents, carriers, or encapsulating agents. Useful adjuvants for use in agricultural formulations include those disclosed in the Compendium of Herbicide Adjuvants, 13^{th} edition (2016), publicly available online at www[dot]herbicide-adjuvants[dot]com.

### Embodiments

Various embodiments of the linear polyribonucleotides, circular polyribonucleotides, DNA molecules, systems, methods, and other compositions described herein are set forth in the following sets of numbered embodiments.
1. A linear polyribonucleotide comprising the following, operably linked in a 5′-to-3′ orientation: (A) a 5′ self-cleaving ribozyme; (B) a 5′ annealing region; (C) a polyribonucleotide cargo; (D) a 3′ annealing region; and (E) a 3′ self-cleaving ribozyme.
2. A linear polyribonucleotide having the formula 5′-(A)-(B)-(C)-(D)-(E)-3′, wherein: (A) comprises a 5′ self-cleaving ribozyme; (B) comprises a 5′ annealing region; (C) comprises a polyribonucleotide cargo; (D) comprises a 3′ annealing region; and (E) comprises a 3′ self-cleaving ribozyme.
3. The linear polyribonucleotide of embodiment 1 or 2, wherein the 5′ self-cleaving ribozyme is capable of self-cleavage at a site that is located within 10 ribonucleotides of the 3′ end of the 5′ self-cleaving ribozyme or that is located at the 3′ end of the 5′ self-cleaving ribozyme.
4. The linear polyribonucleotide of any one of embodiment 1-3, wherein the 5′ self-cleaving ribozyme is a ribozyme selected from Hammerhead, Hairpin, Hepatitis Delta Virus ribozyme (HDV), Varkud Satellite (VS), glmS ribozyme, Twister, Twister sister, Hatchet, and Pistol.
5. The linear polyribonucleotide of embodiment 4, wherein the 5′ self-cleaving ribozyme is a Hammerhead ribozyme.
6. The linear polyribonucleotide of any one of embodiments 1-5, wherein the 5′ self-cleaving ribozyme comprises a region having at least 85% sequence identity with the nucleic acid sequence of SEQ ID NO: 1.
7. The linear polyribonucleotide of embodiment 6, wherein the 5′ self-cleaving ribozyme comprises the nucleic acid sequence of SEQ ID NO: 2.
8. The linear polyribonucleotide of any one of embodiments 1-3, wherein the 5′ self-cleaving ribozyme comprises a nucleic acid sequence having at least 85% sequence identity with any one of SEQ ID NOs: 24-571, or a catalytically-competent fragment thereof.
9. The linear polyribonucleotide of embodiment 8, wherein the 5′ self-cleaving ribozyme comprises the nucleic acid sequence of any one of SEQ ID NOs: 24-571, or a catalytically-competent fragment thereof.
10. The linear polyribonucleotide of any one of embodiments 1-9, wherein the 3′ self-cleaving ribozyme is capable of self-cleavage at a site that is located within 10 ribonucleotides of the 5′ end of the 3′ self-cleaving ribozyme or that is located at the 5′ end of the 3′ self-cleaving ribozyme.
11. The linear polyribonucleotide any one of embodiments 1-10, wherein the 3′ self-cleaving ribozyme is a ribozyme selected from Hammerhead, Hairpin, Hepatitis Delta Virus ribozyme (HDV), Varkud Satellite (VS), glmS ribozyme, Twister, Twister sister, Hatchet, and Pistol.
12. The linear polyribonucleotide of embodiment 11, wherein the 3′ self-cleaving ribozyme is a hepatitis delta virus (HDV) ribozyme.
13. The linear polyribonucleotide of any one of embodiments 1-11, wherein the 3′ self-cleaving ribozyme comprises a region having at least 85% sequence identity with the nucleic acid sequence of SEQ ID NO: 2.
14. The linear polyribonucleotide of embodiment 13, wherein the 3′ self-cleaving ribozyme comprises the nucleic acid sequence of SEQ ID NO: 7.
15. The linear polyribonucleotide of any one of embodiments 1-10, wherein the 3′ self-cleaving ribozyme comprises a nucleic acid sequence having at least 85% sequence identity with any one of SEQ ID NOs: 24-571, or a catalytically-competent fragment thereof.
16. The linear polyribonucleotide of embodiment 15, wherein the 3′ self-cleaving ribozyme comprises the nucleic acid sequence of any one of SEQ ID NOs: 24-571, or a catalytically-competent fragment thereof.
17. The linear polyribonucleotide of any one of embodiments 1-16, wherein cleavage of the 5′ self-cleaving ribozyme and of the 3′ self-cleaving ribozyme produce a ligase-compatible linear polyribonucleotide.
18. The linear polyribonucleotides of any one of embodiments 1-17, wherein cleavage of the 5′ self-cleaving ribozyme produces a free 5′-hydroxyl group and cleavage of 3′ self-cleaving ribozyme produces a free 2′,3′-cyclic phosphate group.
19. The linear polyribonucleotide of embodiment 17 or 18, wherein the ligase is an RNA ligase.
20. The linear polyribonucleotide of embodiment 19, wherein the RNA ligase is a tRNA ligase.
21. The linear polyribonucleotide of embodiment 20, wherein the tRNA ligase is a T4 ligase, an RtcB ligase, a TRL-1 ligase, and Rnl1 ligase, an Rnl2 ligase, a LIG1 ligase, a LIG2 ligase a PNK/PNL ligase, a PF0027 ligase, a thpR ligT ligase, a ytlPor ligase, or a variant thereof.
22. The linear polyribonucleotide of embodiment 19, wherein the RNA ligase is a plant RNA ligase, a chloroplast RNA ligase, an RNA ligase from archaea, a bacterial RNA ligase, a eukaryotic RNA ligase, a viral RNA ligase, or a mitochondrial RNA ligase, or a variant thereof.
23. The linear polyribonucleotide of any one of embodiments 1-22, wherein the 5′ annealing region has 5 to 100 ribonucleotides.
24. The linear polyribonucleotide of any one of embodiments 1-23, wherein the 3′ annealing region has 5 to 100 ribonucleotides.
25. The linear polyribonucleotide of any one of embodiments 1-24, wherein the 5′ annealing region comprises a 5′ complementary region having between 5 and 50 ribonucleotides; and the 3′ annealing region comprises a 3′ complementary region having between 5 and 50 ribonucleotides; and wherein the 5′ complementary region and the 3′ complementary region have between 50% and 100% sequence complementarity; and/or wherein the 5′ complementary region and the 3′ complementary region have a free energy of binding of less than −5 kcal/mol; and/or wherein the 5′ complementary region and the 3′ complementary region have a Tm of binding of at least 10° C.
26. The linear polyribonucleotide of embodiment 25, wherein the 5′ annealing region further comprises a 5′ non-complementary region having between 5 and 50 ribonucleotides and is located 5′ to the 5′ complementary region; and 3′ annealing region further comprises a 3′ non-complementary region having between 5 and 50 ribonucleotides and is located 3′ to the 3′ complementary region; and wherein the 5′ non-complementary region and the 3′ non-complementary region have between 0% and 50% sequence complementarity; and/or wherein the 5′ non-complementary region and the 3′ non-complementary region have a free energy of binding of greater than −5 kcal/mol; and/or wherein the 5′ non-complementary region and the 3′ non-complementary region have a Tm of binding of less than 10° C.
27. The linear polyribonucleotide of any one of embodiments 1-26, wherein the 5′ annealing region comprises a region having at least 85% sequence identity with the nucleic acid sequence of SEQ ID NO: 3.
28. The linear polyribonucleotide of embodiment 27, wherein the 5′ annealing region comprises the nucleic acid sequence of SEQ ID NO: 3.
29. The linear polyribonucleotide of any one of embodiments 1-28, wherein the 3′ annealing region comprises a region having at least 85% sequence identity with the nucleic acid sequence of SEQ ID NO: 4.
30. The linear polyribonucleotide of embodiment 29, wherein the 3′ annealing region comprises the nucleic acid sequence of SEQ ID NO: 4.
31. The linear polyribonucleotide of any one of embodiments 1-30, wherein the polyribonucleotide cargo comprises an expression sequence encoding a polypeptide.
32. The linear polyribonucleotide of any one of embodiments 1-31, wherein the polyribonucleotide cargo comprises an IRES operably linked to an expression sequence encoding a polypeptide.
33. The linear polyribonucleotide of embodiment 31 or 32, wherein the polypeptide is a biologically active polypeptide.
34. The linear polyribonucleotide of any one of embodiments 31-33, wherein the polypeptide is polypeptide for use in therapeutic or agricultural applications.
35. The linear polyribonucleotide of any one of embodiments 31-34, wherein the polypeptide is a polypeptide having a sequence encoded in the genome of a vertebrate, invertebrate, plant, or microbe.
36. The linear polyribonucleotide of any one of embodiments 31-34, wherein the polypeptide has a biological effect when contacted with a vertebrate, invertebrate, or plant, or when contacted with a vertebrate cell, invertebrate cell, microbial cell, or plant cell.
37. The linear polyribonucleotide of embodiment 35 or 36, wherein the vertebrate is selected from a human, a non-human mammal, a reptile, a bird, an amphibian, or a fish.
38. The linear polyribonucleotide of embodiment 35 or 36, wherein the invertebrate is selected from an insect, an arachnid, a nematode, or a mollusk.
39. The linear polyribonucleotide of embodiment 35 or 36, wherein the plant is selected from a monocot, a dicot, a gymnosperm, or a eukaryotic alga.
40. The linear polyribonucleotide of embodiment 35 or 36, wherein the microbe is selected from a bacterium, a fungus, or an archaeon.
41. The linear polyribonucleotide of any one of embodiments 1-40, wherein the linear polyribonucleotide further comprises a spacer region of at least 5 polyribonucleotides in length between the 5′ annealing region and the polyribonucleotide cargo.
42. The linear polyribonucleotide of any one of embodiments 1-41, wherein the linear polyribonucleotide further comprises a spacer region of between 5 and 1000 polyribonucleotides in length between the 5′ annealing region and the polyribonucleotide cargo.
43. The linear polyribonucleotide of embodiment 41 or 42, wherein the spacer region comprises a polyA sequence.
44. The linear polyribonucleotide of embodiment 41 or 42, wherein the spacer region comprises a polyA-C sequence.
45. The linear polyribonucleotide of any one of embodiments 1-44, wherein the linear polyribonucleotide is at least 1 kb.
46. The linear polyribonucleotide of any one of embodiments 1-45, wherein the linear polyribonucleotide is 1 kb to 20 kb.
47. A deoxyribonucleic acid comprising an RNA polymerase promoter operably linked to a sequence encoding the linear polyribonucleotide of any one of embodiments 1-46.
48. The deoxyribonucleic acid of embodiment 47, wherein the RNA polymerase promoter is a T7 promoter, a T6 promoter, a T4 promoter, a T3 promoter, an SP3 promoter, or an SP6 promoter.
49. A circular polyribonucleotide produced from the linear polyribonucleotide of any one of embodiments 1-46 or from the deoxyribonucleic acid of embodiment 47 or 48.
50. The circular polyribonucleotide of embodiment 46, wherein, wherein the circular polyribonucleotide is at least 1 kb.
51. The circular polyribonucleotide of embodiment 50, wherein, wherein the circular polyribonucleotide is 1 kb to 20 kb.
52. A method of producing a circular polyribonucleotide, the method comprising: providing the linear polyribonucleotide of any one of embodiments 1-46 wherein the linear polyribonucleotide is in solution under conditions suitable for cleavage of the 5′ self-cleaving ribozyme and the 3′ self-cleaving ribozyme thereby producing a ligase-compatible linear polyribonucleotide; and contacting the ligase-compatible linear polyribonucleotide with a ligase under conditions suitable for ligation of the 5′ and 3′ ends of the ligase-compatible linear polyribonucleotide; thereby producing a circular polyribonucleotide.
53. The method of embodiment 52, wherein the linear polyribonucleotide is produced from a deoxyribonucleic acid.
54. The method of embodiment 53, wherein the deoxyribonucleic acid comprises an RNA polymerase promoter operably linked to a sequence encoding the linear polyribonucleotide.
55. The method of embodiment 54, wherein the RNA polymerase promoter is a T7 promoter, a T6 promoter, a T4 promoter, a T3 promoter, an SP3 promoter, or an SP6 promoter.
56. The method of any one of embodiments 53-55, wherein the linear polyribonucleotide is transcribed from the deoxyribonucleic acid by transcription in a cell-free system.
57. The method of any one of embodiments 52-56, wherein ligase-compatible linear polyribonucleotide is purified prior to contacting the ligase-compatible linear polyribonucleotide with a ligase.
58. The method of embodiment 57, wherein the ligase-compatible linear polyribonucleotide is purified by enzymatic purification or by chromatography.
59. The method of embodiment 56, wherein the transcription of the linear polyribonucleotide is performed in a solution comprising the ligase.
60. A method of producing a circular polyribonucleotide, the method comprising: providing a deoxyribonucleotide encoding the linear polyribonucleotide of any one of embodiments 1-46; transcribing the deoxyribonucleotide in a cell-free system to produce the linear polyribonucleotide; wherein the transcribing occurs under conditions suitable for cleavage of the 5′ self-cleaving ribozyme and 3′ self-cleaving ribozyme thereby producing a ligase-compatible linear polyribonucleotide; optionally purifying the ligase-compatible linear polyribonucleotide; and contacting the ligase-compatible linear polyribonucleotide with a ligase under conditions suitable for ligation of the 5′ and 3′ ends of the ligase-compatible linear polyribonucleotide, thereby producing a circular polyribonucleotide.
61. A method of producing a circular polyribonucleotide, the method comprising: providing a deoxyribonucleotide encoding the linear polyribonucleotide of any one of embodiments 1-46; transcribing the deoxyribonucleotide in a cell-free system to produce the linear polyribonucleotide; wherein the transcribing occurs under conditions suitable for cleavage of the 5′ self-cleaving ribozyme and 3′ self-cleaving ribozyme thereby producing a ligase-compatible linear polyribonucleotide; and wherein the transcribing occurs in a solution comprising a ligase and under conditions suitable for ligation of the 5′ and 3′ ends of the ligase-compatible linear polyribonucleotide, thereby producing a circular polyribonucleotide.
62. A method of producing a circular polyribonucleotide, the method comprising: providing a deoxyribonucleotide encoding a linear polyribonucleotide; transcribing the deoxyribonucleotide in a cell-free system to produce the linear polyribonucleotide, wherein the transcribing occurs in a solution comprising a ligase and under conditions suitable for ligation of the 5′ and 3′ ends of the linear polyribonucleotide, thereby producing a circular polyribonucleotide.
63. The method of 62, wherein the linear polyribonucleotide comprises a 5′ self-cleaving ribozyme and a 3′ self-cleaving ribozyme.
64. The method of 62, wherein the linear polyribonucleotide comprises a 5′ split-intron and a 3′ split-intron.
65. The method of any one of embodiments 62-64, wherein the linear polyribonucleotide comprises a 5′ annealing region and a 3′ annealing region.
66. The method of any one of embodiments 60-65, wherein the deoxyribonucleic acid comprises an RNA polymerase promoter operably linked to a sequence encoding the linear polyribonucleotide.
67. The method of embodiment 66, wherein the RNA polymerase promoter is a T7 promoter, a T6 promoter, a T4 promoter, a T3 promoter, an SP3 promoter, or an SP6 promoter.
68. The method of any one of embodiments 52-67, wherein the ligase is an RNA ligase.
69. The method of embodiment 68, wherein the RNA ligase is a tRNA ligase.
70. The method of embodiment 69, wherein the tRNA ligase is a T4 ligase, an RtcB ligase, a TRL-1 ligase, and Rnl1 ligase, an Rnl2 ligase, a LIG1 ligase, a LIG2 ligase a PNK/PNL ligase, a PF0027 ligase, a thpR ligT ligase, a ytlPor ligase, or a variant thereof.
71. The method of embodiment 68, wherein the RNA ligase is a plant RNA ligase, a chloroplast RNA ligase, an RNA ligase from archaea, a bacterial RNA ligase, a eukaryotic RNA ligase, a viral RNA ligase, or a mitochondrial RNA ligase, or a variant thereof.
72. A method of producing a circular polyribonucleotide, the method comprising: providing a linear polyribonucleotide comprising the following, operably linked in a 5′ to 3′ orientation: a 5′ self-cleaving ribozyme; a 5′ annealing region comprising a 5′ complementary region; a polyribonucleotide cargo; a 3′ annealing region comprising a 3′ complementary region; and a 3′ self-cleaving ribozyme; wherein the 5′ complementary region and the 3′ complementary region have a free energy of binding of less than −5 kcal/mol, and/or wherein the 5′ complementary region and the 3′ complementary region have a Tm of binding of at least 10° C.; and wherein the linear polyribonucleotide is in solution in a cell-free system under conditions suitable for cleavage of the 5′ self-cleaving ribozyme and the 3′ self-cleaving ribozyme, thereby producing a ligase-compatible linear polyribonucleotide in the cell-free system; and contacting the ligase-compatible linear polyribonucleotide in the cell-free system with a ligase under conditions suitable for ligation of the 5′ and 3′ ends of the ligase-compatible linear polyribonucleotide; thereby producing a circular polyribonucleotide.
73. The method of embodiment 72, wherein the linear polynucleotide is provided by transcription from a deoxyribonucleotide that encodes the linear polynucleotide, optionally wherein the deoxyribonucleotide is in the cell-free system.
74. The method of embodiment 73, wherein the transcription is performed in a solution comprising the ligase.
75. The method of embodiment 72, 73, or 74, wherein the 5′ self-cleaving ribozyme is a ribozyme selected from the group consisting of Hammerhead, Hairpin, Hepatitis Delta Virus ribozyme (HDV), Varkud Satellite (VS), glmS ribozyme, Twister, Twister sister, Hatchet, and Pistol.
76. The method of any one of embodiments 72 to 75, wherein the 3′ self-cleaving ribozyme is a ribozyme selected from the group consisting of Hammerhead, Hairpin, Hepatitis Delta Virus ribozyme (HDV), Varkud Satellite (VS), glmS ribozyme, Twister, Twister sister, Hatchet, and Pistol.
77. The method of any one of embodiments 72 to 76, wherein the 5′ complementary region has between 5 and 50 ribonucleotides and the 3′ complementary region has between 5 and 50 ribonucleotides.
78. The method of any one of embodiments 72 to 77, wherein the 5′ complementary region and the 3′ complementary region have between 50% and 100% sequence complementarity, and optionally wherein the 5′ complementary region and the 3′ complementary region include no more than 10 mismatches between them.
79. The method of any one of embodiments 72 to 78, wherein the 5′ annealing region further comprises a 5′ non-complementary region that has between 5 and 50 ribonucleotides and is located 5′ to the 5′ complementary region; and wherein the 3′ annealing region further comprises a 3′ non-complementary region that has between 5 and 50 ribonucleotides and is located 3′ to the 3′ complementary region; and wherein: the 5′ non-complementary region and the 3′ non-complementary region have between 0% and 50% sequence complementarity; and/or the 5′ non-complementary region and the 3′ non-complementary region have a free energy of binding of greater than −5 kcal/mol; and/or the 5′ non-complementary region and the 3′ non-complementary region have a Tm of binding of less than 10° C.
80. The method of any one of embodiments 72 to 79, wherein the 3′ annealing region and the 5′ annealing region promote association of the free 3′ and 5′ ends.
81. The method of any one of embodiments 72 to 80, wherein the polyribonucleotide cargo comprises: at least one coding sequence encoding a polypeptide; or at least one non-coding sequence; or a combination of at least one coding sequence encoding a polypeptide and at least one non-coding sequence.
82. The method of embodiment 81, wherein the polyribonucleotide cargo comprises at least one coding sequence encoding a polypeptide, and wherein the polypeptide comprises an amino acid sequence encoded in the genome of a vertebrate, invertebrate, plant, or microbe, and/or wherein the polypeptide comprises a therapeutic polypeptide, a plant-modifying polypeptide, or an agricultural polypeptide.
83. The method of embodiment 81, wherein the polyribonucleotide cargo comprises at least one coding sequence encoding a polypeptide, and further comprises an additional element selected from the group consisting of: an internal ribosome entry site (IRES) or a 5′ UTR sequence, located 5′ to and operably linked to the coding sequence, optionally with intervening ribonucleotides between the IRES or 5′ UTR sequence and the coding sequence; a 3′ UTR sequence, located 3′ to and operably linked to the coding sequence, optionally with intervening ribonucleotides between the 3′ UTR and the coding sequence; both (a) and (b).
84. The method of any one of embodiments 72 to 83, wherein the linear polyribonucleotide further comprises a spacer region of at least 5 polyribonucleotides in length between the 5′ annealing region and the polyribonucleotide cargo, optionally wherein the spacer region comprises a polyA sequence or a polyA-C sequence.
85. The method of any one of embodiments 72 to 84, wherein the ligase-compatible linear polyribonucleotide includes a free 5′-hydroxyl group and/or the ligase-compatible linear polyribonucleotide includes a free 2′,3′-cyclic phosphate.
86. The method of any one of embodiments 72 to 85, wherein the ligase is an RNA ligase, optionally wherein the RNA ligase is a tRNA ligase.
87. The method of embodiment 86, wherein the tRNA ligase is (a) a ligase selected from the group consisting of a T4 ligase, an RtcB ligase, a TRL-1 ligase, and Rnl1 ligase, an Rnl2 ligase, a LIG1 ligase, a LIG2 ligase a PNK/PNL ligase, a PF0027 ligase, a thpR ligT ligase, and a ytlPor ligase; or (b) a ligase selected from the group consisting of a plant RNA ligase, a chloroplast RNA ligase, an RNA ligase from archaea, a bacterial RNA ligase, a eukaryotic RNA ligase, a viral RNA ligase, and a mitochondrial RNA ligase.
88. The circular polyribonucleotide produced by the method of any one of embodiments 72 to 87.
89. A linear polyribonucleotide comprising the following, operably linked in a 5′ to 3′ orientation: a 5′ self-cleaving ribozyme; a 5′ annealing region comprising a 5′ complementary region; a polyribonucleotide cargo; a 3′ annealing region comprising a 3′ complementary region; and a 3′ self-cleaving ribozyme; wherein the 5′ complementary region and the 3′ complementary region have a free energy of binding of less than −5 kcal/mol, and/or wherein the 5′ complementary region and the 3′ complementary region have a Tm of binding of at least 10° C.
90. The linear polyribonucleotide of embodiment 89, wherein the 5′ self-cleaving ribozyme is a ribozyme selected from Hammerhead, Hairpin, Hepatitis Delta Virus ribozyme (HDV), Varkud Satellite (VS), glmS ribozyme, Twister, Twister sister, Hatchet, and Pistol.
91. The linear polyribonucleotide of embodiment 89 or 90, wherein the 3′ self-cleaving ribozyme is a ribozyme selected from Hammerhead, Hairpin, Hepatitis Delta Virus ribozyme (HDV), Varkud Satellite (VS), glmS ribozyme, Twister, Twister sister, Hatchet. and Pistol.
92. The linear polyribonucleotide of any one of embodiments 89, 90, or 91, wherein the 5′ complementary region has between 5 and 50 ribonucleotides and the 3′ complementary region has between 5 and 50 ribonucleotides.
93. The linear polyribonucleotide of any one of embodiments 89 to 92, wherein the 5′ complementary region and the 3′ complementary region have between 50% and 100% sequence complementarity, and optionally wherein the 5′ complementary region and the 3′ complementary region include no more than 10 mismatches between them.
94. The linear polyribonucleotide of any one of embodiments 89 to 93, wherein the 5′ annealing region further comprises a 5′ non-complementary region that has between 5 and 50 ribonucleotides and is located 5′ to the 5′ complementary region; and wherein the 3′ annealing region further comprises a 3′ non-complementary region that has between 5 and 50 ribonucleotides and is located 3′ to the 3′ complementary region; and wherein: the 5′ non-complementary region and the 3′ non-complementary region have between 0% and 50% sequence complementarity; and/or the 5′ non-complementary region and the 3′ non-complementary region have a free energy of binding of greater than −5 kcal/mol; and/or the 5′ non-complementary region and the 3′ non-complementary region have a Tm of binding of less than 10° C.
95. The linear polyribonucleotide of any one of embodiments 89 to 94, wherein the polyribonucleotide cargo comprises: at least one coding sequence encoding a polypeptide; or at least one non-coding sequence; or a combination of at least one coding sequence encoding a polypeptide and at least one non-coding sequence.
96. The linear polyribonucleotide of embodiment 95, wherein the polyribonucleotide cargo comprises at least one coding sequence encoding a polypeptide, and wherein the polypeptide comprises an amino acid sequence encoded in the genome of a vertebrate, invertebrate, plant, or microbe.
97. The linear polyribonucleotide of embodiment 95, wherein the polyribonucleotide cargo comprises at least one coding sequence encoding a polypeptide, and wherein the polypeptide is a therapeutic polypeptide, a plant-modifying polypeptide, or an agricultural polypeptide.
98. The linear polyribonucleotide of any one of embodiments 89 to 97, further comprising a spacer region of at least 5 polyribonucleotides in length between the 5′ annealing region and the polyribonucleotide cargo, optionally wherein the spacer region comprises a polyA sequence or a polyA-C sequence.
99. A DNA molecule comprising a DNA sequence encoding the linear polyribonucleotide of any one of embodiments 89 to 97, optionally further comprising a heterologous promoter operably linked to the DNA sequence encoding the linear polyribonucleotide.
100. The DNA molecule of embodiment 99, wherein the heterologous promoter is a promoter selected from the group comprising a T7 promoter, a T6 promoter, a T4 promoter, a T3 promoter, an SP3 promoter, and an SP6 promoter.
101. A cell-free system for generating a circular RNA, the system comprising a solution that comprises: a linear polyribonucleotide, wherein the linear polyribonucleotide comprises the following, operably linked in a 5′ to 3′ orientation: a 5′ self-cleaving ribozyme; a 5′ annealing region comprising a 5′ complementary region; a polyribonucleotide cargo; a 3′ annealing region comprising a 3′ complementary region; and a 3′ self-cleaving ribozyme; wherein the 5′ complementary region and the 3′ complementary region have a free energy of binding of less than −5 kcal/mol, and/or wherein the 5′ complementary region and the 3′ complementary region have a Tm of binding of at least 10° C.; and a ligase; wherein conditions of the solution are suitable for cleavage of the 5′ self-cleaving ribozyme and the 3′ self-cleaving ribozyme and ligation of the 5′ and 3′ ends of the resulting ligase-compatible linear polyribonucleotide by the ligase, thereby generating a circular RNA.
102. The circular RNA generated by the cell-free system of embodiment 101.
103. A method of producing a circular polyribonucleotide, the method comprising: subjecting a linear polyribonucleotide to conditions suitable for cleavage of self-cleaving ribozymes, wherein the linear polyribonucleotide comprises the following, operably linked in a 5′ to 3′ orientation: a 5′ self-cleaving ribozyme; a 5′ annealing region comprising a 5′ complementary region; a polyribonucleotide cargo; a 3′ annealing region comprising a 3′ complementary region; and a 3′ self-cleaving ribozyme; wherein the 5′ complementary region and the 3′ complementary region have a free energy of binding of less than −5 kcal/mol, and/or wherein the 5′ complementary region and the 3′ complementary region have a Tm of binding of at least 10° C.; and whereby the 5′ self-cleaving ribozyme and the 3′ self-cleaving ribozyme are cleaved to produce a ligase-compatible linear polyribonucleotide; optionally purifying the ligase-compatible linear polyribonucleotide; and in a cell-free system, contacting the ligase-compatible linear polyribonucleotide with an RNA ligase under conditions suitable for ligation of the 5′ and 3′ ends of the ligase-compatible linear polyribonucleotide, optionally wherein the RNA ligase is a tRNA ligase; thereby producing a circular polyribonucleotide.
104. The method of embodiment 103, wherein the 5′ complementary region and the 3′ complementary region have between 50% and 100% sequence complementarity, and optionally wherein the 5′ complementary region and the 3′ complementary region include no more than 10 mismatches between them.
105. The method of embodiment 103 or 104, wherein the 5′ annealing region further comprises a 5′ non-complementary region that has between 5 and 50 ribonucleotides and is located 5′ to the 5′ complementary region; and wherein the 3′ annealing region further comprises a 3′ non-complementary region that has between 5 and 50 ribonucleotides and is located 3′ to the 3′ complementary region; and wherein: the 5′ non-complementary region and the 3′ non-complementary region have between 0% and 50% sequence complementarity; and/or the 5′ non-complementary region and the 3′ non-complementary region have a free energy of binding of greater than −5 kcal/mol; and/or the 5′ non-complementary region and the 3′ non-complementary region have a Tm of binding of less than 10° C.
106. The method of any one of embodiments 103, 104, or 105, wherein the ligase-compatible linear polyribonucleotide includes a free 5′-hydroxyl group and/or the ligase-compatible linear polyribonucleotide includes a free 2′,3′-cyclic phosphate.

### EXAMPLES

The following examples are put forth to provide those of ordinary skill in the art with a description of how the compositions and methods described herein can be used, made, and evaluated, and are intended to be purely exemplary of the disclosure and are not intended to limit the scope of what the inventors regard as their invention.

### Example 1: Construct Design

This example describes the design of the DNA construct (SEQ ID NO: 8). A schematic depicting the design of the DNA construct is provided in FIG. 1. The construct encodes, from 5′-to-3′: a promotor capable of recruiting an RNA polymerase for RNA synthesis (SEQ ID NO: 1); a 5′ self-cleaving ribozyme that cleaves at its 3′ end (SEQ ID NO: 17); a 5′ annealing region (SEQ ID NO: 18); an internal ribosome entry site (IRES) (SEQ ID NO: 20); a coding region encoding a polypeptide (SEQ ID NO: 21); a 3′ annealing region (SEQ ID NO: 19); and a 3′ self-cleaving ribozyme that cleaves at its 5′ end (SEQ ID NO: 22).

The DNA construct was transcribed to produce a linear RNA (SEQ ID NO: 9) including, from 5′-to-3′: a 5′ self-cleaving ribozyme that cleaves at its 3′ end (SEQ ID NO: 2); a 5′ annealing region (SEQ ID NO: 3); an internal ribosome entry site (IRES) (SEQ ID NO: 5); a coding region encoding a polypeptide (SEQ ID NO: 6); a 3′ annealing region (SEQ ID NO: 4); and a 3′ self-cleaving ribozyme that cleaves at its 5′ end (SEQ ID NO: 7). Upon expression, the linear RNA self-cleaved to produce a ligase-compatible linear RNA having a free 5′ hydroxyl and a free 3′ monophosphate (SEQ ID NO: 10). The ligase-compatible linear RNA was circularized by addition of an RNA ligase. A schematic depicting the process of circularization is provided in FIG. 2.

### Example 2: Methods for Generating Circular RNA in a Cell-Free System

This example describes a method for generating the circular RNA construct in vitro.

In vitro transcription of ribonucleotides was performed using a T7 in vitro transcription reaction (Lucigen Ampliscribe T7 Flash, ASF3257). Subsequent cleavage of the 5′ and 3′ hammerhead ribozymes yielded a 5′-hydroxyl and a 2′,3′ cyclic phosphate RNA sequence with ends that were joined by a tRNA ligase. RNA product of in vitro transcription was treated with DNase to remove the DNA template. Linear RNA was then column purified (New England Biolabs Monarch 500 ug RNA Cleanup Kit, T2050).

Linear RNA was then circularized by treatment with RNA ligase according to manufacturer's instructions. 200 ug of purified linear template in water was heated to 72° C. for 10 minutes. 10× buffer and MnCl₂ were added, and the mixture was cooled at room temperature for 10 minutes. GTP, ligase, and an RNase inhibitor cocktail were added, and the mixture was incubated at 37° C. for 4 hours in a dry air incubator.

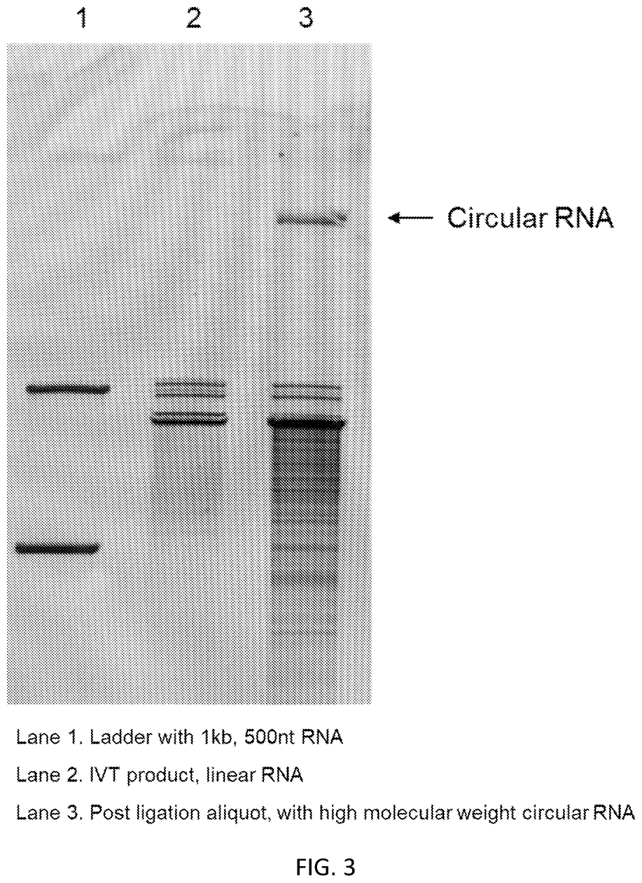

Ligation reaction mixture was purified by ethanol precipitation and resuspended in nuclease-free water. To confirm the purity and quality of ligated RNA, an aliquot was heated to 95° C. for 3 minutes in 50% formamide loading dye and run on a 6% denaturing urea PAGE gel. Linear RNA migrated at expected molecular weight, while circular RNA migrated with high-molecular weight shift confirming that the RNA is circular (see FIG. 3).

In another example, the circular RNA is generated in vitro with modified nucleotides. In vitro transcription of ribonucleotides is performed using a T7 in vitro transcription reaction (Lucigen Ampliscribe T7 Flash, ASF3257) as described in the immediately preceding example, with the following modifications. The manufacturer's instructions are followed, except that the pseudouridine triphosphate (Trilink, N-1019) is used in place of UTP. Quality control of the resulting in vitro transcribed RNA is performed as described above. Briefly, the RNA is separated by gel electrophoresis and stained with ethidium bromide. A band visualized at the expected size indicates that RNA production was successful. The pseudo-uridine substituted RNA is optionally circularized by contacting with RtcB ligase, for example.

### Example 3: Purification of RNA Using Gel Purification

This example describes purification of an RNA. Ligated RNA mixture was purified by PAGE gel purification. One (1) part of RNA sample was mixed with 3 parts of formamide loading buffer (ThermoFisher Scientific, USA), incubated for 3 minutes at 95° C., and chilled on ice. Samples were loaded into 4% urea PAGE gel, with no more than 12 ug of RNA per well. Samples were run for 2-3 hours at 250V and stained with ethidium bromide (ThermoFisher Scientific, USA). High-molecular weight circular bands were cut out and RNA purified by incubating between 3 hours—overnight in elution buffer containing TE buffer, sodium dodecyl sulfate and sodium acetate (ThermoFisher Scientific, USA). Eluted RNA was purified by ethanol precipitation and eluted in 20 ul of nuclease-free water (ThermoFisher Scientific, USA). Quality of purified product was checked by running 200 ng on denaturing PAGE gel and by quantification using a microvolume spectrophotometer.

### Example 4: Confirmation and Quantification of Circular RNA

This example describes the confirmation of the presence of circular RNA and quantification relative to total IVT product. The gel from Example 3 was analyzed using the ImageJ gel analysis tool for pixel intensity and circular band intensity was quantified relative to the intensity of total RNA product. Circular RNA comprised of 75% of total RNA.

### Example 5: RNAs are Functional

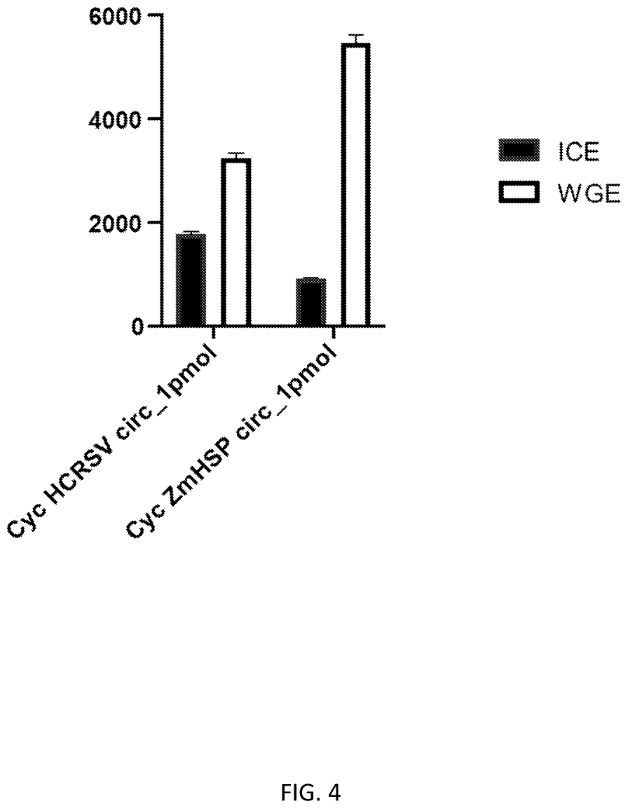

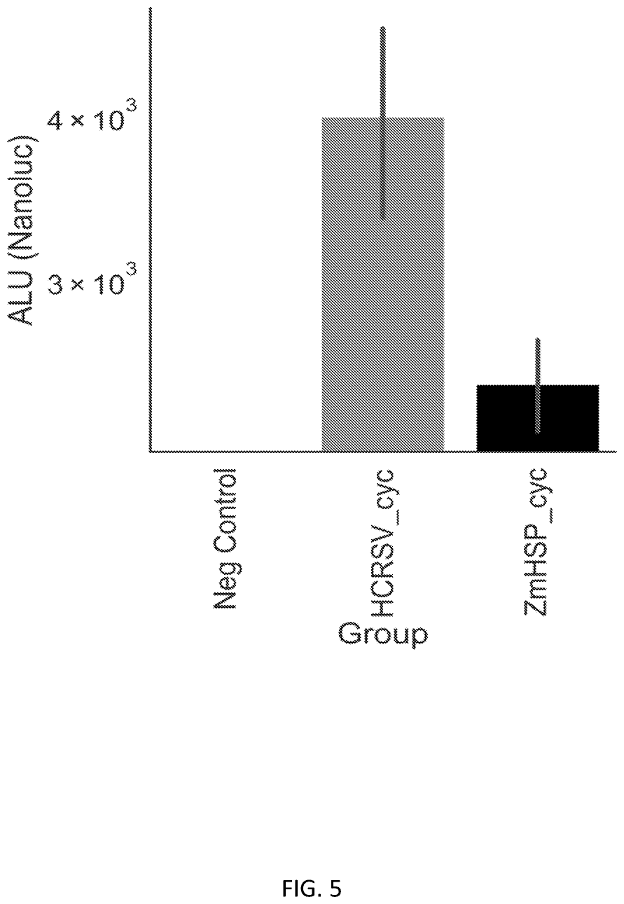

This example describes functional protein expression from circular RNA generated by the methods described herein. To confirm that the circular RNA generated by the methods described herein remains functional, the expression of luciferase was quantified. Wheat germ extract (Promega Corporation), TNT T7 Insect Cell Extract Protein Expression System (Promega Corporation), and Nuclease Treated Rabbit Reticulocyte Lysate (Promega Corporation) were incubated for 1 hour with IRES-luciferase circular RNAs (SEQ ID NOs:10, 15, 16, 23) according to the manufacturer's instructions. Each construct includes an IRES selected from CrTMV (SEQ ID NO:11), HCRSV (SEQ ID NO:12), or ZmHSP (SEQ ID NO:13). Luciferase expression was then measured using Nano-Glo Assay Kit (Promega Corporation). Circular RNAs generated using the methods described herein were able to drive protein expression. 1 pmol HCRSV RNA and ZmHSP RNA drive Nanoluc luciferase expression in insect cell extract (ICE) and wheat germ extract (WGE) (FIG. 4). 2 pmol of RNAs drive Nanoluc luciferase expression in Rabbit Reticulocyte Lysate (FIG. 5).

### Example 6: Methods for Generating Circular RNA with Larger Cargo in a Cell-Free System

This example describes a method for generating RNA constructs for circularization incorporating a larger cargo in a cell-free system. In vitro transcription of ribonucleotides was performed using a T7 in vitro transcription reaction (Lucigen Ampliscribe T7 Flash, ASF3257). Subsequent cleavage of the 5′ and 3′ hammerhead ribozymes yielded a 5′-hydroxyl and a 2′,3′ cyclic phosphate RNA sequence with ends that were joined by a tRNA ligase. RNA product of in vitro transcription was treated with DNase to remove the DNA template. Linear RNA was then column purified (New England Biolabs Monarch 500 ug RNA Cleanup Kit, T2050).

Linear RNA was then circularized by treatment with RNA ligase according to the manufacturer's instructions. 200 micrograms of purified linear template in water was heated to 72° C. for 10 minutes. 10× buffer and MnCl₂ were added, and the mixture was cooled at room temperature for 10 minutes. GTP, ligase, and an RNAse inhibitor cocktail were added, and the mixture was incubated at 37° C. for 4 hours in a dry air incubator.

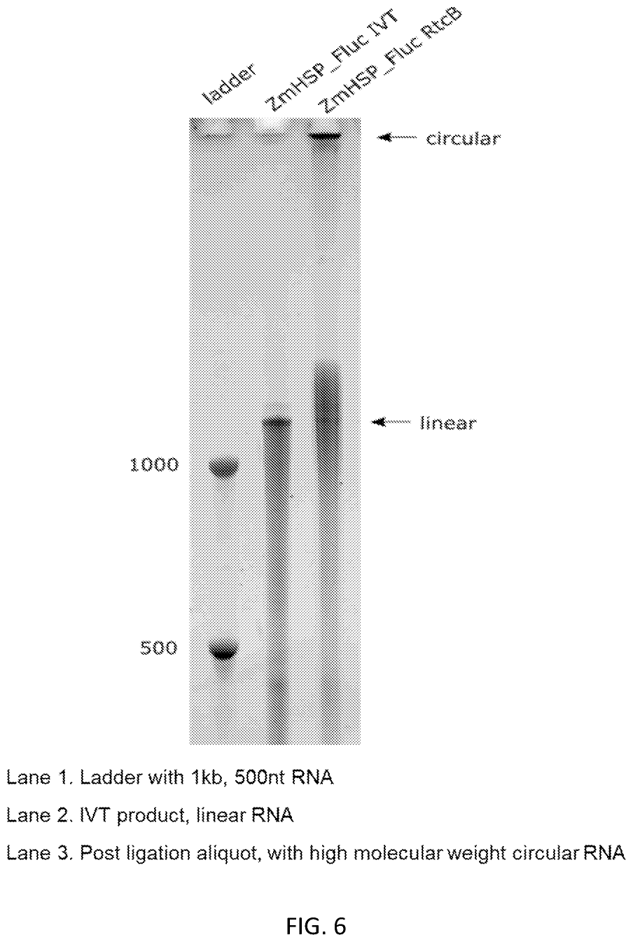

Ligation reaction mixture was purified by ethanol precipitation and resuspended in nuclease-free water. To confirm the purity and quality of ligated RNA, an aliquot was heated to 95° C. for 3 minutes in 50% formamide loading dye and run on a 6% denaturing urea PAGE gel. Linear RNA migrated at expected molecular weight, while circular RNA migrated with high-molecular weight shift (FIG. 6). The final RNA sequence contains an IRES element (ZmHSP, SEQ ID NO: 13) and firefly luciferase (SEQ ID NO: 14), producing a final circular RNA 1850 nucleotides in length (SEQ ID NO: 16).

### Example 7: Generating Circular RNA in a Cell-Free System

This example describes a method of producing a circular polyribonucleotide in a cell-free system from a linear polyribonucleotide precursor. In this example, the linear polynucleotide includes a 5′ annealing region including a 5′ complementary region, and a 3′ annealing region including a 3′ complementary region, wherein fewer than 10 mismatches occur between the 5′ complementary region and the 3′ complementary region, and wherein the 5′ complementary region and the 3′ complementary region have a free energy of binding of less than −5 kcal/mol, and/or wherein the 5′ complementary region and the 3′ complementary region have a Tm of binding of at least 10° C.

More specifically, the linear precursor included, operably linked in 5′ to 3′ direction: (a) a heterologous promoter capable of recruiting an RNA polymerase for RNA synthesis (T7 promoter, SEQ ID: 572); (b) a 5′ self-cleaving ribozyme that cleaves at its 3′ end (a modified P3 Twister U2A ribozyme, SEQ ID: 595); (c) 5′ annealing region (including a nucleotide sequence from the 5′ half of a loop of Eggplant Latent Viroid (ELVd), SEQ ID: 597); (d) a polyribonucleotide cargo comprising a Pepper aptamer sequence (SEQ ID: 599), a ZmHSP101 IRES sequence (SEQ ID: 584), and a Nanoluc open reading frame (SEQ ID: 592); (e) a 3′ annealing region (including a nucleotide sequence from the 3′ half of a loop of Eggplant Latent Viroid (ELVd), SEQ ID: 598); and (f) a 3′ self-cleaving ribozyme that cleaves at its 5′ end (a modified P1 Twister Ribozyme, SEQ ID: 596).

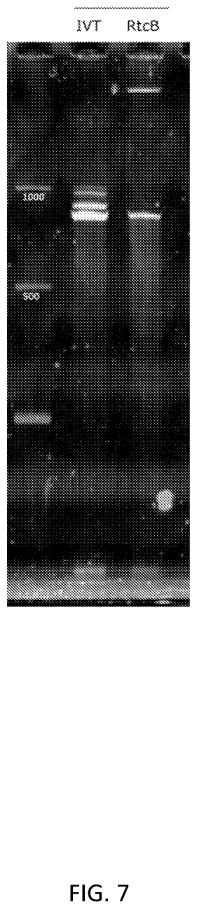

The construct was cloned and sequence verified in E. coli bacteria using standard molecular techniques. PCR was used to generated a linear amplicon comprising the T7 promoter and the entire Cyclone DNA construct. Circular RNA was produced as described in example 2: briefly, the linear amplicon was used as a template for in vitro transcription to produce polyribonucleotides. The polyribonucleotides were contacted with RtcB ligase (New England Biolabs (NEB), Beverly, MA, USA) according to the manufacturer's instructions. Polyribonucleotides were purified using a Monarch® 500 microgram RNA purification column (NEB). Polyribonucleotides were separated by denaturing PAGE. Higher-molecular weight polyribonucleotides (RNAs) indicated successful circularization. Additional quality control steps to verify circular topology of RNA included treatment with exonuclease, which showed that circular RNAs were not digested, confirming their circular topology. Polyribonucleotides and polyacrylamide gels containing separated RNAs were additionally incubated in aptamer buffer containing 100 mM potassium chloride, and stained with HBC525, the ligand for Pepper aptamer. Excitation at 485 nm and detection at 525 nm permitted visualization of the Pepper aptamer after PAGE analysis (FIG. 7. The higher band observed for the linear polynucleotide that had been treated with the RtcB ligase indicated circularization of the linear precursor and functionality of the Pepper aptamer in the resulting circular RNA.

### Example 8: Generating Circular RNA in a Cell-Free System

This example describes additional non-limiting embodiments of methods of producing a circular polyribonucleotide in a cell-free system from a linear polyribonucleotide precursor.

Variations on the methods for generating circular RNA as described in the preceding examples, especially Examples 6 and 7, were developed as follows.

In one embodiment, preparation of sequence-confirmed plasmid DNA was performed using a Monarch Plasmid Miniprep kit according to the manufacturer's instructions, except that RNase A was not added to the neutralization buffer N3. The resulting DNA plasmid was amplified by PCR to generate a linear DNA amplicon free of ribonuclease contamination when used as the template for cell-free (in vitro) transcription. In an example, the linear DNA amplicon was transcribed in vitro overnight in a final volume of 60 microliters. RtcB RNA ligase (NEB) was added directly to the cell-free transcription mixture after DNase treatment. Additional reaction components, except DTT, were additionally added to the final concentration recommended by the manufacturer. The ligation reaction proceeded at 37 degrees C. for 4 hours. The ligation reaction mixture was subjected to ethanol precipitation, resuspended in nuclease-free water, and optionally purified, e.g., by gel purification, by treatment with exonucleases, or by a combination of gel purification and exonuclease treatment; or optionally not further purified.

After RNA production and any optional purification steps, circular RNA production efficiency was measured using denaturing PAGE, e.g., as described in Example 7. The ratio of circular RNA relative to linear RNA precursor was quantified. The ratio of circular:linear RNA was increased after the implementation of the improvements described in this example, relative to the ratio of circular:linear RNA observed using the procedures described in Example 7.

### Example 9. Translation of Coding Sequences Included in a Circular RNA's Polynucleotide Cargo

This example describes embodiments of a circular RNA that includes a polynucleotide cargo including one or more coding or expression sequences.

The circular RNA described in Example 1 included a polyribonucleotide cargo including sequence encoding a polypeptide (Nanoluc luciferase, SEQ ID NO: 592). This circular RNA, when tested in wheat germ or insect cell extracts, provided reproducible, low levels of Nanoluc reporter production. Additional modifications to the circular RNA were tested for increased stability of the circular RNA and/or increased translation efficiency of polypeptides encoded by the polyribonucleotide cargo. The DNA constructs encoding modified linear precursors for these circular RNAs were cloned and sequence verified according to standard molecular techniques.

Examples of these modifications included:
(a) replacement of the internal ribosome entry site (IRES) with a 5′UTR sequence (e.g., any one of SEQ ID NOs:600, 601, 602, 603, 604, or 612) 5′ and operably linked to the coding sequence, either directly or with intervening sequence;
(b) including a 3′ UTR sequence (e.g., any one of SEQ ID NOs: 605, 606, 607, 608, 609, 610, 611, or 613) 3′ and operably linked to the coding sequence, either directly or with intervening sequence, e.g., including a 3′ UTR 3′ to and operably linked to the Nanoluc open reading frame in a construct based on that described in Example 1;
(c) including in the DNA construct DNA sequence encoding an IRES or a 5′ UTR (e.g., any one of SEQ ID NOs: 582, 583, 584, 591, 601, 602, 603, 604, or 612) 5′ and operably linked to the coding sequence, as well as a DNA sequence encoding a 3′UTR selected from SEQ ID NOs:605, 606, 607, 608, 609, 610, 611, or 613 3′ and operably linked to the polynucleotide cargo.

In an example, a linear polyribonucleotide including a polyribonucleotide cargo including the Nanoluc open reading frame was produced, circularized, and purified as described in Examples 1-4. Translation efficiencies were measured using insect cell extract (“ICE”, Promega Corporation) and/or wheat germ extract (“WGE”, Promega Corporation) as described in example 5. Briefly, RNAs were contacted with ICE and WGE for 1 hour according to the manufacturer's instructions and the Nanoluc luciferase assay performed according to the manufacturer's instructions. Luminescence intensity was normalized against a control RNA construct containing the ZmHSP101 IRES operably linked to the Nanoluc ORF and lacking a 3′UTR.

The results of the experiment showed that a circular RNA that included modifications flanking the cargo sequence provided increased translation efficiency of a polypeptide-coding cargo sequence. For example, a circular RNA that included both (a) the sTNV 5′UTR (SEQ ID NO: 600) 5′ and operably linked to the cargo sequence, and (b) the sTNV 3′UTR (SEQ ID NO: 605) 3′ and operably linked to the cargo sequence, had increased translation efficiency compared to the control RNA construct, i.e., ˜5-fold higher translation efficiency than control in wheat germ extract, and ˜1.2-fold higher translation efficiency than the control construct in insect cell extract. In another example, a circular RNA that included both (a) the TCV 5′UTR (SEQ ID NO: 612) 5′ and operably linked to the cargo sequence, and (b) the TCV 3′UTR (SEQ ID NO: 613) 3′ and operably linked to the cargo sequence, had increased translation efficiency compared to the control RNA construct, i.e., ˜1.5-fold higher translation efficiency than control in insect cell extract, and ˜0.9-fold higher translation efficiency than the control construct in wheat germ extract.

TABLE 12
Summary of Sequences used in Examples 7, 8, and 9

| | SEQ | Sequence Type |
|---|---|---|
| Note/name | ID NO | DNA/PRT/RNA |
| T7 promoter | 572 | DNA |
| 5′ hammerhead ribozyme | 573 | RNA |
| 5′ CRC | 574 | RNA |
| 3′ CRC | 575 | RNA |
| EMCV IRES | 576 | RNA |
| Nanoluc ORF | 577 | RNA |
| 3′ hammerhead HDV | 578 | RNA |
| final EMCV Nanoluc construct DNA | 579 | DNA |
| final EMCV Nanoluc construct RNA transcript after IVT | 580 | RNA |
| final EMCV Nanoluc construct RNA, post-ribozyme | 581 | RNA |
| processing | | |
| CrTMV IRES | 582 | RNA |
| HCRSV IRES | 583 | RNA |
| ZmHSP101 IRES | 584 | RNA |
| Firefly luciferase ORF | 585 | RNA |
| final CrTMV Nanoluc construct RNA, post-ribozyme | 586 | RNA |
| processing | | |
| final ZmHSP Fluc construct RNA, post-ribozyme processing | 587 | RNA |
| 5′ hammerhead ribozyme DNA coding | 588 | DNA |
| 5′ CRC DNA coding | 589 | DNA |
| 3′ CRC DNA coding | 590 | DNA |
| EMCV IRES DNA coding | 591 | DNA |
| Nanoluc ORF DNA coding | 592 | DNA |
| 3′ hammerhead HDV DNA coding | 593 | DNA |
| final HCRSV Nanoluc construct RNA, post-ribozyme | 594 | RNA |
| processing | | |
| P3 Twister U2A ribozyme, modified with 5′ ELVd annealing | 595 | DNA |
| region stem, DNA coding | | |
| P1 Twister ribozyme, modified with 3′ ELVd annealing region | 596 | DNA |
| stem, DNA coding | | |
| 5′ annealing region, 5′ half of ELVd loop, DNA coding | 597 | DNA |
| 3′ annealing region, 3′ half of ELVd loop, DNA coding | 598 | DNA |
| Pepper aptamer,, DNA coding | 599 | DNA |
| Satellite tobacco necrosis virus (sTNV) 5′utr, DNA coding | 600 | DNA |
| Maize Necrotic Streak Virus (MNeSV) 5′utr, DNA coding | 601 | DNA |
| Tobacco necrosis virus D (TNV-D) 5′utr, DNA coding | 602 | DNA |
| Barley Yellow Dwarf Virus (BYDV) 5′utr, DNA coding | 603 | DNA |
| 5S0 synthetic 5′utr, DNA coding | 604 | DNA |
| sTNV 3′utr, DNA coding | 605 | DNA |
| MNeSV 3′utr, DNA coding | 606 | DNA |
| TNV-d 3′utr, DNA coding | 607 | DNA |
| BYDV 3′utr, DNA coding | 608 | DNA |
| Cowpea Mosaic Virus (CPMV) 2 3′utr, DNA coding | 609 | PRT |
| Arabidopsis thaliana PsaK (AtPsaK) 3′UTR, DNA coding | 610 | DNA |
| TMV 3′ UTR, DNA coding | 611 | DNA |
| Turnip Crinkle Virus (TCV) 5′UTR, DNA coding | 612 | DNA |
| TCV 3′UTR, DNA coding | 613 | DNA |

All cited patents and patent publications referred to in this application are incorporated herein by reference in their entirety. All the materials and methods disclosed and claimed herein can be made and used without undue experimentation as instructed by the above disclosure and illustrated by the examples. Although the materials and methods related to this invention have been described in terms of embodiments and illustrative examples, it will be apparent to those of skill in the art that substitutions and variations can be applied to the materials and methods described herein without departing from the concept, spirit, and scope of the invention. Thus, the breadth and scope of this invention should not be limited by any of the above-described Examples, but should be defined only in accordance with the preceding embodiments, the following claims, and their equivalents.

1. A method of producing a circular polyribonucleotide, the method comprising:
(a) providing a linear polyribonucleotide comprising the following, operably linked in a 5′ to 3′ orientation:
(i) a 5′ self-cleaving ribozyme;
(ii) a 5′ annealing region comprising a 5′ complementary region;
(iii) a polyribonucleotide cargo;
(iv) a 3′ annealing region comprising a 3′ complementary region; and
(v) a 3′ self-cleaving ribozyme;
wherein the 5′ complementary region and the 3′ complementary region have a free energy of binding of less than −5 kcal/mol, and/or wherein the 5′ complementary region and the 3′ complementary region have a Tm of binding of at least 10° C.; and
wherein the linear polyribonucleotide is in solution in a cell-free system under conditions suitable for cleavage of the 5′ self-cleaving ribozyme and the 3′ self-cleaving ribozyme, thereby producing a ligase-compatible linear polyribonucleotide in the cell-free system; and
(b) contacting the ligase-compatible linear polyribonucleotide in the cell-free system with a ligase under conditions suitable for ligation of the 5′ and 3′ ends of the ligase-compatible linear polyribonucleotide; thereby producing a circular polyribonucleotide.

2. The method of claim 1, wherein the linear polynucleotide is provided by transcription from a deoxyribonucleotide that encodes the linear polynucleotide, optionally wherein the deoxyribonucleotide is in the cell-free system.

3. The method of claim 2, wherein the transcription is performed in a solution comprising the ligase.

4. The method of claim 1, wherein the 5′ and/or 3′ self-cleaving ribozyme is a ribozyme selected from the group consisting of Hammerhead, Hairpin, Hepatitis Delta Virus ribozyme (HDV), Varkud Satellite (VS), glmS ribozyme, Twister, Twister sister, Hatchet, and Pistol.

5. (canceled)

6. The method of claim 1, wherein the 5′ complementary region has between 5 and 50 ribonucleotides and the 3′ complementary region has between 5 and 50 ribonucleotides, and/or wherein the 5′ complementary region and the 3′ complementary region have between 50% and 100% sequence complementarity, optionally wherein the 5′ complementary region and the 3′ complementary region include no more than 10 mismatches between them.

7. (canceled)

8. The method of claim 1, wherein the 5′ annealing region further comprises a 5′ non-complementary region that has between 5 and 50 ribonucleotides and is located 5′ to the 5′ complementary region; and wherein the 3′ annealing region further comprises a 3′ non-complementary region that has between 5 and 50 ribonucleotides and is located 3′ to the 3′ complementary region; and wherein:
(a) the 5′ non-complementary region and the 3′ non-complementary region have between 0% and 50% sequence complementarity; and/or
(b) the 5′ non-complementary region and the 3′ non-complementary region have a free energy of binding of greater than −5 kcal/mol; and/or
(c) the 5′ non-complementary region and the 3′ non-complementary region have a Tm of binding of less than 10° C.

9. The method of claim 1, wherein the 3′ annealing region and the 5′ annealing region promote association of the free 3′ and 5′ ends.

10. The method of claim 1, wherein the polyribonucleotide cargo comprises:
(a) at least one coding sequence encoding a polypeptide, optionally wherein the polypeptide comprises an amino acid sequence encoded in the genome of a vertebrate, invertebrate, plant, or microbe, and/or wherein the polypeptide comprises a therapeutic polypeptide, a plant-modifying polypeptide, or an agricultural polypeptide, and optionally wherein the polyribonucleotide cargo further comprises an additional element selected from the group consisting of: (i) an internal ribosome entry site (IRES) or a 5′ UTR sequence, located 5′ to and operably linked to the coding sequence, optionally with intervening ribonucleotides between the IRES or 5′ UTR sequence and the coding sequence; (ii) a 3′ UTR sequence, located 3′ to and operably linked to the coding sequence, optionally with intervening ribonucleotides between the 3′ UTR and the coding sequence; and (iii) both (i) and (ii); or
(b) at least one non-coding sequence; or
(c) a combination of at least one coding sequence encoding a polypeptide and at least one non-coding sequence.

11. (canceled)

12. (canceled)

13. The method of claim 1, wherein the linear polyribonucleotide further comprises a spacer region of at least 5 polyribonucleotides in length between the 5′ annealing region and the polyribonucleotide cargo, optionally wherein the spacer region comprises a polyA sequence or a polyA-C sequence.

14. The method of claim 1, wherein the ligase-compatible linear polyribonucleotide includes a free 5′-hydroxyl group and/or the ligase-compatible linear polyribonucleotide includes a free 2′,3′-cyclic phosphate.

15. The method of claim 1, wherein the ligase is an RNA ligase, optionally wherein the RNA ligase is a tRNA ligase, optionally wherein the tRNA ligase is (a) a ligase selected from the group consisting of a T4 ligase, an RtcB ligase, a TRL-1 ligase, and Rnl1 ligase, an Rnl2 ligase, a LIG1 ligase, a LIG2 ligase a PNK/PNL ligase, a PF0027 ligase, a thpR ligT ligase, and a ytlPor ligase; or (b) a ligase selected from the group consisting of a plant RNA ligase, a chloroplast RNA ligase, an RNA ligase from archaea, a bacterial RNA ligase, a eukaryotic RNA ligase, a viral RNA ligase, and a mitochondrial RNA ligase.

16. (canceled)

17. The circular polyribonucleotide produced by the method of claim 1.

18. A linear polyribonucleotide comprising the following, operably linked in a 5′ to 3′ orientation:
(a) a 5′ self-cleaving ribozyme;
(b) a 5′ annealing region comprising a 5′ complementary region;
(c) a polyribonucleotide cargo;
(d) a 3′ annealing region comprising a 3′ complementary region; and
(e) a 3′ self-cleaving ribozyme;
wherein the 5′ complementary region and the 3′ complementary region have a free energy of binding of less than −5 kcal/mol, and/or wherein the 5′ complementary region and the 3′ complementary region have a Tm of binding of at least 10° C.

19. The linear polyribonucleotide of claim 18, wherein the 5′ and/or 3′ self-cleaving ribozyme is a ribozyme selected from Hammerhead, Hairpin, Hepatitis Delta Virus ribozyme (HDV), Varkud Satellite (VS), glmS ribozyme, Twister, Twister sister, Hatchet, and Pistol.

20. (canceled)

21. The linear polyribonucleotide of claim 18, wherein the 5′ complementary region has between 5 and 50 ribonucleotides and the 3′ complementary region has between 5 and 50 ribonucleotides, and/or wherein the 5′ complementary region and the 3′ complementary region have between 50% and 100% sequence complementarity, optionally wherein the 5′ complementary region and the 3′ complementary region include no more than 10 mismatches between them.

22. (canceled)

23. The linear polyribonucleotide of claim 18, wherein the 5′ annealing region further comprises a 5′ non-complementary region that has between 5 and 50 ribonucleotides and is located 5′ to the 5′ complementary region; and wherein the 3′ annealing region further comprises a 3′ non-complementary region that has between 5 and 50 ribonucleotides and is located 3′ to the 3′ complementary region; and wherein:
(a) the 5′ non-complementary region and the 3′ non-complementary region have between 0% and 50% sequence complementarity; and/or
(b) the 5′ non-complementary region and the 3′ non-complementary region have a free energy of binding of greater than −5 kcal/mol; and/or
(c) the 5′ non-complementary region and the 3′ non-complementary region have a Tm of binding of less than 10° C.

24. The linear polyribonucleotide of claim 18, wherein the polyribonucleotide cargo comprises:
(a) at least one coding sequence encoding a polypeptide, optionally wherein the polypeptide comprises an amino acid sequence encoded in the genome of a vertebrate, invertebrate, plant, or microbe, and/or wherein the polypeptide comprises a therapeutic polypeptide, a plant-modifying polypeptide, or an agricultural polypeptide, and optionally wherein the polyribonucleotide cargo further comprises an additional element selected from the group consisting of: (i) an internal ribosome entry site (IRES) or a 5′ UTR sequence, located 5′ to and operably linked to the coding sequence, optionally with intervening ribonucleotides between the IRES or 5′ UTR sequence and the coding sequence; (ii) a 3′ UTR sequence, located 3′ to and operably linked to the coding sequence, optionally with intervening ribonucleotides between the 3′ UTR and the coding sequence; and (iii) both (i) and (ii); or
(b) at least one non-coding sequence; or
(c) a combination of at least one coding sequence encoding a polypeptide and at least one non-coding sequence.

25. (canceled)

26. (canceled)

27. The linear polyribonucleotide of claim 18, further comprising a spacer region of at least 5 polyribonucleotides in length between the 5′ annealing region and the polyribonucleotide cargo, optionally wherein the spacer region comprises a polyA sequence or a polyA-C sequence.

28. A DNA molecule comprising a DNA sequence encoding the linear polyribonucleotide of claim 18, optionally further comprising a heterologous promoter operably linked to the DNA sequence encoding the linear polyribonucleotide, optionally wherein the heterologous promoter is a promoter selected from the group comprising a T7 promoter, a T6 promoter, a T4 promoter, a T3 promoter, an SP3 promoter, and an SP6 promoter.

29. (canceled)

30. A cell-free system for generating a circular RNA, the system comprising a solution that comprises:
(a) a linear polyribonucleotide,
wherein the linear polyribonucleotide comprises the following, operably linked in a 5′ to 3′ orientation:
(i) a 5′ self-cleaving ribozyme;
(ii) a 5′ annealing region comprising a 5′ complementary region;
(iii) a polyribonucleotide cargo;
(iv) a 3′ annealing region comprising a 3′ complementary region; and
(v) a 3′ self-cleaving ribozyme;
wherein the 5′ complementary region and the 3′ complementary region have a free energy of binding of less than −5 kcal/mol, and/or wherein the 5′ complementary region and the 3′ complementary region have a Tm of binding of at least 10° C.; and
(b) a ligase;
wherein conditions of the solution are suitable for cleavage of the 5′ self-cleaving ribozyme and the 3′ self-cleaving ribozyme and ligation of the 5′ and 3′ ends of the resulting ligase-compatible linear polyribonucleotide by the ligase, thereby generating a circular RNA.

31. (canceled)

32. A method of producing a circular polyribonucleotide, the method comprising: in a cell free system, contacting a ligase-compatible linear polyribonucleotide with an RNA ligase under conditions suitable for ligation of the 5′ and 3′ ends of the ligase-compatible linear polyribonucleotide, optionally wherein the RNA ligase is a tRNA ligase;
wherein the ligase-compatible linear polyribonucleotide is produced from a linear polyribonucleotide that has been subjected to conditions suitable for cleavage of self-cleaving ribozymes, wherein the linear polyribonucleotide comprises the following, operably linked in a 5′ to 3′ orientation:
(i) a 5′ self-cleaving ribozyme;
(ii) a 5′ annealing region comprising a 5′ complementary region;
(iii) a polyribonucleotide cargo;
(iv) a 3′ annealing region comprising a 3′ complementary region; and
(v) a 3′ self-cleaving ribozyme;
wherein the 5′ complementary region and the 3′ complementary region have a free energy of binding of less than −5 kcal/mol, and/or wherein the 5′ complementary region and the 3′ complementary region have a Tm of binding of at least 10° C.; and
whereby the 5′ self-cleaving ribozyme and the 3′ self-cleaving ribozyme are cleaved to produce a ligase-compatible linear polyribonucleotide; and wherein the ligase-compatible linear polyribonucleotide is optionally purified;
thereby producing a circular polyribonucleotide.

33. The method of claim 32, wherein the 5′ complementary region and the 3′ complementary region have between 50% and 100% sequence complementarity, and optionally wherein the 5′ complementary region and the 3′ complementary region include no more than 10 mismatches between them.

34. The method of claim 32, wherein the 5′ annealing region further comprises a 5′ non-complementary region that has between 5 and 50 ribonucleotides and is located 5′ to the 5′ complementary region; and wherein the 3′ annealing region further comprises a 3′ non-complementary region that has between 5 and 50 ribonucleotides and is located 3′ to the 3′ complementary region; and wherein:
(a) the 5′ non-complementary region and the 3′ non-complementary region have between 0% and 50% sequence complementarity; and/or
(b) the 5′ non-complementary region and the 3′ non-complementary region have a free energy of binding of greater than −5 kcal/mol; and/or
(c) the 5′ non-complementary region and the 3′ non-complementary region have a Tm of binding of less than 10° C.

35. The method of claim 32, wherein the ligase-compatible linear polyribonucleotide includes a free 5′-hydroxyl group and/or the ligase-compatible linear polyribonucleotide includes a free 2′,3′-cyclic phosphate.
